SEQUENCE LISTING (158 sequences)

SEQ ID NO: 1 - DNA, 22 bp, Artificial Sequence
  Synthetic construct
  1018 ISS
tgactgtgaacgttcgagatga

SEQ ID NO: 2 - DNA, 20 bp, Artificial Sequence
  Synthetic construct
  Abetimus, subunit 1
gtgtgtgtgtgtgtgtgtgt

SEQ ID NO: 3 - DNA, 20 bp, Artificial Sequence
  Synthetic construct
  Abetimus, subunit 2
cacacacacacacacacaca

SEQ ID NO: 4 - DNA, 20 bp, Artificial Sequence
  Synthetic construct
  Abetimus, subunit 3
cacacacacacacacacaca

SEQ ID NO: 5 - DNA, 20 bp, Artificial Sequence
  Synthetic construct
  Abetimus, subunit 4
cacacacacacacacacaca

SEQ ID NO: 6 - DNA, 20 bp, Artificial Sequence
  Synthetic construct
  Abetimus, subunit 5
cacacacacacacacacaca

SEQ ID NO: 7 - DNA, 20 bp, Artificial Sequence
  Synthetic construct
  Abetimus, subunit 6
gtgtgtgtgtgtgtgtgtgt

SEQ ID NO: 8 - DNA, 20 bp, Artificial Sequence
  Synthetic construct
  Abetimus, subunit 7
gtgtgtgtgtgtgtgtgtgt

SEQ ID NO: 9 - DNA, 20 bp, Artificial Sequence
  Synthetic construct
  Abetimus, subunit 8
gtgtgtgtgtgtgtgtgtgt

SEQ ID NO: 10 - DNA, 20 bp, Artificial Sequence
  Synthetic construct
  Afovirsen
ttgcttccatcttcctcgtc

SEQ ID NO: 11 - DNA, 25 bp, Artificial Sequence
  Synthetic construct
  Aganirsen
tatccggagggctcgccatgctgct

SEQ ID NO: 12 - DNA, 24 bp, Artificial Sequence
  Synthetic construct
  Agatolimod
tcgtcgttttgtcgttttgtcgtt

SEQ ID NO: 13 - DNA, 20 bp, Artificial Sequence
  Synthetic construct
  Alicaforsen
gcccaagctggcatccgtca

SEQ ID NO: 14 - DNA, 26 bp, Artificial Sequence
  Synthetic construct
  AGRO100
ggtggtggtggttgtggtggtggtgg

SEQ ID NO: 15 - DNA, 20 bp, Artificial Sequence
  Synthetic construct
  Amlivirsen
gcagaggtgaagcgaagugc

SEQ ID NO: 16 - DNA, 31 bp, Artificial Sequence
  Synthetic construct
  Pegnivacigin
guggacuauaccgcguaaugcugccuccact

SEQ ID NO: 17 - RNA, 15 nt, Artificial Sequence
  Synthetic construct
  Anivamersen
cgcgguauaguccac

SEQ ID NO: 18 - DNA, 20 bp, Artificial Sequence
  Synthetic construct
  Apatorsen
gggacgcggcgctcggucau

SEQ ID NO: 19 - DNA, 26 bp, Artificial Sequence
  Synthetic construct
  Aprinocarsen
ggtggtggtggttgtggtggtggtgg

SEQ ID NO: 20 - DNA, 17 bp, Artificial Sequence
  Synthetic construct
  Zintevir
gtggtgggtgggtgggt

SEQ ID NO: 21 - RNA, 32 nt, Artificial Sequence
  Synthetic construct
  ARC19499
ggaauauacuuggcucguuaggugcguauaua

SEQ ID NO: 22 - DNA, 20 bp, Artificial Sequence
  Synthetic construct
  Archexin
gctgcatgatctccttggcg

SEQ ID NO: 23 - DNA, 21 bp, Artificial Sequence
  Synthetic construct
  Asvasiran antisense
cuugacuuugcuaagagcctt

SEQ ID NO: 24 - DNA, 21 bp, Artificial Sequence
  Synthetic construct
  Asvasiran sense
ggcucuuagcaaagucaagtt

SEQ ID NO: 25 - DNA, 20 bp, Artificial Sequence
  Synthetic construct
  Atesidorsen
ucagggcattctttccauuc

SEQ ID NO: 26 - RNA, 23 nt, Artificial Sequence
  Synthetic construct
  ATU-027 antisense
agacuugaggacuuccuggacaa

SEQ ID NO: 27 - RNA, 23 nt, Artificial Sequence
  Synthetic construct
  ATU-027 sense
uuguccaggaaguccucaagucu

SEQ ID NO: 28 - DNA, 39 bp, Artificial Sequence
  Synthetic construct
  ZIMURA
cgccgcggucucaggcgcugagucugaguuuaccugcgt

SEQ ID NO: 29 - DNA, 19 bp, Artificial Sequence
  Synthetic construct
  AVI-7537
gccatggttttttctcagg

SEQ ID NO: 30 - DNA, 16 bp, Artificial Sequence
  Synthetic construct
  Baliforsen
tcccgaatgtccgaca

SEQ ID NO: 31 - DNA, 21 bp, Artificial Sequence
  Synthetic construct
  Bamosiran antisense
cauugugcaugugauccagtt

SEQ ID NO: 32 - DNA, 21 bp, Artificial Sequence
  Synthetic construct
  Bamosiran sense
cuggaucacaugcacaaugtt

SEQ ID NO: 33 - DNA, 18 bp, Artificial Sequence
  Synthetic construct
  Bazlitoran
ctatctgucgttctctgu

SEQ ID NO: 34 - DNA, 16 bp, Artificial Sequence
  Synthetic construct
  Beclanorsen
cucccaacgtgcgcca

SEQ ID NO: 35 - DNA, 20 bp, Artificial Sequence
  Synthetic construct
  Bepirovirsen
gcagaggtgaagcgaagtgc

SEQ ID NO: 36 - DNA, 21 bp, Artificial Sequence
  Synthetic construct
  Bevasiranib antisense
accucaccaaggccagcactt

SEQ ID NO: 37 - DNA, 21 bp, Artificial Sequence
  Synthetic construct
  Bevasiranib sense
gugcuggccuuggugaggutt

SEQ ID NO: 38 - DNA, 23 bp, Artificial Sequence
  Synthetic construct
  Brivoligide strand 1
ctacgcccaccgcccacgcatac

SEQ ID NO: 39 - DNA, 23 bp, Artificial Sequence
  Synthetic construct
  Brivoligide strand 2
gtatgcgtgggcggtgggcgtag

SEQ ID NO: 40 - DNA, 22 bp, Artificial Sequence
  Synthetic construct
  Casimersen
caatgccatcctggagttcctg

SEQ ID NO: 41 - DNA, 24 bp, Artificial Sequence
  Synthetic construct
  Cavrotolimod
tcgtcgttttgtcgttttgtcgtt

SEQ ID NO: 42 - DNA, 25 bp, Artificial Sequence
  Synthetic construct
  Cemdisiran antisense
uauuauaaaaauaucuugcuuuutt

SEQ ID NO: 43 - RNA, 22 nt, Artificial Sequence
  Synthetic construct
  Cemdisiran sense
  misc_feature (22)..(22): n is a, c, g, or u
aagcaagauauuuuuauaauan

SEQ ID NO: 44 - DNA, 20 bp, Artificial Sequence
  Synthetic construct
  Cenersen
ccctgctcccccctggctcc

SEQ ID NO: 45 - DNA, 19 bp, Artificial Sequence
  Synthetic construct
  Cobitolimod
ggaacagttcgtccatggc

SEQ ID NO: 46 - DNA, 30 bp, Artificial Sequence
  Synthetic construct
  NEXAGON
gtaattgcggcaagaagaattgtttctgtc

SEQ ID NO: 47 - DNA, 16 bp, Artificial Sequence
  Synthetic construct
  Cofirasersen
cccgatagctggtugu

SEQ ID NO: 48 - RNA, 19 nt, Artificial Sequence
  Synthetic construct
  Cosdosiran antisense
gccagaauguggaacuccu

SEQ ID NO: 49 - RNA, 19 nt, Artificial Sequence
  Synthetic construct
  Cosdosiran sense
aggaguuccacauucuggc

SEQ ID NO: 50 - DNA, 21 bp, Artificial Sequence
  Synthetic construct
  CPG-8954
gggggggtgtcgcagcagggg

SEQ ID NO: 51 - DNA, 20 bp, Artificial Sequence
  Synthetic construct
  Cupabimod strand 1
ggagggaaatcccttcaagg

SEQ ID NO: 52 - DNA, 20 bp, Artificial Sequence
  Synthetic construct
  Cupabimod strand 2
ccttgaagggatttccctcc

SEQ ID NO: 53 - DNA, 21 bp, Artificial Sequence
  Synthetic construct
  Custirsen
cagcagcagagtcttcaucau

SEQ ID NO: 54 - DNA, 16 bp, Artificial Sequence
  Synthetic construct
  Danvatirsen
cuatttggatgtcagc

SEQ ID NO: 55 - RNA, 21 nt, Artificial Sequence
  Synthetic construct
  Daplusiran antisense
guggacuucucucaauuuucu

SEQ ID NO: 56 - RNA, 21 nt, Artificial Sequence
  Synthetic construct
  Daplusiran sense
agaaaauugagagaaguccac

SEQ ID NO: 57 - RNA, 25 nt, Artificial Sequence
  Synthetic construct
  Dematirsen
guugccuccgguucugaagguguuc

SEQ ID NO: 58 - DNA, 20 bp, Artificial Sequence
  Synthetic construct
  Donidalorsen
tgcaagtctcttggcaaaca

SEQ ID NO: 59 - RNA, 20 nt, Artificial Sequence
  Synthetic construct
  Drisapersen
ucaaggaagauggcauuucu

SEQ ID NO: 60 - DNA, 14 bp, Artificial Sequence
  Synthetic construct
  Edifoligide strand 1
ctagatttcccgcg

SEQ ID NO: 61 - DNA, 14 bp, Artificial Sequence
  Synthetic construct
  Edifoligide strand 2
gatccgcgggaaat

SEQ ID NO: 62 - DNA, 40 bp, Artificial Sequence
  Synthetic construct
  Egaptivon pegol
gcgugcagugccuucggccgtgcggtgccuccgucacgct

SEQ ID NO: 63 - DNA, 20 bp, Artificial Sequence
  Synthetic construct
  EIF-4E
tgttatattcctggatcctt

SEQ ID NO: 64 - RNA, 33 nt, Artificial Sequence
  Synthetic construct
  Eluforsen
aucauaggaaacaccaaagaugauauuuucuuu

SEQ ID NO: 65 - RNA, 40 nt, Artificial Sequence
  Synthetic construct
  Emapticap pegol
gcacgucccucaccggugcaagugaagccguggcucugcg

SEQ ID NO: 66 - DNA, 20 bp, Artificial Sequence
  Synthetic construct
  Eplontersen
ucuuggttacatgaaauccc

SEQ ID NO: 67 - DNA, 30 bp, Artificial Sequence
  Synthetic construct
  Eteplirsen
ctccaacatcaaggaagatggcatttctag

SEQ ID NO: 68 - DNA, 20 bp, Artificial Sequence
  Synthetic construct
  Fesomersen
acggcattggtgcacaguuu

SEQ ID NO: 69 - RNA, 23 nt, Artificial Sequence
  Synthetic construct
  Fitusiran antisense
uugaaguaaaugguguuaaccag

SEQ ID NO: 70 - RNA, 21 nt, Artificial Sequence
  Synthetic construct
  Fitusiran sense
gguuaacaccauuuacuucaa

SEQ ID NO: 71 - DNA, 21 bp, Artificial Sequence
  Synthetic construct
  Fomivirsen
gcgtttgctcttcttcttgcg

SEQ ID NO: 72 - DNA, 18 bp, Artificial Sequence
  Synthetic construct
  Gataparsen
tgtgctattctgtgaatt

SEQ ID NO: 73 - RNA, 23 nt, Artificial Sequence
  Synthetic construct
  Givosiran antisense
aaugaugagacacucuuucuggu

SEQ ID NO: 74 - RNA, 21 nt, Artificial Sequence
  Synthetic construct
  Givosiran sense
cagaaagagugucucaucuua

SEQ ID NO: 75 - DNA, 21 bp, Artificial Sequence
  Synthetic construct
  GNKG-168
tcgtcgacgtcgttcgttctc

SEQ ID NO: 76 - DNA, 25 bp, Artificial Sequence
  Synthetic construct
  Golodirsen
gttgcctccggttctgaaggtgttc

SEQ ID NO: 77 - DNA, 20 bp, Artificial Sequence
  Synthetic construct
  GPI-2A
ggttcttttggtccttgtct

SEQ ID NO: 78 - DNA, 20 bp, Artificial Sequence
  Synthetic construct
  GTI-2040
ggctaaatcgctccaccaag

SEQ ID NO: 79 - DNA, 20 bp, Artificial Sequence
  Synthetic construct
  GTI-2501
ctctagcgtcttaaagccga

SEQ ID NO: 80 - DNA, 24 bp, Artificial Sequence
  Synthetic construct
  IMT-504
tcatcattttgtcattttgtcatt

SEQ ID NO: 81 - DNA, 22 bp, Artificial Sequence
  Synthetic construct
  Inclisiran antisense
  misc_feature (22)..(22): n is a, c, g, t or u
cuagaccugutuugcuuuugun

SEQ ID NO: 82 - RNA, 23 nt, Artificial Sequence
  Synthetic construct
  Inclisiran sense
acaaaagcaaaacaggucuagaa

SEQ ID NO: 83 - DNA, 20 bp, Artificial Sequence
  Synthetic construct
  Inotersen
ucuuggttacatgaaauccc

SEQ ID NO: 84 - DNA, 13 bp, Artificial Sequence
  Synthetic construct
  Imetelstat
tagggttagacaa

SEQ ID NO: 85 - DNA, 19 bp, Artificial Sequence
  Synthetic construct
  Lademirsen
acatcagtctgauaagcta

SEQ ID NO: 86 - RNA, 44 nt, Artificial Sequence
  Synthetic construct
  Lexaptepid pegol
gcgccguaugggauuaaguaaaugaggaguuggaggaagggcgc

SEQ ID NO: 87 - DNA, 26 bp, Artificial Sequence
  Synthetic construct
  Litenimod
taaacgttataacgttatgacgtcat

SEQ ID NO: 88 - RNA, 21 nt, Artificial Sequence
  Synthetic construct
  Lumasiran antisense
gacuuucauccuggaaauaua

SEQ ID NO: 89 - RNA, 23 nt, Artificial Sequence
  Synthetic construct
  Lumasiran sense
uauauuuccaggaugaaagucca

SEQ ID NO: 90 - DNA, 20 bp, Artificial Sequence
  Synthetic construct
  Mipomersen
gccucagtctgcttcgcacc

SEQ ID NO: 91 - DNA, 15 bp, Artificial Sequence
  Synthetic construct
  Miravirsen
ccattgtcacactcc

SEQ ID NO: 92 - DNA, 21 bp, Artificial Sequence
  Synthetic construct
  Mongersen
gtcgccccttctccccgcagc

SEQ ID NO: 93 - RNA, 22 nt, Artificial Sequence
  Synthetic construct
  Nedosiran antisense
ucagauaaaaaggacaacaugg

SEQ ID NO: 94 - RNA, 36 nt, Artificial Sequence
  Synthetic construct
  Nedosiran sense
auguuguccuuuuuaucugagcagccgaaaggcugc

SEQ ID NO: 95 - DNA, 18 bp, Artificial Sequence
  Synthetic construct
  Nusinersen
tcacctttataatgctgg

SEQ ID NO: 96 - DNA, 18 bp, Artificial Sequence
  Synthetic construct
  Oblimersen
tctcccagcgtgcgccat

SEQ ID NO: 97 - RNA, 45 nt, Artificial Sequence
  Synthetic construct
  Olaptesed pegol
gcguggugugaucuagauguauuggcugauccuagucagguacgc

SEQ ID NO: 98 - RNA, 21 nt, Artificial Sequence
  Synthetic construct
  Olpasiran antisense
cagccccuuauuguuauacga

SEQ ID NO: 99 - RNA, 21 nt, Artificial Sequence
  Synthetic construct
  Olpasiran sense
ucguauaacaauaaggggcug

SEQ ID NO: 100 - DNA, 21 bp, Artificial Sequence
  Synthetic construct
  Patisiran antisense
guaaccaagaguauuccautt

SEQ ID NO: 101 - DNA, 21 bp, Artificial Sequence
  Synthetic construct
  Patisiran sense
auggaauacucuugguuactt

SEQ ID NO: 102 - DNA, 28 bp, Artificial Sequence
  Synthetic construct
  Pegaptanib
cggaaucagugaaugcuuauacauccgt

SEQ ID NO: 103 - DNA, 30 bp, Artificial Sequence
  Synthetic construct
  Pegpleranib
caggcuacgcgtagagcaucatgatccugt

SEQ ID NO: 104 - DNA, 20 bp, Artificial Sequence
  Synthetic construct
  Pelacarsen
tgctccgttggtgcttgttc

SEQ ID NO: 105 - DNA, 18 bp, Artificial Sequence
  Synthetic construct
  Prexigebersen
atatttggcgatggcttc

SEQ ID NO: 106 - DNA, 30 bp, Artificial Sequence; identical to SEQ ID NO: 67
  Synthetic construct
  Radavirsen

SEQ ID NO: 107 - RNA, 25 nt, Artificial Sequence
  Synthetic construct
  Remlarsen antisense
uagcaccauuugaaaucaguguuuu

SEQ ID NO: 108 - RNA, 23 nt, Artificial Sequence
  Synthetic construct
  Remlarsen sense
aacacuguuuacaaaugguccua

SEQ ID NO: 109 - RNA, 18 nt, Artificial Sequence
  Synthetic construct
  Renadirsen
ccuaccguaacccgucgc

SEQ ID NO: 110 - DNA, 20 bp, Artificial Sequence
  Synthetic construct
  Resten-MP
acgttgaggggcatcgtcgc

SEQ ID NO: 111 - RNA, 21 nt, Artificial Sequence
  Synthetic construct
  Revusiran antisense
ugggauuucauguaaccaaga

SEQ ID NO: 112 - RNA, 23 nt, Artificial Sequence
  Synthetic construct
  Revusiran sense
ucuugguuacaugaaaucccauc

SEQ ID NO: 113 - RNA, 9 nt, Artificial Sequence
  Synthetic construct
  RGLS-4326
agcacuuug

SEQ ID NO: 114 - RNA, 20 nt, Artificial Sequence
  Synthetic construct
  Rimigorsen
ucagcuucuguuagccacug

SEQ ID NO: 115 - DNA, 24 bp, Artificial Sequence
  Synthetic construct
  Rosomidnar
cacgcacgcgcatccccgcccgtg

SEQ ID NO: 116 - RNA, 20 nt, Artificial Sequence
  Synthetic construct
  Suvodirsen
ucaaggaagauggcauuucu

SEQ ID NO: 117 - DNA, 19 bp, Artificial Sequence
  Synthetic construct
  Temavirsen
acaccautgucacactcca

SEQ ID NO: 118 - RNA, 19 nt, Artificial Sequence
  Synthetic construct
  Teprasiran antisense
ugaagggugaaauauucuc

SEQ ID NO: 119 - RNA, 19 nt, Artificial Sequence
  Synthetic construct
  Teprasiran sense
gagaauauuucacccuuca

SEQ ID NO: 120 - RNA, 19 nt, Artificial Sequence
  Synthetic construct
  Tivanisiran antisense
aagcgcaucuucuacuuca

SEQ ID NO: 121 - RNA, 19 nt, Artificial Sequence
  Synthetic construct
  Tivanisiran sense
ugaaguagaagaugcgcuu

SEQ ID NO: 122 - DNA, 20 bp, Artificial Sequence
  Synthetic construct
  Tofersen
caggatacatttctacagcu

SEQ ID NO: 123 - DNA, 20 bp, Artificial Sequence
  Synthetic construct
  Tominersen
cucagtaacattgacaccac

SEQ ID NO: 124 - DNA, 18 bp, Artificial Sequence
  Synthetic construct
  Trabedersen
cggcatgtctattttgta

SEQ ID NO: 125 - DNA, 25 bp, Artificial Sequence
  Synthetic construct
  Trecovirsen
tcttcctctctctacccacgctctc

SEQ ID NO: 126 - DNA, 30 bp, Artificial Sequence
  Synthetic construct
  Vidutolimod
gggggggggggacgatcgtcgggggggggg

SEQ ID NO: 127 - DNA, 21 bp, Artificial Sequence
  Synthetic construct
  Viltolarsen
cctccggttctgaaggtgttc

SEQ ID NO: 128 - DNA, 20 bp, Artificial Sequence
  Synthetic construct
  Volanesorsen
agcuucttgtccagcuuuau

SEQ ID NO: 129 - DNA, 20 bp, Artificial Sequence
  Synthetic construct
  Vupanorsen
ggacattgccagtaatcgca

SEQ ID NO: 130 - RNA, 21 nt, Artificial Sequence
  Synthetic construct
  Vutrisiran antisense
ugggauuucauguaaccaaga

SEQ ID NO: 131 - RNA, 23 nt, Artificial Sequence
  Synthetic construct
  Vutrisiran sense
ucuugguuacaugaaaucccauc

SEQ ID NO: 132 - RNA, 20 nt, Artificial Sequence
  Synthetic construct
  Rovanersen
ggcacaagggcacagacuuc

SEQ ID NO: 133 - DNA, 20 bp, Artificial Sequence
  Synthetic construct
  Lexanersen
gugcacacagtagatgaggg

SEQ ID NO: 134 - DNA, 16 bp, Artificial Sequence
  Synthetic construct
  Cepadacursen (CIVI 008)
aatgctacaaaaccca

SEQ ID NO: 135 - DNA, 16 bp, Artificial Sequence
  Synthetic construct
  ISIS-863633
aataatctcatgtcag

SEQ ID NO: 136 - DNA, 19 bp, Artificial Sequence
  Synthetic construct
  AEG35156
ugcaccctggataccauuu

SEQ ID NO: 137 - DNA, 26 bp, Artificial Sequence
  Synthetic construct
  AS1411
ggtggtggtggttgtggtggtggtgg

SEQ ID NO: 138 - RNA, 22 nt, Artificial Sequence
  Synthetic construct
  Belcesiran antisense
uuuaagaagacaaaggguuugg

SEQ ID NO: 139 - RNA, 36 nt, Artificial Sequence
  Synthetic construct
  Belcesiran sense
aaacccuuugucuucuuaaagcagccgaaaggcugc

SEQ ID NO: 140 - DNA, 20 bp, Artificial Sequence
  Synthetic construct
  Cimderlirsen
ccacctttgggtgaatagca

SEQ ID NO: 141 - DNA, 16 bp, Artificial Sequence
  Synthetic construct
  Frenlosirsen
agttgtaaatgagucg

SEQ ID NO: 142 - RNA, 20 nt, Artificial Sequence
  Synthetic construct
  LSP-GR3
gcuagguuuacgggaccucu

SEQ ID NO: 143 - DNA, 20 bp, Artificial Sequence
  Synthetic construct
  Monarsen
ctgcgatattttcttgtacc

SEQ ID NO: 144 - DNA, 20 bp, Artificial Sequence
  Synthetic construct
  Olezarsen
agcuucttgtccagcuuuau

SEQ ID NO: 145 - DNA, 25 bp, Artificial Sequence
  Synthetic construct
  PUL-042
tcgtcgtcgttcgaacgacgttgat

SEQ ID NO: 146 - DNA, 20 bp, Artificial Sequence
  Synthetic construct
  Sapablursen
cuuuattccaaagggcagcu

SEQ ID NO: 147 - RNA, 17 nt, Artificial Sequence
  Synthetic construct
  Sepofarsen
gguggaucacgaguuca

SEQ ID NO: 148 - DNA, 18 bp, Artificial Sequence
  Synthetic construct
  Tadnersen
gcccctagcgcgcgacuc

SEQ ID NO: 149 - DNA, 11 bp, Artificial Sequence
  Synthetic construct
  Tilsotolimod chain 1
tcgaacgttcg

SEQ ID NO: 150 - DNA, 11 bp, Artificial Sequence
  Synthetic construct
  Tilsotolimod chain 2
tcgaacgttcg

SEQ ID NO: 151 - RNA, 21 nt, Artificial Sequence
  Synthetic construct
  Tomligisiran antisense
uaccaauuuaugccuacagcg

SEQ ID NO: 152 - RNA, 21 nt, Artificial Sequence
  Synthetic construct
  Tomligisiran sense
cgcuguaggcauaaauuggua

SEQ ID NO: 153 - DNA, 21 bp, Artificial Sequence
  Synthetic construct
  TOP-1731
tcatgagtggcagctgcaatt

SEQ ID NO: 154 - RNA, 25 nt, Artificial Sequence
  Synthetic construct
  Varodarsen
uuugccgcugcccaaugccauccug

SEQ ID NO: 155 - DNA, 21 bp, Artificial Sequence
  Synthetic construct
  VEGLIN3
tggcttgaagatgtactcgat

SEQ ID NO: 156 - RNA, 23 nt, Artificial Sequence
  Synthetic construct
  Zilebesiran antisense
uguacucucauuguggaugacga

SEQ ID NO: 157 - RNA, 21 nt, Artificial Sequence
  Synthetic construct
  Zilebesiran sense
gucauccacaaugagaguaca

SEQ ID NO: 158 - DNA, 13 bp, Artificial Sequence
  Synthetic construct
  Factor VII ASO
gcaccacggtcca

(19) United States
(12) Patent Application Publication
(10) Pub. No.: US 2024/0207409 A1
(43) Pub. Date: Jun. 27, 2024

(54) ORAL DELIVERY OF OLIGONUCLEOTIDES

(71) Applicant: CiVi Biopharma, Inc., Chevy Chase, MD (US)

(72) Inventors: Henrik OERUM; Stewart Alwyl NOBLE, Chevy Chase, MD (US); Charles Lester SHEAR, Chevy Chase, MD (US)

(21) Appl. No.: 18/555,229

(22) PCT Filed: Apr. 21, 2022
(86) PCT No.: PCT/US2022/025807

§ 371 Date: Oct. 12, 2023

Related U.S. Application Data
(60) Provisional application No. 63/178,361, filed on Apr. 22, 2021.
(60) Provisional application No. 63/261,506, filed on Sep. 22, 2021.

(51) Int. Cl. A61K 47/18; A61K 9/00; A61K 9/48; C12N 15/113

(52) U.S. Cl. CPC A61K 47/183; A61K 9/0053; A61K 9/4825; A61K 9/4891; C12N 15/113; C12N 2310/11; C12N 2310/13; C12N 2310/14; C12N 2310/141; C12N 2310/531

(57) ABSTRACT

The present disclosure provides oligonucleotide compositions comprising (1) an oligonucleotide of the present disclosure, e.g., an ASO, siRNA, shRNA, DNA or RNA aptamer, gene therapy vector, miRNA, miRNA mimic, antimiR, DNA or RNA decoy, CpG oligonucleotide, or any therapeutic or diagnostic oligonucleotide known in the art, and (ii) a caprylic acid derivative, e.g., 5-CNAC. In some aspects, the oligonucleotide composition is formulated for delivery to the gastrointestinal tract. Thus, some aspects, the present disclosure provides oligonucleotide compositions for oral delivery comprising a therapeutic or diagnostic oligonucleotide (e.g., an ASO) and a caprylic acid derivative (e.g., 5-CNAC or de derivative thereof).

### CROSS-REFERENCE TO RELATED APPLICATIONS

This PCT application claims the priority benefit of U.S. Provisional Application No. 63/178,361, filed on Apr. 22, 2021, 63/261,506, filed on Sep. 22, 2021, and 63/288,379, filed Dec. 10, 2021, all of which are herein incorporated by reference in their entireties.

### REFERENCE TO SEQUENCE LISTING SUBMITTED ELECTRONICALLY VIA EFS-WEB

The content of the electronically submitted sequence listing (Name: 4009_023PC03_Seqlisting_ST25.txt; Size: 35,805 bytes; and Date of Creation: Apr. 20, 2022) filed with the application is incorporated herein by reference in its entirety.

### FIELD

The present disclosure relates to oral formulations comprising oligonucleotides, e.g., antisense oligonucleotides (ASO), siRNA, shRNA, and at least one delivery agent derived from caprylic acid, e.g., SNAC or 5-CNAC.

### BACKGROUND

Oral delivery of pharmacologically active agents is generally the delivery route of choice since it is convenient, relatively easy and generally painless, resulting in greater patient compliance relative to other modes of delivery. However, biological, chemical and physical barriers such as varying pH in the gastrointestinal tract, powerful digestive enzymes, and active agent impermeable gastrointestinal membranes, makes oral delivery of some pharmacologically active agents to mammals problematic, e.g., the oral delivery of therapeutic nucleic acids, such as antisense oligonucleotides.

Oral delivery of hydrophilic macromolecules with a molecular weight above 1000 Da remains a challenge due to susceptibility to pH and gastric/small intestinal enzymes, as well as low intestinal epithelial membrane permeability. The low permeability results from minimal passive or carrier-mediated transcellular permeation across phospholipid bilayers, as well as restricted paracellular transport via tight junctions.

Accordingly, there is a need to develop systems that allow the oral delivery of therapeutic nucleic acids, such as antisense oligonucleotides.

### BRIEF SUMMARY

The present disclosure provides a method for increasing (i) oral uptake, (ii) biological effect or therapeutic effect, and/or (iii) circulating plasma levels, of a therapeutic oligonucleotide, comprising co-administering the therapeutic oligonucleotide and N-(5-chlorosalicyloyl)-8-aminocaprylic acid (5-CNAC). In some aspects, the (i) oral uptake, (ii) biological effect or therapeutic effect, and/or (ii) circulating plasma levels is increased by at least about 10%, about 20%, about 30%, about 40%, about 50%, about 60%, about 70%, about 80%, about 90%, about 100%, about 125%, about 150%, about 175%, or about 200% compared to oral uptake, biological effect or therapeutic effect and/or circulating plasma levels observed when the therapeutic oligonucleotide is administered without 5-CNAC or co-administered with N-(8-[2-hydroxybenzoyl]amino)caprylic acid (SNAC).

The present disclosure provides an oligonucleotide composition comprising a therapeutic oligonucleotide, and 5-CNAC or a salt thereof, wherein the oligonucleotide composition is formulated for delivery to the gastrointestinal tract. In some aspects, the salt of 5-CNAC is selected from the group consisting of a sodium salt, a potassium salt, a calcium salt, and any combination thereof, and wherein the salt of 5-CNAC is a monosodium salt or a disodium salt. In some aspects, the therapeutic oligonucleotide is an antisense oligonucleotide (ASO), short interference RNA (siRNA), small hairpin RNA (shRNA), DNA and/or RNA aptamer, micro RNA (miRNA), anti-micro RNA (antimiR), CpG oligonucleotide, or DNA and/or RNA decoy.

The present disclosure also provides a method of manufacturing the oligonucleotide composition comprising a therapeutic oligonucleotide, and 5-CNAC or a salt thereof, wherein the method comprises admixing (i) a therapeutic oligonucleotide selected from the group consisting of an ASO, a siRNA, a shRNA, a DNA or RNA aptamer, a miRNA, a miRNA mimic, an antimiR, a DNA or RNA decoy, and CpG oligonucleotide; and, (ii) 5-CNAC or a salt thereof, wherein the salt is a monosodium or disodium salt. In some aspects, the admixing comprises dry blending. In some aspects, the method further comprises encapsulating the resulting dry blend of step in a capsule.

Also provided is a tablet or capsule comprising an oligonucleotide composition comprising (i) a therapeutic oligonucleotide selected from the group consisting of an ASO, a siRNA, a shRNA, a DNA or RNA aptamer, a miRNA, a miRNA mimic, an antimiR, a DNA or RNA decoy, and CpG oligonucleotide; and, (ii) 5-CNAC or a salt thereof, wherein the salt is a monosodium or disodium salt. In some aspects, the tablet or capsule comprises an enteric coating, a pH sensitive coating, or a combination thereof. In some aspects, the tablet or capsule has a weight between 10 mg and 500 mg. In some aspects, the tablet or capsule comprises 1 mg to 500 mg of therapeutic oligonucleotide.

The present disclosure also provides a method of treating a disease or condition in a subject in need thereof comprising administering an effective amount of an oligonucleotide composition disclosed herein or a tablet or capsule disclosed herein to the subject. In some aspects, the oligonucleotide composition, table, or capsule is administered orally. In some aspects, the oligonucleotide composition, tablet, or capsule is administered as a single dose or multiple doses. In some aspects, the oligonucleotide composition, tablet, or capsule is administered at least 5, 10, 15, 20, 25, 30, 35, 40, 45, 50, 55 or 60 minutes prior to a meal. In some aspects of the methods, oligonucleotide compositions, tablets, or capsules disclosed herein, the therapeutic oligonucleotide is selected from the group consisting of 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™), ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimderlirsen, cobitolimod, cobomarsen, CODA-001 (NEXAGON™), cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™), fazisiran, fesomersen, fitusiran, fomivirsen (VITRAVENE™), frenlosirsen, gataparsen, givosiran (GIVLAARI™) GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS-FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX-H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™), oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™) pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SB010, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™), vupanorsen, vutrisiran, WVE-003, WVE-004, WVEN-531, zilebesiran, and zilganersen.

### BRIEF DESCRIPTION OF THE DRAWINGS/FIGURES

FIG. 1A shows the structure of caprylic acid, and two caprylic acid derivatives, SNAC and 5-CNAC.

FIG. 2A shows the structure of exemplary caprylic acid derivatives.

FIG. 2A shows the structure of representative caprylic acid derivatives.

FIG. 2 shows the chemical structures of exemplary oral delivery agents.

FIG. 3 shows the detailed chemical structure and full name of cepadacursen, an antisense oligonucleotide conjugate targeting PCSK9. CIVI 008 is a cepadacursen formulation for oral delivery.

FIG. 4 shows the detailed chemical structure of ISIS 863633, an antisense oligonucleotide conjugate targeting PCSK9.

FIG. 5 shows potential topologies of oligonucleotides comprising partially double-stranded nucleic acids, e.g., RNA sponges or tough decoys. The arrows in the structures represent antisense sequences that can bind to a target RNA, e.g., a microRNA.

FIG. 6 shows the design of the clinical trial presented in Example 1.

FIG. 7 shows plasma concentration levels of CIVI 008 after administration with or without SNAC.

FIG. 8 shows plasma AUC levels in the SQ (subcutaneous) arm (P0001, P0002, P0901, and P0902) and PO (oral) arm of the study of Example 1 (P0301, P0302, P0303, P1201, P1202, P1203, P0401, P0402, P0403, P1301, P1302, P1303, P0501, and P1401).

FIG. 9 shows liver concentration levels of CIVI 008 in the SQ and PO arms of the study presented in Example 1.

FIG. 10 shows mean changes in PCSK9 expression levels with respect to baseline level at days 35 and 42 after oral CIVI 008 administration.

FIGS. 11A and 25B show changes in plasma LDL cholesterol levels with respect to baseline after administration of one or two capsules of CIVI 008 (FIG. 11A) or under control conditions (FIG. 11B).

FIG. 12 shows changes in plasma LDL cholesterol levels with respect to baseline during the study presented in Example 1.

FIG. 13 shows a schematic description of the study presented in Example 3.

FIG. 14 shows plasma concentration levels of CIVI 008 after administration with 5-CNAC.

FIG. 15 shows a comparison between pharmacokinetic parameters (mean AUC₀₋₅ and mean Cₘₐₓ) corresponding to the administration of CIVI 008 capsules comprising either SNAC or 5-CNAC.

FIG. 16 shows plasma concentration levels and pharmacokinetic parameters (mean AUC₀₋₅ and mean Cₘₐₓ) corresponding to administration of CIVI 008 with 5-CNAC in size 4 capsules (Group A) or size 0 capsules (Group B).

FIG. 17 shows the Mean AUC and mean Cₘₐₓ at Days 1 and 3, in monkeys administered from 5 mg to 30 mg of CIVI 008 formulated with 5-CNAC.

FIG. 18A and FIG. 18B show a comparison of mean pharmacokinetic parameters on Day 1 and 3, in monkeys administered similar doses of CIVI 008 in capsules prepared by either dry blending (FIG. 18A) or freeze-drying (FIG. 18B).

FIG. 19 shows the reduction from baseline in PCSK9 and plasma LDL after 22 days of dosing.

FIG. 20 shows the % LDL reduction from baseline in monkeys administered CIVI 008 formulated with either SNAC or 5-CNAC.

FIG. 21 shows plasma concentration levels and pharmacokinetic parameters (mean AUC₀₋₅, mean Cₘₐₓ and Tmax) corresponding to administration of CIVI 008 with or without GalNac and formulated with 5-CNAC in size 0 capsules.

FIG. 22 shows plasma concentration levels and pharmacokinetic parameters (mean AUC₀₋₅, mean Cₘₐₓ and Tmax) corresponding to administration of an oligos against Factor VII with or without GalNac and formulated with 5-CNAC in size 0 capsules.

FIG. 23 as is schematic representation showing exemplary constructs comprising one or more caprylic acid derivatives of the present disclosure, including 5′ covalent and/or 3′ covalent attachment to single or double stranded oligonucleotides, attachment at internal locations (i.e., not 5′ or 3′ end), attachment of multiple concatenated caprylic acid derivatives, attachment of caprylic acid derivatives to loops, attachment of caprylic acid derivatives to overhangs, and attachment of caprylic acid derivatives to non-complementary regions.

### DETAILED DESCRIPTION

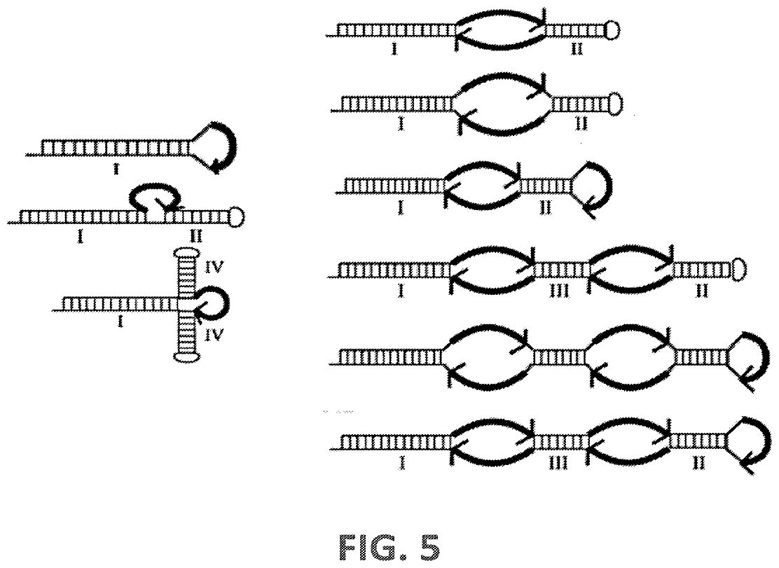

The present disclosure provides compositions comprising an oligonucleotide or combination thereof comprising, e.g., an antisense oligonucleotide (ASO), short interference RNA (siRNA), small hairpin RNA (shRNA), RNA and/or DNA aptamer, micro RNA (miRNA), anti-micro RNA (antimiR), DNA or RNA decoy (see, e.g., FIG. 5), CpG oligonucleotide, or any combination thereof, and a delivery agent comprising a caprylic acid derivative, e.g., SNAC, or 5-CNAC. In some aspects, the oligonucleotide compositions of the present disclosure are formulated for delivery to the gastrointestinal tract, e.g., for oral delivery.

Caprylic acid derivatives, e.g., SNAC or 5-CNAC, work through a number of different mechanisms to improve the absorption of nucleic acids in the gastrointestinal tract. Due to their lipophilic properties, they are able to embed into and modify the composition of plasma membranes. According, intracellular accumulation of therapeutic agents is possible via a transcellular process. Furthermore, caprylic acid derivatives such as SNAC or 5-CNAC can reduce the tendency of therapeutic agents to aggregate (e.g., to form multimers that may affect absorption). Caprylic acid derivatives such as SNAC or 5-CNAC can also act as buffers to neutralize the acidic conditions of the stomach (gastric pH as generally a range between 1 and 2.5), which can enhance the absorption of the therapeutic agent in at least two ways. First, the activity of digestive enzymes is higher at an acidic pH. Accordingly, the local increase in pH neutralizes degradative enzymes and therefore decreases the probability of enzymatic degradation. This extension of the half-life of the therapeutic agent allows more drug to be absorbed into the gastrointestinal tract cells and reach systemic circulation. Second, the buffering activity of caprylic acid derivatives such as SNAC or 5-CNAC can modify (e.g., enhance) the solubility of the therapeutic agent. These mechanisms of action differ from the mechanisms of action of other oral delivery agents such as C₁₀ (sodium caprate). C₁₀ acts via opening epithelial tight junctions, which is the result of inositol 1,4,5-triphosphate (IP3) and calcium-mediated cell signaling events that trigger the contraction of the peri-junctional actomyosin ring (PAMR), permitting increased tight junction permeability. Of important concern about C₁₀ are its antimicrobial effects (see, e.g., Cox et al. 2008, Pharm. Res. 25:114-122; Petschow et al. 1996, Antimicrob. Agents Chemother. 40:302-306; Van Immerseel et al. 2004, Appl. Environ. Microbiol. 70:3582-3587; and Chadeganipour and Haims 2001, Mycoses 44:109-112), which can promote gastrointestinal microbiome changes. These changes in gastrointestinal microbiome may lead to local inflammation. Similar antimicrobial effects have not been observed in caprylic acid derivatives such as SNAC or 5-CNAC.

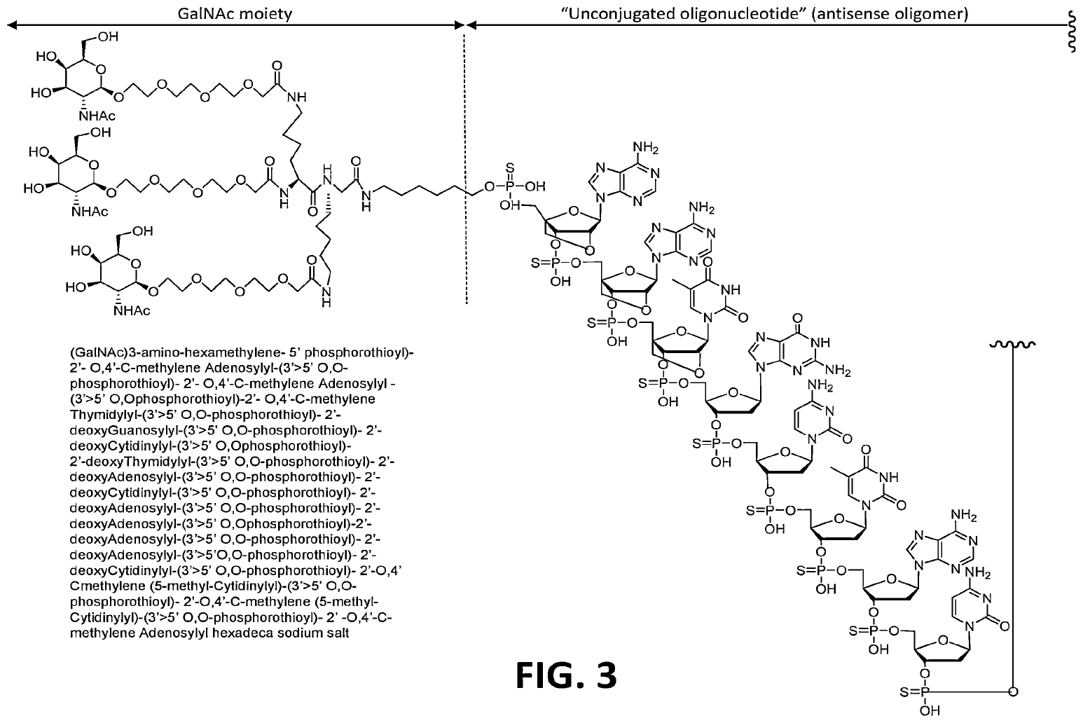

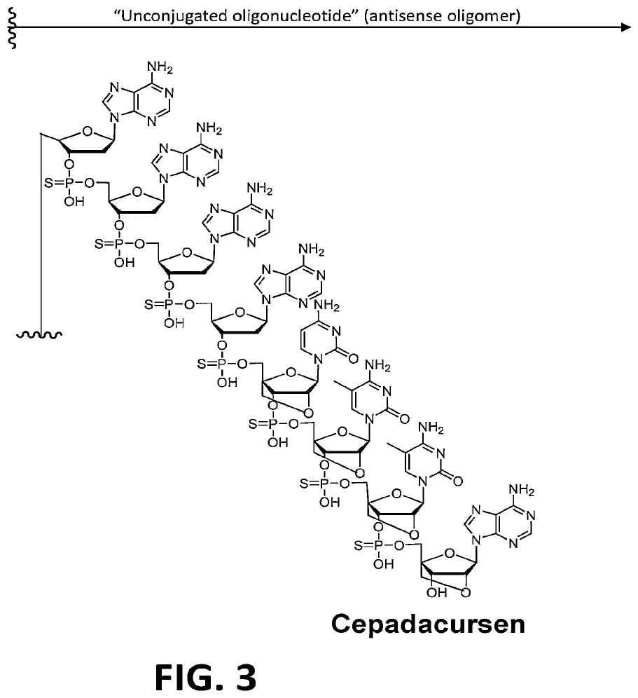

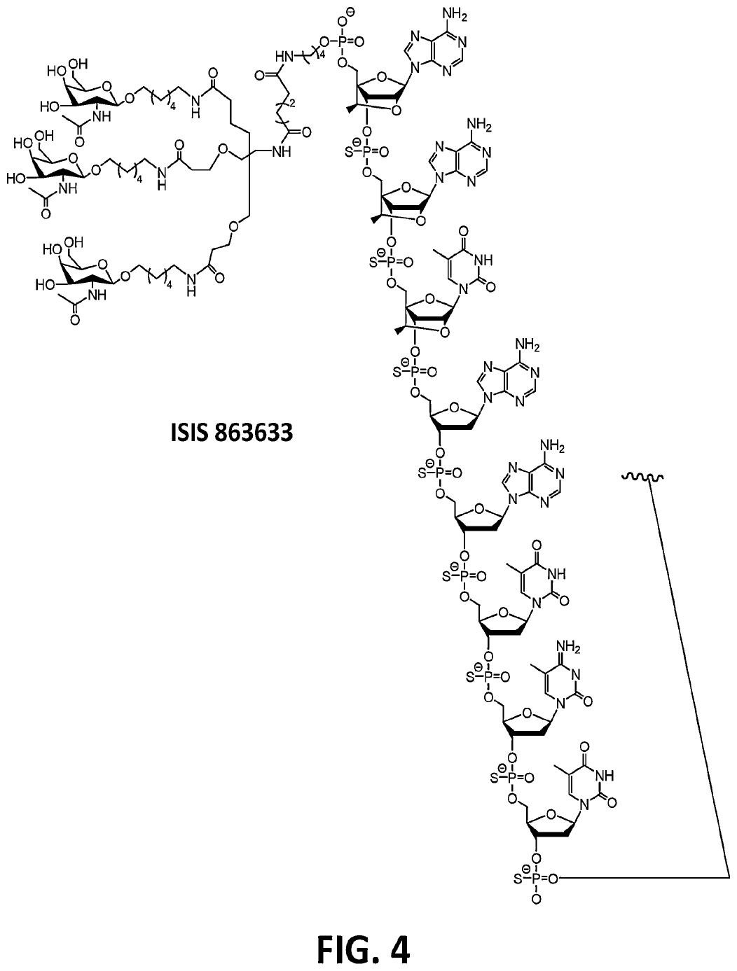

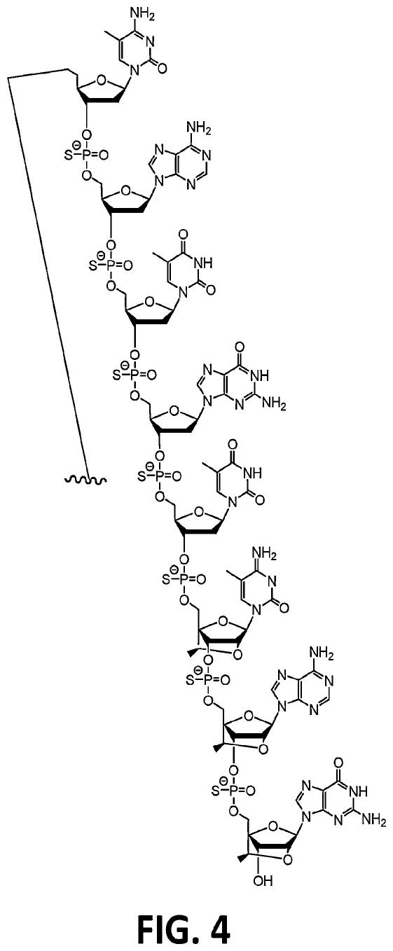

In some aspects, the oligonucleotide used in the compositions and methods of the present disclosure comprises an antisense oligonucleotide, e.g., an antisense oligonucleotide conjugate that targets a nucleic acid, e.g., an mRNA, encoding PCSK9. Specific examples of nucleic acid therapeutic agents that can be delivered in the compositions and methods disclosed herein are CIVI 008 and ISIS 863633 as depicted in FIG. 3 and FIG. 4, respectively.

In some aspects, the oligonucleotide compositions disclosed herein comprise a caprylic acid derivative such as SNAC or 5-CNAC that protect the oligonucleotide from conditions that may lead to the oligonucleotide's degradation and/or aggregation, for example, the conditions found in the gastrointestinal tract, for example, in the stomach. In some aspects, the oligonucleotide compositions disclosed herein can be used to administer therapeutic or diagnostic oligonucleotides to other locations in the body in which specific conditions such as low pH, high pH, or the presence of nucleases may lead to the degradation of the oligonucleotide. For example, the vagina's pH levels is 3.8 to 4.5, the pH level in the bladder is about 6.0 (ranging from 4.5 to about 8), and high nuclease levels are present in fluids such as tears, saliva, mucus, or perspiration. In other words, formulations for the treatment of mucose tissues, the urogenital tract, or for topical administration can also incorporate the caprylic acid derivatives disclosed herein, e.g., SNAC or 5-CNAC.

Experimental data presented herein shows that despite structural similarities, 5-CNAC is unexpectedly efficient in increasing the absorption and bioavailability of therapeutic oligonucleotides, e.g., ASOs, with respect to SNAC. Thus, the formulation of therapeutic oligonucleotides with 5-CNAC results in much higher plasma concentrations when the therapeutic oligonucleotides are orally administered compared to the plasma concentrations observed when the same oligonucleotides are formulated with SNAC. It has also been observed that formulations with 5-CNAC can obviate the need to use delivery moieties such as GalNAc. In other words, it is possible to obviate the conjugation of therapeutic oligonucleotides with GalNAc, and still achieve plasma concentration above those observed when the oligonucleotide is conjugated to GalNAc when the therapeutic oligonucleotide is formulated with 5-CNAC.

The oligonucleotide compositions disclosed herein, e.g., compositions comprising an oligonucleotide, e.g., in pill or capsule form, comprise at least one delivery agent that protects the payload (i.e., nucleic acid therapeutic agent), e.g., during passage through the gastrointestinal tract (e.g., through the stomach and upper portion of the small intestine) following oral administration. In some aspects, the delivery agent (e.g., for oral delivery) is a salt (e.g., a sodium salt) of a fatty acid, such as a caprylic acid derivative, e.g., 8-[2-hydroxybenzoyl]amino) caprylic acid, SNAC, or 5-CNAC, or a combination thereof. In a particular aspect, the delivery agent (e.g., for oral delivery) is a salt, e.g., a sodium salt (e.g., a monosodium or disodium salt) of SNAC or a sodium salt (e.g., a monosodium or disodium salt) of 5-CNAC.

Also provided herein are methods of manufacturing the oligonucleotide compositions and delivery agents disclosed herein. The disclosure also provides methods to treat a subject a subject if need thereof comprising administering the oligonucleotide compositions disclosed herein.

### Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “derivative” as used herein refers to a chemical compound related structurally to a compound disclosed herein (e.g., C8, SNAC, or 5-CNAC), e.g., a compound having the same carbon skeleton, but chemically modified to introduce, e.g., a side chain or group disclosed herein, in one or more positions, and wherein the derivative possesses a biological activity (e.g., the ability to function as an oral delivery agent) that is substantially similar to a biological activity of the entity or molecule it is a derivative.

The term “oligomer” or “oligonucleotide” in the context of the present disclosure are used interchangeably, and refer to a molecule formed by covalent linkage of two or more nucleotides. Herein, a single nucleotide (unit) can also be referred to as a monomer or unit.

The term “nucleotide” as used herein, refers to a glycoside comprising a sugar moiety, a base moiety and a covalently linked group (linkage group), such as a phosphate or phosphorothioate internucleoside linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide can be referred to as a monomer or unit. In certain aspects, the term “nucleotide analogs” refers to nucleotides having modified sugar moieties. Non-limiting examples of the nucleotides having modified sugar moieties (e.g., LNA) are disclosed elsewhere herein. In other aspects, the term “nucleotide analogs” refers to nucleotides having modified nucleobase moieties. The nucleotides having modified nucleobase moieties include, but are not limited to, 5-methyl-cytosine, isocytosine, pseudoisocytosine, 5-bromouracil, 5-propynyluracil, 6-aminopurine, 2-aminopurine, inosine, diaminopurine, and 2-chloro-6-aminopurine. In some aspects, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleoside” as used herein refers to a glycoside comprising a sugar moiety and a base moiety, and can therefore be used when referring to the nucleotide units, which are covalently linked by the internucleoside linkages between the nucleotides of a polynucleotide, e.g., an ASO disclosed herein. In the field of biotechnology, the term “nucleotide” is often used to refer to a nucleic acid monomer or unit. In the context of a polynucleotide, e.g., an ASO disclosed herein, the term “nucleotide” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleoside linkages are implicit. Likewise, particularly in the case of oligonucleotides where one or more of the internucleoside linkage groups are modified, the term “nucleotide” can refer to a “nucleoside.” For example, the term “nucleotide” can be used, even when specifying the presence or nature of the linkages between the nucleosides.

In some aspects, the terms “nucleoside”, “nucleotide”, “unit” and “monomer” are used interchangeably. It will be recognized that when referring to a sequence of nucleotides or monomers, what is referred to is the sequence of bases, such as A, T, G, C or U.

The plural terms “nucleic acids” or “nucleotides” is intended to encompass plural nucleic acids. In some aspects, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In some aspects, “nucleic acids” or “nucleotides” refer to a sequence in an oligonucleotide of the present disclosure, e.g., an ASO, siRNA, shRNA, DNA or RNA aptamer, -, miRNA, miRNA mimic, antimiR, DNA or RNA decoy, CpG oligonucleotide, or any therapeutic or diagnostic oligonucleotides known in the art.

When the term refers to a sequence in a oligonucleotide, e.g., an ASO disclosed herein, the nucleic acids or nucleotides can be non-naturally occurring, i.e., chemically synthesized, enzymatically produced, recombinantly produced, or any combination thereof. In some aspects, the nucleic acids or nucleotides in an oligonucleotide, e.g., an ASO disclosed herein, are produced synthetically or recombinantly, but are not a naturally occurring sequence or a fragment thereof. In some aspects, the nucleic acids or nucleotides in a polynucleotide, e.g., an ASO disclosed herein, are not naturally occurring because they contain at least one nucleoside analog that is not naturally occurring in nature.

The terms “reverse complement”, “reverse complementary” and “reverse complementarity” as used herein are interchangeable with the terms “complement”, “complementary” and “complementarity”.

The term, “complementary” means that two sequences are complementary when the sequence of one can bind to the sequence of the other in an anti-parallel sense wherein the 3-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 90%, preferably 95%, most preferably 100%, complementarity to a defined sequence.

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine. Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine. In some aspects, the nucleobases can be independently selected from the group consisting of adenine, guanine, cytosine, thymidine, uracil, 5-methylcytosine. In some aspects, the nucleobases can be independently selected from the group consisting of adenine, guanine, cytosine, thymidine, and 5-methylcytosine. In some aspects, at least one of the nucleobases present in an oligomer of the present disclosure is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

“Nucleotide analogues” are variants of natural nucleotides, such as DNA or RNA nucleotides, by virtue of modifications in the sugar and/or base moieties. Analogues can, in principle, be merely “silent” or “equivalent” to the natural nucleotides in the context of the oligonucleotide, i.e., they have no functional effect on the way the oligonucleotide works to inhibit target gene expression. Such “equivalent” analogues can nevertheless be useful if, for example, they are easier or cheaper to manufacture, or are more stable to storage or manufacturing conditions, or represent a tag or label.

The term “nucleotide length” as used herein means the total number of the nucleotides (monomers) in a given sequence. As one of ordinary skill in the art would recognize, the 5′ terminal nucleotide of a nucleic acid, e.g., an ASO, does not comprise a 5′ internucleoside linkage group, although it can comprise a 5′ terminal group.

The compounds described herein, e.g., caprylic acid derivatives or oligonucleotides, can contain one or several asymmetric centers, and can be present in the form of optically pure enantiomers or mixtures of enantiomers, for example, racemates, mixtures of diastereoisomers, diastereoisomeric racemates, or mixtures of diastereoisomeric racemates. In some aspects, the asymmetric center can be an asymmetric carbon atom. The term “asymmetric carbon atom” means a carbon atom with four different substituents. According to the Cahn-Ingold-Prelog Convention an asymmetric carbon atom can be of the “R” or “S” configuration.

As used herein, a “coding region,” “coding sequence,” or “open reading frame” is a portion of a polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. In some aspects, an oligonucleotide, e.g., an ASO disclosed herein such as CIVI 008, can target a PCSK9 coding region of a nucleic acid encoding the PCSK9 protein, e.g., an RNA.

The term “non-coding region” as used herein means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript. In some aspects, an oligomer (e.g., an ASO) or antisense oligonucleotide conjugate (e.g., ISIS 863633) disclosed herein can target a PCSK9 non-coding region of a nucleic acid encoding the PCSK9 protein, e.g., an RNA.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “downstream,” when referring to a nucleotide sequence, means that a nucleic acid or a nucleotide sequence is located 3′ to a reference nucleotide sequence. In certain aspects, downstream nucleotide sequences relate to sequences that follow the starting point of transcription. For example, the translation initiation codon of a gene is located downstream of the start site of transcription. In some aspects, an oligonucleotide, e.g., an ASO, disclosed herein can target a region of a nucleic acid encoding a target protein, e.g., an RNA, downstream of the target protein open reading frame (ORF).

The term “upstream” refers to a nucleotide sequence that is located 5′ to a reference nucleotide sequence. In some aspects, an oligonucleotide, e.g., an ASO, disclosed herein can target a region of a nucleic acid encoding a target protein, e.g., an RNA, upstream of the target protein ORF.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, UTRs, and stem-loop structures. If a coding region is intended for expression in a eukaryotic cell, a polyadenylation signal and transcription termination sequence will usually be located 3′ to the coding sequence. In some aspects, an oligonucleotide, e.g., an ASO, disclosed herein can target a regulatory region.

The term “transcript” as used herein can refer to a primary transcript that is synthesized by transcription of DNA and becomes a messenger RNA (mRNA) after processing, i.e., a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms to produce different proteins and RNAs, such as mRNA, tRNA, rRNA, lncRNA, miRNA and others. Thus, the term “transcript” can include exons, introns, 5′ UTRs, and 3′ UTRs.

The term “expression” as used herein refers to a process by which a polynucleotide produces a gene product, for example, an RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide that is translated from a transcript. Gene products described herein further include nucleic acids with post-transcriptional modifications, e.g., polyadenylation or splicing, or polypeptides with post-translational modifications, e.g., methylation, glycosylation, the addition of lipids, association with other protein subunits, or proteolytic cleavage.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal. In some aspects, the subject is a human.

As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The term “pharmaceutical composition” refers to a preparation which is in such form as to permit the biological activity of the active ingredient to be effective, and which contains no additional components which are unacceptably toxic to a subject to which the composition would be administered. Such composition can be sterile. The term “oral pharmaceutical composition” refers to a pharmaceutical composition that can be administered orally. Oral administration is a route of administration where a substance is taken through the mouth. “Per os” abbreviated to P.O. is sometimes used as a direction for medication to be taken orally. Many medications are taken orally because they are intended to have a systemic effect, for example, reaching different parts of the body via the bloodstream.

The term “delivery agent” as used herein refers to a carrier compound or carrier molecule that is useful in the delivery of a nucleic acid therapeutic agent of the present disclosure, e.g., an ASO. The term “oral delivery agent” as used herein refers to a carrier compound or carrier molecule that is useful in the oral delivery of a nucleic acid therapeutic agent of the present disclosure, e.g., an ASO.

In some aspects, the pharmaceutical composition of the present disclosure is administered orally. The term “oral,” as used herein, and grammatical variants thereof (e.g., orally) comprises any kind of oral delivery routes (comprising buccal, sublabial, and sublingual routes). Medications for oral administration can come in various forms, including oral solid dosage (OSD) forms (e.g., tablets to swallow, chew, or dissolve in water or under the tongue; capsules and chewable capsules, e.g., with a coating that dissolves in the stomach or bowel to release the medication there; time-release or sustained-release tablets and capsules, which release the medication gradually; powders; or granules), and oral liquid dosage forms (e.g., teas, drops, liquid medications, suspensions, or syrups).

“Administering,” as used herein, means to give a composition, e.g., an oral pharmaceutical composition comprising an oligonucleotide of the present disclosure, e.g., an ASO, siRNA, shRNA, DNA or RNA aptamer, gene therapy vector, miRNA, miRNA mimic, antimiR, DNA or RNA decoy, CpG oligonucleotide, or any therapeutic or diagnostic oligonucleotide known in the art to a subject via a pharmaceutically acceptable route, e.g., orally. An “effective amount” of, e.g., e.g., an oral pharmaceutical composition comprising an oligonucleotide disclosed herein, is an amount sufficient to carry out a specifically stated purpose, e.g., to treat a disease or condition. An “effective amount” can be determined empirically and in a routine manner, in relation to the stated purpose.

“Treat,” “treatment,” or “treating,” as used herein refers to, e.g., the reduction in severity of a disease or condition; the reduction in the duration of the course of a disease or condition; the amelioration or elimination of one or more symptoms associated with a disease or condition; delay the onset of a disease or condition; or the provision of beneficial effects to a subject with a disease or condition, without necessarily curing the disease or condition. The term also includes prophylaxis or prevention of a disease or condition, or its symptoms or sequelae.

“Prevent” or “preventing,” as used herein, refers to decreasing or reducing the occurrence or severity of a particular outcome. In some aspects, preventing an outcome is achieved through prophylactic treatment. In some aspects, an oral pharmaceutical composition comprising a nucleic acid therapeutic agent disclosed herein is administered to a subject prophylactically. In some aspects, the subject is at risk of developing a disease or condition.

The terms “oligonucleotide composition of the present disclosure,” “therapeutic oligonucleotide of the present disclosure,” or “oligonucleotide of the present disclosure” refers, for example, to 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™), ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimderlirsen, cobitolimod, cobomarsen, CODA-001 (NEXAGON™), cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™), fazisiran, fesomersen, fitusiran, fomivirsen (VITRAVENE™), frenlosirsen, gataparsen, givosiran (GIVLAARI™), GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS-FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX-H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™), oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™), pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SB010, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™) vupanorsen, vutrisiran, WVE-003, WVE-004, WVEN-531, zilebesiran, zilganersen, and non-conjugated forms thereof, i.e., forms of the molecules that comprise the nucleotide oligomer portion but do not comprise a conjugated moiety such as a GalNAc moiety or polyethylene glycol (PEG).

As used herein, the term “CIVI 008” refers to the compound shown in FIG. 3 (cepadacursen) formulated for oral administration.

As used herein, the term “unconjugated form” refers to the oligonucleotide portion of an oligonucleotide conjugate (e.g., cepadacursen, CIVI 008), as exemplified in FIG. 3. As shown in FIG. 3, the unconjugated form of an ASO would correspond to the antisense oligomer portion of the ASO, i.e., the ASO without its GalNAc moiety. In the case of a double stranded therapeutic oligonucleotide, e.g., a siRNA, the unconjugated form would comprise the sense-antisense duplex without delivery moieties (e.g., GalNAc) that may be covalently attached to the sense strand.

### I. Oligonucleotide Compositions Comprising Caprylic Acid Derivatives

The present disclosure provides oligonucleotide compositions comprising: (i) an ASO, siRNA, shRNA, DNA or RNA aptamer, miRNA, miRNA mimic, antimiR, DNA or RNA decoy, CpG oligonucleotide, any therapeutic or diagnostic oligonucleotide known in the art, or a combination thereof; and (ii) a caprylic acid derivative, e.g., SNAC or 5-CNAC, a salt thereof, or any combination thereof.

In some aspects, the oligonucleotide compositions of the present disclosure are delivered to the gastrointestinal tract, e.g., orally. Thus, in some aspects, the present disclosure provides oligonucleotide compositions for oral delivery comprising. e.g., an ASO, siRNA, shRNA, DNA or RNA aptamer, miRNA, miRNA mimic, antimiR, DNA or RNA decoy, CpG oligonucleotide, or any therapeutic or diagnostic oligonucleotide known in the art, and a caprylic acid derivative, e.g., SNAC or 5-CNAC.

As used herein the term “caprylic acid derivative” refers to a molecule comprising a carbocyclic 6-membered ring with a fatty acid substituent, e.g., C8 (caprylic acid), wherein the ring further comprises and at least one polar substituent group, e.g., an —OH or halogen group.

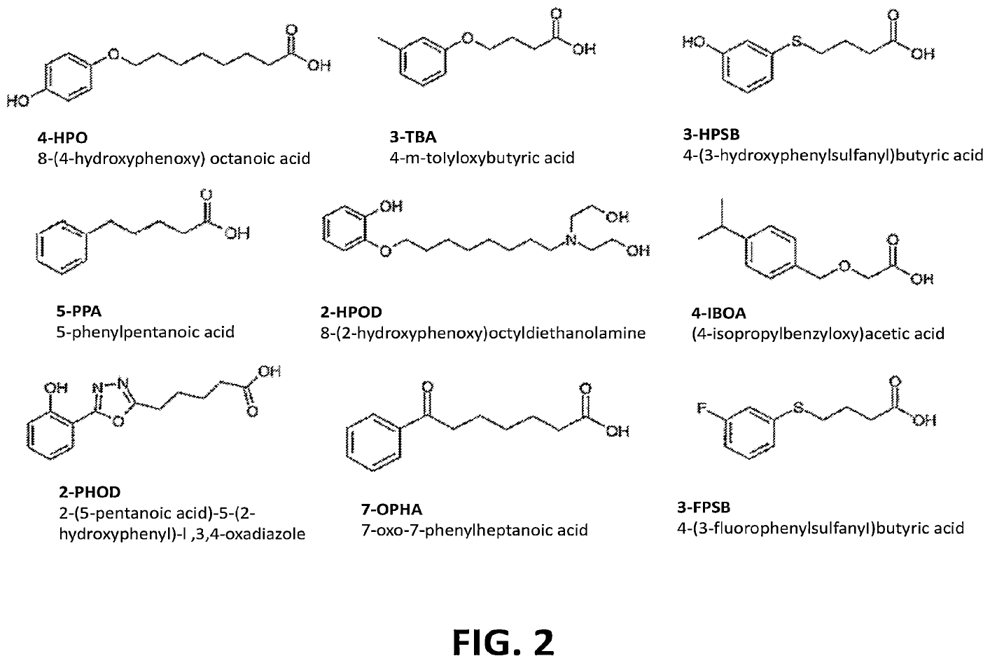

In some aspects, the term caprylic acid derivative broadly encompasses the compounds of FIG. 2, which comprise, e.g., butyric (C4), pentanoic (C5), or heptanoic (C7) fatty acid moieties. In some aspects, the caprylic acid derivatives of the present disclosure (e.g., 5-CNAC) can be used as oligonucleotide delivery agents. In some aspects, the caprylic acid derivatives of the present disclosure (e.g., 5-CNAC) can be used as gastrointestinal tract delivery agents. In some aspects, the caprylic acid derivatives of the present disclosure (e.g., 5-CNAC) can be used as oral delivery agents.

In some aspects, the caprylic acid derivative comprises a compound of the formula presented below

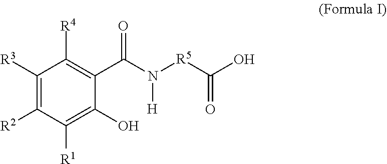

wherein
(i) R¹, R², R³, and R⁴ are independently hydrogen, OH, NR⁶R⁷, halogen, C₁-C₄ alkyl, or C₁-C₄ alkoxy;
(ii) R⁵ is a substituted or unsubstituted C₂-C₁₆ alkylene, substituted or unsubstituted C₂-C₁₆alkenylene, substituted or unsubstituted C₁-C₁₂ alkyl(arylene), or substituted or unsubstituted aryl(C₁-C₄ alkylene); and
(iii) R⁶ and R⁷ are independently hydrogen, oxygen, or C₁-C₄ alkyl.

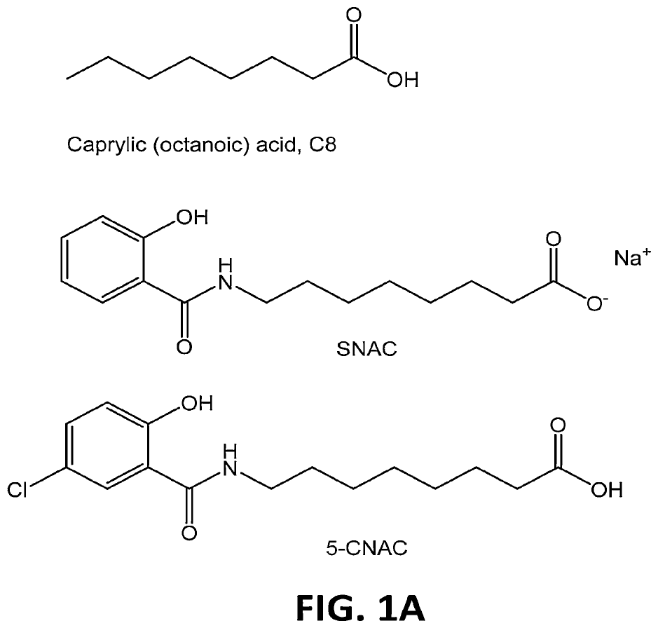

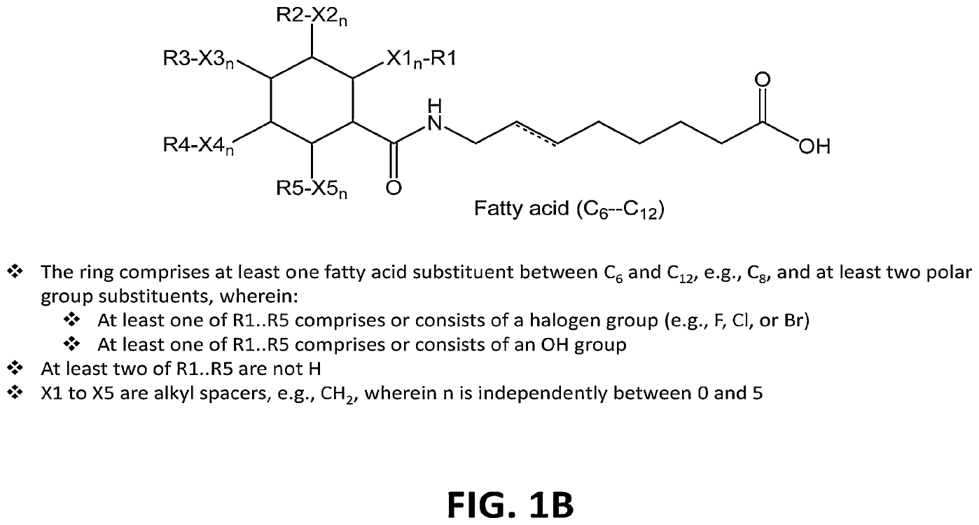

In some aspects, the caprylic acid derivative comprises a compound of the formula presented below (see FIG. 1B)

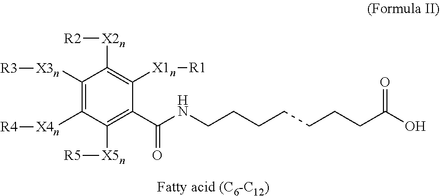

wherein the benzene ring comprises at least one fatty substituent between C2 and C12, e.g., C8, and at least two polar substituents wherein
a. At least one of R1, R2, R3, R4 or R5 independently comprises or consists of a halogen, e.g., a halogen selected from F, Cl, or Br;
b. At least one of R1, R2, R3, R4, or R5 independently comprises or consists of a hydroxy group;
c. At least two of R1, R2, R3, R4, or R5 are not H;
d. X1, X2, X3, X4, and X5 are alkyl spacers, linear or branched, comprising n CH₂ units wherein n is 0 to 5 (e.g., linear groups such as —CH₂—, —CH₂—CH₂—, —CH₂—CH₂—CH₂—).

A “halogen” group refers to fluorine, chlorine, bromine or iodine.

A “hydroxy function” or “hydroxy group” is OH.

In some aspects, the ring portion of a caprylic acid derivative of the present disclosure, e.g., a molecule of Formula I or Formula II, is a benzene ring. However, in other aspects, the ring portion of a caprylic acid derivative of the present disclosure can be a carbocyclic or heterocyclic three to ten-membered ring, which may be saturated, partially unsaturated, or aromatic. In some aspects, the heterocycle comprises between one and four heteroatoms selected from amongst O, S, and N. In some aspects, the ring is optionally fused to between one and four five- or six-membered rings, wherein each ring can be independently saturated, partially unsaturated or aromatic, carbocyclic, or heterocyclic, and wherein each fused heterocycle can independently comprise one or two heteroatoms selected from amongst O, N, and S.

A “carbocycle” refers to a three- to 10-membered carbocyclic ring that can be saturated, partially unsaturated, or aromatic, and which is bound to the rest of the molecule via any available C atom.

A “heterocycle” refers to a three- to 10-membered cyclic ring containing at least one heteroatom selected from amongst N, O and S, that can be saturated, partially unsaturated, or aromatic, and which is bound to the rest of the molecule via any available C atom.

Examples of carbocycles and heterocycles include, amongst others, phenyl, naphthyl, thienyl, furyl, pyrrolyl, thiazolyl, isothiazolyl, imidazolyl, pyrazolyl, 1,2,3-triazolyl, 1,2,4-triazolyl, tetrazolyl, 1,3,4-thiadiazolyl, 1,2,4-thiadiazolyl, pyridyl, pyrazinyl, pyrimidinyl, pyridazinyl, benzimidazolyl, benzofuranyl, isobenzofuranyl, indolyl, isoindolyl, benzothiophenyl, benzothiazolyl, cyclopropyl, cyclobutyl, cyclopentyl, cyclohexyl, cycloheptyl, azetidinyl and aziridinyl.

In some aspects, at least one of the ring substituents is attached to the ring via an ether, thioether, carbon-carbon, or amide bond. In some aspects, at least one non-fatty acid ring substituent is attached to the ring via an ether, thioether, carbon-carbon, or amide bond. In some aspects, the fatty acid ring substituent is attached to the ring via an ether, thioether, carbon-carbon, or amide bond. In some aspects, at least one, two, three, four, or five substituents R¹, R², R³, R⁴, or R⁵ of Formula I or R1, R2, R3, R4, or R5 of Formula II, or substituents of a carbocyclic or heterocyclic three to ten-membered ring disclosed above, can be independently selected from the group consisting of an alkyl chain, an amine function, a hydroxy function, a carboxylic acid function, a carboxylic amide, a halogen, or any combination thereof.

The term “alkyl” or “alkyl chain” in the context of the present disclosure refers to a saturated hydrocarbon moiety, which can be linear, branched, cyclic, or cyclic with linear or branched side chains. The term alkyl includes partially unsaturated hydrocarbons such as propenyl. Examples are methyl, ethyl, n- or isobutyl, n- or cyclohexyl. The term alkyl can extend to alkyl groups linked or bridged by heteroatoms. Heteroatoms in the context of the present invention are nitrogen (N), sulfur (S) and oxygen (O).

An “amine function” or “amine group” is a function NRR′, with R and R′ selected independently, e.g., from hydrogen (—H) and an alkyl group such as a C₁-Cₙ alkyl, wherein n is an integer between 0 and 20.

A “carboxylic acid function” or “carboxylic acid group” is COOH or its anion, COO⁻.

A “carboxylic amide” is CONRR′, with R and R′ selected independently, e.g., from hydrogen (—H) and an alkyl group such as a C₁-Cₙ alkyl, wherein n is and integer between 0 and 20.

The caprylic acid derivatives of the present disclosure can be present in any form commonly used in pharmaceutical technology. Particular aspects include, but are not limited to, sodium salts, magnesium salts, potassium salts, ammonium salts, free acids, or a mixture of the preceding forms. Other pharmaceutically acceptable salts are known to the skilled artisan and can be obtained, inter alia, from Haynes et al. (2005) J. Pharmaceutical Sci. 94:2111-2120.

In some aspects, the caprylic acid derivative comprises a compound of Formula I or Formula II as described above, wherein the caprylic acid derivative is a free acid or a sodium salt, e.g., a monosodium or a disodium salt. In some aspects, the caprylic acid derivative is a hydrate or a solvate. In some aspects, the caprylic acid derivative is an alcohol solvate. In some aspects, the alcohol solvate is an ethanol solvate. In some aspects, the ethanol solvate is a solvate of a salt. In some aspects, the ethanol solvate is an ethanol solvate of a monosodium salt. In some aspects, the ethanol solvate is an ethanol solvate of a disodium salt. In some aspects, the caprylic acid derivative is a hydrate. In some aspects, the hydrate is a hydrate of a salt. In some aspects, the hydrate is a hydrate of a monosodium salt. In some aspects, the hydrate is a hydrate of a disodium salt.

Thus, in some aspects, the caprylic acid derivative can comprises free acid of 5-CNAC, a sodium salt of 5-CNAC, e.g., a monosodium salt of 5-CNAC, a disodium salt of 5-CAN, or a combination thereof. In some aspects, the caprylic acid derivative is a hydrate of 5-CNAC. In some aspects, the caprylic acid derivative is a solvate of 5-CNAC. In some aspects, the caprylic acid derivative is an alcohol solvate of 5-CNAC. In some aspects, the alcohol solvate is an ethanol solvate of CNAC. In some aspects, the ethanol solvate is a solvate of a salt of 5-CNAC. In some aspects, the ethanol solvate is an ethanol solvate of a monosodium salt of 5-CNAC. In some aspects, the ethanol solvate is an ethanol solvate of a disodium salt of 5-CNAC. In some aspects, the caprylic acid derivative is a hydrate of 5-CNAC. In some aspects, the hydrate is a hydrate of a salt of 5-CNAC. In some aspects, the hydrate is a hydrate of a monosodium salt of 5-CNAC. In some aspects, the hydrate is a hydrate of a disodium salt of 5-CNAC.

In some aspects, the caprylic acid derivative comprises a single compound (e.g., SNAC, or 5-CNAC). In other aspects, the caprylic acid derivative comprises a combination of compounds (e.g., a combination of SNAC, or 5-CNAC). In some aspects, the caprylic acid derivative comprises C8, SNAC, 5-CNAC, 4-CNAB, 4-MOAC, SNAD, 4-TPO (8-(4-hydroxyphenoxy) octanoic acid), 5-PPA (5-phenylpentanoic acid), 2-PHOD (8-(2-hydroxyphenoxy)octyldiethanolamine), 3-TBA (4-m-tolyloxybutyric acid), 2-HIPOD (2-(5-pentanoic acid)-5-(2-hydroxyphenyl)-1,3,4-oxadiazole), 7-OPHA (7-oxo-7-phenylheptanoic acid), 3-HPSB (4-(3-hydroxyphenylsulfanyl)butyric acid), 4-IBOA ((4-isopropylbenzyloxy)acetic acid), 3-FPSB (4-(3-fluorophenylsulfanyI)butyric acid), or any combination thereof.

In some aspects, the caprylic acid derivative of the present disclosure is a compound of Formula I or Formula II in which caprylic acid (C8) has been substituted with another fatty acid moiety. Thus, in some aspects of the present disclosure, the C8 moiety of a caprylic acid derivative disclosed herein can optionally be replaced by another fatty acid moiety at least 6 carbon atoms in length, for example, from 6 to 20 carbon atoms in length (C6 to C20), optionally from 6 to 18 carbon atoms in length (i.e., C6 to C18), optionally from 6 to 16 carbon atoms in length (i.e., C6 to C16), optionally from 6 to 14 carbon atoms in length (i.e., C6 to C14), optionally from 6 to 12 carbon atoms in length (i.e., C6 to C12), and optionally from 6 to 10 carbon atoms in length (i.e., C6 to C10).

In some aspects, the C8 moiety of any of the caprylic acid derivatives disclosed herein (e.g., 5-CNAC) can be replaced with a C6, C7, C9, C10, C11, C12, C13, C14, C15, C16, C17, C18, C19, or C20 fatty acid moiety. In some aspects, the C8 moiety of any of the caprylic acid derivatives disclosed herein (e.g., 5-CNAC) can be replaced with a C6, C7, C9, C10, C11, C12, C13, C14, C15, C16, C17, C18, C19, or C20 unsaturated fatty acid moiety. In some aspects, the C8 moiety of any of caprylic acid derivatives disclosed herein (e.g., 5-CNAC) can be replaced with a C6, C7, C8, C9, C10, C11, C12, C13, C14, C15, C16, C17, C18, C19, or C20 saturated fatty acid moiety.

In some aspects, the fatty acid is an essential fatty acid. In view of the beneficial health effects of certain essential fatty acids, the therapeutic benefits of oral formulations can be increased by including such fatty acids in the caprylic acid derivative. In some aspects, the essential fatty acid is an n-6 or n-3 essential fatty acid selected from the group consisting of linolenic acid, gamma-linolenic acid, dihomo-gamma-linolenic acid, arachidonic acid, adrenic acid, docosapentaenoic n-6 acid, alpha-linolenic acid, or stearidonic acid.

Fatty acid chains differ greatly in the length of their chains and may be categorized according to chain length. Medium-chain fatty acids (MCFA) include fatty acids with chains of about 6-12 carbons. In some aspects, the fatty acid is a MCFA. Long-chain fatty acids (LCFA) include fatty acids with chains of 13-20 carbons or longer. In some aspects, the fatty acid is a LCFA.

In some aspects, the fatty acid has a C6 chain. In some aspects, the fatty acid has a C7 chain. In some aspects, the fatty acid has a C8 chain. In some aspects, the fatty acid has a C9 chain. In some aspects, the fatty acid has a C10 chain. In some aspects, the fatty acid has a C11 chain. In some aspects, the fatty acid has a C12 chain. In some aspects, the fatty acid has a C13 chain. In some aspects, the fatty acid has a C14 chain. In some aspects, the fatty acid has a C15 chain. In some aspects, the fatty acid has a C16 chain. In some aspects, the fatty acid has a C17 chain. In some aspects, the fatty acid has a C18 chain. In some aspects, the fatty acid has a C19 chain. In some aspects, the fatty acid has a C20 chain.

In some aspects, the fatty acid has a C6-C7, C6-C8, C6-C9, C6-C10, C6-C11, C6-C12, C6-C13, C6-C14, C6-C15, C6-C16, C6-C17, C6-C18, C6-C19, C6-C20, C7-C8, C7-C9, C7-C10, C7-C11, C7-C12, C7-C13, C7-C14, C7-C15, C7-C16, C7-C17, C7-C18, C7-C19, C7-C20, C8-C9, C8-C10, C8-C11, C8-C12, C8-C13, C8-C14, C8-C15, C8-C16, C8-C17, C8-C18, C8-C19, C8-C20, C9-C10, C9-C11, C9-C12, C9-C13, C9-C14, C9-C15, C9-C16, C9-C17, C9-C18, C9-C19, C9-C20, C10-C11, C10-C12, C10-C13, C10-C14, C10-C15, C10-C16, C10-C17, C10-C18, C10-C19, C10-C20, C11-C12, C11-C13, C11-C14, C11-C15, C11-C16, C11-C17, C11-C18, C11-C19, C11-C20, C12-C13, C12-C14, C12-C15, C12-C16, C12-C17, C12-C18, C12-C19, C12-C20, C13-C14, C13-C15, C13-C16, C13-C17, C13-C18, C13-C19, C13-C20, C14-C15, C14-C16, C14-C17, C14-C18, C14-C19, C14-C20, C15-C16, C15-C17, C15-C18, C15-C19, C15-C20, C16-C17, C16-C18, C16-C19, C16-C20, C17-C18, C17-C19, C17-C20, C18-C19, C18-C20, or C19-C20 chain.

In some aspects, the fatty acid is a linear fatty acid. In other aspects, the fatty acid is a branched fatty acid. Suitable fatty acids include saturated straight-chain fatty acids, saturated branched fatty acids, unsaturated fatty acids, hydroxy fatty acids, and polycarboxylic acids.

Examples of useful saturated straight-chain fatty acids include those having an even number of carbon atoms, such as caproic acid (C6), caprylic acid (C8), capric acid (C10), lauric acid (C12), myristic acid (C14), palmitic acid (C16), or stearic acid (C18), and those having an odd number of carbon atoms, such as propionic acid (C3), n-valeric acid (C5), enanthic acid (C7), pelargonic acid (C9), hendecanoic acid (C11), tridecanoic acid (C13), pentadecanoic acid (C15), or heptadecanoic acid (C17).

Examples of suitable saturated branched fatty acids include isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, or isostearic acid. Suitable saturated odd-carbon branched fatty acids include anteiso fatty acids terminating with an isobutyl group, such as 6-methyl-octanoic acid, 8-methyl-decanoic acid, 10-methyl-dodecanoic acid, 12-methyl-tetradecanoic acid, or 14-methyl-hexadecanoic acid.

Examples of suitable unsaturated fatty acids include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, and the like.

Examples of suitable hydroxy fatty acids include α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, 9-hydroxy-trans-10,12-octadecadienic acid, 9,10-dihydroxystearic acid, 12-hydroxystearic acid and the like.

Examples of suitable polycarboxylic acids include adipic acid, pimelic acid, suberic acid, azelaic acid, sebacic acid, and the like. In some aspects, each fatty acid is independently selected from valeric acid, enanthic acid, pelargonic acid, undecylic acid, lauric acid, tridecylic acid, myristic acid, pentadecylic acid, palmitic acid, margaric acid, or stearic acid. In some aspects, each fatty acid is independently selected from α-linolenic acid, stearidonic acid, eicosapentaenoic acid, docosahexaenoic acid, linoleic acid, gamma-linoleic acid, dihomo-gamma-linoleic acid, arachidonic acid, docosatetraenoic acid, palmitoleic acid, vaccenic acid, paullinic acid, oleic acid, elaidic acid, bosseopentaenoic acid, or another monounsaturated or polyunsaturated fatty acid.

In some aspects, the caprylic acid derivative comprises or consists of N-(8-(2-hydroxybenzoyl)amino)caprylic acid, as shown below.

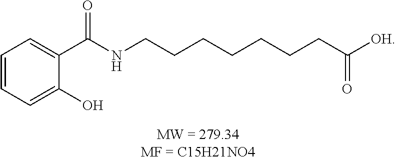 [SMILES: O=C(O)CCCCCCCNC(=O)c1ccccc1O]

In some aspects, the caprylic acid derivative comprises a salt of N-(8-(2-hydroxybenzoyl)amino)caprylic acid. In some aspects, the caprylic acid derivative comprises a solvate of N-(8-(2-hydroxybenzoyl)amino)caprylic acid. In some aspects, the caprylic acid derivative comprises a hydrate of N-(8-(2-hydroxybenzoyl)amino)caprylic acid. In some aspects, the caprylic acid derivative comprises a salt, hydrate, or solvate of N-(8-(2-hydroxybenzoyl)amino)caprylic acid, or any combination thereof.

In some aspects, the salt of N-(8-(2-hydroxybenzoyl)amino)caprylic acid is selected from the group consisting of a sodium salt, a potassium salt, a calcium salt of N-(8-(2-hydroxybenzoyl)amino)caprylic acid, and any combination thereof. In some aspects, the salt of N-(8-(2-hydroxybenzoyl)amino)caprylic acid is a sodium salt. In some aspects, the salt of N-(8-(2-hydroxybenzoyl)amino)caprylic acid is a disodium salt. In some aspects, the salt of N-(8-(2-hydroxybenzoyl)amino)caprylic acid is a monosodium salt (Salcaprozate sodium 203787-91-1, SNAC, sodium 8-(2-hydroxybenzamido)octanoate), as shown below.

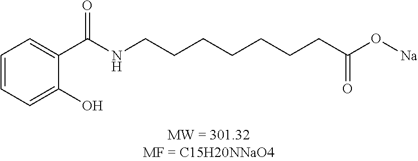

In some aspects, the caprylic acid derivative comprises 5-CNAC (N-(5-chlorosalicyloyl)-8-aminocaprylic acid), as shown below

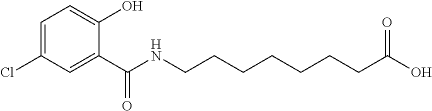 [SMILES: O=C(O)CCCCCCCNC(=O)c1cc(Cl)ccc1O]

In some aspects, the caprylic acid derivative comprises a salt of N-(5-chlorosalicyloyl)-8-aminocaprylic acid. In some aspects, the caprylic acid derivative comprises a solvate of N-(5-chlorosalicyloyl)-8-aminocaprylic acid. In some aspects, the caprylic acid derivative comprises a hydrate of N-(5-chlorosalicyloyl)-8-aminocaprylic acid. In some aspects, the caprylic acid derivative comprises a salt, hydrate, or solvate of N-(5-chlorosalicyloyl)-8-aminocaprylic acid, or any combination thereof.

In some aspects, the salt of N-(5-chlorosalicyloyl)-8-aminocaprylic acid is selected from the group consisting of a sodium salt, a potassium salt, a calcium salt of N-(5-chlorosalicyloyl)-8-aminocaprylic acid, and any combination thereof. In some aspects, the salt of N-(5-chlorosalicyloyl)-8-aminocaprylic acid is a sodium salt. In some aspects, the salt of N-(5-chlorosalicyloyl)-8-aminocaprylic acid is a disodium salt. In some aspects, the salt of N-(5-chlorosalicyloyl)-8-aminocaprylic acid is a monosodium salt.

In some aspects, the caprylic acid derivative comprises 4-CNAB (4-[(4-chloro-2-hydroxy-benzoyl)amino]butanoic acid; Salclobuzate), as shown below

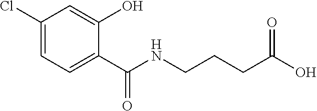 [SMILES: O=C(O)CCCNC(=O)c1ccc(Cl)cc1O]

In some aspects, the caprylic acid derivative comprises a salt of 4-[(4-chloro-2-hydroxy-benzoyl)amino]butanoic acid. In some aspects, the caprylic acid derivative comprises a solvate of 4-[(4-chloro-2-hydroxy-benzoyl)amino]butanoic acid. In some aspects, the caprylic acid derivative comprises a hydrate of 4-[(4-chloro-2-hydroxy-benzoyl)amino]butanoic acid. In some aspects, the caprylic acid derivative comprises a salt, hydrate, or solvate of 4-[(4-chloro-2-hydroxy-benzoyl)amino]butanoic acid, or any combination thereof.

In some aspects, the salt of 4-[(4-chloro-2-hydroxy-benzoyl)amino]butanoic acid is selected from the group consisting of a sodium salt, a potassium salt, a calcium salt of 4-[(4-chloro-2-hydroxy-benzoyl)amino]butanoic acid, and any combination thereof. In some aspects, the salt of 4-[(4-chloro-2-hydroxy-benzoyl)amino]butanoic acid is a sodium salt. In some aspects, the salt of 4-[(4-chloro-2-hydroxy-benzoyl)amino]butanoic acid is a disodium salt. In some aspects, the salt of 4-[(4-chloro-2-hydroxy-benzoyl)amino]butanoic acid is a monosodium salt.

In some aspects, the caprylic acid derivative comprises 4-MOAC (N-(8-[4-methoxy-chloro-2-hydroxybenzoyl-amino) octanoic acid), as shown below

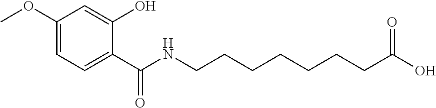 [SMILES: COc1ccc(C(=O)NCCCCCCCC(=O)O)c(O)c1]

In some aspects, the caprylic acid derivative comprises a salt of N-(8-[4-methoxy-chloro-2-hydroxybenzoyl]-amino) octanoic acid. In some aspects, the caprylic acid derivative comprises a solvate of N-(8-[4-methoxy-chloro-2-hydroxybenzoyl]-amino) octanoic acid. In some aspects, the caprylic acid derivative comprises a hydrate of N-(8-[4-methoxy-chloro-2-hydroxybenzoyl]-amino) octanoic acid. In some aspects, the caprylic acid derivative comprises a salt, hydrate, or solvate of N-(8-[4-methoxy-chloro-2-hydroxybenzoyl]-amino) octanoic acid, or any combination thereof.

In some aspects, the salt of N-(8-[4-methoxy-chloro-2-hydroxybenzoyl]-amino) octanoic acid is selected from the group consisting of a sodium salt, a potassium salt, a calcium salt of N-(8-[4-methoxy-chloro-2-hydroxybenzoyl]-amino) octanoic acid, and any combination thereof. In some aspects, the salt of N-(8-[4-methoxy-chloro-2-hydroxybenzoyl]-amino) octanoic acid is a sodium salt. In some aspects, the salt of N-(8-[4-methoxy-chloro-2-hydroxybenzoyl]-amino) octanoic acid is a disodium salt. In some aspects, the salt of N-(8-[4-methoxy-chloro-2-hydroxybenzoyl]-amino) octanoic acid is a monosodium salt.

In some aspects, the caprylic acid derivative comprises SNAD (N-(10-[2-hydroxybenzoyl]-amino) decanoic acid), as shown below

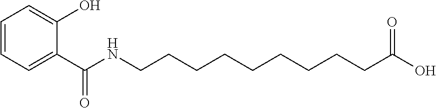 [SMILES: O=C(O)CCCCCCCCCNC(=O)c1ccccc1O]

In some aspects, the caprylic acid derivative comprises a salt of N-(10-[2-hydroxybenzoyl]-amino) decanoic acid. In some aspects, the caprylic acid derivative comprises a solvate of N-(10-[2-hydroxybenzoyl]-amino) decanoic acid. In some aspects, the caprylic acid derivative comprises a hydrate of N-(10-[2-hydroxybenzoyl]-amino) decanoic acid. In some aspects, the caprylic acid derivative comprises a salt, hydrate, or solvate of N-(10-[2-hydroxybenzoyl]-amino) decanoic acid, or any combination thereof.

In some aspects, the salt of N-(10-[2-hydroxybenzoyl]-amino) decanoic acid is selected from the group consisting of a sodium salt, a potassium salt, a calcium salt of N-(10-[2-hydroxybenzoyl]-amino) decanoic acid, and any combination thereof. In some aspects, the salt of N-(10-[2-hydroxybenzoyl]-amino) decanoic acid is a sodium salt. In some aspects, the salt of N-(10-[2-hydroxybenzoyl]-amino) decanoic acid is a disodium salt. In some aspects, the salt of N-(10-[2-hydroxybenzoyl]-amino) decanoic acid is a monosodium salt.

In some aspects, the caprylic acid derivative comprises a compound presented in FIG. 1A, FIG. 1B, or FIG. 2. In some aspects, the caprylic acid derivative comprises a salt of a compound presented in FIG. 1A, FIG. 1B, or FIG. 2. In some aspects, the caprylic acid derivative comprises a solvate of a compound presented in FIG. 1A, FIG. 1B, or FIG. 2. In some aspects, the caprylic acid derivative comprises a hydrate of a compound presented in FIG. 1A, FIG. 1B, or FIG. 2. In some aspects, the caprylic acid derivative comprises a salt, hydrate, or solvate of a compound presented in FIG. 1A, FIG. 1B, or FIG. 2, or any combination thereof.

In some aspects, the salt of a compound presented in FIG. 1A, FIG. 1B, or FIG. 2 is selected from the group consisting of a sodium salt, a potassium salt, a calcium salt of a compound presented in FIG. 1A, FIG. 1B, or FIG. 2, and any combination thereof. In some aspects, the salt of a compound presented in FIG. 1A, FIG. 1B, or FIG. 2 is a sodium salt. In some aspects, the salt of a compound presented in FIG. 1A, FIG. 1B, or FIG. 2 is a disodium salt. In some aspects, the salt of a compound presented in FIG. 1A, FIG. 1B, or FIG. 2 is a monosodium salt. See U.S. Pat. No. 5,650,386A, 6,399,798B2, 7,384,982B2, 7,659,311B2, 8,003,697B2, 8,207,227B2, 8,658,695B2, 7,544,833B2, 7,659,311, 8,003,697, 8,207,227, 8,658,695, 7,384,982, 9,278,123B2, U.S. Ser. No. 10/086,047B2, U.S. Pat. Nos. 8,435,946B2, 8,748,383B2, and 7,569,539B2, and U.S. Patent Appl. Publ. Nos. US20180360918A1, US20110092426A1, and US20150283212A1, all of which are herein incorporated by reference in their entireties.

In some aspects, the caprylic acid derivative comprises a solvate of the salts of SNAC, 5-CNAC, a compound of Formula I or Formula II, or a combination thereof. In some aspects, the caprylic acid derivative is a solvate of a salt 5-CNAC, e.g., solvate of a monosodium or disodium salt of 5-CNAC or a combination thereof. The term “solvate” as used herein includes, but is not limited to, a molecular or ionic complex of molecules or ions of a solvent with molecules or ions of the caprylic acid derivative or salt thereof, or hydrate or solvate thereof.

In some aspects, the caprylic acid derivative comprises a hydrate of the salts of SNAC, 5-CNAC, a compound of Formula I or Formula II, or a combination thereof. In some aspects, the caprylic acid derivative is a hydrate of a salt of 5-CNAC, e.g., a hydrate of a monosodium salt or a disodium salt of 5-CNAC or a combination thereof. The term “hydrate” as used herein includes, but is not limited to, (i) a substance containing water combined in the molecular form and (ii) a crystalline substance containing one or more molecules of water of crystallization or a crystalline material containing free water.

In some aspects, the caprylic acid derivative comprises a solvate of a salt of SNAC, 5-CNAC, or a compound of Formula I or Formula II, wherein the salt is a sodium salt, potassium salt, calcium salt, or a combination thereof. In some aspects, the caprylic acid derivative comprises a solvate of a salt of SNAC, 5-CNAC, or a compound of Formula I or Formula II wherein the salt is a sodium salt.

In some aspects, the caprylic acid derivative comprises a solvate of a salt of SNAC, 5-CNAC, or a compound of Formula I or Formula II, wherein the salt is a monosodium salt. In some aspects, the caprylic acid derivative comprises a sodium salt of a compound for Formula I. In some aspects, the caprylic acid derivative comprises a sodium salt of a compound of Formula II. In some aspects, the caprylic acid derivative comprises a sodium salt of SNAC, e.g., monosodium SNAC. In some aspects, the caprylic acid derivative comprises a sodium salt of 5-CNAC, e.g., monosodium 5-CNAC or a disodium salt of 5-CNAC.

In some aspects, the caprylic acid derivative comprises an alcohol solvate of a salt of C8, C10, SNAC, or 5-CNAC, wherein the salt is a sodium salt. In some aspects, the caprylic acid derivative comprises an alcohol solvate of the salts of C8, C10, SNAC, or 5-CNAC, wherein the salt is a monosodium salt. In some aspects, the caprylic acid derivative comprises a hydrate of a salt of C8, C10, SNAC, or 5-CNAC, wherein the salt is a sodium salt. In some aspects, the caprylic acid derivative comprises a hydrate of a salt of C8, C10, SNAC, or 5-CNAC, wherein the salt is a monosodium salt. In some aspects, the caprylic acid derivative comprises a hydrate of a salt of C8, C10, SNAC, or 5-CNAC, wherein the salt is a sodium salt, and the hydrate is a monohydrate.

Methods to prepare sodium salts, alcohol solvates, and hydrates are described, e.g., in Int'l Publ. WO 00/059863, which is herein incorporated by reference in its entirety. For example, a sodium salt may be prepared from the ethanol solvate by evaporating or drying the ethanol solvate by methods known in the art to form the anhydrous sodium salt. Drying is generally carried out at a temperature of from about 80° C. to about 120° C., e.g., from about 85° C. to about 90° C. In some aspects, drying is conducted at about 85° C. The drying step is generally performed at a pressure of about 660 mm Hg (8.8 kPa) or greater. The anhydrous sodium salt generally contains less than about 5% by weight of ethanol and preferably less than about 2% by weight of ethanol, based on 100% total weight of anhydrous sodium salt.

The sodium salt of a caprylic acid derivative disclosed herein can also be prepared by making a slurry of the delivery agent in water and adding aqueous sodium hydroxide, sodium alkoxide or the like. Suitable sodium alkoxides include, but are not limited to, sodium methoxide, sodium ethoxide, and combinations thereof. A still further method of preparing the sodium salt is by reacting the delivery agent with sodium hydroxide to yield the sodium salt.

The sodium salt can be isolated as a solid by concentrating the solution containing the sodium salt to a thick paste by vacuum distillation. This paste may be dried in a vacuum oven to obtain the sodium salt of the caprylic acid derivative as a solid. The solid can also be isolated by spray drying an aqueous solution of the disodium salt. The caprylic acid derivatives disclosed herein may be prepared by methods known in the art, e.g., as mentioned above, by methods described in U.S. Pat. Nos. 5,773,647 and 5,866,536, which are herein incorporated by reference in their entireties.

Ethanol solvates of the caprylic acid derivatives disclosed herein (e.g., C8, C10, SNAC, 5-CNAC, or any combination thereof) include, but are not limited to, a molecular or ionic complex of molecules or ions of ethanol solvent with molecules or ions of the sodium salt of the caprylic acid derivative. Typically, the ethanol solvate contains about one ethanol molecule or ion for every molecule of sodium salt of the caprylic acid derivative.

Ethanol solvates of sodium salts of the caprylic acid derivative can be prepared by dissolving the caprylic acid derivative in ethanol. The caprylic acid derivative/ethanol solution is then reacted with a molar excess of a sodium containing salt, such as a monosodium containing salt, relative to caprylic acid derivative, i.e., for every mole of caprylic acid derivative there is more than one mole of sodium cations, yielding the ethanol solvate. Suitable monosodium salts include, but are not limited to, sodium hydroxide; sodium alkoxides, such as sodium methoxide and sodium ethoxide; and any combination of the foregoing. Generally, the reaction is performed at or below the reflux temperature of the mixture, such as at ambient temperature. The ethanol solvate is then recovered by methods known is the art, such as, concentration of the resulting slurry at atmospheric distillation, cooling the concentrated slurry and filtering the solid. The recovered solid can then be vacuum dried to obtain the ethanol solvate.

Hydrates of the sodium salts of the caprylic acid derivative may be prepared by drying the ethanol solvate to from an anhydrous disodium salt, as described above, and hydrating the anhydrous sodium salt. In some aspects, the monohydrate of the sodium salt is formed. Since the anhydrous sodium salts are very hygroscopic, the hydrates form upon exposure to atmospheric moisture. Generally, the hydrating step is performed at from about ambient temperature to about 50° C., preferably ambient temperature to about 30° C. and in an environment having at least 50% relative humidity. Alternatively, the anhydrous sodium salt may be hydrated with steam.

The caprylic acid derivatives of the present disclosure typically contain an effective amount of one or more of the caprylic acid derivatives (e.g., SNAC, 5-CNAC, or any combination thereof) disclosed herein, i.e., an amount sufficient to deliver the active agent (e.g., an ASO such as CIVI 008) for the desired effect. Generally, the caprylic acid derivative (e.g., SNAC, 5-CNAC, or any combination thereof) is present in an amount of about 2.5% to about 99.4% by weight. In some aspects, the caprylic acid derivative (e.g., SNAC, 5-CNAC, or any combination thereof) is present in an amount of about 15% to about 75% by weight. In some aspects, the caprylic acid derivative (e.g., SNAC, 5-CNAC, or any combination thereof) is present in an amount of least about 25%, at least about 30%, or at least about 35% but equal to or less than about 60 or about 70% by weight. Accordingly, in some aspects the caprylic acid derivative (e.g., SNAC, 5-CNAC, or any combination thereof) is present in an amount of least about 25%, at least about 30%, at least about 35%, at least about 40%, at least about 45%, at least about 50%, at least about 55%, at least about 60%, at least about 65%, or at least about 70% by weight. In some aspects, the caprylic acid derivative (e.g., SNAC, 5-CNAC, or any combination thereof) is present in an amount of about 25%, about 30%, about 35%, about 40%, about 45%, about 50%, about 55%, about 60%, about 65%, or about 70% by weight.

In some aspects, the caprylic acid derivative (e.g., 5-CNAC) can interact non-covalently with the oligonucleotide. In some aspects, the caprylic acid derivative is (e.g., 5-CNAC) covalently attached to an oligonucleotide disclosed herein. In some aspects, the caprylic acid derivative (e.g., 5-CNAC) is covalently attached to the 5′ end of the oligonucleotide. In some aspects, the caprylic acid derivative (e.g., 5-CNAC) is covalently attached to the 3′ end of the oligonucleotide. In some aspects, a caprylic acid derivative (e.g., 5-CNAC) can be covalently attached to the 5′ end of the oligonucleotide and a second caprylic acid derivative (e.g., 5-CNAC) can be covalently attached to the 3′ end of the oligonucleotide. In some aspects, a caprylic acid derivative (e.g., 5-CNAC) can be covalently attached to an oligonucleotide at a position that is not the 5′ end or the 3′ end of the oligonucleotide. In some aspects, when multiple caprylic acid derivative units are attached to an oligonucleotide, each caprylic acid derivative unit can be the same. In some aspects, when multiple caprylic acid derivative units are attached to an oligonucleotide, at least one caprylic acid derivative unit can be different. In some aspects, when multiple caprylic acid derivative units are attached to an oligonucleotide, all caprylic acid derivative units are different. In some aspects, a caprylic acid derivative (e.g., 5-CNAC) can be covalently attached to an oligonucleotide via a spacer or linker. In some aspects, a caprylic acid derivative (e.g., 5-CNAC) can be covalently attached to an oligonucleotide via a cleavable linker. In some aspects, the cleavable linker is a pH-sensitive cleavable linker. In some aspects, the cleavable linker can be cleaved by enzymes, e.g., enzymes present in the gastrointestinal tract, such as enzymes present in the stomach or small intestine.

In some aspects, the cleavable linkage can comprise a redox cleavable linker (e.g., a disulfide bond), a reactive oxygen species cleavable linker (e.g., a thioketal cleavable linker), a pH dependent cleavable linker (e.g., a low pH-labile hydrazone bond), an enzymatic cleavable linker, a protease cleavable linker, an esterase cleavable linker, a phosphatase cleavable linker, a self-immolative linker (e.g., p-aminobenzyl carbamate, pABC), or any combination thereof.

In some aspects, the cleavable linker comprises or consists of a cinnamyl group, a naphthyl group, a biphenyl group, a heterocyclic ring, a homoaromatic group, coumarin, furan, thiophene, thiazole, oxazole, isoxazole, pyrrole, pyrazole, pyridine, imidazone, triazole, or any combination thereof. In some aspects, the cleavable linker comprises or consists of a dipeptide, a tripeptide, a tetrapeptide, a pentapeptide, or a hexapeptide. In some aspects, the dipeptide is selected from the group consisting of valine-alanine, valine-citrulline, phenylalanine-lysine, N-methylvaline-citrulline, cyclohexylalanine-lysine, and beta-alanine-lysine. In some aspects, the tripeptide is glutamic acid-valine-citrulline. In some aspects, the cleavable linker comprises valine-alanine-p-aminobenzylcarbamate or valine-citrulline-p-aminobenzylcarbamate.

In some aspects, the present disclosure provides constructs exemplified in the schemas below:

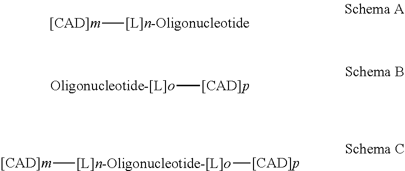

wherein:
CAD is a caprylic acid derivative, e.g., 5-CNAC;
L is a linker, e.g., a cleavable or non-cleavable linker; and,
m, n, o, and p are independently integers between 0 and 5.

In oligonucleotide comprising multiple strands, the oral delivery agent (e.g., 5-CNAC) can be attach at either end of the nucleotide sequence, e.g., the 5′ end or the 3′ end, or both. Accordingly, in some aspects, the present disclosure provides constructs according to the following schemas:

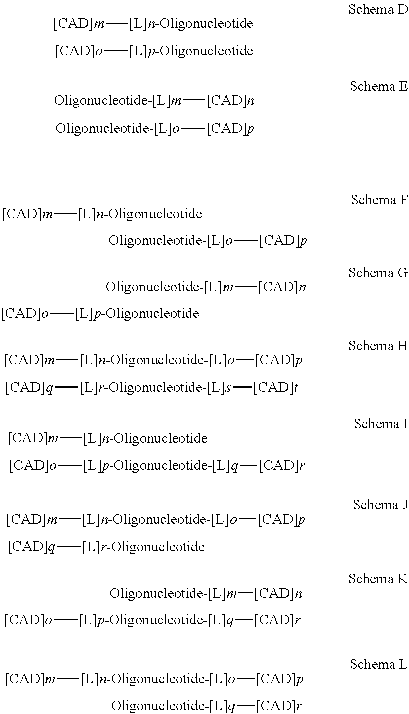

wherein:
CAD is a caprylic acid derivative, e.g., 5-CNAC;
L is a linker, e.g., a cleavable or non-cleavable linker; and,
m, n, o, p, q, r, s, or t are independently integers between 0 and 5.

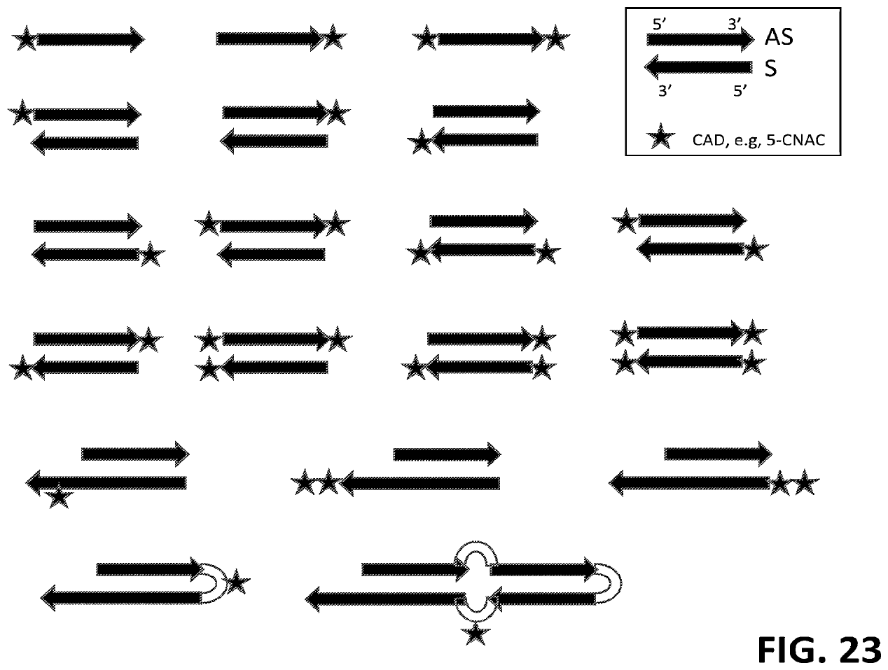

In some aspects, m, n, o, p, q, r, s, or t are 1. In some aspects, m is 0. In some aspects, m is 1. In some aspects, m is higher than 1. In some aspects, n is 0. In some aspects, n is 1. In some aspects, n is higher than 1. In some aspects, o is 0. In some aspects, o is 1. In some aspects, o is higher than 1. In some aspects, p is 0. In some aspects, p is 1. In some aspects, p is higher than 1. In some aspects, q is 0. In some aspects, q is 1. In some aspects, q is higher than 1. In some aspects, r is 0. In some aspects, r is 1. In some aspects, r is higher than 1. In some aspects, s is 0. In some aspects, s is 1. In some aspects, s is higher than 1. In some aspects, t is 0. In some aspects, t is 1. In some aspects, t is higher than 1. Exemplary constructs are depicted in FIG. 23.

When multiple CAD and L are present in a construct, they can be identical or different. Thus, for example, in a construct of Schema C, both CAD units can be the same (e.g., 5-CNAC) or different. In a construct of Schema C, both L units can be the same or different.

In some aspects, a branching unit [B] is interposed between [CAD] and [L] or between [L] and the Oligonucleotide. In some aspects, the branching unit [B] provides 2, 3, or 4 branching points, i.e., the [B] branching unit can be connected with 2 [CAD] units, 3 [CAD] units, or 4 [CAD] units, which are not connected to each other and are connected to the construct via the [B] branching unit.

In some aspects, a caprylic acid derivative, e.g., 5-CNAC, can be covalently attached to a connecting loop, e.g., a loop connecting the antisense and sense strands of a hairpin (e.g., in a shRNA). In some aspects, a caprylic acid derivative, e.g., 5-CNAC, can be attached to an oligonucleotide through a non-terminal location, i.e., a location different from the 5′ end or the 3′ end.

In some aspects, the caprylic acid derivative (e.g., 5-CNAC) is an oral delivery agent. In some aspects, an oral delivery agent. (e.g., 5-CNAC) is covalently attached to an antisense oligonucleotide (e.g., the oligonucleotide moiety of CIVI 008 or ISIS 863633 without a GalNAc moiety) or antisense oligonucleotide conjugate disclosed herein (e.g., CIVI 008 or ISIS 863633), either directly or via a linker or combination of linkers, wherein the linker or combination of linkers can comprise a cleavable linker. In some aspects, the oral delivery agent (e.g., 5-CNAC) is covalently attached to an antisense oligonucleotide directly or via a spacer.

In some aspects, the oral delivery agent (e.g., 5-CNAC) is covalently attached to a non-nucleotide or non-polynucleotide moiety of an antisense oligonucleotide conjugate disclosed herein (e.g., the GalNAc moiety of CIVI 008 or ISIS 863633), directly or via a linker, spacer, or combination thereof. Accordingly, in some aspects, the oral delivery agent (e.g., 5-CNAC) or a combination thereof is attached, e.g., to a GalNAc conjugate moiety comprising a cleavable linker. Cleavage of the cleavable linker or combination thereof can release both the GalNAc and the oral delivery agent (e.g., 5-CNAC) from the conjugate. In other aspects, the GalNAc moiety and the oral delivery agent are attached via two separate cleavable linkers, which can be the same or different. In some aspects, both cleavable linkers may be cleaved according to the same mechanism (e.g., two pH sensitive linkers). In other aspects, each cleavable linker may be cleaved according to a different mechanism (e.g., a linker could be a pH sensitive linker, and the second linker could be enzymatically cleaved, e.g., by esterases).

In some aspects, the oral delivery agent (e.g., 5-CNAC) can be covalently attached to the 5′ of an oligonucleotide disclosed herein, e.g., an ASO. In some aspects, the oral delivery agent (e.g., 5-CNAC) can be covalently attached to the 3′ of an oligonucleotide disclosed herein, e.g., an ASO. In some aspects, the oral delivery agent (e.g., 5-CNAC) can be covalently attached to the 5′ or 3′ of an oligonucleotide disclosed herein, e.g., an ASO, directly (e.g., to the 5′ or 3′ nucleotides). In some aspects, the oral delivery agent (e.g., 5-CNAC) can be covalently attached to the 5′ or 3′ of an oligonucleotide disclosed herein, e.g., an ASO, indirectly to the 5′ or 3′ nucleotide via a linker, a spacer, or a combination thereof.

In some aspects, the oral delivery agent (e.g., 5-CNAC) can be covalently attached to an antisense oligonucleotide conjugate disclosed herein, e.g., CIVI 008. In some aspects, the oral delivery agent (e.g., 5-CNAC) can be covalently attached to the nucleic acid moiety of an antisense oligonucleotide conjugate disclosed herein. In some aspects, the oral delivery agent (e.g., 5-CNAC) can be covalently attached to the non-nucleotide or non-polynucleotide moiety (e.g., GalNAc moiety) of an antisense oligonucleotide conjugate disclosed herein, e.g., CIVI 008 or an unconjugated form thereof, i.e., without a GalNAc moiety.

In some aspects, the oral delivery agent (e.g., 5-CNAC) can be covalently attached to an antisense oligonucleotide conjugate disclosed herein, e.g., an ASO, via a linker, spacer, or a combination thereof. In some aspects, the oral delivery agent (e.g., 5-CNAC) can be covalently attached to the nucleic acid moiety of an antisense oligonucleotide conjugate disclosed herein, e.g., CIVI 008, via a linker, spacer, or a combination thereof. In some aspects, the oral delivery agent (e.g., 5-CNAC) can be covalently attached to the non-nucleotide or non-polynucleotide moiety (e.g., GalNAc moiety) of an antisense oligonucleotide conjugate disclosed herein via a linker, spacer, or a combination thereof.

In some aspects, a nucleic acid therapeutic agent, e.g., an antisense oligonucleotide (ASO), short interference RNA (siRNA), small hairpin RNA (shRNA), DNA or RNA aptamer, gene therapy vector, micro RNA (miRNA), anti-micro RNA (antimiR), DNA or RNA decoy, CpG oligonucleotide, ribozyme, circular RNA (circRNA), or any other therapeutic oligonucleotide known in the art, can be covalently attached to more than one oral delivery agents disclosed herein, e.g., a caprylic acid derivative such as SNAC, 5-CNAC, SNAD, 4-CNAB, 4-MOAC, 4-HPO, 3-TBA, 3-HPSB, 5-PPA, 2-HIPOD, 4-IBOA, 2-PHOD, 7-OPHA, 3-FPSB, a molecule disclosed in FIG. 1A, FIG. 1B, or FIG. 2, a derivative thereof, a pharmaceutically acceptable hydrate, solvate, or salt thereof, or any combination thereof. In some aspects, the nucleic acid therapeutic agent is not conjugated to a GalNAc moiety.

In some aspects, a nucleic acid therapeutic agent, e.g., an ASO, siRNA, shRNA, DNA or RNA aptamer, gene therapy vector, miRNA, miRNA mimic, antimiR, DNA or RNA decoy, CpG oligonucleotide, or any therapeutic or diagnostic oligonucleotide known in the art can have more than one oral delivery agent disclosed herein, e.g., a caprylic acid derivative such as SNAC, 5-CNAC, SNAD, 4-CNAB, 4-MOAC, 4-HPO, 3-TBA, 3-HPSB, 5-PPA, 2-HPOD, 4-IBOA, 2-PHOD, 7-OPHA, 3-FPSB, a molecule disclosed in FIG. 1A, FIG. 1B, or FIG. 2, a derivative thereof, a pharmaceutically acceptable hydrate, solvate, or salt thereof, or any combination thereof, covalently attached at different positions (e.g., one or more oral delivery agents covalently attached to the nucleic acid moiety and an one or more oral delivery agents covalently attached to a heterologous moiety such as a GalNAc moiety; or one or more oral delivery agents covalently attached to a nucleic acid not conjugated to a delivery moiety such as a GalNAc moiety).

Compositions for oral delivery can be manufactured, e.g., by admixing (i) a nucleic acid therapeutic agent, e.g., an ASO, siRNA, shRNA, DNA or RNA aptamer, gene therapy vector, miRNA, miRNA mimic, antimiR, DNA or RNA decoy, CpG oligonucleotide, or any therapeutic or diagnostic oligonucleotide known in the art (e.g., CIVI 008 or an unconjugated form thereof, i.e., without a GalNAc moiety), wherein the nucleic acid therapeutic agent comprises or doesn't comprise a conjugated GalNAc moiety; and, (ii) an oral delivery agent (e.g., 5-CNAC) comprising a caprylic acid derivative such as SNAC, 5-CNAC, SNAD, 4-CNAB, 4-MOAC, 4-HPO, 3-TBA, 3-HPSB, 5-PPA, 2-HPOD, 4-IBOA, 2-PHOD, 7-OPHA, 3-FPSB, a molecule disclosed in FIG. 1A, FIG. 1B, or FIG. 2, a derivative thereof, a pharmaceutically acceptable hydrate, solvate, or salt thereof, or any combination thereof.

Accordingly, the present disclosure provides a method to manufacture a composition for oral delivery (e.g., a pill or a tablet) comprising admixing (i) a nucleic acid therapeutic agent, e.g., an ASO, siRNA, shRNA, DNA or RNA aptamer, gene therapy vector, miRNA, miRNA mimic, antimiR, DNA or RNA decoy, CpG oligonucleotide, or any therapeutic or diagnostic oligonucleotide known in the art (e.g., CIVI 008 or an unconjugated form thereof, i.e., without a GalNAc moiety), wherein the nucleic acid therapeutic agent comprises or doesn't comprise a conjugated GalNAc moiety; and, (ii) an oral delivery agent (e.g., 5-CNAC) comprising a caprylic acid derivative such as SNAC, 5-CNAC, SNAD, 4-CNAB, 4-MOAC, 4-HPO, 3-TBA, 3-HPSB, 5-PPA, 2-HIPOD, 4-IBOA, 2-PHOD, 7-OPHA, 3-FPSB, a molecule disclosed in FIG. 1A, FIG. 1B, or FIG. 2, a derivative thereof, a pharmaceutically acceptable hydrate, solvate, or salt thereof, or any combination thereof.

In some aspects, when a caprylic acid derivative is covalently attached to an oligonucleotide, e.g., a nucleic acid therapeutic agent such as an ASO, siRNA, shRNA, DNA or RNA aptamer, gene therapy vector, miRNA, miRNA mimic, antimiR, DNA or RNA decoy, CpG oligonucleotide, or any therapeutic or diagnostic oligonucleotide known in the art (e.g., CIVI 008), the attachment of the caprylic acid derivative (e.g., 5-CNAC) can be conducted via solid phase synthesis. Thus, in some aspects, the present disclosure provides any of the caprylic acid derivatives disclosed herein, e.g., 5-CNAC or a derivative thereof, or any of the compounds disclosed, e.g., in FIG. 1A, FIG. 1B, FIG. 2, or any combination thereof, in a phosphoramidite form. Accordingly, the present disclosure provides a 5-CNAC phosphoramidite. Also provided are phosphoramidite forms of caprylic acid derivatives such as SNAC, 5-CNAC, SNAD, 4-CNAB, 4-MOAC, 4-HPO, 3-TBA, 3-HPSB, 5-PPA, 2-HIPOD, 4-IBOA, 2-PHOD, 7-OPHA, 3-FPSB, a molecule disclosed in FIG. 1A, FIG. 1B, or FIG. 2. In some aspects, the present disclosure provides a kit comprising a 5-CNAC phosphoramidite and instructions to conjugate such 5-CNAC phosphoramite to an oligonucleotide.

In some aspects, the present disclosure provides a method to manufacture a therapeutic oligonucleotide conjugate comprising covalently attaching at least one caprylic acid derivative disclosed herein (e.g., 5-CNAC) to an oligonucleotide oligomer (e.g., an antisense oligonucleotide). In some aspects, the present disclosure provides a method to manufacture a therapeutic oligonucleotide conjugate comprising attaching covalently or non-covalently at least one caprylic acid derivative disclosed herein (e.g., 5-CNAC) to an oligonucleotide oligomer of SEQ ID NO: 134 (oligonucleotide oligomer in CIVI008, cepadacursen).

In some aspects, the oligonucleotide comprises, consists, or consists essentially of a single chain oligonucleotide 10 to 100 contiguous nucleotides in length. In some aspects, the oligonucleotide comprises a single chain oligonucleotide 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides in length.

In some aspects, the oligonucleotide consists of a single chain oligonucleotide 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides in length.

In some aspects, the oligonucleotide comprises a single chain oligonucleotide at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 contiguous nucleotides in length.

In some aspects, the oligonucleotide consists of a single chain oligonucleotide at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 contiguous nucleotides in length.

In some aspects, the oligonucleotide comprises a single chain oligonucleotide between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, between about 95 and about 100, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 15 and about 25, between about 25 and about 35, between about 35 and about 45, between about 45 and about 55, between about 55 and about 65, between about 65 and about 75, between about 75 and about 85, between about 85 and about 95, between about 10 and about 25, between about 15 and about 30, between about 20 and about 35, between about 25 and about 40, between about 30 and about 45, between about 35 and about 50, between about 40 and about 55, between about 45 and about 60, between about 50 and about 65, between about 55 and about 70, between about 60 and about 75, between about 65 and about 80, between about 70 and about 85, between about 75 and about 90, between about 80 and about 95, between about 85 and about 100, between about 10 and about 30, between about 15 and about 35, between about 20 and about 40, between about 25 and about 45, between about 30 and about 50, between about 35 and about 55, between about 40 and about 60, between about 45 and about 65, between about 50 and about 70, between about 55 and about 75, between about 60 and about 80, between about 65 and about 85, between about 70 and about 90, between about 75 and about 95, or between about 80 and about 100 contiguous nucleotides in length.

In some aspects, the oligonucleotide consists of a single chain oligonucleotide between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, between about 95 and about 100, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 15 and about 25, between about 25 and about 35, between about 35 and about 45, between about 45 and about 55, between about 55 and about 65, between about 65 and about 75, between about 75 and about 85, between about 85 and about 95, between about 10 and about 25, between about 15 and about 30, between about 20 and about 35, between about 25 and about 40, between about 30 and about 45, between about 35 and about 50, between about 40 and about 55, between about 45 and about 60, between about 50 and about 65, between about 55 and about 70, between about 60 and about 75, between about 65 and about 80, between about 70 and about 85, between about 75 and about 90, between about 80 and about 95, between about 85 and about 100, between about 10 and about 30, between about 15 and about 35, between about 20 and about 40, between about 25 and about 45, between about 30 and about 50, between about 35 and about 55, between about 40 and about 60, between about 45 and about 65, between about 50 and about 70, between about 55 and about 75, between about 60 and about 80, between about 65 and about 85, between about 70 and about 90, between about 75 and about 95, or between about 80 and about 100 contiguous nucleotides in length.

In some aspects, the oligonucleotide comprises, consists, or consists essentially of a double stranded nucleic acid, comprising a sense and an antisense strand, wherein the sense strand and/or the antisense strand is 10 to 100 contiguous nucleotides in length.

In some aspects, the oligonucleotide comprises a double stranded nucleic acid, comprising a sense and an antisense strand, wherein the sense strand and/or the antisense strand is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides in length.

In some aspects, the oligonucleotide consists of a double stranded nucleic acid, comprising a sense and an antisense strand, wherein the sense strand and/or the antisense strand is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides in length.

In some aspects, the oligonucleotide comprises a double stranded nucleic acid, comprising a sense and an antisense strand, wherein the sense strand and/or the antisense strand is at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 contiguous nucleotides in length.

In some aspects, the oligonucleotide consists of a double stranded nucleic acid, comprising a sense and an antisense strand, wherein the sense strand and/or the antisense strand is at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 contiguous nucleotides in length.

In some aspects, the oligonucleotide comprises a double stranded nucleic acid, comprising a sense and an antisense strand, wherein the sense strand and/or the antisense strand is between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, between about 95 and about 100, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 15 and about 25, between about 25 and about 35, between about 35 and about 45, between about 45 and about 55, between about 55 and about 65, between about 65 and about 75, between about 75 and about 85, between about 85 and about 95, between about 10 and about 25, between about 15 and about 30, between about 20 and about 35, between about 25 and about 40, between about 30 and about 45, between about 35 and about 50, between about 40 and about 55, between about 45 and about 60, between about 50 and about 65, between about 55 and about 70, between about 60 and about 75, between about 65 and about 80, between about 70 and about 85, between about 75 and about 90, between about 80 and about 95, between about 85 and about 100, between about 10 and about 30, between about 15 and about 35, between about 20 and about 40, between about 25 and about 45, between about 30 and about 50, between about 35 and about 55, between about 40 and about 60, between about 45 and about 65, between about 50 and about 70, between about 55 and about 75, between about 60 and about 80, between about 65 and about 85, between about 70 and about 90, between about 75 and about 95, or between about 80 and about 100 contiguous nucleotides in length.

In some aspects, the oligonucleotide consists of a double stranded nucleic acid, comprising a sense and an antisense strand, wherein the sense strand and/or the antisense strand is between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, between about 95 and about 100, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 15 and about 25, between about 25 and about 35, between about 35 and about 45, between about 45 and about 55, between about 55 and about 65, between about 65 and about 75, between about 75 and about 85, between about 85 and about 95, between about 10 and about 25, between about 15 and about 30, between about 20 and about 35, between about 25 and about 40, between about 30 and about 45, between about 35 and about 50, between about 40 and about 55, between about 45 and about 60, between about 50 and about 65, between about 55 and about 70, between about 60 and about 75, between about 65 and about 80, between about 70 and about 85, between about 75 and about 90, between about 80 and about 95, between about 85 and about 100, between about 10 and about 30, between about 15 and about 35, between about 20 and about 40, between about 25 and about 45, between about 30 and about 50, between about 35 and about 55, between about 40 and about 60, between about 45 and about 65, between about 50 and about 70, between about 55 and about 75, between about 60 and about 80, between about 65 and about 85, between about 70 and about 90, between about 75 and about 95, or between about 80 and about 100 contiguous nucleotides in length.

In some aspects, the oligonucleotide comprises, consists, or consists essentially of a partially double stranded nucleic acid, comprising a sense and an antisense strand, wherein the sense strand and/or the antisense strand is 10 to 100 contiguous nucleotides in length. In some aspects, the partially double stranded molecule comprises a complementarity region between a sense and an antisense strand, and one or more overhangs. In some aspects, an overhang can be present at the 5′ end of a sense strand. In some aspect, and overhang can be present at the 3′ end of a sense strand. In some aspects, an overhang can be present at the 5′ end of an antisense strand. In some aspect, and overhang can be present at the 3′ end of an antisense strand. In some aspects, an overhang can be present at the 5′ end of a sense strand, at the 3′ end of a sense strand, at the 5′ end of an antisense strand, at the 3′ end of an antisense strand, or any combination thereof.

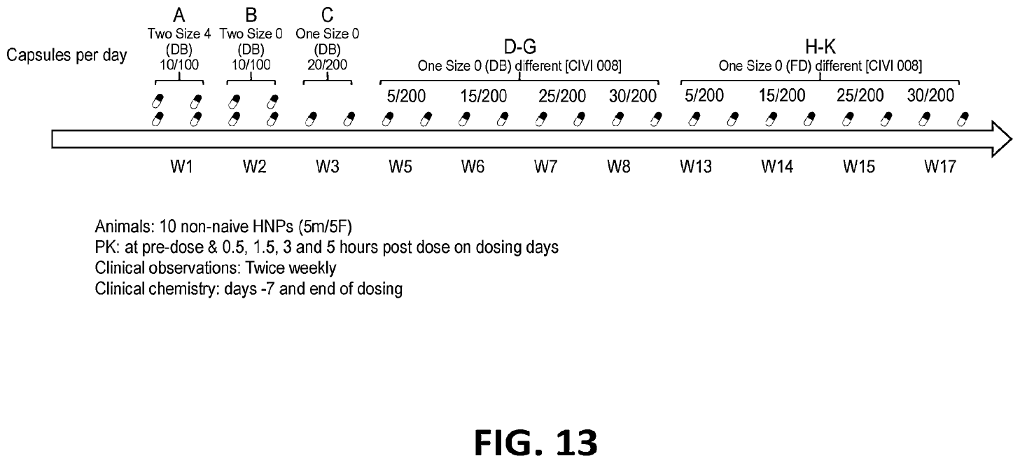

Potential topologies of oligonucleotides comprising partially double stranded nucleic acids, e.g., RNA sponges or tough decoys, are depicted in FIG. 13.

In some aspects, the double stranded nucleic acid is a single chain molecule comprising the sense and antisense strands connected by a loop. In some aspects, the double stranded nucleic acid is a two-chain molecule wherein the sense and antisense strands are not connected by a loop.

In some aspects, the oligonucleotide is an ASO. In some aspects, the ASO is a gapmer. In some aspects, the oligonucleotide is an ASO comprising at least one nucleotide analog, e.g., an LNA unit or 5-MOE unit. In some aspects, the ASO is about 12, 13, 14, 15, 16, 17, 18, 19, 20, 21, or 22 nucleotides in length. In some aspects, the ASO is 12 nucleotides in length. In some aspects, the ASO is 13 nucleotides in length. In some aspects, the ASO is 14 nucleotides in length. In some aspects, the ASO is 15 nucleotides in length. In some aspects, the ASO is 16 nucleotides in length. In some aspects, the ASO is 17 nucleotides in length. In some aspects, the ASO is 18 nucleotides in length. In some aspects, the ASO is 19 nucleotides in length. In some aspects, the ASO is 20 nucleotides in length. In some aspects, the ASO is 21 nucleotides in length. In some aspects, the ASO is 22 nucleotides in length.

In some aspects, the ASO can be conjugated to a moiety capable of targeting a particular tissue, e.g., liver. In some aspects, the ASO conjugate comprises an ASO about 12, 13, 14, 15, 16, 17, 18, 19, 20, 21, or 22 nucleotides in length. In some aspects, the ASO conjugate comprises an ASO 12 nucleotides in length. In some aspects, the ASO conjugate comprises an ASO 13 nucleotides in length. In some aspects, the ASO conjugate comprises an ASO 14 nucleotides in length. In some aspects, the ASO conjugate comprises an ASO 15 nucleotides in length. In some aspects, the ASO conjugate comprises an ASO 16 nucleotides in length. In some aspects, the ASO conjugate comprises an ASO 17 nucleotides in length. In some aspects, the ASO conjugate comprises an ASO 18 nucleotides in length. In some aspects, the ASO conjugate comprises an ASO 19 nucleotides in length. In some aspects, the ASO conjugate comprises an ASO 20 nucleotides in length. In some aspects, the ASO conjugate comprises an ASO 21 nucleotides in length. In some aspects, the ASO conjugate comprises an ASO 22 nucleotides in length.

In some aspects of the present disclosure, the oligonucleotide is a nucleic acid therapeutic agent.

As some specific aspects, the compositions and methods disclosed here related to the following therapeutic agents. In some aspects, the non-conjugated form (e.g., without GalNAc) is formulated with 5-CNAC, e.g., as an oral capsule.

1018 ISS: In some aspects, the nucleic acid therapeutic agent is 1018 ISS. 1018 ISS, also known as ISS-1018, is a CpG oligonucleotide which functions as an immunostimulant. In some aspects, the present disclosure provides a composition comprising 1018 ISS and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising 1018 ISS and 5-CNAC. In one specific aspect, the present disclosure provides a composition comprising 1018 ISS and 5-CNAC disodium salt. In some aspects, the composition is formulated for oral delivery. The present disclosure also provides a method to treat or prevent a disease or condition in a subject in need thereof comprising administering an effective amount of a composition comprising 1018 ISS disclosed herein (e.g., a composition such as a pharmaceutical composition comprising 1018 ISS and a monosodium or disodium salt of 5-CNAC) to the subject. Also provided is a pill or capsule comprising 1018 ISS and a caprylic acid derivative disclosed herein, e.g., a monosodium or disodium salt of 5-CNAC.

The present disclosure also provides a method to treat or prevent a disease or condition in a subject in need thereof comprising administering an effective amount of a composition comprising 1018 ISS disclosed herein (e.g., a composition such as a pharmaceutical composition comprising 1018 ISS and a monosodium or disodium salt of 5-CNAC) to the subject. Also provided is a pill or capsule comprising 1018 ISS and a caprylic acid derivative disclosed herein, e.g., a monosodium or disodium salt of 5-CNAC.

The oligonucleotide sequence of 1018 ISS is TGACTGTGAACGTTCGAGATGA (SEQ ID NO: 1). In some aspects, is 1018 ISS a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

AEG35156 (GEM640): In some aspects, the nucleic acid therapeutic agent is AEG35156. AEG35156 is an antisense oligonucleotide for the treatment of hepatocellular carcinoma, acute myeloid leukemia, B-cell lymphoma, chronic lymphocytic leukemia, multiple sclerosis, non-small cell lung cancer, or pancreatic cancer that targets X-Linked Inhibitor of Apoptosis (XIAP). In some aspects, the present disclosure provides a composition comprising AEG35156 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AEG35156 and 5-CNAC. In one specific aspect, the present disclosure provides a composition comprising AEG35156 and 5-CNAC disodium salt. In some aspects, the composition is formulated for oral delivery.

The present disclosure also provides a method to treat or prevent a disease or condition in a subject in need thereof comprising administering an effective amount of a composition comprising AEG35156 disclosed herein (e.g., a composition such as a pharmaceutical composition comprising AEG35156 and a monosodium or disodium salt of 5-CNAC) to the subject. Also provided is a pill or capsule comprising AEG35156 and a caprylic acid derivative disclosed herein, e.g., a monosodium or disodium salt of 5-CNAC.

The oligonucleotide sequence of AEG35156 is UGCACCCTGGATACCAUUU (SEQ ID NO: 136). In some aspects, AEG35156 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

AB-729: In some aspects, the nucleic acid therapeutic agent is AB-729. AB-729 is an anti-miRNA (antimir) for the treatment of hepatitis B infection that targets hepatitis virus B's HBsAg. In some aspects, the present disclosure provides a composition comprising AB-729 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AB-729 and 5-CNAC. In one specific aspect, the present disclosure provides a composition comprising AB-729 and 5-CNAC disodium salt. In some aspects, the composition is formulated for oral delivery.

The present disclosure also provides a method to treat or prevent a disease or condition in a subject in need thereof comprising administering an effective amount of a composition comprising AB-729 disclosed herein (e.g., a composition such as a pharmaceutical composition comprising AB-729 and a monosodium or disodium salt of 5-CNAC) to the subject. Also provided is a pill or capsule comprising AB-729 and a caprylic acid derivative disclosed herein, e.g., a monosodium or disodium salt of 5-CNAC. In some aspects, AB-729 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Abetimus: In some aspects, the nucleic acid therapeutic agent is abetimus. Abetimus is an immunosuppressant oligonucleotide for the treatment of lupus nephritis. In some aspects, the present disclosure provides a composition comprising abetimus and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising abetimus and 5-CNAC. In one specific aspect, the present disclosure provides a composition comprising abetimus and 5-CNAC disodium salt. In some aspects, the composition is formulated for oral delivery.

The present disclosure also provides a method to treat or prevent a disease or condition in a subject in need thereof comprising administering an effective amount of a composition comprising abetimus disclosed herein (e.g., a composition such as a pharmaceutical composition comprising abetimus and a monosodium or disodium salt of 5-CNAC) to the subject. Also provided is a pill or capsule comprising abetimus and a caprylic acid derivative disclosed herein, e.g., a monosodium or disodium salt of 5-CNAC.

The oligonucleotide sequences of abetimus comprise GTGTGTGTGTGTGTGTGTGT (subunit 1) (SEQ ID NO: 2), CACACACACACACACACACA (subunit 2) (SEQ ID NO: 3), CACACACACACACACACACA (subunit 3) (SEQ ID NO: 4), CACACACACACACACACACA (subunit 4) (SEQ ID NO: 5), CACACACACACACACACACA (subunit 5) (SEQ ID NO: 6), GTGTGTGTGTGTGTGTGTGT (subunit 6) (SEQ ID NO: 7), GTGTGTGTGTGTGTGTGTGT (subunit 7) (SEQ ID NO: 8), and GTGTGTGTGTGTGTGTGTGT (subunit 8) (SEQ ID NO: 9). In some aspects, abetimus is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Afovirsen: In some aspects, the nucleic acid therapeutic agent is afovirsen. Afovirsen is an antisense oligonucleotide for the treatment of human papillomavirus infection that targets the mRNA of human papillomavirus. In some aspects, the present disclosure provides a composition comprising afovirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising afovirsen and 5-CNAC. In one specific aspect, the present disclosure provides a composition comprising afovirsen and 5-CNAC disodium salt. In some aspects, the composition is formulated for oral delivery.

The present disclosure also provides a method to treat or prevent a disease or condition in a subject in need thereof comprising administering an effective amount of a composition comprising afovirsen disclosed herein (e.g., a composition such as a pharmaceutical composition comprising afovirsen and a monosodium or disodium salt of 5-CNAC) to the subject. Also provided is a pill or capsule comprising afovirsen and a caprylic acid derivative disclosed herein, e.g., a monosodium or disodium salt of 5-CNAC.

The oligonucleotide sequence of afovirsen is TTGCTTCCATCTTCCTCGTC (SEQ ID NO: 10). In some aspects, afovirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Aganirsen: In some aspects, the nucleic acid therapeutic agent is aganirsen. Aganirsen, also known as GS101, is an antisense oligonucleotide used for the inhibition of corneal neovascularization, a major risk factor of corneal graft rejection that targets insulin receptor substrate-1 (IRS1). In some aspects, the present disclosure provides a composition comprising aganirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising aganirsen and 5-CNAC. In one specific aspect, the present disclosure provides a composition comprising aganirsen and 5-CNAC disodium salt. In some aspects, the composition is formulated for oral delivery.

The present disclosure also provides a method to treat or prevent a disease or condition in a subject in need thereof comprising administering an effective amount of a composition comprising aganirsen disclosed herein (e.g., a composition such as a pharmaceutical composition comprising aganirsen and a monosodium or disodium salt of 5-CNAC) to the subject. Also provided is a pill or capsule comprising aganirsen and a caprylic acid derivative disclosed herein, e.g., a monosodium or disodium salt of 5-CNAC.

The oligonucleotide sequence of aganirsen is TATCCGGAGGGCTCGCCATGCTGCT (SEQ ID NO: 11). In some aspects, aganirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Agatolimod: In some aspects, the nucleic acid therapeutic agent is agatolimod. Agatolimod, also known as CPG-7909, AMA1-C1 or PF-3512676, is a CpG oligodeoxynucleotide for the treatment of cancers such as basal cell cancer, non-Hodgkin's lymphoma, breast cancer, metastatic or recurrent malignancies, non-small cell lung cancer, infectious diseases, allergies, and asthma that acts as a toll-like receptor 9 (TLR9) agonist. In some aspects, the present disclosure provides a composition comprising agatolimod and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising agatolimod and 5-CNAC. In one specific aspect, the present disclosure provides a composition comprising agatolimod and 5-CNAC disodium salt. In some aspects, the composition is formulated for oral delivery.

The present disclosure also provides a method to treat or prevent a disease or condition in a subject in need thereof comprising administering an effective amount of a composition comprising gatolimod disclosed herein (e.g., a composition such as a pharmaceutical composition comprising gatolimod and a monosodium or disodium salt of 5-CNAC) to the subject. Also provided is a pill or capsule comprising gatolimod and a caprylic acid derivative disclosed herein, e.g., a monosodium or disodium salt of 5-CNAC.

The oligonucleotide sequence of agatolimod is TCGTCGTTTTGTCGTTTTGTCGTT (SEQ ID NO: 12). In some aspects, agatolimod is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Alicaforsen: In some aspects, the nucleic acid therapeutic agent is alicaforsen. Alicaforsen is an antisense oligonucleotide for the treatment of acute distress flares in moderate to severe inflammatory bowel disease that targets ICAM-1. In some aspects, the present disclosure provides a composition comprising alicaforsen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising alicaforsen and 5-CNAC. In one specific aspect, the present disclosure provides a composition comprising alicaforsen and 5-CNAC disodium salt. In some aspects, the composition is formulated for oral delivery.

The present disclosure also provides a method to treat or prevent a disease or condition in a subject in need thereof comprising administering an effective amount of a composition comprising alicaforsen disclosed herein (e.g., a composition such as a pharmaceutical composition comprising alicaforsen and a monosodium or disodium salt of 5-CNAC) to the subject. Also provided is a pill or capsule comprising alicaforsen and a caprylic acid derivative disclosed herein, e.g., a monosodium or disodium salt of 5-CNAC.

The oligonucleotide sequence of alicaforsen is GCCCAAGCTGGCATCCGTCA (SEQ ID NO: 13). In some aspects, alicaforsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

AGRO100: In some aspects, the nucleic acid therapeutic agent is AGRO100. AGRO100, also known as AS1411, is an aptamer used for the treatment of acute myeloid leukemia, advanced solid tumors, metastatic renal cell carcinoma, and myeloid leukemia that targets IKBKG. In some aspects, the present disclosure provides a composition comprising AGRO100 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AGRO100 and 5-CNAC. In one specific aspect, the present disclosure provides a composition comprising AGRO100 and 5-CNAC disodium salt. In some aspects, the composition is formulated for oral delivery.

The present disclosure also provides a method to treat or prevent a disease or condition in a subject in need thereof comprising administering an effective amount of a composition comprising AGRO100 disclosed herein (e.g., a composition such as a pharmaceutical composition comprising AGRO100 and a monosodium or disodium salt of 5-CNAC) to the subject. Also provided is a pill or capsule comprising AGRO100 and a caprylic acid derivative disclosed herein, e.g., a monosodium or disodium salt of 5-CNAC.

The oligonucleotide sequence of AGRO100 is GGTGGTGGTGGTTGTGGTGGTGGTGG (SEQ ID NO: 14). In some aspects, AGRO100 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Amlivirsen: In some aspects, the nucleic acid therapeutic agent is amlivirsen. Amlivirsen is an antiviral antisense oligonucleotide. In some aspects, the present disclosure provides a composition comprising amlivirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising amlivirsen and 5-CNAC. In one specific aspect, the present disclosure provides a composition comprising amlivirsen and 5-CNAC disodium salt. In some aspects, the composition is formulated for oral delivery.

The present disclosure also provides a method to treat or prevent a disease or condition in a subject in need thereof comprising administering an effective amount of a composition comprising amlivirsen disclosed herein (e.g., a composition such as a pharmaceutical composition comprising amlivirsen and a monosodium or disodium salt of 5-CNAC) to the subject. Also provided is a pill or capsule comprising amlivirsen and a caprylic acid derivative disclosed herein, e.g., a monosodium or disodium salt of 5-CNAC.

The oligonucleotide sequence of amlivirsen is GCAGAGGTGAAGCGAAGUGC (SEQ ID NO: 15). In some aspects, amlivirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Anivamersen/PEGnivacogin: In some aspects, the nucleic acid therapeutic agent is anivamersen and/or pegnivacogin. Anivamersen and pegnivacogin are components of the REG1 anticoagulation system. Pegnivacogin is an RNA aptamer inhibitor of coagulation factor IXa and anivamersen is a complementary sequence reversal oligonucleotide. In some aspects, the present disclosure provides a composition comprising anivamersen or pegnivacogin and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising anivamersen or pegnivacogin and 5-CNAC. In one specific aspect, the present disclosure provides a composition comprising anivamersen or pegnivacogin and 5-CNAC disodium salt. In some aspects, the composition is formulated for oral delivery.

The present disclosure also provides a method to treat or prevent a disease or condition in a subject in need thereof comprising administering an effective amount of a composition comprising anivamersen or pegnivacogin disclosed herein (e.g., a composition such as a pharmaceutical composition comprising anivamersen or pegnivacogin and a monosodium or disodium salt of 5-CNAC) to the subject. Also provided is a pill or capsule comprising anivamersen or pegnivacogin and a caprylic acid derivative disclosed herein, e.g., a monosodium or disodium salt of 5-CNAC.

The oligonucleotide sequence of pegnivacogin is GUGGACUAUACCGCGUAAUGCUGCCUCCACT (SEQ ID NO: 16). The oligonucleotide sequence of anivamersen is CGCGGUAUAGUCCAC (SEQ ID NO: 17). In some aspects, anivamersen is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

Apatorsen: In some aspects, the nucleic acid therapeutic agent is apatorsen. Apatorsen, also known as OGX-427, is an antisense oligonucleotide used for the treatment of advanced squamous cell lung cancers that targets Hsp27. In some aspects, the present disclosure provides a composition comprising apatorsen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising apatorsen and 5-CNAC. In one specific aspect, the present disclosure provides a composition comprising apatorsen and 5-CNAC disodium salt. In some aspects, the composition is formulated for oral delivery.

The present disclosure also provides a method to treat or prevent a disease or condition in a subject in need thereof comprising administering an effective amount of a composition comprising apatorsen disclosed herein (e.g., a composition such as a pharmaceutical composition comprising apatorsen and a monosodium or disodium salt of 5-CNAC) to the subject. Also provided is a pill or capsule comprising apatorsen and a caprylic acid derivative disclosed herein, e.g., a monosodium or disodium salt of 5-CNAC.

The oligonucleotide sequence of apatorsen is GGGACGCGGCGCTCGGUCAU (SEQ ID NO: 18). In some aspects, apatorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Aprinocarsen: In some aspects, the nucleic acid therapeutic agent is aprinocarsen. Aprinocarsen is an antisense oligonucleotide for the treatment of cancer that targets PKC-α. In some aspects, the present disclosure provides a composition comprising aprinocarsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising aprinocarsen and 5-CNAC. In one specific aspect, the present disclosure provides a composition comprising aprinocarsen and 5-CNAC disodium salt. In some aspects, the composition is formulated for oral delivery.

The present disclosure also provides a method to treat or prevent a disease or condition in a subject in need thereof comprising administering an effective amount of a composition comprising aprinocarsen disclosed herein (e.g., a composition such as a pharmaceutical composition comprising aprinocarsen and a monosodium or disodium salt of 5-CNAC) to the subject. Also provided is a pill or capsule comprising aprinocarsen and a caprylic acid derivative disclosed herein, e.g., a monosodium or disodium salt of 5-CNAC.

The oligonucleotide sequence of aprinocarsen is GGTGGTGGTGGTTGTGGTGGTGGTGG (SEQ ID NO: 19). In some aspects, aprinocarsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

APTA-16: In some aspects, the nucleic acid therapeutic agent is APTA-16. APTA-16 is an aptamer for the treatment of acute myeloid leukemia, myelodysplastic syndromes, or liver cancer, that targets histone methyltransferase. In some aspects, the present disclosure provides a composition comprising APTA-16 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising APTA-16 and 5-CNAC. In one specific aspect, the present disclosure provides a composition comprising APTA-16 and 5-CNAC disodium salt. In some aspects, the composition is formulated for oral delivery.

The present disclosure also provides a method to treat or prevent a disease or condition in a subject in need thereof comprising administering an effective amount of a composition comprising APTA-16 disclosed herein (e.g., a composition such as a pharmaceutical composition comprising APTA-16 and a monosodium or disodium salt of 5-CNAC) to the subject. Also provided is a pill or capsule comprising APTA-16 and a caprylic acid derivative disclosed herein, e.g., a monosodium or disodium salt of 5-CNAC. In some aspects, APTA-16 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ZINTEVIR™: In some aspects, the nucleic acid therapeutic agent is AR-177. AR-177, also known as ZINTEVIR™, is an oligonucleotide analogue which functions as an integrase inhibitor and can be used for the treatment of HIV-1 infection. In some aspects, the present disclosure provides a composition comprising AR-177 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AR-177 and 5-CNAC. In one specific aspect, the present disclosure provides a composition comprising AR-177 and 5-CNAC disodium salt. In some aspects, the composition is formulated for oral delivery.

The present disclosure also provides a method to treat or prevent a disease or condition in a subject in need thereof comprising administering an effective amount of a composition comprising AR-177 disclosed herein (e.g., a composition such as a pharmaceutical composition comprising AR-177 and a monosodium or disodium salt of 5-CNAC) to the subject. Also provided is a pill or capsule comprising AR-177 and a caprylic acid derivative disclosed herein, e.g., a monosodium or disodium salt of 5-CNAC.

The oligonucleotide sequence of AR-177 is GTGGTGGGTGGGTGGGT (SEQ ID NO: 20). In some aspects, AR-177 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ARC19499: In some aspects, the nucleic acid therapeutic agent is ARC19499. ARC19499, also known as BAX-499, is an RNA aptamer for the treatment of hemophilia that targets TFPI. In some aspects, the present disclosure provides a composition comprising ARC19499 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising ARC19499 and 5-CNAC. In one specific aspect, the present disclosure provides a composition comprising ARC19499 and 5-CNAC disodium salt. In some aspects, the composition is formulated for oral delivery.

The present disclosure also provides a method to treat or prevent a disease or condition in a subject in need thereof comprising administering an effective amount of a composition comprising ARC19499 disclosed herein (e.g., a composition such as a pharmaceutical composition comprising ARC19499 and a monosodium or disodium salt of 5-CNAC) to the subject. Also provided is a pill or capsule comprising ARC19499 and a caprylic acid derivative disclosed herein, e.g., a monosodium or disodium salt of 5-CNAC.

The oligonucleotide sequence of ARC19499 is GGAAUAUACUUGGCUCGUUAGGUGCGUAUAUA (SEQ ID NO: 21). In some aspects, ARC19499 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Archexin: In some aspects, the nucleic acid therapeutic agent is archexin. Archexin, also known as RX-201, is an antisense oligonucleotide for the treatment of metastatic renal cancer, ovarian cancer, renal cell carcinoma, glioblastoma, stomach cancer, pancreatic cancer, lung cancer, or cervical carcinomas that targets the AKT-1 protein kinase. In some aspects, the present disclosure provides a composition comprising archexin and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising archexin and 5-CNAC. In one specific aspect, the present disclosure provides a composition comprising archexin and 5-CNAC disodium salt. In some aspects, the composition is formulated for oral delivery.

The present disclosure also provides a method to treat or prevent a disease or condition in a subject in need thereof comprising administering an effective amount of a composition comprising archexin disclosed herein (e.g., a composition such as a pharmaceutical composition comprising archexin and a monosodium or disodium salt of 5-CNAC) to the subject. Also provided is a pill or capsule comprising archexin and a caprylic acid derivative disclosed herein, e.g., a monosodium or disodium salt of 5-CNAC.

The oligonucleotide sequence of archexin is GCTGCATGATCTCCTTGGCG (SEQ ID NO: 22). In some aspects, archexin is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Asvasiran: In some aspects, the nucleic acid therapeutic agent is asvasiran. Asvasiran is a siRNA for the treatment of respiratory syncytial virus infection that targets the RSV N gene. In some aspects, the present disclosure provides a composition comprising asvasiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising asvasiran and 5-CNAC. In one specific aspect, the present disclosure provides a composition comprising asvasiran and 5-CNAC disodium salt. In some aspects, the composition is formulated for oral delivery.

The present disclosure also provides a method to treat or prevent a disease or condition in a subject in need thereof comprising administering an effective amount of a composition comprising asvasiran disclosed herein (e.g., a composition such as a pharmaceutical composition comprising asvasiran and a monosodium or disodium salt of 5-CNAC) to the subject. Also provided is a pill or capsule comprising asvasiran and a caprylic acid derivative disclosed herein, e.g., a monosodium or disodium salt of 5-CNAC.

The oligonucleotide sequence of asvasiran is a duplex RNA comprising the antisense sequence CUUGACUUUGCUAAGAGCCTT (SEQ ID NO: 23) and the sense sequence GGCUCUUAGCAAAGUCAAGTT (SEQ ID NO: 24). In some aspects, asvasiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Atesidorsen: In some aspects, the nucleic acid therapeutic agent is atesidorsen. Atesidorsen, also known as ATL1103, is an antisense oligonucleotide for the treatment of acromegaly, cancer, or diabetic retinopathy that targets somatotropin receptors. In some aspects, the present disclosure provides a composition comprising atesidorsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising atesidorsen and 5-CNAC. In one specific aspect, the present disclosure provides a composition comprising atesidorsen and 5-CNAC disodium salt. In some aspects, the composition is formulated for oral delivery.

The present disclosure also provides a method to treat or prevent a disease or condition in a subject in need thereof comprising administering an effective amount of a composition comprising atesidorsen disclosed herein (e.g., a composition such as a pharmaceutical composition comprising atesidorsen and a monosodium or disodium salt of 5-CNAC) to the subject. Also provided is a pill or capsule comprising atesidorsen and a caprylic acid derivative disclosed herein, e.g., a monosodium or disodium salt of 5-CNAC.

The oligonucleotide sequence of atesidorsen is UCAGGGCATTCTTTCCAUUC (SEQ ID NO: 25). In some aspects, atesidorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ATU-027: In some aspects, the nucleic acid therapeutic agent is ATU-027. ATU-027 is a siRNA targeting protein kinase N3 that inhibits cancer progression, e.g., in prostate and pancreatic cancer. In some aspects, the present disclosure provides a composition comprising ATU-027 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising ATU-027 and 5-CNAC. In one specific aspect, the present disclosure provides a composition comprising ATU-027 and 5-CNAC disodium salt. In some aspects, the composition is formulated for oral delivery.

The present disclosure also provides a method to treat or prevent a disease or condition in a subject in need thereof comprising administering an effective amount of a composition comprising ATU-027 disclosed herein (e.g., a composition such as a pharmaceutical composition comprising ATU-027 and a monosodium or disodium salt of 5-CNAC) to the subject. Also provided is a pill or capsule comprising ATU-027 and a caprylic acid derivative disclosed herein, e.g., a monosodium or disodium salt of 5-CNAC.

The oligonucleotide sequence of ATU-027 is a RNA duple comprising the antisense sequence AGACUUGAGGACUUCCUGGACAA (SEQ ID NO: 26) and the sense sequence UUGUCCAGGAAGUCCUCAAGUCU (SEQ ID NO: 27). In some aspects, ATU-027 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

AVT-02: In some aspects, the nucleic acid therapeutic agent is AVT-02 developed by Avontec GmbH. AVT-02 is a short, double stranded oligonucleotide decoy for the treatment of psoriasis vulgaris that targets STAT-1. In some aspects, the present disclosure provides a composition comprising AVT-02 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AVT-02 and 5-CNAC. In one specific aspect, the present disclosure provides a composition comprising AVT-02 and 5-CNAC disodium salt. In some aspects, the composition is formulated for oral delivery.

The present disclosure also provides a method to treat or prevent a disease or condition in a subject in need thereof comprising administering an effective amount of a composition comprising AVT-02 disclosed herein (e.g., a composition such as a pharmaceutical composition comprising AVT-02 and a monosodium or disodium salt of 5-CNAC) to the subject. Also provided is a pill or capsule comprising AVT-02 and a caprylic acid derivative disclosed herein, e.g., a monosodium or disodium salt of 5-CNAC. In some aspects, AVT-02 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ZIMURA™: In some aspects, the nucleic acid therapeutic agent is avacincaptad pegol (ZIMURA™). Avacincaptad pegol is PEG-conjugated oligonucleotide for the treatment of polyploidal choroidal vasculopathy, Stargardt disease, or wet age-related macular degeneration that functions as a complement C5 inhibitor. In some aspects, the present disclosure provides a composition comprising avacincaptad pegol and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising avacincaptad pegol and 5-CNAC. In one specific aspect, the present disclosure provides a composition comprising avacincaptad pegol and 5-CNAC disodium salt. In some aspects, the composition is formulated for oral delivery.

The present disclosure also provides a method to treat or prevent a disease or condition in a subject in need thereof comprising administering an effective amount of a composition comprising avacincaptad pegol disclosed herein (e.g., a composition such as a pharmaceutical composition comprising avacincaptad pegol and a monosodium or disodium salt of 5-CNAC) to the subject. Also provided is a pill or capsule comprising avacincaptad pegol and a caprylic acid derivative disclosed herein, e.g., a monosodium or disodium salt of 5-CNAC.

The oligonucleotide sequence of avacincaptad pegol is CGCCGCGGUCUCAGGCGCUGAGUCUGAGUUUACCUGCGT (SEQ ID NO: 28). In some aspects, avacincaptad is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

AVI-7537: In some aspects, the nucleic acid therapeutic agent is AVI-7537. AVI-7537 is a morpholino antisense oligonucleotide that targets the VP24 gene of Ebola virus. In some aspects, the present disclosure provides a composition comprising AVI-7537 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AVI-7537 and 5-CNAC. In one specific aspect, the present disclosure provides a composition comprising AVI-7537 and 5-CNAC disodium salt. In some aspects, the composition is formulated for oral delivery.

The present disclosure also provides a method to treat or prevent a disease or condition in a subject in need thereof comprising administering an effective amount of a composition comprising AVI-7537 disclosed herein (e.g., a composition such as a pharmaceutical composition comprising AVI-7537 and a monosodium or disodium salt of 5-CNAC) to the subject. Also provided is a pill or capsule comprising AVI-7537 and a caprylic acid derivative disclosed herein, e.g., a monosodium or disodium salt of 5-CNAC.

The oligonucleotide sequence of AVI-7537 is GCCATGGTTTTTTCTCAGG (SEQ ID NO: 29). In some aspects, AVI-7537 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

AVI-7288: In some aspects, the nucleic acid therapeutic agent is AVI-7288. AVI-7288 is a morpholino antisense oligonucleotide that targets Marburg virus nucleoprotein (NP). In some aspects, the present disclosure provides a composition comprising AVI-7288 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising AVI-7288 and 5-CNAC. In one specific aspect, the present disclosure provides a composition comprising AVI-7288 and 5-CNAC disodium salt. In some aspects, the composition is formulated for oral delivery.

The present disclosure also provides a method to treat or prevent a disease or condition in a subject in need thereof comprising administering an effective amount of a composition comprising AVI-7288 disclosed herein (e.g., a composition such as a pharmaceutical composition comprising AVI-7288 and a monosodium or disodium salt of 5-CNAC) to the subject. Also provided is a pill or capsule comprising AVI-7288 and a caprylic acid derivative disclosed herein, e.g., a monosodium or disodium salt of 5-CNAC. In some aspects, AVI-7288 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Baliforsen: In some aspects, the nucleic acid therapeutic agent is baliforsen. Baliforsen, also known as IONIS-598769, is an antisense oligonucleotide for the treatment of myotonic dystrophy. In some aspects, the present disclosure provides a composition comprising baliforsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising baliforsen and 5-CNAC. In one specific aspect, the present disclosure provides a composition comprising baliforsen and 5-CNAC disodium salt. In some aspects, the composition is formulated for oral delivery.

The present disclosure also provides a method to treat or prevent a disease or condition in a subject in need thereof comprising administering an effective amount of a composition comprising baliforsen disclosed herein (e.g., a composition such as a pharmaceutical composition comprising baliforsen and a monosodium or disodium salt of 5-CNAC) to the subject. Also provided is a pill or capsule comprising baliforsen and a caprylic acid derivative disclosed herein, e.g., a monosodium or disodium salt of 5-CNAC.

The oligonucleotide sequence of baliforsen is TCCCGAATGTCCGACA (SEQ ID NO: 30). In some aspects, baliforsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Bamosiran: In some aspects, the nucleic acid therapeutic agent is bamosiran. Bamosiran, also known as SYL-040012, is a siRNA for the treatment of glaucoma or ocular hypertension that targets beta 2 adrenergic receptors. In some aspects, the present disclosure provides a composition comprising bamosiran and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising bamosiran and 5-CNAC. In one specific aspect, the present disclosure provides a composition comprising bamosiran and 5-CNAC disodium salt. In some aspects, the composition is formulated for oral delivery.

The present disclosure also provides a method to treat or prevent a disease or condition in a subject in need thereof comprising administering an effective amount of a composition comprising bamosiran disclosed herein (e.g., a composition such as a pharmaceutical composition comprising bamosiran and a monosodium or disodium salt of 5-CNAC) to the subject. Also provided is a pill or capsule comprising bamosiran and a caprylic acid derivative disclosed herein, e.g., a monosodium or disodium salt of 5-CNAC.

The oligonucleotide sequence of bamosiran is a duplex RNA comprising an antisense strand of sequence CAUUGUGCAUGUGAUCCAGTT (SEQ ID NO: 31) and a sense strand of sequence CUGGAUCACAUGCACAAUGTT (SEQ ID NO: 32). In some aspects, bamosiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Bazlitoran: In some aspects, the nucleic acid therapeutic agent is bazlitoran. Bazlitoran, also known as IMO-8400, is a DNA oligonucleotide for the treatment of Waldenstrom's macroglobulinemia that targets toll-like receptors TLR7, TLR8 and TLR9. In some aspects, the present disclosure provides a composition comprising bazlitoran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising bazlitoran and 5-CNAC. In one specific aspect, the present disclosure provides a composition comprising bazlitoran and 5-CNAC disodium salt. In some aspects, the composition is formulated for oral delivery.

The present disclosure also provides a method to treat or prevent a disease or condition in a subject in need thereof comprising administering an effective amount of a composition comprising bazlitoran disclosed herein (e.g., a composition such as a pharmaceutical composition comprising bazlitoran and a monosodium or disodium salt of 5-CNAC) to the subject. Also provided is a pill or capsule comprising bazlitoran and a caprylic acid derivative disclosed herein, e.g., a monosodium or disodium salt of 5-CNAC.

The oligonucleotide sequence of bazlitoran is CTATCTGUCGTTCTCTGU (SEQ ID NO: 33). In some aspects, bazlitoran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

BC007: In some aspects, the nucleic acid therapeutic agent is BC007. BC007 is a non-modified DNA aptamer out of a family of aptamers that bind to and lead to the neutralization of autoantibodies that are directed against G-protein-coupled receptors (GPCR-AABs). BC007 binds to β1-adrenergic-receptor-autoantibodies. BC007 can be used for the treatment of dilated cardiomyopathy or chronic fatigue syndrome. In some aspects, the present disclosure provides a composition comprising BC007 and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising BC007 and 5-CNAC. In one specific aspect, the present disclosure provides a composition comprising BC007 and 5-CNAC disodium salt. In some aspects, the composition is formulated for oral delivery.

The present disclosure also provides a method to treat or prevent a disease or condition in a subject in need thereof comprising administering an effective amount of a composition comprising BC007 disclosed herein (e.g., a composition such as a pharmaceutical composition comprising BC007 and a monosodium or disodium salt of 5-CNAC) to the subject. Also provided is a pill or capsule comprising BC007 and a caprylic acid derivative disclosed herein, e.g., a monosodium or disodium salt of 5-CNAC. In some aspects, BC007 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Beclanorsen: In some aspects, the nucleic acid therapeutic agent is beclanorsen. Beclanorsen, also known as SPC-2996, is an antisense oligonucleotide for the treatment of lymphoid leukemias that targets Bcl-2. In some aspects, the present disclosure provides a composition comprising beclanorsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising beclanorsen and 5-CNAC. In one specific aspect, the present disclosure provides a composition comprising beclanorsen and 5-CNAC disodium salt. In some aspects, the composition is formulated for oral delivery.

The present disclosure also provides a method to treat or prevent a disease or condition in a subject in need thereof comprising administering an effective amount of a composition comprising beclanorsen disclosed herein (e.g., a composition such as a pharmaceutical composition comprising beclanorsen and a monosodium or disodium salt of 5-CNAC) to the subject. Also provided is a pill or capsule comprising beclanorsen and a caprylic acid derivative disclosed herein, e.g., a monosodium or disodium salt of 5-CNAC.

The oligonucleotide sequence of beclanorsen is CUCCCAACGTGCGCCA (SEQ ID NO: 34). In some aspects, beclanorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Bepirovirsen: In some aspects, the nucleic acid therapeutic agent is bepirovirsen. Bepirovirsen, also known as ISIS-505358, ISIS-GSK3RX, GSK-3228836 or IONIS HBVRX, is an antisense oligonucleotide for the treatment of hepatitis B. In some aspects, the present disclosure provides a composition comprising bepirovirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising bepirovirsen and 5-CNAC. In one specific aspect, the present disclosure provides a composition comprising bepirovirsen and 5-CNAC disodium salt. In some aspects, the composition is formulated for oral delivery.

The present disclosure also provides a method to treat or prevent a disease or condition in a subject in need thereof comprising administering an effective amount of a composition comprising bepirovirsen disclosed herein (e.g., a composition such as a pharmaceutical composition comprising bepirovirsen and a monosodium or disodium salt of 5-CNAC) to the subject. Also provided is a pill or capsule comprising bepirovirsen and a caprylic acid derivative disclosed herein, e.g., a monosodium or disodium salt of 5-CNAC.

The oligonucleotide sequence of bepirovirsen is GCAGAGGTGAAGCGAAGTGC (SEQ ID NO: 35). In some aspects, bepirovirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Bevasiranib: In some aspects, the nucleic acid therapeutic agent is bevasiranib. Bevasiranib is a siRNA for the treatment of exudative age-related macular degeneration that targets VEGF. In some aspects, the present disclosure provides a composition comprising bevasiranib and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising bevasiranib and 5-CNAC. In one specific aspect, the present disclosure provides a composition comprising bevasiranib and 5-CNAC disodium salt. In some aspects, the composition is formulated for oral delivery.

The present disclosure also provides a method to treat or prevent a disease or condition in a subject in need thereof comprising administering an effective amount of a composition comprising bevasiranib disclosed herein (e.g., a composition such as a pharmaceutical composition comprising bevasiranib and a monosodium or disodium salt of 5-CNAC) to the subject. Also provided is a pill or capsule comprising bevasiranib and a caprylic acid derivative disclosed herein, e.g., a monosodium or disodium salt of 5-CNAC.

The oligonucleotide sequence of bevasiranib is an RNA duplex comprising an antisense strand of sequence ACCUCACCAAGGCCAGCACTT (SEQ ID NO: 36) and a sense strand of sequence GUGCUGGCCUUGGUGAGGUTT (SEQ ID NO: 37). In some aspects, bevasiranib is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

BMN 044: In some aspects, the nucleic acid therapeutic agent is BMN 044. BMN 044, also known as PRO44, is an antisense oligonucleotide for the treatment of Duchenne muscular dystrophy that targets mRNA encoding dystrophin. In some aspects, the present disclosure provides a composition comprising BMN 044 and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising BMN 044 and 5-CNAC. In one specific aspect, the present disclosure provides a composition comprising BMN 044 and 5-CNAC disodium salt. In some aspects, the composition is formulated for oral delivery.

The present disclosure also provides a method to treat or prevent a disease or condition in a subject in need thereof comprising administering an effective amount of a composition comprising BMN 044 disclosed herein (e.g., a composition such as a pharmaceutical composition comprising BMN 044 and a monosodium or disodium salt of 5-CNAC) to the subject. Also provided is a pill or capsule comprising BMN 044 and a caprylic acid derivative disclosed herein, e.g., a monosodium or disodium salt of 5-CNAC. In some aspects, BMN 044 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

BMN 053: In some aspects, the nucleic acid therapeutic agent is BMN 053. BMN 053, also known as PRO53, is an antisense oligonucleotide for the treatment of Duchenne muscular dystrophy that targets dystrophin. In some aspects, the present disclosure provides a composition comprising BMN 053 and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising BMN 053 and 5-CNAC. In one specific aspect, the present disclosure provides a composition comprising BMN 053 and 5-CNAC disodium salt. In some aspects, the composition is formulated for oral delivery.

The present disclosure also provides a method to treat or prevent a disease or condition in a subject in need thereof comprising administering an effective amount of a composition comprising BMN 053 disclosed herein (e.g., a composition such as a pharmaceutical composition comprising BMN 053 and a monosodium or disodium salt of 5-CNAC) to the subject. Also provided is a pill or capsule comprising BMN 053 and a caprylic acid derivative disclosed herein, e.g., a monosodium or disodium salt of 5-CNAC. In some aspects, BMN 053 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Brivoligide: In some aspects, the nucleic acid therapeutic agent is brivoligide. Brivoligide is a 23 bp decoy DNA that functions an early growth response protein 1 inhibitor. Brivoligide can be used to treat pain, e.g., postoperative pain. In some aspects, the present disclosure provides a composition comprising brivoligide and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising brivoligide and 5-CNAC. In one specific aspect, the present disclosure provides a composition comprising brivoligide and 5-CNAC disodium salt. In some aspects, the composition is formulated for oral delivery.

The present disclosure also provides a method to treat or prevent a disease or condition in a subject in need thereof comprising administering an effective amount of a composition comprising brivoligide disclosed herein (e.g., a composition such as a pharmaceutical composition comprising brivoligide and a monosodium or disodium salt of 5-CNAC) to the subject. Also provided is a pill or capsule comprising brivoligide and a caprylic acid derivative disclosed herein, e.g., a monosodium or disodium salt of 5-CNAC.

The oligonucleotide sequence of brivoligide is a duplex DNA comprising CTACGCCCACCGCCCACGCATAC (SEQ ID NO: 38) and GTATGCGTGGGCGGTGGGCGTAG (SEQ ID NO: 39). In some aspects, brivoligide is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Casimersen: In some aspects, the nucleic acid therapeutic agent is casimersen. Casimersen is a morpholino antisense oligonucleotide for the treatment of Duchenne muscular dystrophy that targets dystrophin's exon 45. In some aspects, the present disclosure provides a composition comprising casimersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising casimersen and 5-CNAC. In one specific aspect, the present disclosure provides a composition comprising casimersen and 5-CNAC disodium salt. In some aspects, the composition is formulated for oral delivery.

The present disclosure also provides a method to treat or prevent a disease or condition in a subject in need thereof comprising administering an effective amount of a composition comprising casimersen disclosed herein (e.g., a composition such as a pharmaceutical composition comprising casimersen and a monosodium or disodium salt of 5-CNAC) to the subject. Also provided is a pill or capsule comprising casimersen and a caprylic acid derivative disclosed herein, e.g., a monosodium or disodium salt of 5-CNAC.

The oligonucleotide sequence of casimersen is CAATGCCATCCTGGAGTTCCTG (SEQ ID NO: 40). In some aspects, casimersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cavrotolimod: In some aspects, the nucleic acid therapeutic agent is cavrotolimod. Cavrotolimod is an immunostimulant oligonucleotide that functions as a TLR9 agonists and can be used for the treatment of hematological malignancies, Merkel cell carcinoma, solid tumors, or squamous cell cancer. In some aspects, the present disclosure provides a composition comprising cavrotolimod and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cavrotolimod and 5-CNAC. In one specific aspect, the present disclosure provides a composition comprising cavrotolimod and 5-CNAC disodium salt. In some aspects, the composition is formulated for oral delivery.

The present disclosure also provides a method to treat or prevent a disease or condition in a subject in need thereof comprising administering an effective amount of a composition comprising cavrotolimod disclosed herein (e.g., a composition such as a pharmaceutical composition comprising cavrotolimod and a monosodium or disodium salt of 5-CNAC) to the subject. Also provided is a pill or capsule comprising cavrotolimod and a caprylic acid derivative disclosed herein, e.g., a monosodium or disodium salt of 5-CNAC.

The oligonucleotide sequence of cavrotolimod is TCGTCGTTTTGTCGTTTTGTCGTT (SEQ ID NO: 41). In some aspects, cavrotolimod is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cemdisiran: In some aspects, the nucleic acid therapeutic agent is cemdisiran. Cemdisiran, also known as AD062643, is a siRNA for the treatment of hemolytic uremic syndrome, IgA nephropathy, paroxysmal nocturnal hemoglobinuria, or myasthenia gravis that targets complement C5. In some aspects, the present disclosure provides a composition comprising cemdisiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cemdisiran and 5-CNAC. In one specific aspect, the present disclosure provides a composition comprising cemdisiran and 5-CNAC disodium salt. In some aspects, the composition is formulated for oral delivery.

The present disclosure also provides a method to treat or prevent a disease or condition in a subject in need thereof comprising administering an effective amount of a composition comprising cemdisiran disclosed herein (e.g., a composition such as a pharmaceutical composition comprising cemdisiran and a monosodium or disodium salt of 5-CNAC) to the subject. Also provided is a pill or capsule comprising cemdisiran and a caprylic acid derivative disclosed herein, e.g., a monosodium or disodium salt of 5-CNAC.

The oligonucleotide sequence of cemdisiran is an RNA duplex comprising an antisense strand of sequence UAUUAUAAAAAUAUCUUGCUUUUTT (SEQ ID NO: 42) and a sense strand of sequence AAGCAAGAUAUUUUUAUAAUAN (SEQ ID NO: 43). In some aspects, cemdisiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cenersen: In some aspects, the nucleic acid therapeutic agent is cenersen. Cenersen is an antisense oligonucleotide for the treatment of myelodysplastic syndromes, acute myeloid leukemia, or chronic lymphocytic leukemia that targets p53. In some aspects, the present disclosure provides a composition comprising cenersen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising cenersen and 5-CNAC. In one specific aspect, the present disclosure provides a composition comprising cenersen and 5-CNAC disodium salt. In some aspects, the composition is formulated for oral delivery.

The present disclosure also provides a method to treat or prevent a disease or condition in a subject in need thereof comprising administering an effective amount of a composition comprising cenersen disclosed herein (e.g., a composition such as a pharmaceutical composition comprising cenersen and a monosodium or disodium salt of 5-CNAC) to the subject. Also provided is a pill or capsule comprising cenersen and a caprylic acid derivative disclosed herein, e.g., a monosodium or disodium salt of 5-CNAC.

The oligonucleotide sequence of cenersen is CCCTGCTCCCCCCTGGCTCC (SEQ ID NO: 44). In some aspects, cenersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cobitolimod: In some aspects, the nucleic acid therapeutic agent is cobitolimod. Cobitolimod is an oligodeoxyribonucleotide for the treatment of ulcerative colitis or brain edema that is an agonist of Toll-like 9 receptors. In some aspects, the present disclosure provides a composition comprising cobitolimod and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cobitolimod and 5-CNAC. In one specific aspect, the present disclosure provides a composition comprising cobitolimod and 5-CNAC disodium salt. In some aspects, the composition is formulated for oral delivery.

The present disclosure also provides a method to treat or prevent a disease or condition in a subject in need thereof comprising administering an effective amount of a composition comprising cobitolimod disclosed herein (e.g., a composition such as a pharmaceutical composition comprising cobitolimod and a monosodium or disodium salt of 5-CNAC) to the subject. Also provided is a pill or capsule comprising cobitolimod and a caprylic acid derivative disclosed herein, e.g., a monosodium or disodium salt of 5-CNAC.

The oligonucleotide sequence of cobitolimod is GGAACAGTTCGTCCATGGC (SEQ ID NO: 45). In some aspects, cobitolimod is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cobomarsen: In some aspects, the nucleic acid therapeutic agent is cobomarsen. Cobomarsen, also known as MRG-106 or M11667, is an anti-miRNA (antimir) for the treatment of cutaneous T cell lymphoma, adult T-cell leukemia-lymphoma, chronic lymphocytic leukemia, diffuse large B cell lymphoma, or amyotrophic lateral sclerosis that targets miR-155. In some aspects, the present disclosure provides a composition comprising cobomarsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cobomarsen and 5-CNAC. In one specific aspect, the present disclosure provides a composition comprising cobomarsen and 5-CNAC disodium salt. In some aspects, the composition is formulated for oral delivery.

The present disclosure also provides a method to treat or prevent a disease or condition in a subject in need thereof comprising administering an effective amount of a composition comprising cobomarsen disclosed herein (e.g., a composition such as a pharmaceutical composition comprising cobomarsen and a monosodium or disodium salt of 5-CNAC) to the subject. Also provided is a pill or capsule comprising cobomarsen and a caprylic acid derivative disclosed herein, e.g., a monosodium or disodium salt of 5-CNAC. In some aspects, cobomarsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

NEXAGON™: In some aspects, the nucleic acid therapeutic agent is CODA-001. CODA-001, also known as NEXAGON™, is an antisense oligonucleotide for the treatment of wounds, leg ulcers, diabetic foot ulcers, or corneal injuries that targets gap junctions. NEXAGON™ is a natural, unmodified oligonucleotide (30-mer) that downregulates expression of the key gap junction protein Cx43. In some aspects, the present disclosure provides a composition comprising CODA-001 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising CODA-001 and 5-CNAC. In one specific aspect, the present disclosure provides a composition comprising CODA-001 and 5-CNAC disodium salt. In some aspects, the composition is formulated for oral delivery.

The present disclosure also provides a method to treat or prevent a disease or condition in a subject in need thereof comprising administering an effective amount of a composition comprising CODA-001 disclosed herein (e.g., a composition such as a pharmaceutical composition comprising CODA-001 and a monosodium or disodium salt of 5-CNAC) to the subject. Also provided is a pill or capsule comprising CODA-001 and a caprylic acid derivative disclosed herein, e.g., a monosodium or disodium salt of 5-CNAC.

The oligonucleotide sequence of CODA-001 is GTAATTGCGGCAAGAAGAATTGTTTCTGTC (SEQ ID NO: 46). In some aspects, CODA-001 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cofirasersen: In some aspects, the nucleic acid therapeutic agent is cofirasersen. Cofirasersen, also known as is an IONIS-ENACRX and ION-827359, is an antisense oligonucleotide for the treatment of pulmonary disease, chronic bronchitis, or cystic fibrosis that targets ENaC. In some aspects, the present disclosure provides a composition comprising cofirasersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cofirasersen and 5-CNAC. In one specific aspect, the present disclosure provides a composition comprising cofirasersen and 5-CNAC disodium salt. In some aspects, the composition is formulated for oral delivery.

The present disclosure also provides a method to treat or prevent a disease or condition in a subject in need thereof comprising administering an effective amount of a composition comprising cofirasersen disclosed herein (e.g., a composition such as a pharmaceutical composition comprising cofirasersen and a monosodium or disodium salt of 5-CNAC) to the subject. Also provided is a pill or capsule comprising cofirasersen and a caprylic acid derivative disclosed herein, e.g., a monosodium or disodium salt of 5-CNAC.

The oligonucleotide sequence of cofirasersen is CCCGATAGCTGGTUGU (SEQ ID NO: 47). In some aspects, cofirasersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cosdosiran: In some aspects, the nucleic acid therapeutic agent is cosdosiran. Cosdosiran, also known as QPI-1007, is a neuroprotective siRNA for the treatment of nonarteritic anterior ischemic optic neuropathy that inhibits caspase 2 synthesis. In some aspects, the present disclosure provides a composition comprising cosdosiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cosdosiran and 5-CNAC. In one specific aspect, the present disclosure provides a composition comprising cosdosiran and 5-CNAC disodium salt. In some aspects, the composition is formulated for oral delivery.

The present disclosure also provides a method to treat or prevent a disease or condition in a subject in need thereof comprising administering an effective amount of a composition comprising cosdosiran disclosed herein (e.g., a composition such as a pharmaceutical composition comprising cosdosiran and a monosodium or disodium salt of 5-CNAC) to the subject. Also provided is a pill or capsule comprising cosdosiran and a caprylic acid derivative disclosed herein, e.g., a monosodium or disodium salt of 5-CNAC.

The oligonucleotide sequence of cosdosiran is an RNA duplex comprising an antisense strand of sequence GCCAGAAUGUGGAACUCCU (SEQ ID NO: 48) and a sense strand of sequence AGGAGUUCCACAUUCUGGC (SEQ ID NO: 49). In some aspects, cosdosiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

CPG-8954: In some aspects, the nucleic acid therapeutic agent is CPG-8954. CPG-8954 is a CpG oligonucleotide for the treatment of cancer and viral infections. In some aspects, the present disclosure provides a composition comprising CPG-8954 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising CPG-8954 and 5-CNAC. In one specific aspect, the present disclosure provides a composition comprising CPG-8954 and 5-CNAC disodium salt. In some aspects, the composition is formulated for oral delivery.

The present disclosure also provides a method to treat or prevent a disease or condition in a subject in need thereof comprising administering an effective amount of a composition comprising CPG-8954 disclosed herein (e.g., a composition such as a pharmaceutical composition comprising CPG-8954 and a monosodium or disodium salt of 5-CNAC) to the subject. Also provided is a pill or capsule comprising CPG-8954 and a caprylic acid derivative disclosed herein, e.g., a monosodium or disodium salt of 5-CNAC.

The oligonucleotide sequence of CPG-8954 is GGGGGGGTGTCGCAGCAGGGG (SEQ ID NO: 50). In some aspects, CPG-8954 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cupabimod: In some aspects, the nucleic acid therapeutic agent is cupabimod. Cupabimod, also known as AMG-0103, is an oligonucleotide for the treatment of pain, e.g., chronic discogenic lumbar back pain. In some aspects, the present disclosure provides a composition comprising cupabimod and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising cupabimod and 5-CNAC. In one specific aspect, the present disclosure provides a composition comprising cupabimod and 5-CNAC disodium salt. In some aspects, the composition is formulated for oral delivery.

The present disclosure also provides a method to treat or prevent a disease or condition in a subject in need thereof comprising administering an effective amount of a composition comprising cupabimod disclosed herein (e.g., a composition such as a pharmaceutical composition comprising cupabimod and a monosodium or disodium salt of 5-CNAC) to the subject. Also provided is a pill or capsule comprising cupabimod and a caprylic acid derivative disclosed herein, e.g., a monosodium or disodium salt of 5-CNAC.

The oligonucleotide sequence of cupabimod is a double stranded DNA comprising the sequences GGAGGGAAATCCCTTCAAGG (SEQ ID NO: 51) and CCTTGAAGGGATTTCCCTCC (SEQ ID NO: 52). In some aspects, cupabimod is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Custirsen: In some aspects, the nucleic acid therapeutic agent is custirsen. Custirsen, also known as OGX-011 and ISIS-112989, is an antisense oligonucleotide for the treatment of metastatic castrate resistant prostate cancer that targets clusterin. In some aspects, the present disclosure provides a composition comprising custirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising custirsen and 5-CNAC. In one specific aspect, the present disclosure provides a composition comprising custirsen and 5-CNAC disodium salt. In some aspects, the composition is formulated for oral delivery.

The present disclosure also provides a method to treat or prevent a disease or condition in a subject in need thereof comprising administering an effective amount of a composition comprising custirsen disclosed herein (e.g., a composition such as a pharmaceutical composition comprising custirsen and a monosodium or disodium salt of 5-CNAC) to the subject. Also provided is a pill or capsule comprising custirsen and a caprylic acid derivative disclosed herein, e.g., a monosodium or disodium salt of 5-CNAC.

The oligonucleotide sequence of custirsen is CAGCAGCAGAGTCTTCAUCAU (SEQ ID NO: 53). In some aspects, custirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Danvatirsen: In some aspects, the nucleic acid therapeutic agent is danvatirsen. Danvatirsen, also known as AZD 9150 and ISIS-481464, is an antisense oligonucleotide for the treatment of bladder cancer, colorectal cancer, head and neck cancer, malignant ascites, non-small cell lung cancer, pancreatic cancer, solid tumors, liver cancer, non-Hodgkin's lymphoma, or diffuse large B cell lymphoma, and targets the STAT3 transcription factor. In some aspects, the present disclosure provides a composition comprising danvatirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising danvatirsen and 5-CNAC. In one specific aspect, the present disclosure provides a composition comprising danvatirsen and 5-CNAC disodium salt. In some aspects, the composition is formulated for oral delivery.

The present disclosure also provides a method to treat or prevent a disease or condition in a subject in need thereof comprising administering an effective amount of a composition comprising danvatirsen disclosed herein (e.g., a composition such as a pharmaceutical composition comprising danvatirsen and a monosodium or disodium salt of 5-CNAC) to the subject. Also provided is a pill or capsule comprising danvatirsen and a caprylic acid derivative disclosed herein, e.g., a monosodium or disodium salt of 5-CNAC.

The oligonucleotide sequence of danvatirsen is CUATTTGGATGTCAGC (SEQ ID NO: 54). In some aspects, danvatirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Daplusiran: In some aspects, the nucleic acid therapeutic agent is daplusiran. Daplusiran is an antiviral siRNA. In some aspects, the present disclosure provides a composition comprising daplusiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising daplusiran and 5-CNAC. In one specific aspect, the present disclosure provides a composition comprising daplusiran and 5-CNAC disodium salt. In some aspects, the composition is formulated for oral delivery.

The present disclosure also provides a method to treat or prevent a disease or condition in a subject in need thereof comprising administering an effective amount of a composition comprising daplusiran disclosed herein (e.g., a composition such as a pharmaceutical composition comprising daplusiran and a monosodium or disodium salt of 5-CNAC) to the subject. Also provided is a pill or capsule comprising daplusiran and a caprylic acid derivative disclosed herein, e.g., a monosodium or disodium salt of 5-CNAC.

The oligonucleotide sequence of daplusiran is an RNA duplex comprising an antisense strand of sequence GUGGACUUCUCUCAAUUUUCU (SEQ ID NO: 55) and a sense strand of sequence AGAAAAUUGAGAGAAGUCCAC (SEQ ID NO: 56). In some aspects, daplusiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Defibrotide (DEFITELIO™): In some aspects, the nucleic acid therapeutic agent is defibrotide (DEFITELIO™). Defibrotide, also known as DASOVAS™, NORAVID™, or PROCICLIDE™, is a heparanase inhibitor that functions as an angiogenesis and platelet aggregation inhibitor. Defibrotide is a mixture of single-stranded oligonucleotides that is purified from the intestinal mucosa of pigs. Defibrotide can be used for the treatment of veno-occlusive disorders, graft-versus-host disease, neurological disorders, thrombotic microangiopathies, deep vein thrombosis, thrombosis, diabetic nephropathies, or multiple myeloma. In some aspects, the present disclosure provides a composition comprising defibrotide and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising defibrotide and 5-CNAC. In one specific aspect, the present disclosure provides a composition comprising defibrotide and 5-CNAC disodium salt. In some aspects, the composition is formulated for oral delivery.

The present disclosure also provides a method to treat or prevent a disease or condition in a subject in need thereof comprising administering an effective amount of a composition comprising defibrotide disclosed herein (e.g., a composition such as a pharmaceutical composition comprising defibrotide and a monosodium or disodium salt of 5-CNAC) to the subject. Also provided is a pill or capsule comprising defibrotide and a caprylic acid derivative disclosed herein, e.g., a monosodium or disodium salt of 5-CNAC. In some aspects, defibrotide is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Dematirsen: In some aspects, the nucleic acid therapeutic agent is the antisense oligonucleotide dematirsen. In some aspects, the present disclosure provides a composition comprising dematirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising dematirsen and 5-CNAC. In one specific aspect, the present disclosure provides a composition comprising dematirsen and 5-CNAC disodium salt. In some aspects, the composition is formulated for oral delivery.

The present disclosure also provides a method to treat or prevent a disease or condition in a subject in need thereof comprising administering an effective amount of a composition comprising dematirsen disclosed herein (e.g., a composition such as a pharmaceutical composition comprising dematirsen and a monosodium or disodium salt of 5-CNAC) to the subject. Also provided is a pill or capsule comprising dematirsen and a caprylic acid derivative disclosed herein, e.g., a monosodium or disodium salt of 5-CNAC.

The oligonucleotide sequence of dematirsen is GUUGCCUCCGGUUCUGAAGGUGUUC (SEQ ID NO: 57). In some aspects, dematirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Donidalorsen: In some aspects, the nucleic acid therapeutic agent is the antisense oligonucleotide donidalorsen. Donidalorsen, also known as ISIS-721744, is a plasma kallikrein inhibitor that can be used for the treatment of COVID 2019 infections, hereditary angioedema, or acute respiratory disease. In some aspects, the present disclosure provides a composition comprising donidalorsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising donidalorsen and 5-CNAC. In one specific aspect, the present disclosure provides a composition comprising donidalorsen and 5-CNAC disodium salt. In some aspects, the composition is formulated for oral delivery.

The present disclosure also provides a method to treat or prevent a disease or condition in a subject in need thereof comprising administering an effective amount of a composition comprising donidalorsen disclosed herein (e.g., a composition such as a pharmaceutical composition comprising donidalorsen and a monosodium or disodium salt of 5-CNAC) to the subject. Also provided is a pill or capsule comprising donidalorsen and a caprylic acid derivative disclosed herein, e.g., a monosodium or disodium salt of 5-CNAC.

The oligonucleotide sequence of donidalorsen is TGCAAGTCTCTTGGCAAACA (SEQ ID NO: 58). In some aspects, donidalorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Drisapersen (KYNDRISA™): In some aspects, the nucleic acid therapeutic agent is drisapersen (KYNDRISA™). Drisapersen, also known as GSK 2402968A, is an antisense oligonucleotide for the treatment of Duchenne muscular dystrophy that targets mRNA encoding dystrophin. In some aspects, the present disclosure provides a composition comprising drisapersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising drisapersen and 5-CNAC. In one specific aspect, the present disclosure provides a composition comprising drisapersen and 5-CNAC disodium salt. In some aspects, the composition is formulated for oral delivery.

The present disclosure also provides a method to treat or prevent a disease or condition in a subject in need thereof comprising administering an effective amount of a composition comprising drisapersen disclosed herein (e.g., a composition such as a pharmaceutical composition comprising drisapersen and a monosodium or disodium salt of 5-CNAC) to the subject. Also provided is a pill or capsule comprising drisapersen and a caprylic acid derivative disclosed herein, e.g., a monosodium or disodium salt of 5-CNAC.

The oligonucleotide sequence of drisapersen is UCAAGGAAGAUGGCAUUUCU (SEQ ID NO: 59). In some aspects, drisapersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Edifoligide: In some aspects, the nucleic acid therapeutic agent is edifoligide. Edifoligide is a 14 bp decoy DNA that functions as a CDC2 kinase inhibitor and can be used for the treatment of coronary artery restenosis or vascular graft occlusion. In some aspects, the present disclosure provides a composition comprising edifoligide and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising edifoligide and 5-CNAC. In one specific aspect, the present disclosure provides a composition comprising edifoligide and 5-CNAC disodium salt. In some aspects, the composition is formulated for oral delivery.

The present disclosure also provides a method to treat or prevent a disease or condition in a subject in need thereof comprising administering an effective amount of a composition comprising edifoligide disclosed herein (e.g., a composition such as a pharmaceutical composition comprising edifoligide and a monosodium or disodium salt of 5-CNAC) to the subject. Also provided is a pill or capsule comprising edifoligide and a caprylic acid derivative disclosed herein, e.g., a monosodium or disodium salt of 5-CNAC.

The oligonucleotide sequence of edifoligide is a double stranded DNA comprising the sequences CTAGATTTCCCGCG (SEQ ID NO: 60) and GATCCGCGGGAAAT (SEQ ID NO: 61). In some aspects, edifoligide is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Egaptivon pegol: In some aspects, the nucleic acid therapeutic agent is egaptivon pegol. Egaptivon pegol, also known as ARC1779, is an aptamer for the treatment of intracranial embolism, cerebral thromboembolism, carotid stenosis, von Willebrand disease, thrombotic thrombocytopenic purpura, thrombotic microangiopathy, thrombosis, or acute myocardial infarction that targets VWF GP1BA. In some aspects, the present disclosure provides a composition comprising egaptivon pegol and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising egaptivon pegol and 5-CNAC. In one specific aspect, the present disclosure provides a composition comprising egaptivon pegol and 5-CNAC disodium salt. In some aspects, the composition is formulated for oral delivery.

The present disclosure also provides a method to treat or prevent a disease or condition in a subject in need thereof comprising administering an effective amount of a composition comprising egaptivon pegol disclosed herein (e.g., a composition such as a pharmaceutical composition comprising egaptivon pegol and a monosodium or disodium salt of 5-CNAC) to the subject. Also provided is a pill or capsule comprising egaptivon pegol and a caprylic acid derivative disclosed herein, e.g., a monosodium or disodium salt of 5-CNAC.

The oligonucleotide sequence of egaptivon pegol is GCGUGCAGUGCCUUCGGCCGTGCGGTGCCUCCGUCACGCT (SEQ ID NO: 62). In some aspects, egaptivon is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

EIF-4E: In some aspects, the nucleic acid therapeutic agent is EIF-4E ASO. EIF-4E ASO is an antisense oligonucleotide for the treatment of prostate cancer disclosed in US20140323543A1, which is herein incorporated by reference. In some aspects, the present disclosure provides a composition comprising EIF-4E ASO and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising EIF-4E ASO and 5-CNAC. In one specific aspect, the present disclosure provides a composition comprising the EIF-4E ASO and 5-CNAC disodium salt. In some aspects, the composition is formulated for oral delivery.

The present disclosure also provides a method to treat or prevent a disease or condition in a subject in need thereof comprising administering an effective amount of a composition comprising EIF-4E ASO disclosed herein (e.g., a composition such as a pharmaceutical composition comprising EIF-4E ASO and a monosodium or disodium salt of 5-CNAC) to the subject. Also provided is a pill or capsule comprising EIF-4E ASO and a caprylic acid derivative disclosed herein, e.g., a monosodium or disodium salt of 5-CNAC.

The oligonucleotide sequence of EIF-4E ASO is TGTTATATTCCTGGATCCTT (SEQ ID NO: 63). In some aspects, EIF-4E is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Eluforsen: In some aspects, the nucleic acid therapeutic agent is eluforsen. Eluforsen, also known as QR-010, is an oligonucleotide partly complementary to Phe508del-CFTR RNA. Eluforsen, also known as QR-010 is designed to repair CFTR-encoded mRNA. In some aspects, the present disclosure provides a composition comprising eluforsen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising eluforsen and 5-CNAC. In one specific aspect, the present disclosure provides a composition comprising eluforsen and 5-CNAC disodium salt. In some aspects, the composition is formulated for oral delivery.

The present disclosure also provides a method to treat or prevent a disease or condition in a subject in need thereof comprising administering an effective amount of a composition comprising eluforsen disclosed herein (e.g., a composition such as a pharmaceutical composition comprising eluforsen and a monosodium or disodium salt of 5-CNAC) to the subject. Also provided is a pill or capsule comprising eluforsen and a caprylic acid derivative disclosed herein, e.g., a monosodium or disodium salt of 5-CNAC.

The oligonucleotide sequence of eluforsen is AUCAUAGGAAACACCAAAGAUGAUAUUUUCUUU (SEQ ID NO: 64). In some aspects, eluforsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Emapticap pegol: In some aspects, the nucleic acid therapeutic agent is emapticap pegol. Emapticap pegol, also known as NOX-E36, is an aptamer for the treatment of systemic lupus erythematosus type 2, diabetes mellitus, chronic inflammatory diseases, albuminuria, and renal impairment that targets CCL2. In some aspects, the present disclosure provides a composition comprising emapticap pegol and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising emapticap pegol and 5-CNAC. In one specific aspect, the present disclosure provides a composition comprising emapticap pegol and 5-CNAC disodium salt. In some aspects, the composition is formulated for oral delivery.

The present disclosure also provides a method to treat or prevent a disease or condition in a subject in need thereof comprising administering an effective amount of a composition comprising emapticap pegol disclosed herein (e.g., a composition such as a pharmaceutical composition comprising emapticap pegol and a monosodium or disodium salt of 5-CNAC) to the subject. Also provided is a pill or capsule comprising emapticap pegol and a caprylic acid derivative disclosed herein, e.g., a monosodium or disodium salt of 5-CNAC.

The oligonucleotide sequence of emapticap pegol is GCACGUCCCUCACCGGUGCAAGUGAAGCCGUGGCUCUGCG (SEQ ID NO: 65). In some aspects, emapticap is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

Eplontersen: In some aspects, the nucleic acid therapeutic agent is eplontersen. Eplontersen, also known as ION-TTR-LRX or AKCEA-TTR-LRX, is an antisense oligonucleotide that functions as a prealbumin expression inhibitor and can be used to treat amyloidosis or transthyretin-related hereditary amyloidosis. In some aspects, the present disclosure provides a composition comprising eplontersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising eplontersen and 5-CNAC. In one specific aspect, the present disclosure provides a composition comprising eplontersen and 5-CNAC disodium salt. In some aspects, the composition is formulated for oral delivery.

The present disclosure also provides a method to treat or prevent a disease or condition in a subject in need thereof comprising administering an effective amount of a composition comprising eplontersen disclosed herein (e.g., a composition such as a pharmaceutical composition comprising eplontersen and a monosodium or disodium salt of 5-CNAC) to the subject. Also provided is a pill or capsule comprising eplontersen and a caprylic acid derivative disclosed herein, e.g., a monosodium or disodium salt of 5-CNAC.

The oligonucleotide sequence of eplontersen is UCUUGGTTACATGAAAUCCC (SEQ ID NO: 66). In some aspects, eplontersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Eteplirsen (EXONDYS 51™): In some aspects, the nucleic acid therapeutic agent is eteplirsen (EXONDYS 51™). Eteplirsen, also known, AVI-4658, is an antisense oligonucleotide for the treatment of Duchenne muscular dystrophy that targets DMD exon 51. In some aspects, the present disclosure provides a composition comprising eteplirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising eteplirsen and 5-CNAC. In one specific aspect, the present disclosure provides a composition comprising eteplirsen and 5-CNAC disodium salt. In some aspects, the composition is formulated for oral delivery.

The present disclosure also provides a method to treat or prevent a disease or condition in a subject in need thereof comprising administering an effective amount of a composition comprising eteplirsen disclosed herein (e.g., a composition such as a pharmaceutical composition comprising eteplirsen and a monosodium or disodium salt of 5-CNAC) to the subject. Also provided is a pill or capsule comprising eteplirsen and a caprylic acid derivative disclosed herein, e.g., a monosodium or disodium salt of 5-CNAC.

The oligonucleotide sequence of eteplirsen is CTCCAACATCAAGGAAGATGGCATTTCTAG (SEQ ID NO: 67). In some aspects, eteplirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Fesomersen: In some aspects, the nucleic acid therapeutic agent is the antisense oligonucleotide fesomersen. In some aspects, the present disclosure provides a composition comprising fesomersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising fesomersen and 5-CNAC. In one specific aspect, the present disclosure provides a composition comprising fesomersen and 5-CNAC disodium salt. In some aspects, the composition is formulated for oral delivery.

The present disclosure also provides a method to treat or prevent a disease or condition in a subject in need thereof comprising administering an effective amount of a composition comprising fesomersen disclosed herein (e.g., a composition such as a pharmaceutical composition comprising fesomersen and a monosodium or disodium salt of 5-CNAC) to the subject. Also provided is a pill or capsule comprising fesomersen and a caprylic acid derivative disclosed herein, e.g., a monosodium or disodium salt of 5-CNAC.

The oligonucleotide sequence of fesomersen is ACGGCATTGGTGCACAGUUU (SEQ ID NO: 68). In some aspects, fesomersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Fitusiran: In some aspects, the nucleic acid therapeutic agent is fitusiran. Fitusiran, also known as ALN-57213, is a siRNA for the treatment of hemophilia A and B that targets SERPINC1. In some aspects, the present disclosure provides a composition comprising fitusiran and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising fitusiran and 5-CNAC. In one specific aspect, the present disclosure provides a composition comprising fitusiran and 5-CNAC disodium salt. In some aspects, the composition is formulated for oral delivery.

The present disclosure also provides a method to treat or prevent a disease or condition in a subject in need thereof comprising administering an effective amount of a composition comprising fitusiran disclosed herein (e.g., a composition such as a pharmaceutical composition comprising fitusiran and a monosodium or disodium salt of 5-CNAC) to the subject. Also provided is a pill or capsule comprising fitusiran and a caprylic acid derivative disclosed herein, e.g., a monosodium or disodium salt of 5-CNAC.

The oligonucleotide sequence of fitusiran is a RNA duplex comprising an antisense strand of sequence UUGAAGUAAAUGGUGUUAACCAG (SEQ ID NO: 69) and a sense strand of sequence GGUUAACACCAUUUACUUCAA (SEQ ID NO: 70). In some aspects, fitusiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Fomivirsen (VITRAVENE™): In some aspects, the nucleic acid therapeutic agent is fomivirsen (VITRAVENE™). Fomivirsen is an antisense oligonucleotide for the treatment of cytomegalovirus-induced retinitis and HIV infections that targets cytomegalovirus mRNA. In some aspects, the present disclosure provides a composition comprising fomivirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising fomivirsen and 5-CNAC. In one specific aspect, the present disclosure provides a composition comprising fomivirsen and 5-CNAC disodium salt. In some aspects, the composition is formulated for oral delivery.

The present disclosure also provides a method to treat or prevent a disease or condition in a subject in need thereof comprising administering an effective amount of a composition comprising fomivirsen disclosed herein (e.g., a composition such as a pharmaceutical composition comprising fomivirsen and a monosodium or disodium salt of 5-CNAC) to the subject. Also provided is a pill or capsule comprising fomivirsen and a caprylic acid derivative disclosed herein, e.g., a monosodium or disodium salt of 5-CNAC.

The oligonucleotide sequence of fomivirsen is GCGTTTGCTCTTCTTCTTGCG (SEQ ID NO: 71). In some aspects, fomivirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Gataparsen: In some aspects, the nucleic acid therapeutic agent is gataparsen. Gataparsen is an antisense oligonucleotide for the treatment of acute myeloid leukemia, non-small cell lung cancer, or prostate cancer that targets BIRC5. In some aspects, the present disclosure provides a composition comprising gataparsen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising gataparsen and 5-CNAC. In one specific aspect, the present disclosure provides a composition comprising gataparsen and 5-CNAC disodium salt. In some aspects, the composition is formulated for oral delivery.

The present disclosure also provides a method to treat or prevent a disease or condition in a subject in need thereof comprising administering an effective amount of a composition comprising gataparsen disclosed herein (e.g., a composition such as a pharmaceutical composition comprising gataparsen and a monosodium or disodium salt of 5-CNAC) to the subject. Also provided is a pill or capsule comprising gataparsen and a caprylic acid derivative disclosed herein, e.g., a monosodium or disodium salt of 5-CNAC.

The oligonucleotide sequence of gataparsen is TGTGCTATTCTGTGAATT (SEQ ID NO: 72). In some aspects, gataparsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Givosiran (GIVLAARI™): In some aspects, the nucleic acid therapeutic agent is givosiran (GIVLAARI™). Givosiran is a siRNA for the treatment of acute hepatic porphyria that targets 5-aminolevulinate synthetase (ALAS1). In some aspects, the present disclosure provides a composition comprising givosiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising givosiran and 5-CNAC. In one specific aspect, the present disclosure provides a composition comprising givosiran and 5-CNAC disodium salt. In some aspects, the composition is formulated for oral delivery.

The present disclosure also provides a method to treat or prevent a disease or condition in a subject in need thereof comprising administering an effective amount of a composition comprising givosiran disclosed herein (e.g., a composition such as a pharmaceutical composition comprising givosiran and a monosodium or disodium salt of 5-CNAC) to the subject. Also provided is a pill or capsule comprising givosiran and a caprylic acid derivative disclosed herein, e.g., a monosodium or disodium salt of 5-CNAC.

The oligonucleotide sequence of givosiran is antisense AAUGAUGAGACACUCUUUCUGGU (SEQ ID NO: 73) sense CAGAAAGAGUGUCUCAUCUUA (SEQ ID NO: 74). In some aspects, givosiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

GNKG-168: In some aspects, the nucleic acid therapeutic agent is GNKG-168. GNKG-168, also known as CPG-685, is an oligonucleotide that functions as a TLR9 agonist. GNKG-168 can be used for the treatment of chronic lymphocytic leukemia. In some aspects, the present disclosure provides a composition comprising GNKG-168 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising GNKG-168 and 5-CNAC. In one specific aspect, the present disclosure provides a composition comprising GNKG-168 and 5-CNAC disodium salt. In some aspects, the composition is formulated for oral delivery.

The present disclosure also provides a method to treat or prevent a disease or condition in a subject in need thereof comprising administering an effective amount of a composition comprising GNKG-168 disclosed herein (e.g., a composition such as a pharmaceutical composition comprising GNKG-168 and a monosodium or disodium salt of 5-CNAC) to the subject. Also provided is a pill or capsule comprising GNKG-168 and a caprylic acid derivative disclosed herein, e.g., a monosodium or disodium salt of 5-CNAC.

The oligonucleotide sequence of GNKG-168 is TCGTCGACGTCGTTCGTTCTC (SEQ ID NO: 75). In some aspects, GNKG-168 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Golodirsen (VYONDYS 53™): In some aspects, the nucleic acid therapeutic agent is golodirsen (VYONDYS 53™). Golodirsen, also known as SRP-4053 and VYONDYS 53™, is an antisense oligonucleotide used to treat Duchenne muscular dystrophy via splicing modulation that targets DMD exon 53. In some aspects, the present disclosure provides a composition comprising golodirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising golodirsen and 5-CNAC. In one specific aspect, the present disclosure provides a composition comprising golodirsen and 5-CNAC disodium salt. In some aspects, the composition is formulated for oral delivery.

The present disclosure also provides a method to treat or prevent a disease or condition in a subject in need thereof comprising administering an effective amount of a composition comprising golodirsen disclosed herein (e.g., a composition such as a pharmaceutical composition comprising golodirsen and a monosodium or disodium salt of 5-CNAC) to the subject. Also provided is a pill or capsule comprising golodirsen and a caprylic acid derivative disclosed herein, e.g., a monosodium or disodium salt of 5-CNAC.

The oligonucleotide sequence of golodirsen is GTTGCCTCCGGTTCTGAAGGTGTTC (SEQ ID NO: 76). In some aspects, golodirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

GPI-2A: In some aspects, the nucleic acid therapeutic agent is GPI-2A. GPI-2A is an antisense oligonucleotide for the treatment of HIV that inhibits the expression of human immunodeficiency virus type 1 capsid. In some aspects, the present disclosure provides a composition comprising GPI-2A and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising GPI-2A and 5-CNAC. In one specific aspect, the present disclosure provides a composition comprising GPI-2A and 5-CNAC disodium salt. In some aspects, the composition is formulated for oral delivery.

The present disclosure also provides a method to treat or prevent a disease or condition in a subject in need thereof comprising administering an effective amount of a composition comprising GPI-2A disclosed herein (e.g., a composition such as a pharmaceutical composition comprising GPI-2A and a monosodium or disodium salt of 5-CNAC) to the subject. Also provided is a pill or capsule comprising GPI-2A and a caprylic acid derivative disclosed herein, e.g., a monosodium or disodium salt of 5-CNAC.

The oligonucleotide sequence of GPI-2A is GGTTCTTTTGGTCCTTGTCT (SEQ ID NO: 77). In some aspects, GPI-2A is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

GTI-2040: In some aspects, the nucleic acid therapeutic agent is GTI-2040. GTI-2040, also known as LOR-2040, is an antisense oligonucleotide for the treatment of renal cell carcinoma that functions as a DNA synthesis inhibitor. GTI-2040 can also be used for the treatment of acute myeloid leukemia, bladder cancer, breast cancer, chronic myeloid leukemia, colorectal cancer, myelodysplastic syndromes, non-small cell lung cancer, or prostate cancer. In some aspects, the present disclosure provides a composition comprising GTI-2040 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising GTI-2040 and 5-CNAC. In one specific aspect, the present disclosure provides a composition comprising GTI-2040 and 5-CNAC disodium salt. In some aspects, the composition is formulated for oral delivery.

The present disclosure also provides a method to treat or prevent a disease or condition in a subject in need thereof comprising administering an effective amount of a composition comprising GTI-2040 disclosed herein (e.g., a composition such as a pharmaceutical composition comprising GTI-2040 and a monosodium or disodium salt of 5-CNAC) to the subject. Also provided is a pill or capsule comprising GTI-2040 and a caprylic acid derivative disclosed herein, e.g., a monosodium or disodium salt of 5-CNAC.

The oligonucleotide sequence of GTI-2040 is GGCTAAATCGCTCCACCAAG (SEQ ID NO: 78). In some aspects, GTI-2040 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

GTI-2501: In some aspects, the nucleic acid therapeutic agent is GTI-2501. GTI-2501 is an antisense oligonucleotide for the treatment of renal cell carcinoma that functions as a DNA synthesis inhibitor by targeting the ribonucleoside-diphosphate reductase large subunit. GTI-2501 can also be used for the treatment of acute myeloid leukemia, bladder cancer, breast cancer, chronic myeloid leukemia, colorectal cancer, myelodysplastic syndromes, non-small cell lung cancer, or prostate cancer. In some aspects, the present disclosure provides a composition comprising GTI-2501 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising GTI-2501 and 5-CNAC. In one specific aspect, the present disclosure provides a composition comprising GTI-2501 and 5-CNAC disodium salt. In some aspects, the composition is formulated for oral delivery.

The present disclosure also provides a method to treat or prevent a disease or condition in a subject in need thereof comprising administering an effective amount of a composition comprising GTI-2501 disclosed herein (e.g., a composition such as a pharmaceutical composition comprising GTI-2501 and a monosodium or disodium salt of 5-CNAC) to the subject. Also provided is a pill or capsule comprising GTI-2501 and a caprylic acid derivative disclosed herein, e.g., a monosodium or disodium salt of 5-CNAC.

The oligonucleotide sequence of GTI-2501 is CTCTAGCGTCTTAAAGCCGA (SEQ ID NO: 79). In some aspects, GTI-2501 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

HBVAXPRO: In some aspects, the nucleic acid therapeutic agent is HBVAXPRO. HBVAXPRO is a decoy for the treatment of Hepatitis B that targets HBV. In some aspects, the present disclosure provides a composition comprising HBVAXPRO and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising HBVAXPRO and 5-CNAC. In one specific aspect, the present disclosure provides a composition comprising HBVAXPRO and 5-CNAC disodium salt. In some aspects, the composition is formulated for oral delivery.

The present disclosure also provides a method to treat or prevent a disease or condition in a subject in need thereof comprising administering an effective amount of a composition comprising HBVAXPRO disclosed herein (e.g., a composition such as a pharmaceutical composition comprising HBVAXPRO and a monosodium or disodium salt of 5-CNAC) to the subject. Also provided is a pill or capsule comprising HBVAXPRO and a caprylic acid derivative disclosed herein, e.g., a monosodium or disodium salt of 5-CNAC. In some aspects, HBVAXPRO is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IMT-504: In some aspects, the nucleic acid therapeutic agent is IMT-504. IMT-504 is a B cell immunostimulant oligonucleotide for the treatment of diabetes mellitus, rabies, breast cancer, chronic lymphocytic leukemia, hepatitis B, influenza virus infections, neuropathic pain, osteoporosis, or sepsis. In some aspects, the present disclosure provides a composition comprising IMT-504 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IMT-504 and 5-CNAC. In one specific aspect, the present disclosure provides a composition comprising IMT-504 and 5-CNAC disodium salt. In some aspects, the composition is formulated for oral delivery.

The present disclosure also provides a method to treat or prevent a disease or condition in a subject in need thereof comprising administering an effective amount of a composition comprising IMT-504 disclosed herein (e.g., a composition such as a pharmaceutical composition comprising IMT-504 and a monosodium or disodium salt of 5-CNAC) to the subject. Also provided is a pill or capsule comprising IMT-504 and a caprylic acid derivative disclosed herein, e.g., a monosodium or disodium salt of 5-CNAC.

The oligonucleotide sequence of IMT-504 is TCATCATTTTGTCATTTTGTCATT (SEQ ID NO: 80). In some aspects, IMT-504 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Inclisiran: In some aspects, the nucleic acid therapeutic agent is inclisiran. Inclisiran, also known as ALN-60212, is a siRNA for the treatment of hypercholesterolemia that targets PCSK9. In some aspects, the present disclosure provides a composition comprising inclisiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising inclisiran and 5-CNAC. In one specific aspect, the present disclosure provides a composition comprising inclisiran and 5-CNAC disodium salt. In some aspects, the composition is formulated for oral delivery.

The present disclosure also provides a method to treat or prevent a disease or condition in a subject in need thereof comprising administering an effective amount of a composition comprising inclisiran disclosed herein (e.g., a composition such as a pharmaceutical composition comprising inclisiran and a monosodium or disodium salt of 5-CNAC) to the subject. Also provided is a pill or capsule comprising inclisiran and a caprylic acid derivative disclosed herein, e.g., a monosodium or disodium salt of 5-CNAC.

The oligonucleotide sequence of inclisiran is an RNA duplex comprising an antisense strand of sequence CUAGACCUGUTUUGCUUUUGUN (SEQ ID NO: 81) and a sense strand of sequence ACAAAAGCAAAACAGGUCUAGAA (SEQ ID NO: 82). In some aspects, inclisiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Inotersen: In some aspects, the nucleic acid therapeutic agent is inotersen (TEGSEDI™). Inotersen is an antisense oligonucleotide for the treatment of hereditary transthyretin-mediated amyloidosis or polyneuropathy that targets TTR. In some aspects, the present disclosure provides a composition comprising inotersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising inotersen and 5-CNAC. In one specific aspect, the present disclosure provides a composition comprising inotersen and 5-CNAC disodium salt. In some aspects, the composition is formulated for oral delivery.

The present disclosure also provides a method to treat or prevent a disease or condition in a subject in need thereof comprising administering an effective amount of a composition comprising inotersen disclosed herein (e.g., a composition such as a pharmaceutical composition comprising inotersen and a monosodium or disodium salt of 5-CNAC) to the subject. Also provided is a pill or capsule comprising inotersen and a caprylic acid derivative disclosed herein, e.g., a monosodium or disodium salt of 5-CNAC.

The oligonucleotide sequence of inotersen is UCUUGGTTACATGAAAUCCC (SEQ ID NO: 83). In some aspects, inotersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Imetelstat: In some aspects, the nucleic acid therapeutic agent is imetelstat. Imetelstat is a telomerase inhibitor oligonucleotide for the treatment of myelodysplastic syndromes, myelofibrosis, multiple myeloma, acute myeloid leukemia, chronic myeloid leukemia, breast cancer, essential thrombocythaemia, lymphoproliferative disorders, non-small cell lung cancer, polycythaemia vera, or solid tumors. In some aspects, the present disclosure provides a composition comprising imetelstat and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising imetelstat and 5-CNAC. In one specific aspect, the present disclosure provides a composition comprising imetelstat and 5-CNAC disodium salt. In some aspects, the composition is formulated for oral delivery.

The present disclosure also provides a method to treat or prevent a disease or condition in a subject in need thereof comprising administering an effective amount of a composition comprising imetelstat disclosed herein (e.g., a composition such as a pharmaceutical composition comprising imetelstat and a monosodium or disodium salt of 5-CNAC) to the subject. Also provided is a pill or capsule comprising imetelstat and a caprylic acid derivative disclosed herein, e.g., a monosodium or disodium salt of 5-CNAC.

The oligonucleotide sequence of imetelstat is TAGGGTTAGACAA (SEQ ID NO: 84). In some aspects, imetelstat is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-APO(a)-Rx: In some aspects, the nucleic acid therapeutic agent is IONIS-APO(a)-Rx. IONIS-APO(a)-Rx is an antisense oligonucleotide for the treatment of high lipoprotein levels that targets apolipoprotein A. In some aspects, the present disclosure provides a composition comprising IONIS-APO(a)-Rx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-APO(a)-Rx and 5-CNAC. In one specific aspect, the present disclosure provides a composition comprising IONIS-APO(a)-Rx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-APO(a)-Rx disclosed herein (e.g., a composition such as a pharmaceutical composition comprising IONIS-APO(a)-Rx and a monosodium or disodium salt of 5-CNAC) to the subject. Also provided is a pill or capsule comprising IONIS-APO(a)-Rx and a caprylic acid derivative disclosed herein, e.g., a monosodium or disodium salt of 5-CNAC. In some aspects, IONIS-APO(a)-Rx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-C9Rx: In some aspects, the nucleic acid therapeutic agent is IONIS-C9Rx. IONIS-C9Rx is an antisense oligonucleotide for the treatment of ALS that targets C9ORF72. In some aspects, the present disclosure provides a composition comprising IONIS-C9Rx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-C9Rx and 5-CNAC. In one specific aspect, the present disclosure provides a composition comprising IONIS-C9Rx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-C9Rx disclosed herein (e.g., a composition such as a pharmaceutical composition comprising IONIS-C9Rx and a monosodium or disodium salt of 5-CNAC) to the subject. Also provided is a pill or capsule comprising IONIS-C9Rx and a caprylic acid derivative disclosed herein, e.g., a monosodium or disodium salt of 5-CNAC. In some aspects, IONIS-C9Rx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-DNM2-2.5Rx: In some aspects, the nucleic acid therapeutic agent is IONIS-DNM2-2.5Rx. IONIS-DNM2-2.5Rx is antisense oligonucleotide for the treatment of centronuclear myopathy that targets DNM2. In some aspects, the present disclosure provides a composition comprising IONIS-DNM2-2.5Rx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-DNM2-2.5Rx and 5-CNAC. In one specific aspect, the present disclosure provides a composition comprising IONIS-DNM2-2.5Rx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-DNM2-2.5Rx disclosed herein (e.g., a composition such as a pharmaceutical composition comprising IONIS-DNM2-2.5Rx and a monosodium or disodium salt of 5-CNAC) to the subject. Also provided is a pill or capsule comprising IONIS-DNM2-2.5Rx and a caprylic acid derivative disclosed herein, e.g., a monosodium or disodium salt of 5-CNAC. In some aspects, IONIS-DNM2-2.5Rx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-FXIRx: In some aspects, the nucleic acid therapeutic agent is IONIS-FXIRx. IONIS-FXIRx is an antisense oligonucleotide for the treatment of total knee arthroplasty that targets Factor XI. In some aspects, the present disclosure provides a composition comprising IONIS-FXIRx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-FXIRx and 5-CNAC. In one specific aspect, the present disclosure provides a composition comprising IONIS-FXIRx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-FXIRx disclosed herein (e.g., a composition such as a pharmaceutical composition comprising IONIS-FXIRx and a monosodium or disodium salt of 5-CNAC) to the subject. Also provided is a pill or capsule comprising IONIS-FXIRx and a caprylic acid derivative disclosed herein, e.g., a monosodium or disodium salt of 5-CNAC. In some aspects, IONIS-FXIRx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-GCGRRx: In some aspects, the nucleic acid therapeutic agent is IONIS-GCGRRx. IONIS-GCGRRx is an antisense oligonucleotide for the treatment of type 2 diabetes that targets glucagon receptor. In some aspects, the present disclosure provides a composition comprising IONIS-GCGRRx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-GCGRRx and 5-CNAC. In one specific aspect, the present disclosure provides a composition comprising IONIS-GCGRRx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-GCGRRx disclosed herein (e.g., a composition such as a pharmaceutical composition comprising IONIS-GCGRRx and a monosodium or disodium salt of 5-CNAC) to the subject. Also provided is a pill or capsule comprising IONIS-GCGRRx and a caprylic acid derivative disclosed herein, e.g., a monosodium or disodium salt of 5-CNAC. In some aspects, IONIS-GCGRRx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-MAPTRx: In some aspects, the nucleic acid therapeutic agent is IONIS-MAPTRx. IONIS-MAPTRx is an antisense oligonucleotide for the treatment of Alzheimer disease that targets MAPT. In some aspects, the present disclosure provides a composition comprising IONIS-MAPTRx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-MAPTRx and 5-CNAC. In one specific aspect, the present disclosure provides a composition comprising IONIS-MAPTRx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-MAPTRx disclosed herein (e.g., a composition such as a pharmaceutical composition comprising IONIS-MAPTRx and a monosodium or disodium salt of 5-CNAC) to the subject. Also provided is a pill or capsule comprising IONIS-MAPTRx and a caprylic acid derivative disclosed herein, e.g., a monosodium or disodium salt of 5-CNAC. In some aspects, IONIS-MAPTRx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

IONIS-TTRRx: In some aspects, the nucleic acid therapeutic agent is IONIS-TTRRx. IONIS-TTRRx is an antisense oligonucleotide for the treatment of familial amyloid polyneuropathy (FAP) that targets transthyretin. In some aspects, the present disclosure provides a composition comprising IONIS-TTRRx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising IONIS-TTRRx and 5-CNAC. In one specific aspect, the present disclosure provides a composition comprising IONIS-TTRRx and 5-CNAC disodium salt. In some aspects, the composition is formulated for oral delivery.

The present disclosure also provides a method to treat or prevent a disease or condition in a subject in need thereof comprising administering an effective amount of a composition comprising IONIS-TTRRx disclosed herein (e.g., a composition such as a pharmaceutical composition comprising IONIS-TTRRx and a monosodium or disodium salt of 5-CNAC) to the subject. Also provided is a pill or capsule comprising IONIS-TTRRx and a caprylic acid derivative disclosed herein, e.g., a monosodium or disodium salt of 5-CNAC. In some aspects, IONIS-TTRRx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ISIS EIF4E Rx: In some aspects, the nucleic acid therapeutic agent is ISIS EIF4E Rx. ISIS EIF4E Rx is an antisense oligonucleotide for the treatment of castrate-resistant prostate cancer that targets eIF-4E. In some aspects, the present disclosure provides a composition comprising ISIS EIF4E Rx and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising ISIS EIF4E Rx and 5-CNAC. In one specific aspect, the present disclosure provides a composition comprising ISIS EIF4E Rx and 5-CNAC disodium salt. In some aspects, the composition is formulated for oral delivery.

The present disclosure also provides a method to treat or prevent a disease or condition in a subject in need thereof comprising administering an effective amount of a composition comprising ISIS EIF4E Rx disclosed herein (e.g., a composition such as a pharmaceutical composition comprising ISIS EIF4E Rx and a monosodium or disodium salt of 5-CNAC) to the subject. Also provided is a pill or capsule comprising ISIS EIF4E Rx and a caprylic acid derivative disclosed herein, e.g., a monosodium or disodium salt of 5-CNAC. In some aspects, ISIS EIF4E Rx is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ISIS-104838, ISIS-1082, ISIS-2503, ISIS-333611, ISIS-113715, ISIS-426115, ISIS-449884, ISIS-463588, ISIS-5132, ISIS-702843, and ISIS-757456: In some aspects, the nucleic acid therapeutic agent is ISIS-104838, ISIS-1082, ISIS-2503, ISIS-333611, ISIS-113715, ISIS-426115, ISIS-449884, ISIS-463588, ISIS-5132, ISIS-702843, or ISIS-757456. In some aspects, the present disclosure provides a composition comprising ISIS-104838, ISIS-1082, ISIS-2503, ISIS-333611, ISIS-113715, ISIS-426115, ISIS-449884, ISIS-463588, ISIS-5132, ISIS-702843, or ISIS-757456 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising ISIS-104838, ISIS-1082, ISIS-2503, ISIS-333611, ISIS-113715, ISIS-426115, ISIS-449884, ISIS-463588, ISIS-5132, ISIS-702843, or ISIS-757456 and 5-CNAC. In one specific aspect, the present disclosure provides a composition comprising ISIS-104838, ISIS-1082, ISIS-2503, ISIS-333611, ISIS-113715, ISIS-426115, ISIS-449884, ISIS-463588, ISIS-5132, ISIS-702843, or ISIS-757456 and 5-CNAC disodium salt. In some aspects, the composition is formulated for oral delivery.

The present disclosure also provides a method to treat or prevent a disease or condition in a subject in need thereof comprising administering an effective amount of a composition comprising ISIS-104838, ISIS-1082, ISI-2503, ISIS-333611, ISIS-113715, ISIS-426115, ISIS-449884, ISIS-463588, ISIS-5132, ISIS-702843, or ISIS-757456 disclosed herein (e.g., a composition such as a pharmaceutical composition comprising ISIS-104838, ISIS-1082, ISI-2503, ISIS-333611, ISIS-113715, ISIS-426115, ISIS-449884, ISIS-463588, ISIS-5132, ISIS-702843, or ISIS-757456 and a monosodium or disodium salt of 5-CNAC) to the subject. Also provided is a pill or capsule comprising ISIS-104838, ISIS-1082, ISI-2503, ISIS-333611, ISIS-113715, ISIS-426115, ISIS-449884, ISIS-463588, ISIS-5132, ISIS-702843, or ISIS-757456 and a caprylic acid derivative disclosed herein, e.g., a monosodium or disodium salt of 5-CNAC. In some aspects, ISIS-104838, ISIS-1082, ISI-2503, ISIS-333611, ISIS-113715, ISIS-426115, ISIS-449884, ISIS-463588, ISIS-5132, ISIS-702843, or ISIS-757456 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Lademirsen: In some aspects, the nucleic acid therapeutic agent is lademirsen. Lademirsen is an antisense oligonucleotide that targets miR-21. In some aspects, lademirsen can be used to treat Alport syndrome. In some aspects, the present disclosure provides a composition comprising lademirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising lademirsen and 5-CNAC. In one specific aspect, the present disclosure provides a composition comprising lademirsen and 5-CNAC disodium salt. In some aspects, the composition is formulated for oral delivery.

The present disclosure also provides a method to treat or prevent a disease or condition in a subject in need thereof comprising administering an effective amount of a composition comprising lademirsen disclosed herein (e.g., a composition such as a pharmaceutical composition comprising lademirsen and a monosodium or disodium salt of 5-CNAC) to the subject. Also provided is a pill or capsule comprising lademirsen and a caprylic acid derivative disclosed herein, e.g., a monosodium or disodium salt of 5-CNAC.

The oligonucleotide sequence of lademirsen is ACATCAGTCTGAUAAGCTA (SEQ ID NO: 85). In some aspects, lademirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Lexaptepid pegol: In some aspects, the nucleic acid therapeutic agent is lexaptepid pegol. Lexaptepid pegol, also known as NOX-H94, is an aptamer for the treatment of anemia, end stage renal disease, anemia of chronic disease, chronic diseases, or inflammation that targets hepcidin. In some aspects, the present disclosure provides a composition comprising lexaptepid pegol and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising lexaptepid pegol and 5-CNAC. In one specific aspect, the present disclosure provides a composition comprising lexaptepid pegol and 5-CNAC disodium salt. In some aspects, the composition is formulated for oral delivery.

The present disclosure also provides a method to treat or prevent a disease or condition in a subject in need thereof comprising administering an effective amount of a composition comprising lexaptepid pegol disclosed herein (e.g., a composition such as a pharmaceutical composition comprising lexaptepid pegol and a monosodium or disodium salt of 5-CNAC) to the subject. Also provided is a pill or capsule comprising lexaptepid pegol and a caprylic acid derivative disclosed herein, e.g., a monosodium or disodium salt of 5-CNAC.

The oligonucleotide sequence of lexaptepid pegol is GCGCCGUAUGGGAUUAAGUAAAUGAGGAGUUGGAGGAAGGGCGC (SEQ ID NO: 86). In some aspects, lexaptepid is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

Litenimod: In some aspects, the nucleic acid therapeutic agent is litenimod. Litenimod is a 26-mer modified oligodeoxynucleotides (ODN) that functions as a TLR9 agonist. In some aspects, the present disclosure provides a composition comprising litenimod and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising litenimod and 5-CNAC. In one specific aspect, the present disclosure provides a composition comprising litenimod and 5-CNAC disodium salt. In some aspects, the composition is formulated for oral delivery.

The present disclosure also provides a method to treat or prevent a disease or condition in a subject in need thereof comprising administering an effective amount of a composition comprising litenimod disclosed herein (e.g., a composition such as a pharmaceutical composition comprising litenimod and a monosodium or disodium salt of 5-CNAC) to the subject. Also provided is a pill or capsule comprising litenimod and a caprylic acid derivative disclosed herein, e.g., a monosodium or disodium salt of 5-CNAC.

The oligonucleotide sequence of litenimod is TAAACGTTATAACGTTATGACGTCAT (SEQ ID NO: 87). In some aspects, liternimod is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Lumasiran: In some aspects, the nucleic acid therapeutic agent is lumasiran. Lumasiran is a siRNA for the treatment of primary hyperoxaluria type 1 that targets HAO1. In some aspects, the present disclosure provides a composition comprising lumasiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising lumasiran and 5-CNAC. In one specific aspect, the present disclosure provides a composition comprising lumasiran and 5-CNAC disodium salt. In some aspects, the composition is formulated for oral delivery.

The present disclosure also provides a method to treat or prevent a disease or condition in a subject in need thereof comprising administering an effective amount of a composition comprising lumasiran disclosed herein (e.g., a composition such as a pharmaceutical composition comprising lumasiran and a monosodium or disodium salt of 5-CNAC) to the subject. Also provided is a pill or capsule comprising lumasiran and a caprylic acid derivative disclosed herein, e.g., a monosodium or disodium salt of 5-CNAC.

The oligonucleotide sequence of lumasiran is a double stranded RNA (dsRNA) comprising an antisense strand of sequence GACUUUCAUCCUGGAAAUAUA (SEQ ID NO: 88) and a sense strand of sequence UAUAUUUCCAGGAUGAAAGUCCA (SEQ ID NO: 89). In some aspects, lumasiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Mipomersen: In some aspects, the nucleic acid therapeutic agent is mipomersen (KYNAMRO™). Mipomersen is antisense oligonucleotide for the treatment of familial hypercholesterolemia that targets APOB. In some aspects, the present disclosure provides a composition comprising mipomersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising mipomersen and 5-CNAC. In one specific aspect, the present disclosure provides a composition comprising mipomersen and 5-CNAC disodium salt. In some aspects, the composition is formulated for oral delivery.

The present disclosure also provides a method to treat or prevent a disease or condition in a subject in need thereof comprising administering an effective amount of a composition comprising mipomersen disclosed herein (e.g., a composition such as a pharmaceutical composition comprising mipomersen and a monosodium or disodium salt of 5-CNAC) to the subject. Also provided is a pill or capsule comprising mipomersen and a caprylic acid derivative disclosed herein, e.g., a monosodium or disodium salt of 5-CNAC.

The oligonucleotide sequence of mipomersen is GCCUCAGTCTGCTTCGCACC (SEQ ID NO: 90). In some aspects, mipomersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Miravirsen: In some aspects, the nucleic acid therapeutic agent is miravirsen. Miravirsen, also known as SPC3649, is an antisense oligonucleotide for the treatment of chronic hepatitis C (CHC) infection that targets miRNA-122. In some aspects, the present disclosure provides a composition comprising miravirsen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising miravirsen and 5-CNAC. In one specific aspect, the present disclosure provides a composition comprising miravirsen and 5-CNAC disodium salt. In some aspects, the composition is formulated for oral delivery.

The present disclosure also provides a method to treat or prevent a disease or condition in a subject in need thereof comprising administering an effective amount of a composition comprising miravirsen disclosed herein (e.g., a composition such as a pharmaceutical composition comprising miravirsen and a monosodium or disodium salt of 5-CNAC) to the subject. Also provided is a pill or capsule comprising miravirsen and a caprylic acid derivative disclosed herein, e.g., a monosodium or disodium salt of 5-CNAC.

The oligonucleotide sequence of miravirsen is CCATTGTCACACTCC (SEQ ID NO: 91). In some aspects, mirasvirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Mongersen: In some aspects, the nucleic acid therapeutic agent is mongersen. Mongersen, also known as GED-0301, is an antisense oligonucleotide for the treatment of Crohn's disease that targets SMAD7. In some aspects, the present disclosure provides a composition comprising mongersen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising mongersen and 5-CNAC. In one specific aspect, the present disclosure provides a composition comprising mongersen and 5-CNAC disodium salt. In some aspects, the composition is formulated for oral delivery.

The present disclosure also provides a method to treat or prevent a disease or condition in a subject in need thereof comprising administering an effective amount of a composition comprising mongersen disclosed herein (e.g., a composition such as a pharmaceutical composition comprising mongersen and a monosodium or disodium salt of 5-CNAC) to the subject. Also provided is a pill or capsule comprising mongersen and a caprylic acid derivative disclosed herein, e.g., a monosodium or disodium salt of 5-CNAC.

The oligonucleotide sequence of mongersen is GTCGCCCCTTCTCCCCGCAGC (SEQ ID NO: 92). In some aspects, mongersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

MTL-CEBPA: In some aspects, the nucleic acid therapeutic agent is MTL-CEBPA. MTL-CEBPA is a small activating RNA (saRNA) designed to reduce immune suppression of myeloid cells by restoring C/EBP-α protein to normal levels using the RNA activation mechanism. MTL-CEBPA can be used for the treatment of liver cancer, solid tumors, colorectal cancer, hepatocellular carcinoma, or liver disorders. In some aspects, the present disclosure provides a composition comprising MTL-CEBPA and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising MTL-CEBPA and 5-CNAC. In one specific aspect, the present disclosure provides a composition comprising MTL-CEBPA and 5-CNAC disodium salt. In some aspects, the composition is formulated for oral delivery.

The present disclosure also provides a method to treat or prevent a disease or condition in a subject in need thereof comprising administering an effective amount of a composition comprising MTL-CEBPA disclosed herein (e.g., a composition such as a pharmaceutical composition comprising MTL-CEBPA and a monosodium or disodium salt of 5-CNAC) to the subject. Also provided is a pill or capsule comprising MTL-CEBPA and a caprylic acid derivative disclosed herein, e.g., a monosodium or disodium salt of 5-CNAC. See, e.g., Setten et al. (2018) “Development of MTL-CEBPA: Small Activating RNA Drug for Hepatocellular Carcinoma” Curr. Pharm. Biotechnol. 19(8):611-621. In some aspects, MTL-CEBPA is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

ND-L02-s0201: In some aspects, the nucleic acid therapeutic agent is ND-L02-s0201. ND-L02-s0201, also known as BMS-986263, is a siRNA for the treatment of extensive hepatic fibrosis that targets HSP47. In some aspects, the present disclosure provides a composition comprising ND-L02-s0201 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising ND-L02-s0201 and 5-CNAC. In one specific aspect, the present disclosure provides a composition comprising ND-L02-s0201 and 5-CNAC disodium salt. In some aspects, the composition is formulated for oral delivery.

The present disclosure also provides a method to treat or prevent a disease or condition in a subject in need thereof comprising administering an effective amount of a composition comprising ND-L02-s0201 disclosed herein (e.g., a composition such as a pharmaceutical composition comprising ND-L02-s0201 and a monosodium or disodium salt of 5-CNAC) to the subject. Also provided is a pill or capsule comprising ND-L02-s0201 and a caprylic acid derivative disclosed herein, e.g., a monosodium or disodium salt of 5-CNAC. In some aspects, ND-L02-s0201 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Nedosiran: In some aspects, the nucleic acid therapeutic agent is nedosiran. Nedosiran is a siRNA for the treatment of primary hyperoxaluria that targets LDHA. In some aspects, the present disclosure provides a composition comprising nedosiran and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising nedosiran and 5-CNAC. In one specific aspect, the present disclosure provides a composition comprising nedosiran and 5-CNAC disodium salt. In some aspects, the composition is formulated for oral delivery.

The present disclosure also provides a method to treat or prevent a disease or condition in a subject in need thereof comprising administering an effective amount of a composition comprising nedosiran disclosed herein (e.g., a composition such as a pharmaceutical composition comprising nedosiran and a monosodium or disodium salt of 5-CNAC) to the subject. Also provided is a pill or capsule comprising nedosiran and a caprylic acid derivative disclosed herein, e.g., a monosodium or disodium salt of 5-CNAC.

The oligonucleotide sequence of nedosiran is a double stranded RNA (dsRNA) comprising an antisense strand of sequence UCAGAUAAAAAGGACAACAUGG (SEQ ID NO: 93) and a sense strand of sequence AUGUUGUCCUUUUUAUCUGAGCAGCCGAAAGGCUGC (SEQ ID NO: 94). In some aspects, nedosiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Nusinersen: In some aspects, the nucleic acid therapeutic agent is nusinersen (SPINRAZA™). Nusinersen is an antisense oligonucleotide (splice modulator) for the treatment of infantile-onset spinal muscular atrophy that targets exon 7 of the Survival of Motor Neuron 2 (SMN2) splicing modulator. In some aspects, the present disclosure provides a composition comprising nusinersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising nusinersen and 5-CNAC. In one specific aspect, the present disclosure provides a composition comprising nusinersen and 5-CNAC disodium salt. In some aspects, the composition is formulated for oral delivery.

The present disclosure also provides a method to treat or prevent a disease or condition in a subject in need thereof comprising administering an effective amount of a composition comprising nusinersen disclosed herein (e.g., a composition such as a pharmaceutical composition comprising nusinersen and a monosodium or disodium salt of 5-CNAC) to the subject. Also provided is a pill or capsule comprising nusinersen and a caprylic acid derivative disclosed herein, e.g., a monosodium or disodium salt of 5-CNAC.

The oligonucleotide sequence of nusinersen is TCACCTTTATAATGCTGG (SEQ ID NO: 95). In some aspects, nusinersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Oblimersen: In some aspects, the nucleic acid therapeutic agent is oblimersen (GENASENSE™). Oblimersen is an antisense oligonucleotide for the treatment of melanoma
that targets Bcl-2. In some aspects, the present disclosure provides a composition comprising oblimersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising oblimersen and 5-CNAC. In one specific aspect, the present disclosure provides a composition comprising oblimersen and 5-CNAC disodium salt. In some aspects, the composition is formulated for oral delivery.

The present disclosure also provides a method to treat or prevent a disease or condition in a subject in need thereof comprising administering an effective amount of a composition comprising oblimersen disclosed herein (e.g., a composition such as a pharmaceutical composition comprising oblimersen and a monosodium or disodium salt of 5-CNAC) to the subject. Also provided is a pill or capsule comprising oblimersen and a caprylic acid derivative disclosed herein, e.g., a monosodium or disodium salt of 5-CNAC.

The oligonucleotide sequence of oblimersen is TCTCCCAGCGTGCGCCAT (SEQ ID NO: 96). In some aspects, oblimersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Olaptesed pegol: In some aspects, the nucleic acid therapeutic agent is olaptesed pegol. Olaptesed pegol, also known as NOX-A12, is an aptamer for the treatment of chronic lymphocytic leukemia, multiple myeloma, hematopoietic stem cell transplantation, autologous stem cell transplantation, glioblastoma, metastatic colorectal cancer, or metastatic pancreatic cancer that targets CXCL12. In some aspects, the present disclosure provides a composition comprising olaptesed pegol and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising olaptesed pegol and 5-CNAC. In one specific aspect, the present disclosure provides a composition comprising olaptesed pegol and 5-CNAC disodium salt. In some aspects, the composition is formulated for oral delivery.

The present disclosure also provides a method to treat or prevent a disease or condition in a subject in need thereof comprising administering an effective amount of a composition comprising olaptesed pegol disclosed herein (e.g., a composition such as a pharmaceutical composition comprising olaptesed pegol and a monosodium or disodium salt of 5-CNAC) to the subject. Also provided is a pill or capsule comprising olaptesed pegol and a caprylic acid derivative disclosed herein, e.g., a monosodium or disodium salt of 5-CNAC.

The oligonucleotide sequence of olaptesed pegol is GCGUGGUGUGAUCUAGAUGUAUUGGCUGAUCCUAGUCAGGUACGC (SEQ ID NO: 97). In some aspects, olaptesed is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

Olpasiran: In some aspects, the nucleic acid therapeutic agent is olpasiran. Olpasiran is a siRNA for the treatment of cardiovascular disorders that targets lipoprotein(a) (Lp(a). In some aspects, the present disclosure provides a composition comprising olpasiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising olpasiran and 5-CNAC. In one specific aspect, the present disclosure provides a composition comprising olpasiran and 5-CNAC disodium salt. In some aspects, the composition is formulated for oral delivery.

The present disclosure also provides a method to treat or prevent a disease or condition in a subject in need thereof comprising administering an effective amount of a composition comprising olpasiran disclosed herein (e.g., a composition such as a pharmaceutical composition comprising olpasiran and a monosodium or disodium salt of 5-CNAC) to the subject. Also provided is a pill or capsule comprising olpasiran and a caprylic acid derivative disclosed herein, e.g., a monosodium or disodium salt of 5-CNAC.

The oligonucleotide sequence of olpasiran is a double stranded RNA (dsRNA) comprising an antisense strand of sequence CAGCCCCUUAUUGUUAUACGA (SEQ ID NO: 98) and a sense strand of sequence UCGUAUAACAAUAAGGGGCUG (SEQ ID NO: 99). In some aspects, olpasiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Patisiran: In some aspects, the nucleic acid therapeutic agent is patisiran (ONPATTRO™). Patisiran is a siRNA for the treatment of hereditary transthyretin-mediated amyloidosis and neuropathy that targets transthyretin. In some aspects, the present disclosure provides a composition comprising patisiran and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising patisiran and 5-CNAC. In one specific aspect, the present disclosure provides a composition comprising patisiran and 5-CNAC disodium salt. In some aspects, the composition is formulated for oral delivery.

The present disclosure also provides a method to treat or prevent a disease or condition in a subject in need thereof comprising administering an effective amount of a composition comprising patisiran disclosed herein (e.g., a composition such as a pharmaceutical composition comprising patisiran and a monosodium or disodium salt of 5-CNAC) to the subject. Also provided is a pill or capsule comprising patisiran and a caprylic acid derivative disclosed herein, e.g., a monosodium or disodium salt of 5-CNAC.

The oligonucleotide sequence of patisiran is a double stranded RNA (dsRNA) comprising an antisense strand of sequence GUAACCAAGAGUAUUCCAUTT (SEQ ID NO: 100) and a sense strand of sequence AUGGAAUACUCUUGGUUACTT (SEQ ID NO: 101). In some aspects, patisiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Pegaptanib: In some aspects, the nucleic acid therapeutic agent is pegaptanib (MACUGEN™). Pegaptanib is an aptamer for the treatment of wet macular degeneration neovascular age-related macular degeneration that targets VEGF. In some aspects, the present disclosure provides a composition comprising pegaptanib and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising pegaptanib and 5-CNAC. In one specific aspect, the present disclosure provides a composition comprising pegaptanib and 5-CNAC disodium salt. In some aspects, the composition is formulated for oral delivery.

The present disclosure also provides a method to treat or prevent a disease or condition in a subject in need thereof comprising administering an effective amount of a composition comprising pegaptanib disclosed herein (e.g., a composition such as a pharmaceutical composition comprising pegaptanib and a monosodium or disodium salt of 5-CNAC) to the subject. Also provided is a pill or capsule comprising pegaptanib and a caprylic acid derivative disclosed herein, e.g., a monosodium or disodium salt of 5-CNAC.

The oligonucleotide sequence of pegaptanib is CGGAAUCAGUGAAUGCUUAUACAUCCGT (SEQ ID NO: 102). In some aspects, pegaptanib is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

Pegpleranib: In some aspects, the nucleic acid therapeutic agent is pegpleranib (FOVISTA™). Pegpleranib is an aptamer for the treatment of subfoveal neovascular age-related macular degeneration that targets PDGF-B. In some aspects, the present disclosure provides a composition comprising pegpleranib and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising pegpleranib and 5-CNAC. In one specific aspect, the present disclosure provides a composition comprising pegpleranib and 5-CNAC disodium salt. In some aspects, the composition is formulated for oral delivery.

The present disclosure also provides a method to treat or prevent a disease or condition in a subject in need thereof comprising administering an effective amount of a composition comprising pegpleranib disclosed herein (e.g., a composition such as a pharmaceutical composition comprising pegpleranib and a monosodium or disodium salt of 5-CNAC) to the subject. Also provided is a pill or capsule comprising pegpleranib and a caprylic acid derivative disclosed herein, e.g., a monosodium or disodium salt of 5-CNAC.

The oligonucleotide sequence of pegpleranib is CAGGCUACGCGTAGAGCAUCATGATCCUGT (SEQ ID NO: 103). In some aspects, pegpleranib is a non-conjugated form, i.e., the oligonucleotide is not conjugated to a delivery moiety such as GalNAc or PEG. In some aspects, the non-conjugated form (e.g., without GalNAc) is formulated with 5-CNAC, e.g., as an oral capsule.

Pelacarsen: In some aspects, the nucleic acid therapeutic agent is pelacarsen. Pelacarsen, also known as IONIC-APO(a)-LRX and ISIS-681257, is an antisense oligonucleotide form the treatment of hyperlipoproteinemia that targets apolipoprotein A. In some aspects, the present disclosure provides a composition comprising pelacarsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising pelacarsen and 5-CNAC. In one specific aspect, the present disclosure provides a composition comprising pelacarsen and 5-CNAC disodium salt. In some aspects, the composition is formulated for oral delivery.

The present disclosure also provides a method to treat or prevent a disease or condition in a subject in need thereof comprising administering an effective amount of a composition comprising pelacarsen disclosed herein (e.g., a composition such as a pharmaceutical composition comprising pelacarsen and a monosodium or disodium salt of 5-CNAC) to the subject. Also provided is a pill or capsule comprising pelacarsen and a caprylic acid derivative disclosed herein, e.g., a monosodium or disodium salt of 5-CNAC.

The oligonucleotide sequence of pelacarsen is TGCTCCGTTGGTGCTTGTTC (SEQ ID NO: 104). In some aspects, pelacarsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Prexigebersen: In some aspects, the nucleic acid therapeutic agent is prexigebersen. Prexigebersen is an antisense oligonucleotide for the treatment of acute myeloid leukemia, myelodysplastic syndromes, chronic myeloid leukemia, precursor cell lymphoblastic leukemia-lymphoma, colorectal cancer, head and neck cancer, lymphoma, solid tumors, thyroid cancer, or breast cancer that targets GRB2. In some aspects, the present disclosure provides a composition comprising prexigebersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising prexigebersen and 5-CNAC. In one specific aspect, the present disclosure provides a composition comprising prexigebersen and 5-CNAC disodium salt. In some aspects, the composition is formulated for oral delivery.

The present disclosure also provides a method to treat or prevent a disease or condition in a subject in need thereof comprising administering an effective amount of a composition comprising prexigebersen disclosed herein (e.g., a composition such as a pharmaceutical composition comprising prexigebersen and a monosodium or disodium salt of 5-CNAC) to the subject. Also provided is a pill or capsule comprising prexigebersen and a caprylic acid derivative disclosed herein, e.g., a monosodium or disodium salt of 5-CNAC.

The oligonucleotide sequence of prexigebersen is ATATTTGGCGATGGCTTC (SEQ ID NO: 105). In some aspects, prexigebersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Radavirsen: In some aspects, the nucleic acid therapeutic agent is radavirsen. Radavirsen, also known as AVI-7100, is an antisense oligonucleotide for the treatment of influenza A virus infections. In some aspects, the present disclosure provides a composition comprising radavirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising radavirsen and 5-CNAC. In one specific aspect, the present disclosure provides a composition comprising radavirsen and 5-CNAC disodium salt. In some aspects, the composition is formulated for oral delivery.

The present disclosure also provides a method to treat or prevent a disease or condition in a subject in need thereof comprising administering an effective amount of a composition comprising radavirsen disclosed herein (e.g., a composition such as a pharmaceutical composition comprising radavirsen and a monosodium or disodium salt of 5-CNAC) to the subject. Also provided is a pill or capsule comprising radavirsen and a caprylic acid derivative disclosed herein, e.g., a monosodium or disodium salt of 5-CNAC.

The oligonucleotide sequence of radavirsen is CTCCAACATCAAGGAAGATGGCATTTCTAG (SEQ ID NO: 106). In some aspects, radavirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Remlarsen: In some aspects, the nucleic acid therapeutic agent is remlarsen. Remlarsen is a miRNA mimic for the treatment of cutaneous fibrosis. Remlarsen mimics miR-29. In some aspects, the present disclosure provides a composition comprising remlarsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising remlarsen and 5-CNAC. In one specific aspect, the present disclosure provides a composition comprising remlarsen and 5-CNAC disodium salt. In some aspects, the composition is formulated for oral delivery.

The present disclosure also provides a method to treat or prevent a disease or condition in a subject in need thereof comprising administering an effective amount of a composition comprising remlarsen disclosed herein (e.g., a composition such as a pharmaceutical composition comprising remlarsen and a monosodium or disodium salt of 5-CNAC) to the subject. Also provided is a pill or capsule comprising remlarsen and a caprylic acid derivative disclosed herein, e.g., a monosodium or disodium salt of 5-CNAC.

The oligonucleotide sequence of remlarsen is a double stranded RNA (dsRNA) comprising an antisense strand of sequence UAGCACCAUUUGAAAUCAGUGUUUU (SEQ ID NO: 107) and a sense strand of sequence AACACUGUUUACAAAUGGUCCUA (SEQ ID NO: 108). In some aspects, remlarsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Renadirsen: In some aspects, the nucleic acid therapeutic agent is renadirsen. Renadirsen, also known as renapersen, is an antisense oligonucleotide for the treatment of Duchenne muscular dystrophy that functions by stimulating the expression of dystrophin. In some aspects, the present disclosure provides a composition comprising renadirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising renadirsen and 5-CNAC. In one specific aspect, the present disclosure provides a composition comprising renadirsen and 5-CNAC disodium salt. In some aspects, the composition is formulated for oral delivery.

The present disclosure also provides a method to treat or prevent a disease or condition in a subject in need thereof comprising administering an effective amount of a composition comprising renadirsen disclosed herein (e.g., a composition such as a pharmaceutical composition comprising renadirsen and a monosodium or disodium salt of 5-CNAC) to the subject. Also provided is a pill or capsule comprising renadirsen and a caprylic acid derivative disclosed herein, e.g., a monosodium or disodium salt of 5-CNAC.

The oligonucleotide sequence of renadirsen is CCUACCGUAACCCGUCGC (SEQ ID NO: 109). In some aspects, renadirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Resten-MP™: In some aspects, the nucleic acid therapeutic agent is Resten-MP™ Resten-MP™, also known as AVI-4126, is an antisense oligonucleotide for the treatment of de novo native coronary artery lesions that targets c-myc In some aspects, the present disclosure provides a composition comprising Resten-MP and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising Resten-MP and 5-CNAC. In one specific aspect, the present disclosure provides a composition comprising Resten-MP and 5-CNAC disodium salt. In some aspects, the composition is formulated for oral delivery.

The present disclosure also provides a method to treat or prevent a disease or condition in a subject in need thereof comprising administering an effective amount of a composition comprising Resten-MP disclosed herein (e.g., a composition such as a pharmaceutical composition comprising Resten-MP and a monosodium or disodium salt of 5-CNAC) to the subject. Also provided is a pill or capsule comprising Resten-MP and a caprylic acid derivative disclosed herein, e.g., a monosodium or disodium salt of 5-CNAC.

The oligonucleotide sequence of AVI-4126 is ACGTTGAGGGGCATCGTCGC (SEQ ID NO: 110). In some aspects, AVI-4126 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Revusiran: In some aspects, the nucleic acid therapeutic agent is revusiran. Revusiran, also known as AD-51547, is a siRNA for the treatment of hereditary amyloidosis that targets TTR. In some aspects, the present disclosure provides a composition comprising revusiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising revusiran and 5-CNAC. In one specific aspect, the present disclosure provides a composition comprising revusiran and 5-CNAC disodium salt. In some aspects, the composition is formulated for oral delivery.

The present disclosure also provides a method to treat or prevent a disease or condition in a subject in need thereof comprising administering an effective amount of a composition comprising revusiran disclosed herein (e.g., a composition such as a pharmaceutical composition comprising revusiran and a monosodium or disodium salt of 5-CNAC) to the subject. Also provided is a pill or capsule comprising revusiran and a caprylic acid derivative disclosed herein, e.g., a monosodium or disodium salt of 5-CNAC.

The oligonucleotide sequence of revusiran is a double stranded RNA (dsRNA)_comprising an antisense strand of sequence UGGGAUUUCAUGUAACCAAGA (SEQ ID NO: 111) and a sense strand of sequence UCUUGGUUACAUGAAAUCCCAUC (SEQ ID NO: 112). In some aspects, revusiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

RG-012: In some aspects, the nucleic acid therapeutic agent is RG-012. RG-012 is an antisense oligonucleotide antimir for the treatment of Aport syndrome that target miR-21. In some aspects, the present disclosure provides a composition comprising RG-012 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising RG-012 and 5-CNAC. In one specific aspect, the present disclosure provides a composition comprising RG-012 and 5-CNAC disodium salt. In some aspects, the composition is formulated for oral delivery.

The present disclosure also provides a method to treat or prevent a disease or condition in a subject in need thereof comprising administering an effective amount of a composition comprising RG-012 disclosed herein (e.g., a composition such as a pharmaceutical composition comprising RG-012 and a monosodium or disodium salt of 5-CNAC) to the subject. Also provided is a pill or capsule comprising RG-012 and a caprylic acid derivative disclosed herein, e.g., a monosodium or disodium salt of 5-CNAC. In some aspects, RG-012 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

RGLS-4326: In some aspects, the nucleic acid therapeutic agent is RGLS-4326. RGLS-4326 is antisense oligonucleotide antimir for the treatment of autosomal dominant polycystic kidney disease that targets miR-17. In some aspects, the present disclosure provides a composition comprising RGLS 4326 and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising RGLS-4326 and 5-CNAC. In one specific aspect, the present disclosure provides a composition comprising RGLS-4326 and 5-CNAC disodium salt. In some aspects, the composition is formulated for oral delivery.

The present disclosure also provides a method to treat or prevent a disease or condition in a subject in need thereof comprising administering an effective amount of a composition comprising RGLS-4326 disclosed herein (e.g., a composition such as a pharmaceutical composition comprising RGLS-4326 and a monosodium or disodium salt of 5-CNAC) to the subject. Also provided is a pill or capsule comprising RGLS-4326 and a caprylic acid derivative disclosed herein, e.g., a monosodium or disodium salt of 5-CNAC.

The oligonucleotide sequence of RGLS-4326 is AGCACUUUG (SEQ ID NO: 113). In some aspects, RGLS-4326 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Rimigorsen: In some aspects, the nucleic acid therapeutic agent is rimigorsen. Rimigorsen is an antisense oligonucleotide for the treatment of Duchenne muscular dystrophy that promotes the synthesis of functional dystrophin. In some aspects, the present disclosure provides a composition comprising rimigorsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising rimigorsen and 5-CNAC. In one specific aspect, the present disclosure provides a composition comprising rimigorsen and 5-CNAC disodium salt. In some aspects, the composition is formulated for oral delivery.

The present disclosure also provides a method to treat or prevent a disease or condition in a subject in need thereof comprising administering an effective amount of a composition comprising rimigorsen disclosed herein (e.g., a composition such as a pharmaceutical composition comprising rimigorsen and a monosodium or disodium salt of 5-CNAC) to the subject. Also provided is a pill or capsule comprising rimigorsen and a caprylic acid derivative disclosed herein, e.g., a monosodium or disodium salt of 5-CNAC.

The oligonucleotide sequence of rimigorsen is UCAGCUUCUGUUAGCCACUG (SEQ ID NO: 114). In some aspects, rimigorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Rosomidnar: In some aspects, the nucleic acid therapeutic agent is rosomidnar. Rosomidnar, also known as PNT-100, is an oligonucleotide inhibitor of apoptosis regulator Bcl2 that can be used for the treatment of diffuse large B cell lymphoma, Richter's syndrome, non-Hodgkin's lymphoma, or solid tumors. In some aspects, the present disclosure provides a composition comprising rosomidnar and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising rosomidnar and 5-CNAC. In one specific aspect, the present disclosure provides a composition comprising rosomidnar and 5-CNAC disodium salt. In some aspects, the composition is formulated for oral delivery.

The present disclosure also provides a method to treat or prevent a disease or condition in a subject in need thereof comprising administering an effective amount of a composition comprising rosomidnar disclosed herein (e.g., a composition such as a pharmaceutical composition comprising rosomidnar and a monosodium or disodium salt of 5-CNAC) to the subject. Also provided is a pill or capsule comprising rosomidnar and a caprylic acid derivative disclosed herein, e.g., a monosodium or disodium salt of 5-CNAC.

The oligonucleotide sequence of rosomidnar is CACGCACGCGCATCCCCGCCCGTG (SEQ ID NO: 115). In some aspects, rosomidnar is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

SB010: In some aspects, the nucleic acid therapeutic agent is SB010. SB010 is an antisense oligonucleotide for the treatment of mild allergic asthma that targets GATA-3. In some aspects, the present disclosure provides a composition comprising SB010 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising Sf010 and 5-CNAC. In one specific aspect, the present disclosure provides a composition comprising Sf010 and 5-CNAC disodium salt. In some aspects, the composition is formulated for oral delivery.

The present disclosure also provides a method to treat or prevent a disease or condition in a subject in need thereof comprising administering an effective amount of a composition comprising SB010 disclosed herein (e.g., a composition such as a pharmaceutical composition comprising SB010 and a monosodium or disodium salt of 5-CNAC) to the subject. Also provided is a pill or capsule comprising SB010 and a caprylic acid derivative disclosed herein, e.g., a monosodium or disodium salt of 5-CNAC. See, e.g., clinicaltrials.gov/ct2/show/NCT01743768. In some aspects, SB-10 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

SLN124: In some aspects, the nucleic acid therapeutic agent is SLN124. SLN124 is a siRNA for the treatment of 0-thalassemia that targets TMPRSS6. In some aspects, the present disclosure provides a composition comprising SLN124 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising SLN124 and 5-CNAC. In one specific aspect, the present disclosure provides a composition comprising SLN124 and 5-CNAC disodium salt. In some aspects, the composition is formulated for oral delivery.

The present disclosure also provides a method to treat or prevent a disease or condition in a subject in need thereof comprising administering an effective amount of a composition comprising SLN124 disclosed herein (e.g., a composition such as a pharmaceutical composition comprising SLN124 and a monosodium or disodium salt of 5-CNAC) to the subject. Also provided is a pill or capsule comprising SLN124 and a caprylic acid derivative disclosed herein, e.g., a monosodium or disodium salt of 5-CNAC. See Altamura et al. (2019) “SLN124, a GalNAc-siRNA Conjugate Targeting TMPRSS6, Efficiently Prevents Iron Overload in Hereditary Haemochromatosis Type 1” Hemanshere 3(6):e301. In some aspects, SLN124 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

SRP-5051: In some aspects, the nucleic acid therapeutic agent is SRP-5051. SRP-5051 is a PPMO antisense oligonucleotide for the treatment of Duchenne muscular dystrophy that target DMD exon 51. SRP-5051 is next generation eteplirsen, in that it targets the same population, those amenable to exon 51 skipping, but the compound is “charged”, meaning that its cell-penetrating capacity is increased. In some aspects, the present disclosure provides a composition comprising SRP-5051 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising SRP-5051 and 5-CNAC. In one specific aspect, the present disclosure provides a composition comprising SRP-5051 and 5-CNAC disodium salt. In some aspects, the composition is formulated for oral delivery.

The present disclosure also provides a method to treat or prevent a disease or condition in a subject in need thereof comprising administering an effective amount of a composition comprising SRP-5051 disclosed herein (e.g., a composition such as a pharmaceutical composition comprising SRP-5051 and a monosodium or disodium salt of 5-CNAC) to the subject. Also provided is a pill or capsule comprising SRP-5051 and a caprylic acid derivative disclosed herein, e.g., a monosodium or disodium salt of 5-CNAC. In some aspects, SRP-5051 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Suvodirsen: In some aspects, the nucleic acid therapeutic agent is suvodirsen. Suvodirsen, also known as WVE-210201, is an antisense oligonucleotide for the treatment of Duchenne muscular dystrophy that targets DMD exon 51. In some aspects, the present disclosure provides a composition comprising suvodirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising suvodirsen and 5-CNAC. In one specific aspect, the present disclosure provides a composition comprising suvodirsen and 5-CNAC disodium salt. In some aspects, the composition is formulated for oral delivery.

The present disclosure also provides a method to treat or prevent a disease or condition in a subject in need thereof comprising administering an effective amount of a composition comprising suvodirsen disclosed herein (e.g., a composition such as a pharmaceutical composition comprising suvodirsen and a monosodium or disodium salt of 5-CNAC) to the subject. Also provided is a pill or capsule comprising suvodirsen and a caprylic acid derivative disclosed herein, e.g., a monosodium or disodium salt of 5-CNAC.

The oligonucleotide sequence of suvodirsen is UCAAGGAAGAUGGCAUUUCU (SEQ ID NO: 116). In some aspects, suvodirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Temavirsen: In some aspects, the nucleic acid therapeutic agent is temavirsen. Temavirsen, also known as RG-101 and RG-2459, is an antiviral antisense oligonucleotide that targets the hepatitis C virus. In some aspects, the present disclosure provides a composition comprising temavirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising temavirsen and 5-CNAC. In one specific aspect, the present disclosure provides a composition comprising temavirsen and 5-CNAC disodium salt. In some aspects, the composition is formulated for oral delivery.

The present disclosure also provides a method to treat or prevent a disease or condition in a subject in need thereof comprising administering an effective amount of a composition comprising temavirsen disclosed herein (e.g., a composition such as a pharmaceutical composition comprising temavirsen and a monosodium or disodium salt of 5-CNAC) to the subject. Also provided is a pill or capsule comprising temavirsen and a caprylic acid derivative disclosed herein, e.g., a monosodium or disodium salt of 5-CNAC.

The oligonucleotide sequence of temavirsen is ACACCAUTGUCACACTCCA (SEQ ID NO: 117). In some aspects, temavirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Teprasiran: In some aspects, the nucleic acid therapeutic agent is teprasiran. Teprasiran, also known as QPI-1002, is a siRNA inhibitor of tumor suppressor protein p53. Teprasiran can be used for the treatment of acute kidney injury or delayed graft function. In some aspects, the present disclosure provides a composition comprising teprasiran and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising teprasiran and 5-CNAC. In one specific aspect, the present disclosure provides a composition comprising teprasiran and 5-CNAC disodium salt. In some aspects, the composition is formulated for oral delivery.

The present disclosure also provides a method to treat or prevent a disease or condition in a subject in need thereof comprising administering an effective amount of a composition comprising teprasiran disclosed herein (e.g., a composition such as a pharmaceutical composition comprising teprasiran and a monosodium or disodium salt of 5-CNAC) to the subject. Also provided is a pill or capsule comprising teprasiran and a caprylic acid derivative disclosed herein, e.g., a monosodium or disodium salt of 5-CNAC.

The oligonucleotide sequence of teprasiran is a double stranded RNA (dsRNA) comprising an antisense strand of sequence UGAAGGGUGAAAUAUUCUC (SEQ ID NO: 118) and a sense strand of sequence GAGAAUAUUUCACCCUUCA (SEQ ID NO: 119). In some aspects, teprasiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Tivanisiran: In some aspects, the nucleic acid therapeutic agent is tivanisiran (SYLENTIS™). Tivanisiran, also known as SYL-1001, is a siRNA that targets the Transient Receptor Potential Vanilloid-1 (TRPV1) channel family. Tivanisiran can be used to treat ocular pain. In some aspects, the present disclosure provides a composition comprising tivanisiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising tivanisiran and 5-CNAC. In one specific aspect, the present disclosure provides a composition comprising tivanisiran and 5-CNAC disodium salt. In some aspects, the composition is formulated for oral delivery.

The present disclosure also provides a method to treat or prevent a disease or condition in a subject in need thereof comprising administering an effective amount of a composition comprising tivanisiran disclosed herein (e.g., a composition such as a pharmaceutical composition comprising tivanisiran and a monosodium or disodium salt of 5-CNAC) to the subject. Also provided is a pill or capsule comprising tivanisiran and a caprylic acid derivative disclosed herein, e.g., a monosodium or disodium salt of 5-CNAC.

The oligonucleotide sequence of tivanisiran is a double stranded RNA (dsRNA) comprising an antisense strand of sequence AAGCGCAUCUUCUACUUCA (SEQ ID NO: 120) and a sense strand of sequence UGAAGUAGAAGAUGCGCUU (SEQ ID NO: 121). In some aspects, tivanisiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Tofersen: In some aspects, the nucleic acid therapeutic agent is tofersen. Tofersen, also known as IONIS-SOD1Rx and BIIB-067, is an antisense oligonucleotide for the treatment of Amyotrophic Lateral Sclerosis (ALS) that targets SOD1. In some aspects, the present disclosure provides a composition comprising tofersen and a caprylic acid derivative disclosed herein, e.g., 5-CNAC or a derivative thereof, or any of the compounds disclosed, e.g., in FIG. 1A, FIG. 1, FIG. 2, or any combination thereof. In some aspects, the caprylic acid derivative disclosed herein is a disodium salt. In one specific aspect, the present disclosure provides a composition comprising tofersen and 5-CNAC. In one specific aspect, the present disclosure provides a composition comprising tofersen and 5-CNAC disodium salt. In some aspects, the composition is formulated for oral delivery.

The present disclosure also provides a method to treat or prevent a disease or condition in a subject in need thereof comprising administering an effective amount of a composition comprising tofersen disclosed herein (e.g., a composition such as a pharmaceutical composition comprising tofersen and a monosodium or disodium salt of 5-CNAC) to the subject. Also provided is a pill or capsule comprising tofersen and a caprylic acid derivative disclosed herein, e.g., a monosodium or disodium salt of 5-CNAC.

The oligonucleotide sequence of tofersen is CAGGATACATTTCTACAGCU (SEQ ID NO: 122). In some aspects, tofersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Tominersen: In some aspects, the nucleic acid therapeutic agent is tominersen. Tominersen, also known as IONIS-HTTRx, RG-6042 and ISIS-443139, is an antisense oligonucleotide for the treatment of Huntington's disease that targets HTT. In some aspects, the present disclosure provides a composition comprising tominersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising tominersen and 5-CNAC. In one specific aspect, the present disclosure provides a composition comprising tominersen and 5-CNAC disodium salt. In some aspects, the composition is formulated for oral delivery.

The present disclosure also provides a method to treat or prevent a disease or condition in a subject in need thereof comprising administering an effective amount of a composition comprising tominersen disclosed herein (e.g., a composition such as a pharmaceutical composition comprising tominersen and a monosodium or disodium salt of 5-CNAC) to the subject. Also provided is a pill or capsule comprising tominersen and a caprylic acid derivative disclosed herein, e.g., a monosodium or disodium salt of 5-CNAC.

The oligonucleotide sequence of tominersen is CUCAGTAACATTGACACCAC (SEQ ID NO: 123). In some aspects, tominersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Trabedersen: In some aspects, the nucleic acid therapeutic agent is trabedersen. Trabedersen, also known as AP-12009 and A-12009, is an antisense oligonucleotide which is an inhibitor of transforming growth factor beta 2. Trabedersen can be used to treat glioblastoma, malignant melanoma, pancreatic cancer, COVID 2019 infections, COVID-19 pneumonia, ovarian cancer, colorectal cancer, or anaplastic astrocytoma. In some aspects, the present disclosure provides a composition comprising trabedersen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising trabedersen and 5-CNAC. In one specific aspect, the present disclosure provides a composition comprising trabedersen and 5-CNAC disodium salt. In some aspects, the composition is formulated for oral delivery.

The present disclosure also provides a method to treat or prevent a disease or condition in a subject in need thereof comprising administering an effective amount of a composition comprising trabedersen disclosed herein (e.g., a composition such as a pharmaceutical composition comprising trabedersen and a monosodium or disodium salt of 5-CNAC) to the subject. Also provided is a pill or capsule comprising trabedersen and a caprylic acid derivative disclosed herein, e.g., a monosodium or disodium salt of 5-CNAC.

The oligonucleotide sequence of trabedersen is CGGCATGTCTATTTTGTA (SEQ ID NO: 124). In some aspects, trabedersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Trecovirsen: In some aspects, the nucleic acid therapeutic agent is trecovirsen. Trecovirsen is an antisense oligonucleotide for the treatment of AIDS that targets GAG. In some aspects, the present disclosure provides a composition comprising trecovirsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising trecovirsen and 5-CNAC. In one specific aspect, the present disclosure provides a composition comprising trecovirsen and 5-CNAC disodium salt. In some aspects, the composition is formulated for oral delivery.

The present disclosure also provides a method to treat or prevent a disease or condition in a subject in need thereof comprising administering an effective amount of a composition comprising trecovirsen disclosed herein (e.g., a composition such as a pharmaceutical composition comprising trecovirsen and a monosodium or disodium salt of 5-CNAC) to the subject. Also provided is a pill or capsule comprising trecovirsen and a caprylic acid derivative disclosed herein, e.g., a monosodium or disodium salt of 5-CNAC.

The oligonucleotide sequence of trecovirsen is TCTTCCTCTCTCTACCCACGCTCTC (SEQ ID NO: 125). In some aspects, trecovirsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Vidutolimod: In some aspects, the nucleic acid therapeutic is vidutolimod. Vidutolimod, also known as CMP-001 is an immunostimulant oligonucleotide for the treatment of malignant melanoma, head and neck cancer, lymphoma, solid tumors, squamous cell cancer, colorectal cancer, non-small cell lung cancer, allergic asthma, atopic dermatitis, hepatitis B, perennial allergic rhinitis, or seasonal allergic rhinitis. Vidutolimod is a TLR9 agonist. In some aspects, the present disclosure provides a composition comprising vidutolimod and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising vidutolimod and 5-CNAC. In one specific aspect, the present disclosure provides a composition comprising vidutolimod and 5-CNAC disodium salt. In some aspects, the composition is formulated for oral delivery.

The present disclosure also provides a method to treat or prevent a disease or condition in a subject in need thereof comprising administering an effective amount of a composition comprising vidutolimod disclosed herein (e.g., a composition such as a pharmaceutical composition comprising vidutolimod and a monosodium or disodium salt of 5-CNAC) to the subject. Also provided is a pill or capsule comprising vidutolimod and a caprylic acid derivative disclosed herein, e.g., a monosodium or disodium salt of 5-CNAC.

The oligonucleotide sequence of vidutolimod is GGGGGGGGGGGACGATCGTCGGGGGGGGGG (SEQ ID NO: 126). In some aspects, vidutolimod is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Viltolarsen: In some aspects, the nucleic acid therapeutic agent is viltolarsen (VILTEPSO™). Viltolarsen is an antisense oligonucleotide for the treatment of Duchenne's muscular dystrophy that targets DMD exon 53. In some aspects, the present disclosure provides a composition comprising viltolarsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising viltolarsen and 5-CNAC. In one specific aspect, the present disclosure provides a composition comprising viltolarsen and 5-CNAC disodium salt. In some aspects, the composition is formulated for oral delivery.

The present disclosure also provides a method to treat or prevent a disease or condition in a subject in need thereof comprising administering an effective amount of a composition comprising viltolarsen disclosed herein (e.g., a composition such as a pharmaceutical composition comprising viltolarsen and a monosodium or disodium salt of 5-CNAC) to the subject. Also provided is a pill or capsule comprising viltolarsen and a caprylic acid derivative disclosed herein, e.g., a monosodium or disodium salt of 5-CNAC.

The oligonucleotide sequence of viltolarsen is CCTCCGGTTCTGAAGGTGTTC (SEQ ID NO: 127). In some aspects, viltolarsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Volanesorsen: In some aspects, the nucleic acid therapeutic agent is volanesorsen (WAYLIVRA™). Volanesorsen, also known as ISIS-304801, is an antisense oligonucleotide for the treatment of hypertriglyceridemia, familial chylomicronemia syndrome, or familial partial lipodystrophy that targets ApoC-III. In some aspects, the present disclosure provides a composition comprising volanesorsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising volanesorsen and 5-CNAC. In one specific aspect, the present disclosure provides a composition comprising volanesorsen and 5-CNAC disodium salt. In some aspects, the composition is formulated for oral delivery.

The present disclosure also provides a method to treat or prevent a disease or condition in a subject in need thereof comprising administering an effective amount of a composition comprising volanesorsen disclosed herein (e.g., a composition such as a pharmaceutical composition comprising volanesorsen and a monosodium or disodium salt of 5-CNAC) to the subject. Also provided is a pill or capsule comprising volanesorsen and a caprylic acid derivative disclosed herein, e.g., a monosodium or disodium salt of 5-CNAC.

The oligonucleotide sequence of volanesorsen is AGCUUCTTGTCCAGCUUUAU (SEQ ID NO: 128). In some aspects, volanesorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Vupanorsen: In some aspects, the nucleic acid therapeutic agent is vupanorsen. Vupanorsen, also known as IONIS-ANGPTL3-LRx, AKCEA-ANGPTL3-LRx and ISIS-703802, is an antisense oligonucleotide conjugate (GalNAc3) for the treatment of cardiovascular disease and reduce triglyceride and cholesterol levels, that targets angiopoietin-like 3 (ANGPTL3). In some aspects, the present disclosure provides a composition comprising vupanorsen and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising vupanorsen and 5-CNAC. In one specific aspect, the present disclosure provides a composition comprising vupanorsen and 5-CNAC disodium salt. In some aspects, the composition is formulated for oral delivery.

The present disclosure also provides a method to treat or prevent a disease or condition in a subject in need thereof comprising administering an effective amount of a composition comprising vupanorsen disclosed herein (e.g., a composition such as a pharmaceutical composition comprising vupanorsen and a monosodium or disodium salt of 5-CNAC) to the subject. Also provided is a pill or capsule comprising vupanorsen and a caprylic acid derivative disclosed herein, e.g., a monosodium or disodium salt of 5-CNAC.

The oligonucleotide sequence of vupanorsen is GGACATTGCCAGTAATCGCA (SEQ ID NO: 129). In some aspects, vupanorsen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Vutrisiran: In some aspects, the nucleic acid therapeutic agent is vutrisiran. Vutrisiran, also known as ALN-TTRsc02 and ALN-65492, is a siRNA for the treatment of hereditary amyloidosis that targets TTR. In some aspects, the present disclosure provides a composition comprising vutrisiran and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising vutrisiran and 5-CNAC. In one specific aspect, the present disclosure provides a composition comprising vutrisiran and 5-CNAC disodium salt. In some aspects, the composition is formulated for oral delivery.

The present disclosure also provides a method to treat or prevent a disease or condition in a subject in need thereof comprising administering an effective amount of a composition comprising vutrisiran disclosed herein (e.g., a composition such as a pharmaceutical composition comprising vutrisiran and a monosodium or disodium salt of 5-CNAC) to the subject. Also provided is a pill or capsule comprising vutrisiran and a caprylic acid derivative disclosed herein, e.g., a monosodium or disodium salt of 5-CNAC.

The oligonucleotide sequence of vutrisiran is a double stranded RNA (dsRNA) comprising an antisense strand of sequence UGGGAUUUCAUGUAACCAAGA (SEQ ID NO: 130) and a sense strand of sequence UCUUGGUUACAUGAAAUCCCAUC (SEQ ID NO: 131). In some aspects, vutrisiran is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Rovanersen/Lexanersen: In some aspects, the nucleic acid therapeutic agent is WVE-120101 (rovanersen, also known as WV-1092) or WVE-120102 (lexanersen, also known as WV-2603). WVE-120101 and WVE-120102 are antisense oligonucleotides for the treatment of Huntington's disease that target mutant HTT. WVE-120101 and WVE-120102 interfere with the mutant mRNA copy of the HTT gene. In some aspects, the present disclosure provides a composition comprising WVE-120101 or WVE-120102 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising WVE-120101 or WVE-120102 and 5-CNAC. In one specific aspect, the present disclosure provides a composition comprising WVE-120101 or WVE-120102 and 5-CNAC disodium salt. In some aspects, the composition is formulated for oral delivery.

The present disclosure also provides a method to treat or prevent a disease or condition in a subject in need thereof comprising administering an effective amount of a composition comprising WVE-120101 or WVE-120102 disclosed herein (e.g., a composition such as a pharmaceutical composition comprising WVE-120101 or WVE-120102 and a monosodium or disodium salt of 5-CNAC) to the subject. Also provided is a pill or capsule comprising WVE-120101 or WVE-120102 and a caprylic acid derivative disclosed herein, e.g., a monosodium or disodium salt of 5-CNAC.

The oligonucleotide sequence of WVE-120101 (rovanersen) is GGCACAAGGGCACAGACUUC (SEQ ID NO: 132). In some aspects, rovanersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

The oligonucleotide sequence of WVE-120102 (lexanersen) is GUGCACACAGTAGATGAGGG (SEQ ID NO: 133). In some aspects, lexanersen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

Cepadacursen (CIVI 008): In some aspects, the nucleic acid therapeutic agent is CIVI 008 (oral cepadacursen) (see FIG. 3). The oligonucleotide sequence of CIVI 008 is AATGCTACAAAACCCA (SEQ ID NO: 134). In some aspects, the present disclosure provides a composition comprising CIVI 008 (oral cepadacursen) and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising CIVI 008 (oral cepadacursen) and 5-CNAC. In one specific aspect, the present disclosure provides a composition comprising CIVI 008 (oral cepadacursen) and 5-CNAC disodium salt. In some aspects, the composition is formulated for oral delivery. The present disclosure also provides a method to treat or prevent a disease or condition in a subject in need thereof comprising administering an effective amount of a composition comprising CIVI 008 (oral cepadacursen) disclosed herein (e.g., a composition such as a pharmaceutical composition comprising CIVI 008 and a monosodium or disodium salt of 5-CNAC) to the subject. Also provided is a pill or capsule comprising CIVI 008 (oral cepadacursen) and a caprylic acid derivative disclosed herein, e.g., a monosodium or disodium salt of 5-CNAC.

In some aspects, the compositions disclosed herein comprise cepadacursen without its triantennary GalNAc moiety. Thus, in some aspects, the CIVI 008 oral compositions of the present disclosure comprise 4′-C-methylene Adenosylyl-(3′>5′ O,Ophosphorothioyl)-2′-O,4′-C-methylene Thymidylyl-(3′>5′ O,O-phosphorothioyl)-2′-deoxyGuanosylyl-(3′>5′ O,O-phosphorothioyl)-2′-deoxyCytidinylyl-(3′>5′ O,Ophosphorothioyl)-2′-deoxyThymidylyl-(3′>5′ O,O-phosphorothioyl)-2′-deoxyAdenosylyl-(3′>5′ O,O-phosphorothioyl)-2′-deoxyCytidinylyl-(3′>5′ O,O-phosphorothioyl)-2′-deoxyAdenosylyl-(3′>5′ O,O-phosphorothioyl)-2′-deoxyAdenosylyl-(3′>5′ O,Ophosphorothioyl)-2′-deoxyAdenosylyl-(3′>5′ O,O-phosphorothioyl)-2′-deoxyAdenosylyl-(3′>5′O,O-phosphorothioyl)-2′-deoxyCytidinylyl-(3′>5′ O,O-phosphorothioyl)-2′-O,4′ Cmethylene (5-methyl-Cytidinylyl)-(3′>5′ O,O-phosphorothioyl)-2′-O,4′-C-methylene (5-methyl-Cytidinylyl)-(3′>5′ O,O-phosphorothioyl)-2′-O,4′-C-methylene Adenosylyl hexadeca sodium salt. In some aspects, instead of a hexadeca sodium salt, the composition is another salt (e.g., potassium salt, sodium/potassium salt, etc.), a hydrate, a solvate, an alcoholate, or a combination thereof.

The oligonucleotide sequence of cepadacursen is AATGCTACAAAACCCA (SEQ ID NO: 134). See U.S. U.S. application Ser. No. 17/547,879 and International Appl. No. PCT/US21/62831, which are herein incorporated by reference in their entireties. In some aspects, cepadacursen is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

In some aspects, CIVI 008 (oral cepadacursen) can be used to treat diseases or conditions caused by abnormal expression levels and/or activity of PCSK9. Accordingly, the present disclosure provides methods of treating a disease or condition caused by abnormal expression levels and/or activity of PCSK9 in a subject in need thereof comprising administering an effective amount of an oral pharmaceutical composition disclosed herein comprising CIVI 008 to the subject, wherein the administration of the pharmaceutical composition reduces the level of serum PCSK9 and/or reduces the level of serum LDL cholesterol in the subject. In some aspects, the disease or condition is selected from the group consisting of atherosclerosis, hypercholesterolemia (e.g., familiar hypercholesterolemia or statin resistant hypercholesterolemia), HDL/LDL cholesterol imbalance, dyslipidemia (e.g., familial hyperlipidemia (FCHL) or acquired hyperlipidemia), coronary artery disease (CAD), and coronary heart disease (CHD). Accordingly, the present disclosure provides a method of treating a disease or condition selected from the group consisting of atherosclerosis, hypercholesterolemia (e.g., familiar hypercholesterolemia or statin resistant hypercholesterolemia), HDL/LDL cholesterol imbalance, dyslipidemia (e.g., familial hyperlipidemia (FCHL) or acquired hyperlipidemia), coronary artery disease (CAD), and coronary heart disease (CHD) in a subject in need thereof, the method comprising administering an effective amount of an oral pharmaceutical composition disclosed herein comprising CIVI 008 and an oral delivery agent such as 5-CNAC.

In some aspects of the present disclosure, CIVI 008 is formulated in a capsule form, wherein the capsule is a hard shell gelatin capsule. In some aspects, the capsule is a size 0 capsule (Closed Length 21.7 mm×External Diameter 7.6 mm). In some aspects, the capsule is a size 4 capsule (Closed Length 14.3 mm×External Diameter 5.05 mm). In some aspects, the capsule contains between about 5 mg and about 30 mg of CIVI 008 (cepadacursen sodium), e.g., about 5 mg, about 10 mg, about 15 mg, about 20 mg, about 25 mg, or about 30 mg of CIVI 008. In some aspects, the capsule contains about 100 mg or about 200 mg of 5-CNAC, e.g., about 100 mg, about 125 mg, about 150 mg, about 175 mg, or about 200 mg of 5-CNAC. In some aspects, the filling of the capsule is manufactured by dry blending the ingredients (i.e., dry CIVI 008 and dry 5-CNAC). In some aspects, the filling of the capsule is manufactured by freeze-drying a co-dissolved mixture of the ingredients (i.e., CIVI 008 and 5-CNAC). In some aspects, the capsule comprises about 10 mg CIVI 008 and about 100 mg 5-CNAC. In some aspects, the capsule comprises about 20 mg CIVI 008 and about 200 mg 5-CNAC. In some aspects, the capsule comprises about 5 mg CIVI 008 and about 200 mg 5-CNAC. In some aspects, the capsule comprises about 25 mg CIVI 008 and about 200 mg 5-CNAC. In some aspects, the capsule comprises about 30 mg CIVI 008 and about 200 mg 5-CNAC.

In some, the present disclosure provides a pharmaceutical composition, e.g., in a capsule form, comprising, e.g., about 10 mg CIVI 008 and about 100 mg 5-CNAC, about 20 mg CIVI 008 and about 200 mg 5-CNAC, about 5 mg CIVI 008 and about 200 mg 5-CNAC, about 25 mg CIVI 008 and about 200 mg 5-CNAC, or about 30 mg CIVI 008 and about 200 mg 5-CNAC, wherein the administration of the pharmaceutical composition to a subject results in an increase of the mean AUC₀₋₅₀ of at least about 10%, at least about 20%, at least about 30%, at least about 40%, at least about 50%, at least about 60%, at least about 70%, at least about 80%, at least about 90%, or at least about 100% with respect to the mean AUC₀₋₅₀ measured when a corresponding pharmaceutical composition comprising SNAC instead of 5-CNAC is administered to the subject. In a specific aspect, the increase of the mean AUC₀₋₅₀ with respect to the mean AUC₀₋₅₀ measured when a corresponding pharmaceutical composition comprising SNAC instead of 5-CNAC is administered to the subject is about 80%.

The term “a corresponding pharmaceutical composition comprising SNAC instead of 5-CNAC” as used herein refers to a reference pharmaceutical composition that comprises the same components as a test pharmaceutical compositions, wherein the only different between the reference pharmaceutical composition and the test composition is the substitution of SNAC present in the reference pharmaceutical composition with 5-CNAC. For example, if the test pharmaceutical composition was in a size 4 capsule containing 10 mg CIVI 008 and 100 mg 5-CNAC, the corresponding reference pharmaceutical composition would be also in a size 4 capsule and would contain 10 mg CIVI 008 and 100 mg SNAC.

In some aspects, the present disclosure provides a pharmaceutical composition, e.g., in a capsule form, comprising, e.g., about 10 mg CIVI 008 and about 100 mg 5-CNAC, about 20 mg CIVI 008 and about 200 mg 5-CNAC, about 5 mg CIVI 008 and about 200 mg 5-CNAC, about 25 mg CIVI 008 and about 200 mg 5-CNAC, or about 30 mg CIVI 008 and about 200 mg 5-CNAC, wherein the administration of the pharmaceutical composition to a subject results in an increase of the mean Cₘₐₓ of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150% with respect to the mean Cₘₐₓ measured when a corresponding pharmaceutical composition comprising SNAC instead of 5-CNAC is administered to the subject. In a specific aspect, the increase of the mean Cₘₐₓ with respect to the mean Cₘₐₓ measured when a corresponding pharmaceutical composition comprising SNAC instead of 5-CNAC is administered to the subject is about 110%.

ISIS-863633: In some aspects, the nucleic acid therapeutic agent is ISIS-863633 (see FIG. 4). See U.S. Pat. No. 10,517,953, which is herein incorporated by reference in its entirety. In some aspects, the present disclosure provides a composition comprising ISIS-863633 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising ISIS-863633 and 5-CNAC. In one specific aspect, the present disclosure provides a composition comprising ISIS-863633 and 5-CNAC disodium salt. In some aspects, the composition is formulated for oral delivery. The present disclosure also provides a method to treat or prevent a disease or condition in a subject in need thereof comprising administering an effective amount of a composition comprising ISIS-863633 disclosed herein (e.g., a composition such as a pharmaceutical composition comprising ISIS-863633 and a monosodium or disodium salt of 5-CNAC) to the subject. Also provided is a pill or capsule comprising ISIS-863633 and a caprylic acid derivative disclosed herein, e.g., a monosodium or disodium salt of 5-CNAC.

The oligonucleotide sequence of ISIS-863633 is AATAATCTCATGTCAG (SEQ ID NO: 135). In some aspects, ISIS-863633 is a non-conjugated form, i.e., the oligonucleotide is not conjugated to a delivery moiety such as GalNAc. In some aspects, the non-conjugated form (e.g., without GalNAc) is formulated with 5-CNAC, e.g., as an oral capsule.

In some aspects, the nucleic acid therapeutic agent is an oligonucleotide (e.g., an ASO or aptamer) or combination thereof (e.g., a siRNA) disclosed in TABLE 1. In some aspects, the present disclosure provides a composition comprising an oligonucleotide (e.g., an ASO or aptamer) or combination thereof (e.g., a siRNA) disclosed in TABLE 1 and a caprylic acid derivative disclosed herein, e.g., 5-CNAC or a derivative thereof, or any of the compounds disclosed, e.g., in FIG. 1A, FIG. 1B, FIG. 2, or any combination thereof. In some aspects, the caprylic acid derivative disclosed herein is a disodium salt. In one specific aspect, the present disclosure provides a composition comprising an oligonucleotide (e.g., an ASO or aptamer) or combination thereof (e.g., a siRNA) disclosed in TABLE 1 and 5-CNAC. In one specific aspect, the present disclosure provides a composition comprising an oligonucleotide (e.g., an ASO or aptamer) or combination thereof (e.g., a siRNA) disclosed in TABLE 1 and 5-CNAC disodium salt. In some aspects, the composition is formulated for oral delivery. The present disclosure also provides a method to treat or prevent a disease or condition in a subject in need thereof comprising administering an effective amount of a composition comprising an oligonucleotide (e.g., an ASO or aptamer) or combination thereof (e.g., a siRNA) disclosed in TABLE 1 (e.g., a composition such as a pharmaceutical composition comprising an oligonucleotide (e.g., an ASO or aptamer) or combination thereof (e.g., a siRNA) disclosed in TABLE 1 and a monosodium or disodium salt of 5-CNAC) to the subject. Also provided is a pill or capsule comprising an oligonucleotide (e.g., an ASO or aptamer) or combination thereof (e.g., a siRNA) disclosed in TABLE 1 and a caprylic acid derivative disclosed herein, e.g., a monosodium or disodium salt of 5-CNAC. See Moumne et al. (2022) Pharmaceutics 14: 260; Crooke et al. (2021) J. Biol. Chem. 296: 100416 which are herein incorporated by reference in their entireties.

In some aspects, an oligonucleotide (e.g., an ASO or aptamer) or combination thereof (e.g., a siRNA) disclosed in TABLE 1 is a non-conjugated form, e.g., the oligonucleotide is not conjugated to a delivery moiety such as GalNAc or PEG. In some aspects, the non-conjugated form (e.g., without GalNAc) is formulated with 5-CNAC, e.g., as an oral capsule.

TABLE 1
Exemplary therapeutic oligonucleotides.

| Oligonucleotide | Target | Sequence |
|---|---|---|
| ALNAAT-02 | SERPINA1 | |
| AROANG-3 | ANGPTL3 | |
| AROAPOC-3 | APOC3 | |
| ARO-HSD | HSD17B13 | |
| AS1411 | Nucleolin | GGTGGTGGTGGTTGTGGTGGTGGTGG (SEQ |
| | | ID NO: 137) |
| ASM-8 | CCR4 and | |
| | CSF2RB | |
| ATL-1102 | ITGA4 | |
| AZD-8233 | PCSK9 | |
| AZD-8701 | FOXP3 | |
| Belcesiran | SERPINA1 | UUUAAGAAGACAAAGGGUUUGG (SEQ ID |
| | | NO: 138) |
| | | AAACCCUUUGUCUUCUUAAAGCAGCCGAAA |
| | | GGCUGC (SEQ ID NO: 139) |
| BIIB-080 | MAPT | |
| Cimderlirsen | GHR | CCACCTTTGGGTGAATAGCA (SEQ ID NO: |
| | | 140) |
| CpG 7909 | TLR9 | |
| DYN-101 | DYN2 | |
| Fazisiran | SERPINA1 | |
| Frenlosirsen | IRF4 | AGTTGTAAATGAGUCG (SEQ ID NO: 141) |
| GTX-102 | UBE2A | |
| ION-224 | DGAT2 | |
| ION-253 | Undisclosed | |
| ION-363 | FUS | |
| ION-464 | SNCA | |
| ION-541 | ATXN2 | |
| ION-859 | LRRK2 | |
| IONIS-AGTLRx | AGT | |
| IONISAR-2.5Rx | AR | |
| IONISENAC-2.5Rx | SCNN1A | |
| IONIS-FB-LRx | CFB | |
| IONIS-FXILRx | F11 | |
| IONIS-HBVLRX | Viral HBV | |
| IONIS-PKKRx | KLKB1 | |
| IONISTMPRSS- | TMPRSS6 | |
| 6LRx | | |
| ISTH-0036 | TGFB2 | |
| JNJ-3989 | Viral HBV | |
| LSP-GR3 | GR3 | GCUAGGUUUACGGGACCUCU (SEQ ID NO: |
| | | 142) |
| Monarsen | AchE | CTGCGATATTTTCTTGTACC (SEQ ID NO: |
| | | 143) |
| MT-5745 | CHST15 | |
| NS-089 | DMD | |
| Olezarsen | APOC3 | AGCUUCTTGTCCAGCUUUAU (SEQ ID NO: |
| | | 144) |
| OLX-101 | CTGF | |
| PUL-042 | TLR2/TLR6/ | TCGTCGTCGTTCGAACGACGTTGAT (SEQ |
| | TLR9 | ID NO: 145) |
| QPI-1007 | CASP2 | |
| QR-1123 | RHO | |
| QRX-421a | USH2A | |
| RG-101 | miR-122 | |
| RG-6346 | HBsAg | |
| Sapablursen | TMPRSS6 | CUUUATTCCAAAGGGCAGCU (SEQ ID NO: |
| | | 146) |
| Sepofarsen | CEP290 | GGUGGAUCACGAGUUCA (SEQ ID NO: |
| | | 147) |
| siG-12D-LODER | KRAS | |
| SR-063 | AR | |
| STK-001 | SVN1A | |
| STP-705 | PTGS2/TGFB1 | |
| Tadnersen | C9orf72 | GCCCCTAGCGCGCGACUC (SEQ ID NO: |
| | | 148) |
| Tilsotolimod | TLR9 | TCGAACGTTCG (SEQ ID NO: 149) |
| | | TCGAACGTTCG (SEQ ID NO: 150) |
| Tomligisiran | | UACCAAUUUAUGCCUACAGCG (SEQ ID NO: |
| | | 151) |
| | | CGCUGUAGGCAUAAAUUGGUA (SEQ ID NO: |
| | | 152) |
| TOP-1731 | | TCATGAGTGGCAGCTGCAATT (SEQ ID NO: |
| | | 153) |
| Varodarsen | | UUUGCCGCUGCCCAAUGCCAUCCUG (SEQ |
| | | ID NO: 154) |
| VEGLIN 3 | VEGF | TGGCTTGAAGATGTACTCGAT (SEQ ID NO: |
| | | 155) |
| VIR-2218 | HBsAg | |
| WVE-003 | HTT | |
| WVE-004 | C9orf72 | |
| WVEN-531 | DMD | |
| Zilebesiran | AGT | UGUACUCUCAUUGUGGAUGACGA (SEQ ID |
| | | NO: 156) |
| | | GUCAUCCACAAUGAGAGUACAX (SEQ ID |
| | | NO: 157) |
| Zilganersen | GFAP | |

In some aspects, the present disclosure provides a pharmaceutical composition or dose form (e.g., a tablet or capsule) comprising (i) 1018 ISS (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AB-729 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) abetimus (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AEG35156 (GEM640) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) afovirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) aganirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) agatolimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) alicaforsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) ALNAAT-02, and (ii) 5-CNAC. In some aspects, the present disclosure provides a pharmaceutical composition or dose form (e.g., a tablet or capsule) comprising (i) amlivirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) anivamersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) apatorsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) aprinocarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) APTA-16 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) AR-177 (ZINTEVIR™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ARC19499 (BAX-499) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) archexin (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AROANG-3 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AROAPOC-3 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) ARO-HSD (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AS1411 (AGRO100) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ASM-8 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) asvasiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) atesidorsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ATL-1102 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) ATU-027 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) avacincaptad pegol (ZIMURA™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AVI-4126 (Resten-MP™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AVI-7288 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AVI-7537 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) AVT-02 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AZD-8233 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) AZD-8701 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) baliforsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) bamosiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) bazlitoran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) BC007 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) beclanorsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) belcesiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) bepirovirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) bevasiranib (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) BIIB-080 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) BMN 044 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) BMN 053 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) brivoligide (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) casimersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cavrotolimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cemdisiran (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) cenersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cepadacursen (CIVI 008) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cimderlirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cobitolimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cobomarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) CODA-001 (NEXAGON™) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) cofirasersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cosdosiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) CpG 7909 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) CPG-8954 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) cupabimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) custirsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) danvatirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) daplusiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) defibrotide (DEFITELIO™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) dematirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) donidalorsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) drisapersen (KYNDRISA™) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) DYN-101 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) edifoligide (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) egaptivon pegol (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) EIF-4E (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) eluforsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) emapticap pegol (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) eplontersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) eteplirsen (EXONDYS 51™) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) fazisiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) fesomersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) fitusiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) fomivirsen (VITRAVENE™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) frenlosirsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) gataparsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) givosiran (GIVLAARI™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) GNKG-168 (CPG-685) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) golodirsen (SRP-4053, VYONDYS 53™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) GPI-2A (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) GTI-2040 (LOR-2040) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) GTI-2501 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) GTX-102 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) HBVAXPRO (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) imetelstat (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IMT-504 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) inclisiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) inotersen (TEGSEDI™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ION-224 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ION-253 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ION-363 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ION-464 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ION-541 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ION-859 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) IONIS-AGTLRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-APO(a)-Rx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONISAR-2.5Rx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-C9Rx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-DNM2-2.5Rx (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) IONISENAC-2.5Rx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-FB-LRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-FXILRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-FXIRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-GCGRRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-HBVLRX (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-MAPTRx (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) IONIS-PKKRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONISTMPRSS-6LRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) IONIS-TTRRx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS EIF4E Rx (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-104838 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-1082 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) ISIS-113715 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-2503 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-333611 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-426115 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-449884 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-463588 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) ISIS-5132 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-702843 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-757456 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISIS-863633 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) ISTH-0036 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) JNJ-3989 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) lademirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) lexanersen (WVE-120102) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) lexaptepid pegol (NOX-H94) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) litenimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) LSP-GR3 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) lumasiran (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) mipomersen (KYNAMRO™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) miravirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) monarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) mongersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) MT-5745 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) MTL-CEBPA (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) ND-L02-s0201 (BMS-986263) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) nedosiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) NS-089 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) nusinersen (SPINRAZA™) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) oblimersen (SPC2996, GENASENSE™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) olaptesed pegol (NOX-A12) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) olezarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) olpasiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) OLX-101 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) patisiran (ONPATTRO™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) pegaptanib (MACUGEN™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) PEGnivacogin (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) pegpleranib (FOVISTA™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) pelacarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) prexigebersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) PUL-042 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) QPI-1007 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) QR-1123 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) QRX-421a (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) radavirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) remlarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) renadirsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) revusiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) RG-012 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) RG-101 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) RG-6346 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) RGLS-4326 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) rimigorsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) rosomidnar (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) rovanersen (WVE-120101) (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) sapablursen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) SB010 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) sepofarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) siG-12D-LODER (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) SLN124 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) SR-063 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) SRP-5051 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) STK-001 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) STP-705 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) suvodirsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) tadnersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) temavirsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) teprasiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) tilsotolimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) tivanisiran (SYLENTIS™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) tofersen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) tominersen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) tomligisiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) TOP-1731 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) trabedersen (AP-12009) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) trecovirsen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) varodarsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) VEGLIN³ (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) vidutolimod (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) viltolarsen (VILTEPSO™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) VIR-2218 (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) volanesorsen (WAYLIVRA™) (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) vupanorsen (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) vutrisiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) WVE-003 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) WVE-004 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) WVEN-531 (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) zilebesiran (or an unconjugated form thereof, i.e., solely the oligonucleotide portion of a conjugate), and (ii) 5-CNAC. In some aspects, the present disclosure provides a pharmaceutical composition or dose form (e.g., a tablet or capsule) comprising (i) zilganersen (or an unconjugated form thereof, i.e., solely the oligonucleotide portion of a conjugate), and (ii) 5-CNAC.

In some aspects, the present disclosure provides a pharmaceutical composition or dose form (e.g., a tablet or capsule) comprising (i) a therapeutic agent selected from the group consisting of 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™), ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimderlirsen, cobitolimod, cobomarsen, CODA-001 (NEXAGON™), cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™), fazisiran, fesomersen, fitusiran, fomivirsen (VITRAVENE™), frenlosirsen, gataparsen, givosiran (GIVLAARI™), GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS-FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX-H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™), oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™), pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SB010, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™) vupanorsen, vutrisiran, WVE-003, WVE-004, WVEN-531, zilebesiran, and zilganersen (or an unconjugated form thereof, i.e., solely the oligonucleotide portion of a conjugate), and (ii) 5-CNAC.

The term “oral composition of the present disclosure” refers to a composition comprising (i) an oligonucleotide, e.g., any of the nucleic acid therapeutic agents disclosed above and (ii) a caprylic acid derivative disclosed herein (e.g., 5-CNAC) as an oral unit-dose form. In some aspects, an oral pharmaceutical composition of the present disclosure is administered as a single dose. In some aspects, the oral pharmaceutical composition of the present disclosure is administered as multiple doses, e.g., it is administered to a human or animal patient in at least two doses in accordance with the dosing interval appropriate for that composition.

As used herein, the term “oral unit-dose form” refers to a physically discrete unit suitable for human and animal consumption and packaged individually as is known in the art. It is contemplated for purposes of the present disclosure that a dosage form comprising an effective amount (e.g., a therapeutically effective amount) of an oligonucleotide composition comprising (i) an oligonucleotide, e.g., any of the nucleic acid therapeutic agents disclosed above (e.g., CIVI 008), and (ii) a caprylic acid derivative disclosed herein (e.g., 5-CNAC) may include one or more unit doses (e.g., tablets, capsules) to achieve the therapeutic effect.

Oral dosage forms (e.g., tablets or capsules) of the oligonucleotide compositions of the present disclosure (e.g., oral pharmaceutical compositions comprising an oligonucleotide such as CIVI 008 combined with a caprylic acid derivate functioning as an oral delivery agent such as 5-CNAC) can be administered from about 5 minutes to about 60 minutes prior to a meal. In some aspects, oral dosage forms of the oligonucleotide compositions of the present disclosure can be administered from about 30 minutes to about 60 minutes prior to a meal. In some aspects, oral dosage forms of the oligonucleotide compositions of the present disclosure can be administered from about 45 minutes to about 90 minutes prior to a meal. In some aspects, oral dosage forms of the oligonucleotide compositions of the present disclosure can be administered from about 60 minutes (1 hour) to about 120 minutes (2 hours) prior to a meal.

In some aspects, oral dosage forms of the oligonucleotide compositions of the present disclosure can be administered at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, or at least about 120 minutes prior to a meal.

In some aspects, oral dosage forms of the oligonucleotide compositions of the present disclosure can be administered about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, or about 120 minutes prior to a meal.

In some aspects, oral dosage forms of the oligonucleotide compositions of the present disclosure can be administered at least about 30 minutes before the intake of food. In some aspects, oral dosage forms of the oligonucleotide compositions of the present disclosure can be administered at least about 45 minutes before the intake of food. In some aspects, oral dosage forms of the oligonucleotide compositions of the present disclosure can be administered at least about 60 minutes before the intake of food. In some aspects, oral dosage forms of the oligonucleotide compositions of the present disclosure can be administered at least about 2 hours before the intake of food.

In some aspects, oral dosage forms of the oligonucleotide compositions of the present disclosure can be provided in a solid form. In some aspects, the solid form is a capsule, e.g., a soft-gel capsule or liquid filled capsule (liquid capsule). In some aspects, oral dosage forms of the oligonucleotide compositions of the present disclosure can also be provided as a tablet, caplet or other solid oral dosage form, all of which can be prepared by methods well known in the art.

In some aspects, the oral dosage form (e.g., a tablet or a capsule) can have a weight between about 5 mg and about 1000 mg, about 10 mg and about 500 mg, about 10 mg and about 250 mg, about 100 mg and about 200 mg, or about 250 mg and about 500 mg. In some aspects, the weight of the oral dosage form (e.g., a tablet or a capsule) is about 5 mg, about 10 mg, about 20 mg, about 25 mg, about 50 mg, about 100 mg, about 200 mg, about 250 mg, about 375 mg, about 500 mg, about 750 mg, or about 1000 mg.

In some aspects, the amount of oligonucleotide, e.g., the amount of ASO, in the oral dosage form (e.g., a tablet or a capsule) is in the range of about 1 mg to about 100 mg, about 5 mg to about 100 mg, about 10 mg to about 100 mg, about 20 mg to about 100 mg, or about 20 mg and about 50 mg. In some aspects, the amount of oligonucleotide of the present disclosure is about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, or about 500 mg. In some aspects, the amount of caprylic acid derivative, e.g., 5-CNAC, is about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, or about 1000 mg.

In some aspects, the amount of oligonucleotide, e.g., the amount of ASO, in the oral dosage form (e.g., a tablet or a capsule) is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 73, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mg.

As used herein, the term “pharmaceutical composition of the present disclosure” refers to a pharmaceutical composition comprising an oligonucleotide composition comprising (i) an oligonucleotide, e.g., any of the nucleic acid therapeutic agents disclosed above (e.g., CIVI 008), (ii) a caprylic acid derivative disclosed herein (e.g., 5-CNAC), and (iii) optionally at least one pharmaceutically acceptable excipient or combination thereof.

In some aspects, the oral pharmaceutical composition of the present disclosure can comprise, in addition to an oligonucleotide (e.g., an ASO such as CIVI 008) disclosed herein and an oral delivery agent (e.g., a caprylic acid derivative such as 5-CNAC), at least one pharmaceutically acceptable excipient or combination thereof. In some aspects, the at least one pharmaceutically acceptable excipient or combination thereof, e.g., in amounts customarily employed, is selected from the group consisting of, but not limited to, a pH adjuster, a preservative, a flavorant, a taste-masking agent, a fragrance, a humectant, a tonicifier, a colorant, a surfactant, a plasticizer, a lubricant such as magnesium stearate, a flow aid, a compression aid, a solubilizer, an excipient, a diluent such as microcrystalline cellulose (e.g., Avicel PH 102), or any combination thereof.

In some aspects, the oral pharmaceutical composition of the present disclosure comprises microcrystalline cellulose. In some aspects, the oral pharmaceutical composition of the present disclosure comprises phosphate buffer salts, citric acid, glycols, other dispersing agents, or any combination thereof.

In some aspects, the oral pharmaceutical composition of the present disclosure can include a diluent, e.g., as microcrystalline cellulose (e.g., Avicel), and a lubricant, e.g., magnesium stearate. In some aspects, the oral pharmaceutical composition of the present disclosure can comprise povidone and/or crospovidone. The crospovidone can be any crospovidone. Crospovidone is a synthetic crosslinked homopolymer of N-vinyl-2-pyrrolidone, also called 1-ethenyl-2-pyrrolidinone, having a molecular weight of 1,000,000 or more. Commercially available crospovidones include Polyplasdone XL, Polyplasdone XL-10, Polyplasdone INF-10 available from ISP, Kollidon CL, available from BASF Corporation. In some aspect, the crospovidone is Polyplasdone XL. Povidone is a synthetic polymer consisting of linear 1-vinyl-2-pyrrolidinone groups having a molecular weight generally between 2,500 and 3,000,000. Commercially available povidones include Kollidon K-30, Kollidon K-90F available from BASF Corporation and Plasdone K-30 and Plasdone K-29/32, available from ISP. As mentioned above, the crospovidones and povidones are commercially available. Alternatively, they may be synthesized by known processes. The crospovidone, povidone or combination thereof can be present in the oral pharmaceutical composition of the present disclosure in an amount of from 0.5 to 50 percent by weight relative to the total weight of the overall oral pharmaceutical composition, e.g., from about 2 to about 25 percent, or from about 5 to about 20 percent by weight relative to the total weight of the oral pharmaceutical composition.

In some aspects, an oral dosage form (e.g., a tablet or a capsule) comprising an oral pharmaceutical composition of the present disclosure (e.g., an antisense oligonucleotide conjugate such as CIVI 008 and an oral delivery agent such as 5-CNAC, and optionally a statin) can comprise a coating, e.g., an enteric coatings and/or a pH sensitive coating, and optionally comprise enzyme-inhibiting agents. Accordingly, in some aspects, the solid oral dosage form does not substantially disintegrate or dissolve in the stomach, but does substantially disintegrate or dissolve in the intestine. In some aspect, the oral pharmaceutical composition of the present disclosure (e.g., an antisense oligonucleotide conjugate such as CIVI 008 and an oral delivery agent such as 5-CNAC, and optionally a statin) can further comprise one or more enzyme-inhibiting agents that prevent enzymatic degradation of active agents in the pharmaceutical formulation, for example, an oligonucleotide (e.g., CIVI 008) and/or an optional therapeutic agent such as a statin, in the stomach or the upper intestine.

In some aspects, an oral pharmaceutical composition of the present disclosure or an oral dosage form disclosed herein (e.g., a tablet or a capsule) is enterically coated to retard disintegration in the stomach. Enteric coatings include, but are not limited to, hydroxypropyl methylcellulose phthalate, hydroxypropyl methylcellulose acetate succinate, polyvinyl acetate phthalate, cellulose acetate trimellitate, cellulose acetate phthalate, poly(methacrylic acid-ethylacrylate), poly(methacrylic acid-methyl methacrylate), and combinations thereof. In yet another aspect, the oral pharmaceutical formulations may be formulated to erode from the surface of oral the dosage form, rather than disintegrate.

In some aspects, an oral pharmaceutical composition of the present disclosure or an oral dosage form disclosed herein (e.g., a tablet or a capsule) further comprises a pH sensitive coating, e.g., a pH-sensitive polymer, which protects the oral pharmaceutical composition or oral dosage form thereof from the acidic environment in the stomach. In some aspects, the pH-sensitive polymer comprises cellulose, acrylic acid, or a derivative thereof. In some aspects, the pH sensitive coating comprises a pH-sensitive hydrogel, pH-activated drug delivery system, pH-sensitive liposome, micelle or lipid nanoparticle, pH-sensitive microsphere, pH-sensitive nanoparticle, or any combination thereof. Enteric (gastro-resistant) coatings, pH sensitive coatings, enzyme inhibiting agent, and gelatin based formulations used, for example, in liquid or gel capsules are described more in detail below.

In some aspects, oral pharmaceutical compositions of the present disclosure can comprise, in addition to an oligonucleotide (e.g., an ASO such as CIVI 008) disclosed herein and an oral delivery agent (e.g., 5-CNAC), a second therapeutically active compound (therapeutic agent).

In some aspects, oral pharmaceutical compositions of the present disclosure can comprise, in addition to an oligonucleotide (e.g., an ASO such as CIVI 008) to treat hypercholesterolemia disclosed herein and an oral delivery agent (e.g., 5-CNAC), a second therapeutically active compound (therapeutic agent) selected from the group consisting of a statin (e.g., lovastatin, cerivastatin, pravastatin, atorvastatin, simvastatin, rosuvastatin, fluvastatin, or a combination thereof), ezetimibe, a bile sequestering resin, nicotinic acid, a fibric acid derivative, probucol, neomycin, dextrothyroxine, a plant stanol ester, a cholesterol absorption inhibitor, implitapide, an inhibitor of bile acid transporters, a regulator of hepatic CYP7a, an estrogen replacement therapeutic, and an anti-inflammatory.

In some aspects, the oral pharmaceutical compositions of the present disclosure can comprise, in addition to an oligonucleotide that targets PCSK9 and reduces its activity disclosed herein (e.g., an ASO such as CIVI 008) and an oral delivery agent (e.g., 5-CNAC), a second therapeutically active compound used in the art to treat a disease or condition associated with an increase in PCSK9 expression and/or PCSK9 activity.

The oral pharmaceutical compositions of the present disclosure can be prepared by conventional methods e.g. by blending a mixture of the active agent or active agents, the oral delivery agent, and other ingredients, kneading, and filling into capsules or, instead of filling into capsules, molding followed by further tableting or compression-molding to give tablets. In addition, a solid dispersion may be formed by known methods followed by further processing to form a tablet or capsule. In some aspects, the ingredients of the oral pharmaceutical compositions of the present disclosure are homogeneously or uniformly mixed throughout the solid dosage form.

The term “capsule” as used herein is intended to mean a pharmaceutical preparation comprising a hard or soft shell (e.g., a gelatin shell) typically containing a single dose of active substance (e.g., and ASO such as CIVI 008). In one aspect, the capsule is intended for oral administration. In some aspects, the capsule shell (also known as capsule body) will disintegrate in the stomach after ingestion (e.g., swallowing) to release the capsule contents (e.g., a dry blend of disclosed herein comprising, e.g., as ASO such as CIVI 008 and 5-CNAC).

As used herein, the term dry blending the term “dry blending” means thoroughly mixing several components together (e.g., an ASO such as CIVI 008 or the unconjugated form thereof and 5-CNAC) in the absence of a liquid medium. In some aspects, a component of the dry blend (e.g., an ASO such as CIVI 008 or the unconjugated form thereof, 5-CNAC, or both) can be in powder form. In some aspects, a component of the dry blend (e.g., an ASO such as CIVI 008 or the unconjugated form thereof and 5-CNAC) can be in a particulate form, e.g., granulated.

In some aspects, the present disclosure provides a pharmaceutical composition comprising 10 mg of an active agent or active agents disclosed herein (e.g., an ASO such as CIVI 008, cepadacursen; or the unconjugated form thereof) and 100 mg of 5-CNAC, wherein both components are in a dry blend. In some aspects, the present disclosure provides a pharmaceutical composition comprising 20 mg of an active agent or active agents disclosed herein (e.g., an ASO such as CIVI 008, cepadacursen) and 200 mg of 5-CNAC, wherein both components are in a dry blend. In some aspects, the present disclosure provides a pharmaceutical composition comprising 5 mg of an active agent or active agents disclosed herein (e.g., an ASO such as CIVI 008, cepadacursen) and 200 mg of 5-CNAC, wherein both components are in a dry blend. In some aspects, the present disclosure provides a pharmaceutical composition comprising 10 mg of an active agent or active agents disclosed herein (e.g., an ASO such as CIVI 008, cepadacursen) and 200 mg of 5-CNAC, wherein both components are in a dry blend. In some aspects, the present disclosure provides a pharmaceutical composition comprising 25 mg an active agent or active agents disclosed herein (e.g., an ASO such as CIVI 008, cepadacursen) and 200 mg of 5-CNAC, wherein both components are in a dry blend. In some aspects, the present disclosure provides a pharmaceutical composition comprising 30 mg of an active agent or active agents disclosed herein (e.g., an ASO such as CIVI 008, cepadacursen) and 200 mg of 5-CNAC, wherein both components are in a dry blend.

In some aspects, the present disclosure provides a capsule (e.g., a hard shell gelatin capsule enterically coated) comprising 10 mg of an active agent or active agents disclosed herein (e.g., an ASO such as CIVI 008, cepadacursen) and 100 mg of 5-CNAC, wherein both components are in a dry blend. In some aspects, the present disclosure provides a capsule comprising 20 mg an active agent or active agents disclosed herein (e.g., an ASO such as CIVI 008, cepadacursen) and 200 mg of 5-CNAC, wherein both components are in a dry blend. In some aspects, the present disclosure provides a capsule comprising 5 mg an active agent or active agents disclosed herein (e.g., an ASO such as CIVI 008, cepadacursen) and 200 mg of 5-CNAC, wherein both components are in a dry blend. In some aspects, the present disclosure provides a capsule comprising 10 mg of an active agent or active agents disclosed herein (e.g., an ASO such as CIVI 008, cepadacursen) and 200 mg of 5-CNAC, wherein both components are in a dry blend. In some aspects, the present disclosure provides a capsule comprising 25 mg of an active agent or active agents disclosed herein (e.g., an ASO such as CIVI 008, cepadacursen) and 200 mg of 5-CNAC, wherein both components are in a dry blend. In some aspects, the present disclosure provides a capsule comprising 30 mg of an active agent or active agents disclosed herein (e.g., an ASO such as CIVI 008, cepadacursen) and 200 mg of 5-CNAC, wherein both components are in a dry blend.

In some aspects, the present disclosure provides a method of treating a disease or condition caused by high expression levels and/or activity of a target gene, e.g., PCSK9, in a subject in need thereof comprising administering an effective amount of a pharmaceutical composition comprising 10 mg of an active agent or active agents disclosed herein (e.g., an ASO such as CIVI 008, cepadacursen) and 100 mg of 5-CNAC, wherein both components are in a dry blend, and optionally wherein the components are in a capsule (e.g., a hard shell gelatin capsule enterically coated). In some aspects, the present disclosure provides a method of treating a disease or condition caused by high expression levels and/or activity of a target gene, e.g., PCSK9, in a subject in need thereof comprising administering an effective amount of a pharmaceutical composition comprising 20 mg of an active agent or active agents disclosed herein (e.g., an ASO such as CIVI 008, cepadacursen) and 200 mg of 5-CNAC, wherein both components are in a dry blend, and optionally wherein the components are in a capsule. In some aspects, the present disclosure provides a method of treating a disease or condition caused by high expression levels and/or activity of a target gene, e.g., PCSK9, in a subject in need thereof comprising administering an effective amount of a pharmaceutical composition comprising 5 mg of an active agent or active agents disclosed herein (e.g., an ASO such as CIVI 008, cepadacursen; or the unconjugated form thereof) and 200 mg of 5-CNAC, wherein both components are in a dry blend, and optionally wherein the components are in a capsule. In some aspects, the present disclosure provides a method of treating a disease or condition caused by high expression levels and/or activity of a target gene, e.g., PCSK9, in a subject in need thereof comprising administering an effective amount of a pharmaceutical composition comprising 10 mg of an active agent or active agents disclosed herein (e.g., an ASO such as CIVI 008, cepadacursen) and 200 mg of 5-CNAC, wherein both components are in a dry blend, and optionally wherein the components are in a capsule. In some aspects, the present disclosure provides a method of treating a disease or condition caused by high expression levels and/or activity of a target gene, e.g., PCSK9, in a subject in need thereof comprising administering an effective amount of a pharmaceutical composition comprising 25 mg of an active agent or active agents disclosed herein (e.g., an ASO such as CIVI 008, cepadacursen; or the unconjugated form thereof) and 200 mg of 5-CNAC, wherein both components are in a dry blend, and optionally wherein the components are in a capsule. In some aspects, the present disclosure provides a method of treating a disease or condition caused by high expression levels and/or activity of a target gene, e.g., PCSK9, in a subject in need thereof comprising administering an effective amount of a pharmaceutical composition comprising 30 mg of an active agent or active agents disclosed herein (e.g., an ASO such as CIVI 008, cepadacursen; or the unconjugated form thereof) and 200 mg of 5-CNAC, wherein both components are in a dry blend, and optionally wherein the components are in a capsule.

In some aspects, the present disclosure provides a pharmaceutical composition comprising an active agent or active agents disclosed herein (e.g., an ASO such as CIVI 008, cepadacursen; or the unconjugated form thereof) and 5-CNAC at a ratio of 1:5, 1:6, 1:7, 1:8, 1:9, 1:10, 1:15, 1:20, 1:30, 1:40, or 1:50 wherein both components are in a dry blend. In some aspects, the present disclosure provides a pharmaceutical composition comprising an active agent or active agents disclosed herein (e.g., an ASO such as CIVI 008, cepadacursen; or the unconjugated form thereof) and 5-CNAC at a ratio of 1:5 wherein both components are in a dry blend. In some aspects, the present disclosure provides a pharmaceutical composition comprising an active agent or active agents disclosed herein (e.g., an ASO such as CIVI 008, cepadacursen; or the unconjugated form thereof) and 5-CNAC at a ratio of 1:10 wherein both components are in a dry blend. In some aspects, the present disclosure provides a pharmaceutical composition comprising an active agent or active agents disclosed herein (e.g., an ASO such as CIVI 008, cepadacursen; or the unconjugated form thereof) and 5-CNAC at a ratio of 1:20 wherein both components are in a dry blend. In some aspects, the present disclosure provides a pharmaceutical composition comprising an active agent or active agents disclosed herein (e.g., an ASO such as CIVI 008, cepadacursen; or the unconjugated form thereof) and 5-CNAC at a ratio of 1:40 wherein both components are in a dry blend.

In some aspects, the present disclosure provides a capsule comprising an active agent or active agents disclosed herein (e.g., an ASO such as CIVI 008, cepadacursen; or the unconjugated form thereof) and 5-CNAC at a ratio of 1:5, 1:6, 1:7, 1:8, 1:9, 1:10, 1:15, 1:20, 1:30, 1:40, or 1:50 wherein both components are in a dry blend. In some aspects, the present disclosure provides a capsule comprising an active agent or active agents disclosed herein (e.g., an ASO such as CIVI 008, cepadacursen) and 5-CNAC at a ratio of 1:5 wherein both components are in a dry blend. In some aspects, the present disclosure provides a capsule comprising an active agent or active agents disclosed herein (e.g., an ASO such as CIVI 008, cepadacursen) and 5-CNAC at a ratio of 1:10 wherein both components are in a dry blend. In some aspects, the present disclosure provides a capsule comprising an active agent or active agents disclosed herein (e.g., an ASO such as CIVI 008, cepadacursen) and 5-CNAC at a ratio of 1:20 wherein both components are in a dry blend. In some aspects, the present disclosure provides a capsule comprising an active agent or active agents disclosed herein (e.g., an ASO such as CIVI 008, cepadacursen) and 5-CNAC at a ratio of 1:40 wherein both components are in a dry blend.

In some aspects, the present disclosure provides a method of treating a disease or condition caused by high expression levels and/or activity of a target gene, e.g., PCSK9, in a subject in need thereof comprising administering an effective amount of a pharmaceutical composition comprising an active agent or active agents disclosed herein (e.g., an ASO such as CIVI 008, cepadacursen) and 5-CNAC at a ratio of 1:5, 1:6, 1:7, 1:8, 1:9, 1:10, 1:15, 1:20, 1:30, 1:40, or 1:50 wherein both components are in a dry blend, and optionally wherein the components are in a capsule.

In some aspects, the present disclosure provides a method of treating a disease or condition caused by high expression levels and/or activity of a target gene, e.g., PCSK9, in a subject in need thereof comprising administering an effective amount of a pharmaceutical composition comprising an active agent or active agents disclosed herein (e.g., an ASO such as CIVI 008, cepadacursen) and 5-CNAC at a ratio of 1:5 wherein both components are in a dry blend, and optionally wherein the components are in a capsule.

In some aspects, the present disclosure provides a method of treating a disease or condition caused by high expression levels and/or activity of a target gene, e.g., PCSK9, in a subject in need thereof comprising administering an effective amount of a pharmaceutical composition comprising an active agent or active agents disclosed herein (e.g., an ASO such as CIVI 008, cepadacursen) and 5-CNAC at a ratio of 1:10 wherein both components are in a dry blend, and optionally wherein the components are in a capsule.

In some aspects, the present disclosure provides a method of treating a disease or condition caused by high expression levels and/or activity of a target gene, e.g., PCSK9, in a subject in need thereof comprising administering an effective amount of a pharmaceutical composition comprising an active agent or active agents disclosed herein (e.g., an ASO such as CIVI 008, cepadacursen) and 5-CNAC at a ratio of 1:20 wherein both components are in a dry blend, and optionally wherein the components are in a capsule.

In some aspects, the present disclosure provides a method of treating a disease or condition caused by high expression levels and/or activity of a target gene, e.g., PCSK9, in a subject in need thereof comprising administering an effective amount of a pharmaceutical composition comprising an active agent or active agents disclosed herein (e.g., an ASO such as CIVI 008, cepadacursen) and 5-CNAC at a ratio of 1:40 wherein both components are in a dry blend, and optionally wherein the components are in a capsule.

The present disclosure provides a method for increasing oral uptake of a therapeutic oligonucleotide of the present disclosure, comprising co-administering the therapeutic oligonucleotide and a delivery agent comprises a caprylic acid (C8) derivative, wherein the caprylic acid derivative is

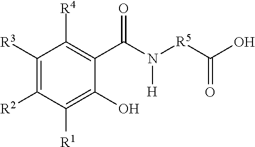

wherein
R¹, R², R³, and R⁴ are independently hydrogen, OH, NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆alkenylene, substituted or unsubstituted C₁-C₁₂ alkyl(arylene), or substituted or unsubstituted aryl(C₁-C₄ alkylene); and
R⁶ and R⁷ are independently hydrogen, oxygen, or C₁-C₄ alkyl.

The term “oral uptake” is synonymous with oral bioavailability, which is the percentage of compound, e.g., therapeutic oligonucleotide, that accesses the bloodstream after oral consumption.

The present disclosure provides a method for increasing oral uptake of a therapeutic oligonucleotide, comprising co-administering the therapeutic oligonucleotide and a delivery agent comprising a caprylic acid (C8) derivative, wherein the caprylic acid derivative is selected from the group consisting of N-(8-[2-hydroxybenzoyl]amino)caprylic acid (SNAC), N-(5-chlorosalicyloyl)-8-aminocaprylic acid (5-CNAC), N-(10-[2-hydroxybenzoly]amino)decanoic acid (SNAD), 4-[(4-chloro-2-hydroxy-benzoyl)amino]butanoic acid (4-CNAB), N-(8-[4-methoxy-chloro-2-hydroxybenzoylj-amino) octanoic acid (4-MOAC), 8-(4-hydroxyphenoxy) octanoic acid (4-HPO), 4-m-tolyloxybutyric acid (3-TBA), 4-(3-hydroxyphenylsulfanyl)butyric acid (3-HPSB), 5-phenylpentanoic acid (5-PPA), 8-(2-hydroxyphenoxy)octyldiethanolamine (2-HPOD), (4-isopropylbenzyloxy)acetic acid (4-IBOA), 2-(5-pentanoic acid)-5-(2-hydroxyphenyl)-1,3,4-oxadiazole (2-PHOD), 7-oxo-7-phenylheptanoic acid (7-OPHA), 4-(3-fluorophenylsulfanyI)butyric acid (3-FPSB), or any combination thereof. In some aspects, the caprylic acid (C8) derivative is not SNAC. In some aspects, the caprylic acid (C8) derivative is 5-CNAC.

The present disclosure provides a method for increasing oral uptake of a therapeutic oligonucleotide, comprising co-administering the therapeutic oligonucleotide and 5-CNAC. In some aspects, the therapeutic nucleotide is a therapeutic oligonucleotide of the present disclosure, e.g., cepadacursen (CIVI 008) or an unconjugated form thereof. In some aspects, the therapeutic oligonucleotide (e.g., CIVI 008) and the delivery agent (e.g., 5-CAN) are co-administered as a formulation in pill, tablet, or capsule form. In some aspects, the oral uptake of a therapeutic oligonucleotide of the present disclosure (e.g., CIVI 008) co-administered with a caprylic acid (C8) derivative (e.g., 5-CNAC) is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, or at least about 200% compared to the oral uptake of the therapeutic oligonucleotide when administered without a caprylic acid (C8) derivative, e.g., without 5-CNAC.

In some aspects, the oral uptake of a therapeutic oligonucleotide of the present disclosure (e.g., CIVI 008) co-administered with a caprylic acid (C8) derivative (e.g., 5-CNAC) is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, or at least about 200% compared to the oral uptake of the therapeutic oligonucleotide when co-administered with SNAC (i.e., replacing 5-CNAC with an equivalent amount of SNAC).

The present disclosure provides a method for increasing the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of a therapeutic oligonucleotide of the present disclosure (e.g., CIVI 008), comprising co-administering the therapeutic oligonucleotide and a delivery agent comprises a caprylic acid (C8) derivative, wherein the caprylic acid derivative is

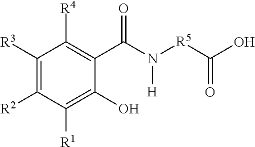

wherein
R¹, R², R³, and R⁴ are independently hydrogen, OH, NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆alkenylene, substituted or unsubstituted C₁-C₁₂ alkyl(arylene), or substituted or unsubstituted aryl(C₁-C₄ alkylene); and
R⁶ and R⁷ are independently hydrogen, oxygen, or C₁-C₄ alkyl.

The present disclosure provides a method for increasing the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of a therapeutic oligonucleotide of the present disclosure (e.g., CIVI 008), comprising co-administering the therapeutic oligonucleotide and a delivery agent comprising a caprylic acid (C8) derivative, wherein the caprylic acid derivative is selected from the group consisting of N-(8-[2-hydroxybenzoyl]amino)caprylic acid (SNAC), N-(5-chlorosalicyloyl)-8-aminocaprylic acid (5-CNAC), N-(10-[2-hydroxybenzoly]amino)decanoic acid (SNAD), 4-[(4-chloro-2-hydroxy-benzoyl)amino]butanoic acid (4-CNAB), N-(8-[4-methoxy-chloro-2-hydroxybenzoylj-amino) octanoic acid (4-MOAC), 8-(4-hydroxyphenoxy) octanoic acid (4-HPO), 4-m-tolyloxybutyric acid (3-TBA), 4-(3-hydroxyphenylsulfanyl)butyric acid (3-HPSB), 5-phenylpentanoic acid (5-PPA), 8-(2-hydroxyphenoxy)octyldiethanolamine (2-HPOD), (4-isopropylbenzyloxy)acetic acid (4-IBOA), 2-(5-pentanoic acid)-5-(2-hydroxyphenyl)-1,3,4-oxadiazole (2-PHOD), 7-oxo-7-phenylheptanoic acid (7-OPHA), 4-(3-fluorophenylsulfanyI)butyric acid (3-FPSB), or any combination thereof. In some aspects, the caprylic acid (C8) derivative is not SNAC. In some aspects, the caprylic acid (C8) derivative is 5-CNAC.

The present disclosure provides a method for increasing the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of a therapeutic oligonucleotide of the present disclosure, comprising co-administering the therapeutic oligonucleotide and 5-CNAC. In some aspects, the therapeutic nucleotide is a therapeutic oligonucleotide of the present disclosure, e.g., cepadacursen (CIVI 008) or an unconjugated form thereof. In some aspects, the therapeutic oligonucleotide (e.g., CIVI 008) and the delivery agent (e.g., 5-CAN) are co-administered as a formulation in pill, tablet, or capsule form. In some aspects, the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of a therapeutic oligonucleotide of the present disclosure (e.g., CIVI 008) co-administered with a caprylic acid (C8) derivative (e.g., 5-CNAC) is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, or at least about 200% compared to the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of the therapeutic oligonucleotide when administered without a caprylic acid (C8) derivative. In some aspects, the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of a therapeutic oligonucleotide of the present disclosure (e.g., CIVI 008) co-administered with a caprylic acid (C8) derivative (e.g., 5-CNAC) is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, or at least about 200% compared to the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, of the therapeutic oligonucleotide when co-administered with SNAC (i.e., replacing 5-CNAC with an equivalent amount of SNAC). In a specific aspects, the therapeutic effect (e.g., reduction in LDL % with respect to baseline when the oligonucleotide is an anti PCSK9 antisense such as CIVI 008) is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, or at least about 200% compare to the biological effect observed with a corresponding oligonucleotide composition comprising SNAC instead of 5-CNAC.

In some aspects, the increase or improvement in the (i) biological effect (e.g., reduction in expression of a target protein) or therapeutic effect (e.g., treatment or a disease or condition or reduction of at least one symptom), (ii) circulating plasma levels, (iii) target tissue levels, (iv) bioavailability, or (v) a combination thereof, when the therapeutic oligonucleotide is co-administered with 5-CNAC with respect to a corresponding composition comprising SNAC is observed at least about 5 days, at least about 10 days, at least about 15 days, at least about 20 days, at least about 25 days, at least about 30 days, at least about 35 days, at least about 40 days, at least about 45 days, at least about 50 days, at least about 55 days, at least about 60 days, at least about 65 days, at least about 70 days, at least about 75 days, at least about 80 days, at least about 85 days, or at least about 90 days after administration.

The present disclosure also provides a method for improved targeting of therapeutic oligonucleotides to non-hepatic tissues comprising co-administering a therapeutic oligonucleotide without a GalNAc moiety and a caprylic acid (C8) derivative disclosed herein, e.g., 5-CNAC.

In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 30 minutes after administration wherein the concentration of therapeutic oligonucleotide is at least about 10%, at least about 20%, at least about 30%, at least about 40%, at least about 50%, at least about 60%, at least about 70%, at least about 80%, at least about 90%, or at least about 100% higher than the concentration of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 30 minutes after administration wherein the concentration of therapeutic oligonucleotide is at least about 2-fold, at least about 3-fold, at least about 4-fold, at least about 5-fold, at least about 6-fold, at least about 7-fold, at least about 8-fold, at least about 9-fold, at least about 10-fold higher than the concentration of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 1 hour after administration wherein the concentration of therapeutic oligonucleotide is at least about 10%, at least about 20%, at least about 30%, at least about 40%, at least about 50%, at least about 60%, at least about 70%, at least about 80%, at least about 90%, or at least about 100% higher than the concentration of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 1 hour after administration wherein the concentration of therapeutic oligonucleotide is at least about 2-fold, at least about 3-fold, at least about 4-fold, at least about 5-fold, at least about 6-fold, at least about 7-fold, at least about 8-fold, at least about 9-fold, at least about 10-fold higher than the concentration of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 2 hours after administration wherein the concentration of therapeutic oligonucleotide is at least about 10%, at least about 20%, at least about 30%, at least about 40%, at least about 50%, at least about 60%, at least about 70%, at least about 80%, at least about 90%, or at least about 100% higher than the concentration of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 2 hours after administration wherein the concentration of therapeutic oligonucleotide is at least about 2-fold, at least about 3-fold, at least about 4-fold, at least about 5-fold, at least about 6-fold, at least about 7-fold, at least about 8-fold, at least about 9-fold, at least about 10-fold higher than the concentration of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 3 hours after administration wherein the concentration of therapeutic oligonucleotide is at least about 10%, at least about 20%, at least about 30%, at least about 40%, at least about 50%, at least about 60%, at least about 70%, at least about 80%, at least about 90%, or at least about 100% higher than the concentration of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 3 hours after administration wherein the concentration of therapeutic oligonucleotide is at least about 2-fold, at least about 3-fold, at least about 4-fold, at least about 5-fold, at least about 6-fold, at least about 7-fold, at least about 8-fold, at least about 9-fold, at least about 10-fold higher than the concentration of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 4 hours after administration wherein the concentration of therapeutic oligonucleotide is at least about 10%, at least about 20%, at least about 30%, at least about 40%, at least about 50%, at least about 60%, at least about 70%, at least about 80%, at least about 90%, or at least about 100% higher than the concentration of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 4 hours after administration wherein the concentration of therapeutic oligonucleotide is at least about 2-fold, at least about 3-fold, at least about 4-fold, at least about 5-fold, at least about 6-fold, at least about 7-fold, at least about 8-fold, at least about 9-fold, at least about 10-fold higher than the concentration of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 5 hours after administration wherein the concentration of therapeutic oligonucleotide is at least about 10%, at least about 20%, at least about 30%, at least about 40%, at least about 50%, at least about 60%, at least about 70%, at least about 80%, at least about 90%, or at least about 100% higher than the concentration of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the concentration of therapeutic oligonucleotide in plasma about 5 hours after administration wherein the concentration of therapeutic oligonucleotide is at least about 2-fold, at least about 3-fold, at least about 4-fold, at least about 5-fold, at least about 6-fold, at least about 7-fold, at least about 8-fold, at least about 9-fold, at least about 10-fold higher than the concentration of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 30 minutes after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean AUC₀₋₅ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 30 minutes after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean AUC₀₋₅ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 1 hour after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean AUC₀₋₅ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 1 hour after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean AUC₀₋₅ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 2 hours after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean AUC₀₋₅ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 2 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean AUC₀₋₅ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 3 hours after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean AUC₀₋₅ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 3 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean AUC₀₋₅ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 4 hours after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean AUC₀₋₅ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 4 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean AUC₀₋₅ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 5 hours after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean AUC₀₋₅ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 5 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean AUC₀₋₅ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 30 minutes after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean Cₘₐₓ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 30 minutes after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean Cₘₐₓ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 1 hour after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean Cₘₐₓ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 1 hour after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean Mean Cₘₐₓ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 2 hours after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean Cₘₐₓ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 2 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean Cₘₐₓ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 3 hours after administration wherein the Mean AUC₀₋₅ is least about 10%, at least about 20%, at least about 30%, at least about 40%, at least about 50%, at least about 60%, at least about 70%, at least about 80%, at least about 90%, or at least about 100% higher than the Mean Cₘₐₓ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 3 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean Cₘₐₓ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 4 hours after administration wherein the Mean Cₘₐₓ is least about 10%, at least about 20%, at least about 30%, at least about 40%, at least about 50%, at least about 60%, at least about 70%, at least about 80%, at least about 90%, or at least about 100% higher than the Mean Cₘₐₓ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 4 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean Cₘₐₓ of the corresponding conjugated therapeutic oligonucleotide (e.g., CIVI 008).

In some aspects, the co-administration of a therapeutic oligonucleotide of the present disclosure without a liver-targeting moiety (e.g., GalNAc), such as the unconjugated form of CIVI 008, with 5-CNAC results in an increase in the Mean AUC₀₋₅ of therapeutic oligonucleotide in plasma about 5 hours after administration wherein the Mean Cₘₐₓ is least about 10%, at least about 20%, at least about 30%, at least about 40%, at least about 50%, at least about 60%, at least about 70%, at least about 80%, at least about 90%, or at least about 100% higher than the Mean Cₘₐₓ of the corresponding conjugated therapeutic oligonucleotide (e.g., CIVI 008). In some aspects, the co-administration of a therapeutic oligonucleotide of the present disclosure without a liver-targeting moiety (e.g., GalNAc), such as the unconjugated form of CIVI 008, with 5-CNAC results in an increase in the Mean Cₘₐₓ of therapeutic oligonucleotide in plasma about 5 hours after administration wherein the Mean AUC₀₋₅ is least about 2-fold, at least about 3-fold, at least about 4-fold, at least about 5-fold, at least about 6-fold, at least about 7-fold, at least about 8-fold, at least about 9-fold, at least about 10-fold higher than the Mean Cₘₐₓ of the corresponding conjugated therapeutic oligonucleotide (e.g., CIVI 008).

### II. Controlled Release Formulations

In some aspects, the oligonucleotide compositions of the present disclosure comprise components to facilitate the transit through the stomach and upper intestine, e.g., enteric coatings, pH sensitive materials, and enzyme inhibitors. In some aspects, the oligonucleotide compositions of the present disclosure can also comprise gelatin, e.g., as a coating or a viscosity-increasing agent.

The enteric (gastro-resistant) coating material, e.g. polymer, can be one that will dissolve in intestinal juices at a pH level higher than that of the stomach, e.g. a pH of greater than 4.5, such as within the small intestine, and therefore permit release of the active substance in the regions of the small intestine and substantially not in the upper portion of the gastrointestinal tract. In one aspect, the enteric material begins to dissolve in an aqueous solution at pH between about 4.5 and about 5.5. In another aspect, the enteric material rapidly dissolves in an aqueous solution at pH of about 5. In another aspect, the enteric material rapidly dissolves in an aqueous solution at pH of about 5.5.

Suitable enteric (gastro-resistant) materials include, but are not limited to, cross-linked polyvinyl pyrrolidone; non-crosslinked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylate divinylbenzene copolymer; polyvinyl alcohols; polyoxyethylene glycols; polyethylene glycol; sodium alginate; galactomannan; carboxypolymethylene; sodium carboxymethyl starch; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g. EUDRAGIT™-L and -S series, including L 100-55, L 30 D-55, L 100, S 100, L 12.5, and S 12.5, available from Evonik Industries; polyvinyl acetate; fats; oils; waxes; fatty alcohols; shellac; zein; gluten; ethylacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane.

A combination of enteric materials may also be used. In some aspects, the enteric material rapidly dissolves at pH 5.5 and higher, to provide fast dissolution in the upper bowel. For example, the enteric material can be selected from a copolymer of methacrylic acid and methyl methacrylate, and a copolymer of methacrylic acid and ethyl acrylate. For example, an enteric polymer is poly(methacrylic acid co-ethyl acrylate)1:1 (EUDRAGIT™ L 30 D-55 and EUDRAGIT™ L 100-55).

Other suitable examples of enteric coating coatings include beeswax and glyceryl monostearate; beeswax, shellac and cellulose; and cetyl alcohol, mastic and shellac, and shellac and stearic acid; polyvinyl acetate and ethyl cellulose; and neutral copolymer of polymethacrylic acid esters (EUDRAGIT™ L 30D); copolymers of methacrylic acid and methacrylic acid methylester, or a neutral copolymer of polymethacrylic acid esters containing metallic stearates. Such coatings comprise mixtures of fats and fatty acids, shellac and shellac derivatives and the cellulose acid phthalates, e.g., those having a free carboxyl content.

One or more plasticizers can be added to enteric polymers to increase their pliability and reduce brittleness, as known in the art. Suitable plasticizers include, for example, butyl citrates, triethyl citrate, diethyl phthalate, dibutyl sebacate, polyethylene glycols (PEGs, such as PEG 6000), acetyl triethyl citrate, and triacetin. In one aspect, the plasticizer is triethyl citrate. While some enteric materials are flexible and do not require plasticizers, more brittle polymers (e.g., EUDRAGIT™ L/S types, EUDRAGIT™ RL/RS, and EUDRAGIT™ FS 30 D) benefit from plasticizers, for example ranging from between 5 wt. % and 30 wt. % based on the dry polymer mass, between about 8 wt. % and about 12 wt. % triethyl citrate with poly(methacrylic acid co-ethyl acrylate) 1:1.

In certain aspects, the enteric coatings comprise one or more anti-tacking agents (antiadherents) to reduce the tackiness of the film and prevent agglomeration, as it is known in the art. Suitable anti-tacking agents include, but are not limited to talc, glyceryl monostearate, fumed silica (e.g., AEROSIL™ 200), precipitated silica (e.g., SIPERNAT™ PQ), and magnesium stearate.

Anti-tacking agents can be used in any suitable quantity, for example ranging between about 10 wt. % and 100 wt. % based on dry mass, between about 10 wt. % and about 50 wt. %, between about 10 wt. % and about 30 wt. %, or between about 15 wt. % and about 30 wt. %. For example, in one aspect, in ranges between 15 wt. % and about 30 wt. % based on dry polymer mass. In some aspects, anti-tacking agents can be used in a quantity of about 10 wt. %, about 20 wt. %, about 30 wt. %, about 40 wt. %, about 50 wt. %, about 60 wt. %, about 70 wt., about 80 wt. %, about 90 wt. %, or about 100 wt. % based on dry mass

One or more surfactants can also be added to an enteric coating mixture to increase substrate wettability and/or stabilize suspensions, as it is known in the art. Surfactants include polysorbates (e.g., Polysorbate 80), sorbitan monooleate, and sodium dodecyl sulfate, and other surfactants described herein.

The enteric coating can be formed by any suitable process. Coating processes include pan coating, fluid bed coating, and dry coating (e.g., heat dry coating and electrostatic dry coating), for example. Pan coating and fluid bed coating using solvent are well-established processes. In liquid coating, the enteric material and optional excipients (e.g. pigments, plasticizers, anti-tacking agents) are mixed in an organic solvent or water to form a solution or dispersion. The coating solution or dispersion is sprayed into solid dosage forms in a pan coater or a fluid bed dryer and dried by hot air. For example, in a Wurster fluid bed coating process, the coating fluid is sprayed from the bottom of the fluid bed apparatus. Alternatively, the coating fluid is applied by top spraying. In certain aspects, a tangential spray is applied.

The amount of enteric material applied is sufficient to achieve desired acid resistance and release characteristics. For example, in one aspect the amount of enteric coating meets USP <711> requirements (USP 36-NF 31) for delayed-release dosage forms, thereby not releasing 10.0 wt. % of drug after 2 hours in 0.1 N HCL. In certain aspects, the formulation releases at least 80% of the active in 20 minutes in pH 6.8 buffer solution, e.g. using a dissolution method of USP 36-NF 31 section <711>.

In one aspect, the enteric coating is present in an amount in a range between about 10% and 40%, or between 25% and about 35% as measured by the weight gain compared to the uncoated particle cores, or ranging between about 25% and about 31% weight gain, between about 27% and about 31% weight gain, or between about 28.5% and about 31% weight gain, based on the weight of the uncoated particle cores. In one aspect, the enteric coating is present in an amount about 10%, about 15%, about 20%, about 25%, about 30%, about 35%, about 40%, about 45%, or about 50% as measured by the weight gain compared to the uncoated particle cores

The formulation can include a capsule shell. Soft and hard capsule shells are known. In one aspect, the capsule shell is a hard-capsule shell, e.g. a gelatin capsule shell or a vegetable-based hard capsule shell. In certain aspects, the capsule shell comprises one or more enteric coatings described herein. During accelerated storage, gelatin capsules may collapse. Thus, in certain aspects, the formulation can include hydroxypropyl methylcellulose capsule shell.

The solid dosage forms of the present invention may be formulated so as to prevent or retard break down in the stomach. Controlled release formulations suitable for use in the present invention may, for example, include an enteric coating or may be formulated to erode from the surface.

According to one aspect, the solid oral dosage forms comprises a therapeutically effective amount of an oral pharmaceutical composition of the present disclosure, wherein the solid oral dosage form has a disintegration time of about 250 seconds to about 650 seconds when orally administered. In another aspect, the disintegration time is about 350 to about 550 seconds when orally administered. In one aspect the disintegration time is greater than 60 seconds when orally administered. In another aspect, the disintegration time is greater than 400 seconds when orally administered. In some aspects, the solid oral dosage form has a disintegration time of about 60 seconds, about 70 seconds, about 80 seconds, about 90 seconds, about 100 seconds, about 110 seconds, about 120 seconds, about 130 seconds, about 140 seconds, about 150 seconds, about 160 seconds, about 170 seconds, about 180 seconds, about 190 seconds, about 200 seconds, about 210 seconds, about 220 seconds, about 230 seconds, about 240 seconds, about 250 seconds, about 260 seconds, about 270 seconds, about 280 seconds, about 290 seconds, about 300 seconds, about 310 seconds, about 320 seconds, about 330 seconds, about 340 seconds, about 350 seconds, about 360 seconds, about 370 seconds, about 380 seconds, about 390 seconds, about 400 seconds, about 410 seconds, about 420 seconds, about 430 seconds, about 440 seconds, about 450 seconds, about 460 seconds, about 470 seconds, about 480 seconds, about 490 seconds, about 500 seconds, about 510 seconds, about 520 seconds, about 530 seconds, about 540 seconds, about 550 seconds, about 560 seconds, about 570 seconds, about 580 seconds, about 590 seconds, about 600 seconds, about 610 seconds, about 620 seconds, about 630 seconds, about 640 seconds, or about 650 seconds when orally administered. Disintegration time can be determined in water at 37±2° C. using the method described in USP <701>.

The solid dosage forms of the present disclosure (e.g., tablets or capsules) may be covered by an enteric coating. The enteric coating may serve as the primary control for delaying the release of the drug composition or compositions in the solid dosage form. The enteric coating stays intact in the stomach and prevents or retards release into the stomach in the solid dosage form. Release of the active agent is delayed until the solid dosage form reaches the intestine. Once in the intestine, the higher pH causes release of the active agent. Enteric coatings include, but are not limited to, hydroxypropyl methylcellulose phthalate, hydroxypropyl methylcellulose acetate succinate, polyvinyl acetate phthalate, cellulose acetate trimellitate, cellulose acetate phthalate, poly(methacrylic acid-ethylacrylate), and poly(methacrylic acid-methyl methacrylate). Other enteric coatings which may be used in accordance with the present invention are described in U.S. Pat. No. 5,851,579, which is hereby incorporated by reference.

In one aspect of the present disclosure, the enteric coating is applied to the entire tablet, or other dosage form. In one aspect the enteric coating is applied to a multi-particulate system, such as a system comprising microparticles and/or nanoparticles.

The solid dosage forms of the present disclosure may be formulated to erode from the surface of the tablet (or other dosage uniform), or at the surface of the multi-particulate system (e.g. a system comprising microparticles). These surface erosion formulations slowly dissolve from the surface rather than disintegrate. By controlling the rate of surface erosion, release of the active agent and drug composition of the solid dosage form can be delayed. The surface erosion formulations can be formulated such that substantial release of the active agents or drug compositions do not occur until the solid oral dosage form reaches the intestines.

In some aspects, the solid dosage forms of the present disclosure can also comprise a protective agent like a nuclease inhibitor. In some aspects, the nuclease inhibitor comprises aurintricarboxylic acid. In some aspects, the nuclease inhibitor comprises a broad specificity nuclease inhibitor such as RNAsin. In some aspects, the nuclease inhibitor comprises GS-6620, IDX184, PSI-7777, PSI-938, RG7128, TMC649128, or ABT-072.

In some aspects, the solid dosage forms of the present disclosure can also comprise a protective agent that prevents or reduces the degradation of a conjugate moiety, e.g., GalNAc, attached covalently or non-covalently to a nucleic acid therapeutic agent of the present disclosure, e.g., an ASO, siRNA, shRNA, DNA or RNA aptamer, miRNA, antimiR, or DNA or RNA decoy. In some aspects, the protective agent prevents or reduces the cleavage of the conjugate moiety, e.g., GalNAc, from the nucleic acid therapeutic agent. In some aspects, the protective agent prevents or reduces the cleavage or degradation of one or more conjugate moieties or portions thereof, e.g., of the N-acetylgalactosamine units in a GalNAc conjugate moiety.

In some aspects, the solid dosage forms of the present disclosure can also comprise an antiacid compound. The term “antacid compound” refers to any pharmaceutically acceptable compound capable of neutralizing stomach acid (e.g., HCl in aqueous solution), preferably wherein one mole of antacid compound is capable of neutralizing at least 0.5 mole of HCl, and more preferably capable of neutralizing at least 1 mole of HCl. The therapeutically active agents (e.g., CIVI 008 alone or in combination with a second agent such as a statin), oral delivery agents (e.g., SNAC or 5-CNAC) and protease inhibitors described herein are excluded from the scope of the phrase “antacid compound”, even though they may exhibit some ability to neutralize stomach acid, in some embodiments of the invention.

Examples of antacid compounds which may be used in any one of the aspects described herein relating to one or more antacid compounds (in accordance with any of the aspects of the disclosure described herein), include, without limitation, calcium carbonate, calcium gluconate, calcium citrate, sodium carbonate, sodium bicarbonate, sodium gluconate, sodium citrate, sodium hydroxide, potassium carbonate, potassium bicarbonate, potassium gluconate, potassium citrate, potassium hydroxide, magnesium carbonate, magnesium gluconate, magnesium citrate, magnesium hydroxide, magnesium oxide, aluminum carbonate, aluminum gluconate, aluminum citrate, and aluminum hydroxide.

In some aspects, solid dosage forms of the present disclosure may also include gastric acid secretion inhibitors. The term “gastric acid secretion inhibitor” refers to any agent which reduces secretion of acid into the stomach, although it does not necessarily have any effect on acid which has already been secreted. Examples of gastric acid secretion inhibitors which may be used in any of the aspects described herein relating to an antacid composition include, without limitation, H₂receptor antagonists, such as cimetidine, famotidine, nizatidine, and ranitidine; and proton pump inhibitors, such as omeprazole, lansoprazole, dexlansoprazole, esomeprazole, rabeprazole and ilaprazole.

The solid dosage forms of the present disclosure may also include enzyme inhibiting agents. Enzyme inhibiting agents incorporated into the solid dosage unit forms may prevent the breakdown of the oligomers or other active agents that may be sensitive to enzymatic degradation. Enzyme inhibiting agents are described in U.S. Pat. No. 6,458,383, which is hereby incorporated by reference. The choice and levels of the enzyme inhibitor are based, e.g., on (1) toxicity and (2) the potency of inhibition, and will be apparent to those skilled in the art. Without wishing to be bound by theory, it is believed that an inhibitor can function solely or in combination as: a competitive inhibitor, by binding at the substrate binding site of the enzyme, thereby preventing the access to the substrate; a non-competitive inhibitor that can be simultaneously bound to the enzyme site along with the substrate, as their binding sites are not identical; and/or a complexing agent due to loss in enzymatic activity caused by deprivation of essential metal ions out of the enzyme structure.

In some aspect, the protease inhibitor included in any of the compositions (including composition unit dosage forms) described herein comprises at least one trypsin inhibitor. In some aspects, the protease inhibitor consists essentially of one or more trypsin inhibitor(s).

Examples of trypsin inhibitor which may be utilized include, without limitation, lima bean trypsin inhibitor, aprotinin, soybean trypsin inhibitor, ovomucoid trypsin inhibitor and any combination thereof. In some aspects, the trypsin inhibitor comprises soybean trypsin inhibitor (SBTI). In some aspects, the trypsin inhibitor (an optionally the at least one protease inhibitor) consists essentially of SBTI.

In some aspects, the protease inhibitor comprises at least one serpin. In some aspects, the protease inhibitor consists essentially of one or more serpin(s). Examples of serpins which may be utilized in any one of the aspects described herein, include, without limitation, alpha 1-antitrypsin, antitrypsin-related protein, alpha 1-antichymotrypsin, kallistatin, protein C inhibitor, cortisol binding globulin, thyroxine-binding globulin, angiotensinogen, centerin, protein Z-related protease inhibitor, vaspin, monocyte/neutrophil elastase inhibitor, plasminogen activator inhibitor-2, squamous cell carcinoma antigen-1 (SCCA-1), squamous cell carcinoma antigen-2 (SCCA-2), maspin, proteinase inhibitor 6 (PI-6), megsin, serpin B8 (PI-8), serpin B9 (PI-9), bomapin, yukopin, hurpin/headpin, antithrombin, heparin cofactor II, plasminogen activator inhibitor 1, glia-derived nexin, pigment epithelium derived factor, alpha 2-antiplasmin, complement 1-inhibitor, 47 kDa heat shock protein (HSP47), neuroserpin and pancpin.

In some aspects, the protease inhibitor comprises at least one cysteine protease inhibitor. In some aspects, the protease inhibitor consists essentially of one or more cysteine protease inhibitor(s). Examples of cysteine protease inhibitors which may be utilized in any one of the aspects described herein include, without limitation, type 1 cystatins, type 2 cystatins, human cystatins C, D, S, SN, and SA, cystatin E/M, cystatin F, and type 3 cystatins (including kininogens).

In some aspects, the protease inhibitor comprises at least one threonine protease inhibitor. In some aspects, the protease inhibitor consists essentially of one or more threonine protease inhibitor(s). Examples of threonine protease inhibitors which may be utilized in any one of the aspects described herein include, without limitation, bortezomib, MLN-519, ER-807446 and TMC-95A.

In some aspects, the protease inhibitor comprises at least one aspartic protease inhibitor. In some aspects, the protease inhibitor consists essentially of one or more aspartic protease inhibitor(s). Examples of aspartic protease inhibitors which may be utilized in any one of the aspects described herein, include, without limitation, α2-macroglobulin, pepstatin A, aspartic protease inhibitor 11, aspartic protease inhibitor 1, aspartic protease inhibitor 2, aspartic protease inhibitor 3, aspartic protease inhibitor 4, aspartic protease inhibitor 5, aspartic protease inhibitor 6, aspartic protease inhibitor 7, aspartic protease inhibitor 8, aspartic protease inhibitor 9, pepsin inhibitor Dit33, and protease A inhibitor 3.

In some aspects, the protease inhibitor comprises at least one metalloprotease inhibitor. In some aspects, the protease inhibitor consists essentially of one or more metalloprotease inhibitor(s). Examples of metalloprotease inhibitors which may be utilized in any one of the aspects described herein, include, without limitation, angiotensin-1-converting enzyme inhibitory peptide, antihemorrhagic factor BJ46a, beta-casein, proteinase inhibitor CeKI, venom metalloproteinase inhibitor DM43, carboxypeptidase A inhibitor, smpl, IMPI, alkaline proteinase, latexin, carboxypeptidase inhibitor, antihemorrhagic factor HSF, testican-3, SPOCK3, TIMP1, metalloproteinase inhibitor 1, metalloproteinase inhibitor 2, TIMP2, metalloproteinase inhibitor 3, TIMP3, metalloproteinase inhibitor 4, TIMP4, putative metalloproteinase inhibitor tag-225, tissue inhibitor of metalloprotease, WAP, kazal inhibitor, immunoglobulin, and kunitz and NTR domain-containing protein 1.

Examples of protease inhibitors which may be utilized in any one of the aspects described herein also include, without limitation, AEBSF—HCl, ε-aminocaproic acid, α1-antichymotypsin, antipain, antithrombin III, α1-antitrypsin, APMSF (4-amidinophenyl-methane sulfonyl-fluoride), sprotinin, benzamidine, chymostatin, DFP (diisopropylfluoro-phosphate), leupeptin, 4-(2-Aminoethyl)-benzenesulfonyl fluoride hydrochloride, PMSF (phenylmethyl sulfonyl fluoride), TLCK (1-chloro-3-tosylamido-7-amino-2-heptanone), TPCK (1-chloro-3-tosylamido-4-phenyl-2-butanone), pentamidine isothionate, pepstatin, guanidium, α2-macroglobulin, a chelating agent of zinc, and iodoacetate.

In certain aspects, the tablet or capsule might be coated with a pH-sensitive coating so that they do not dissolve in the low pH of the stomach. For example, pH-sensitive materials do not significantly dissolve until the dosage form has emptied from the stomach. The pH of the small intestine gradually increases from about 4.5 to about 6.5 in the duodenal bulb to about 7.2 in the distal portions of the small intestine (ileum). To provide predictable dissolution corresponding to the small intestine transit time of about 3 hours (e.g., 2-3 hours) and permit reproducible release therein, the coating should begin to dissolve within the pH range of the duodenum, and continue to dissolve at the pH range within the small intestine. Therefore, the amount (thickness) of enteric coating should be sufficient to be substantially dissolved during the about three-hour transit time within the small intestine (e.g., the proximal and mid-small intestine).

In some aspects, the pharmaceutical dosage form of the present disclosure releases its active compound(s) in the jejunum, e.g., in the terminal jejunum, of a subject, e.g., a human subject, through a specific design of a pH sensitive coating. The coating substantially degrades and/or dissolves in the jejunum by specific selection of the enteric coating which is preferably chosen from pH sensitive polymers substantially degrading and/or dissolving at a pH value of about 5.5 to about 7.5, preferably about 7.2 to about 7.3. Such pH sensitive polymers are preferably selected from hydroxypropylmethyl celluloses (also called hereinafter “hypromelloses”) and anionic copolymers of methacrylic acid and methacrylmethacrylate. In some aspects, the pH sensitive enteric coating containing or being made of hydroxypropylmethyl cellulose is hydroxypropylmethyl cellulose acetate succinate. A commercially available product of this kind is AQOAT®, e.g., AQOAT®-HF (Shin-Etsu Chemical Co., Chiyoda, Japan). In other aspects of the type of anionic copolymers of methacrylic acid and methacrylmethacrylate various forms of EUDRAGIT® polymers may also be used. EUDRAGIT® is commercially available from Evonik Healthcare & Nutrition GmbH, Essen, Germany. In some aspects, EUDRAGIT® FS30D is used as the pH sensitive polymer of the coating, or at least a part thereof.

In further aspects of the disclosure, different coatings can be applied in combination. According to one aspect, the coating comprises or is made of a combination of a hydroxypropylmethyl cellulose and an anionic copolymer of methacrylic acid and methacrylmethacrylate. In some aspects, a combination of coatings is applied such that typically a sub-coating of one pH sensitive polymer is applied as a first layer and a coating of a second pH sensitive polymer is applied on the sub-coating as a second layer. For example, the pH sensitive coating can comprise a sub-coating of or comprising, respectively, a hydroxypropylmethyl cellulose as a first layer, and a second coating comprising or being made of an anionic copolymer of methacrylic acid and methacrylmethacrylate provided as a second layer on the sub-coating. In a further aspect, the coating of the pharmaceutical oral dosage form of the present disclosure comprises a coating comprising a first layer (sub-coating) comprising or being made of an anionic polymer of methacrylic acid and methacrylmethacrylate such as an EUDRAGIT®, e.g., EUDRAGIT® FS30D, and a second layer comprising or being made of a hydroxypropylmethyl cellulose such as AQOAT®, more preferably AQOAT®-HF. More preferably, the anionic copolymer of methacrylic acid and methacrylmethacrylate, e.g., an EUDRAGIT® such as EURDRAGIT® FS30D, is present in less amount than the hydroxypropylmethyl cellulose such as AQOAT®, e.g., AQOAT®-HF. In other words, the thickness of the first layer of this type of combination is lower than the thickness of the second layer in this combination. More specifically, the ratio of amount or thickness, respectively, between first layer and second layer typically ranges from about 1:10 to about 1:50, e.g., from about 1:20 to about 1:30.

In one aspect, the present disclosure provides specific pharmaceutical dosage forms as outlined above which are small in dimension, preferably below 3 mm in the largest dimension, more preferably about 0.6 mm to about 1.7 mm in the largest dimension. Such small dosage forms may conveniently take the form of granules or pellets. Small dosage forms of the present disclosure have the benefit of behaving like a fluid in a subject's stomach causing a fast and constant entry of the pharmaceutical oral dosage form of the invention into the intestinal tract, and therefore to more evenly transport it to the targeted burst release area in the subject's jejunum, preferably the subject's terminal (i.e. distal part) jejunum.

In other aspects of the present disclosure, it may also be convenient that the pharmaceutical oral dosage form is of larger size, i.e. forms wherein the largest dimension of the dosage form is about 3 mm or more, the upper size limit being conveniently selected by the skilled person such that the dosage form can be well swallowed by the subject. A typical range for pharmaceutical oral dosage forms of the present disclosure are dosage forms having a largest dimension of about 3 mm to about 10 mm. It is to be understood that this range includes all integers of mm, namely, 3, 4, 5, 6, 7, 8, 9 and 10 mm as well as any sub-proportions thereof.

Gelatin is a mixture of purified protein fractions that may be obtained by partial hydrolysis of animal collagen by an acid or an alkaline. The process of acid hydrolysis is referred to as Type A and that by alkaline hydrolysis is referred to as Type B. Gelatin is a linear polymer that is comprised of amino acids which could result in a molecular weight ranging from 15,000 to 250,000. As used herein, the term gelatin includes acid and alkaline hydrolysates of animal collagen.

Gelatin may be applied in formulations of the present disclosure to serve many functions, such as a coating, a suspending agent, tablet binder and/or as a viscosity-increasing agent. In water, gelatin swells and softens and it can absorb between 5-10 times its own weight of water. There are several hydrophilic natural and synthetic polymers may be applied, in certain aspects, in place of gelatin. For example, (a) anionic polymers, such as alginic acid, dextran sulfate, or pectin; (b) cationic acids, such as chitosan or polylysine; (c) amphiphatic polymers such as carboxylmethyl chitin or fibrin; or (d) neutral polymers such as dextran, agarose, or pullulan.

As used herein, the term gelatin includes gelatin and gelatin alternatives disclosed in Remington's Pharmaceutical Sciences, 16th ed., Mack Publishing Company, Easton, Pa. (1980), page 1245 and pages 1576-1582, which is hereby incorporated by reference in its entirety. The term gelatin also includes compositions disclosed in U.S. Pat. Nos. 6,090,915, 4,043,996, 4,064,008, 4,176,117, 4,889,920, 4,374,063, 5,210,182, 4,232,425, 4,402,873, 4,427,583, 5,093,474, 5,288,408 and 5,459,241, each of which is hereby incorporated by reference in their entirety.

The term gelatin, as used herein also includes gelatin substitutes and alternatives. Generally, such a gelatin alternative can be made from easily obtainable (e.g. vegetable) materials having a homogeneous composition and having all the essential characteristics of gelatin. In the manufacture of soft gel films and capsules, the soft gel composition preferably possesses the properties of good wet and dry film strength, insolubility in cold water, oil, and alcohol, solubility in hot water, temperature and pressure sealability, film clarity, film flexibility, edibility, inertness to drugs or other materials to be encapsulated, and rapid setting from a hot liquid to form a gel.

One gelatin alternative is a film-forming composition that comprises starch material selected from modified starch and waxy starch; gum; and plasticizer as disclosed in U.S. Pat. No. 6,375,981, which is hereby incorporated by reference. The modified starch or waxy starch preferably has a dextrose equivalent (DE) of less than about 1, and more preferably has no measurable DE. This composition can be, but is not required to be, 100% gelatin-free. Thus, the composition can be used as a gelatin replacement, or as an extender in gelatin formulations.

Another gelatin alternative is wheat fiber gel as disclosed in U.S. Pat. No. 6,440,480, which is hereby incorporated by reference. Wheat fiber gel is made by thermal/physical processing of wheat fiber. A special milling technique is used for treating wheat material resulting in a product containing a large proportion of microfine particles. Specific improvements are obtained by mixing the product with maltodextrin. The product so obtained is sold under the tradename VITACEL®, by FMC Biopolymer of Philadelphia, Pa. This product is a dry powder, which readily disperses in water. Upon stirring of the dispersion the gel forms through shear forces. It is reported that wheat fiber gel can be used as a gelatin replacer in yogurt or ice cream. (I. I. Bollinger, Food Marketing & Techn. October 1995, 4-6).

Carrageenan is yet another gelatin alternative. Carrageenan is a natural hydrocolloid, a polysaccharide hydrocolloid, which is derived from seaweed. It comprises a carbohydrate polymer of repeating sugar units, which is linear, without significant numbers of branches or substitutions.

### III. Kits and Articles of Manufacture

The present disclosure also provides kits and articles of manufacture comprising the oligonucleotides and caprylic acid derivatives disclosed herein, e.g., 5-CNAC. In some aspects, the present disclosure provides a kit or article manufacture comprising (i) one or more oligonucleotides disclosed herein, e.g., an ASO (e.g., CIVI 008) targeting a particular target (e.g., PCSK9), (ii) one or more caprylic acid derivatives (e.g., 5-CNAC) for the oral delivery of the oligonucleotide, (iii) optionally a solvent, and (iv) instructions explaining, e.g., how to admix the oligonucleotides and caprylic acid derivatives, and how to deliver the resulting mixture (e.g., at least 30 minutes prior to a meal).

Such kits and articles of manufacture can comprise containers, each with one or more of the various components (e.g., in concentrated form or solid form) utilized in the methods of treatment disclosed herein, including, for example, one or more oligonucleotides disclosed herein and one or more caprylic acid derivatives disclosed herein. In some aspects, the oligonucleotide and caprylic acid derivative (e.g., CIVI 008 and 5-CNAC) are in the same container. In some aspects, the oligonucleotide (e.g., CIVI 008) and the caprylic acid derivative (e.g., 5-CNAC) are in separate containers. In some aspects, the kit or article of manufacture comprises the oligonucleotide (e.g., CIVI 008) and caprylic acid derivative (e.g., 5-CNAC) in a pill, capsule, or powder form. In some aspects, the pills or capsules can be packaged in a blister pack. In some aspects, the power form composition is packaged in a bag or envelope. In some aspects, the pills or capsules are packaged in a bottle. In some aspects, the blister pack(s), bags, or envelopes are packaged in a box. In some aspects, the box comprises printed instructions. Thus, a kit provided according to this disclosure can also comprise brochures or instructions. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EMBODIMENTS

E1. An oligonucleotide composition comprising an oligonucleotide and a delivery agent, wherein the delivery agent is covalently or non-covalently attached to the oligonucleotide, and wherein the delivery agent comprises a caprylic acid (C8) derivative.
E2. The oligonucleotide composition of embodiment E1, wherein the caprylic acid derivative is

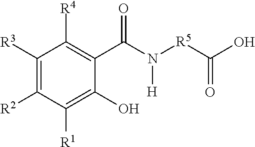

wherein
R¹, R², R³, and R⁴ are independently hydrogen, OH, NR⁶R⁷, halogen, C₁-C₄ alkyl, or C₁-C₄ alkoxy;
R⁵ is a substituted or unsubstituted C₂-C₁₆ alkylene, substituted or unsubstituted C₂-C₁₆ alkenylene, substituted or unsubstituted C₁-C₁₂ alkyl(arylene), or substituted or unsubstituted aryl(C₁-C₄ alkylene); and
R⁶ and R⁷ are independently hydrogen, oxygen, or C₁-C₄ alkyl.
E3. The oligonucleotide composition of embodiment E1 or embodiment E2, wherein the caprylic acid derivative is selected from the group consisting of N-(8-[2-hydroxybenzoyl]amino)caprylic acid (SNAC), N-(5-chlorosalicyloyl)-8-aminocaprylic acid (5-CNAC), N-(10-[2-hydroxybenzoly]amino)decanoic acid (SNAD), 4-[(4-chloro-2-hydroxy-benzoyl)amino]butanoic acid (4-CNAB), N-(8-[4-methoxy-chloro-2-hydroxybenzoylj-amino) octanoic acid (4-MOAC), 8-(4-hydroxyphenoxy) octanoic acid (4-HPO), 4-m-tolyloxybutyric acid (3-TBA), 4-(3-hydroxyphenylsulfanyl)butyric acid (3-HPSB), 5-phenylpentanoic acid (5-PPA), 8-(2-hydroxyphenoxy)octyldiethanolamine (2-HPOD), (4-isopropylbenzyloxy)acetic acid (4-IBOA), 2-(5-pentanoic acid)-5-(2-hydroxyphenyl)-1,3,4-oxadiazole (2-PHOD), 7-oxo-7-phenylheptanoic acid (7-OPHA), 4-(3-fluorophenylsulfanyI)butyric acid (3-FPSB), or any combination thereof.
E4. The oligonucleotide composition of any one of embodiment E1 to E3, wherein the caprylic acid derivative is a salt, hydrate, or solvate of SNAC, or a combination thereof.
E5. The oligonucleotide composition of embodiment E4, wherein the salt of SNAC is selected from the group consisting of a sodium salt, a potassium salt, a calcium salt, and any combination thereof.
E6. The oligonucleotide composition of embodiment E4, wherein the salt of SNAC is a sodium salt.
E7. The oligonucleotide composition of embodiment E4, wherein the salt of SNAC is a monosodium salt.
E8. The oligonucleotide composition of embodiment E4, wherein the salt of SNAC is a disodium salt.
E9. The oligonucleotide composition of any one of embodiments E1 to E8, wherein the caprylic acid derivative is a salt, hydrate, or solvate of 5-CNAC, or a combination thereof.
E10. The oligonucleotide composition of embodiment E9, wherein the salt of 5-CNAC is selected from the group consisting of a sodium salt, a potassium salt, a calcium salt, and any combination thereof.
E11. The oligonucleotide composition of embodiment E9, wherein the salt of 5-CNAC is a sodium salt.
E12. The oligonucleotide composition of embodiment E9, wherein the salt of 5-CNAC is a monosodium salt.
E13. The oligonucleotide composition of embodiment E9, wherein the salt of 5-CNAC is a disodium salt.
E14. The oligonucleotide composition of any one of embodiments E1 to E13, wherein the oligonucleotide is an antisense oligonucleotide (ASO), short interference RNA (siRNA), small hairpin RNA (shRNA), DNA and/or RNA aptamer, micro RNA (miRNA), anti-micro RNA (antimiR), CpG oligonucleotide, or DNA and/or RNA decoy.
E15. The oligonucleotide composition of embodiment E14, wherein the ASO is CIVI 008 or ISIS 863633.
E16. The oligonucleotide composition of any one of embodiments E1 to E15, wherein the oligonucleotide composition is solid.
E17. The oligonucleotide composition of any one of embodiments E1 to E16, wherein the oligonucleotide composition is formulated for delivery to the gastrointestinal tract.
E18. The oligonucleotide composition of any one of embodiments E1 to E17, wherein the oligonucleotide composition is formulated for oral delivery.
E19. The oligonucleotide composition of any one of embodiments E1 to E18, wherein the oligonucleotide composition is in the form of a tablet or a capsule.
E20. The oligonucleotide composition of embodiment E19, wherein the capsule is a liquid capsule.
E21. The oligonucleotide composition of any one of embodiments E1 to E20, wherein the oligonucleotide composition is enterically coated.
E22. The oligonucleotide composition of embodiment E19, wherein the tablet or capsule is enterically coated.
E23. The oligonucleotide composition of any one of embodiments E1 to E22, wherein the oligonucleotide composition further comprises a pH sensitive coating.
E24. The oligonucleotide composition of embodiment E23, wherein the pH sensitive coating is a pH-sensitive polymer.
E25. The oligonucleotide composition of embodiment E24, wherein the pH-sensitive polymer comprises cellulose, acrylic acid, or a derivative thereof.
E26. The oligonucleotide composition of embodiments E23 to E25, wherein the pH sensitive coating comprises a pH-sensitive hydrogel, pH-activated drug delivery system, pH-sensitive liposome, micelle or lipid nanoparticle, pH-sensitive microsphere, pH-sensitive nanoparticle, or any combination thereof.
E27. The oligonucleotide composition of embodiments E19 to E26, wherein the tablet or capsule has a weight between 5 mg and 1000 mg, 10 mg and 500 mg, 10 mg and 250 mg, 100 mg and 200 mg, or 250 mg and 500 mg.
E28. The oligonucleotide composition of any one of embodiments E19 to E27, wherein the amount of nucleic acid therapeutic agent in the tablet or capsule is in the range of 1 mg to 100 mg, 5 mg to 100 mg, 10 mg to 100 mg, 20 mg to 100 mg, 20 mg and 50 mg.
E29. The oligonucleotide composition of any one of embodiment E1 to E28, further comprising at least one pharmaceutically acceptable excipient or combination thereof.
E30. The oligonucleotide composition of embodiment E29, wherein the at least one pharmaceutically acceptable excipient or combination thereof is selected from the group consisting of a pH adjuster, a preservative, a flavorant; a taste-masking agent; a fragrance; a humectant; a tonicifier a colorant; a surfactant; a plasticiser; a lubricant; a flow aid; a compression aid; a solubilizer; an excipient; a diluent; a phosphate buffer salt; citric acid, glycol, a dispersing agent, crospovidone, povidone, or any combination thereof.
E31. A method of manufacturing an oligonucleotide composition for oral delivery comprising admixing (i) an oligonucleotide; and, (ii) an oral delivery agent, wherein the oral delivery agent is a caprylic acid derivative.
E32. The method of embodiment E31, wherein the oligonucleotide is an ASO, siRNA, shRNA, DNA or RNA aptamer, miRNA, miRNA mimic, antimiR, DNA or RNA decoy, or CpG oligonucleotide.
E33. The method of embodiment E31 or E32, wherein the caprylic acid derivative is selected from the group consisting of SNAC, 5-CNAC, SNAD, 4-CNAB, 4-MOAC, 4-HPO, 3-TBA, 3-HPSB, 5-PPA, 2-HPOD, 4-IBOA, 2-PHOD, 7-OPHA, 3-FPSB, and any combination thereof.
E34. The method of embodiment E31, wherein the oligonucleotide is an ASO and the caprylic acid derivative is SNAC or 5-CNAC.
E35. The method of embodiment E34, wherein the SNAC is a monosodium salt.
E36. The method of embodiment E34, wherein the SNAC is a disodium salt.
E37. The method of embodiment E34, wherein 5-CNAC is a monosodium salt.
E38. The method of embodiment E34, wherein 5-CNAC is a disodium salt.
E39. A tablet or capsule comprising an oligonucleotide composition comprising (i) an oligonucleotide selected from the group consisting of ASO, siRNA, shRNA, DNA and/or RNA aptamer, miRNA, antimiR, and DNA and/or RNA decoy; and, (ii) a caprylic acid derivative selected from the group consisting of SNAC, 5-CNAC, SNAD, 4-CNAB, 4-MOAC, 4-HPO, 3-TBA, 3-HPSB, 5-PPA, 2-HPOD, 4-IBOA, 2-PHOD, 7-OPHA, 3-FPSB, and any combination thereof.
E40. The tablet or capsule of embodiment E39, wherein the caprylic acid derivative is SNAC.
E41. The tablet or capsule of embodiment E40, wherein the SNAC is a monosodium salt.
E42. The tablet or capsule of embodiment E40, wherein the SNAC is a disodium salt.
E43. The tablet or capsule of embodiment E39, wherein the caprylic acid derivative is 5-CNAC.
E44. The tablet or capsule of embodiment E43, wherein 5-CNAC is a monosodium salt.
E45. The tablet or capsule of embodiment E43, wherein 5-CNAC is a disodium salt.
E46. A method of treating a disease or condition in a subject in need thereof comprising administering an effective amount of a oligonucleotide composition of any one of embodiments E1 to E30 or the tablet or capsule of embodiments E39 to E45 to the subject.
E47. The method of embodiment E46, wherein the oligonucleotide composition, table, or capsule is administered orally.
E48. The method of embodiment E46 or E47, wherein the oligonucleotide composition, tablet, or capsule is administered as a single dose.
E49. The method of embodiment E46 or E47, wherein the oligonucleotide composition is administered as multiple doses.
E50. The method of any one of embodiments E46 to E49, wherein the oligonucleotide composition, tablet, or capsule is administered from 5 minutes to 60 minutes prior to a meal.
E51. The method of any one of embodiments E46 to E50, wherein the oligonucleotide composition, tablet, or capsule is administered from at least 5, 10, 15, 20, 25, 30, 35, 40, 45, 50, 55 or 60 minutes prior to a meal.
E52. The oligonucleotide compositions of embodiments E1 to E30, method of embodiments E31 to E38, tablet or capsule of embodiment E39 to E45, or method of embodiment E46 to E51, wherein the oligonucleotide is selected from the group consisting of 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™) ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimderlirsen, cobitolimod, cobomarsen, CODA-001 (NEXAGON™), cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™) fazisiran, fesomersen, fitusiran, fomivirsen (VITRAVENE™), frenlosirsen, gataparsen, givosiran (GIVLAARI™), GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS-FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX-H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™) oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™), pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SB010, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™), vupanorsen, vutrisiran, WVE-003, WVE-004, WVEN-531, zilebesiran, and zilganersen.
E53. A capsule comprising
(i) an ASO, siRNA, shRNA, DNA or RNA aptamer, miRNA, miRNA mimic, antimiR, DNA or RNA decoy, or CpG oligonucleotide;
(ii) a 5-CNAC.
E54. The capsule of embodiment E53, where the ASO, siRNA, shRNA, DNA or RNA aptamer, miRNA, miRNA mimic, antimiR, DNA or RNA decoy, or CpG oligonucleotide and 5-CNAC are in a dry blend.
E55. The capsule of embodiment E53 or E54, wherein the capsule is a gelatin capsule.
E56. The capsule of embodiment E55, wherein the gel capsule is a hard-shell gelatin capsule.
E57. The capsule of any one of embodiment E53 to E56, wherein the capsule is enterically coated.
E58. The capsule of any one of embodiment E53 to E57, wherein the capsule comprises between 5 and 30 mg of the ASO, siRNA, shRNA, DNA or RNA aptamer, miRNA, miRNA mimic, antimiR, DNA or RNA decoy, or CpG oligonucleotide and between 100 and 200 mg of 5-CNAC.
E59. The capsule of embodiment E58, wherein the capsule comprises about 5 mg, about 10 mg, about 20 mg, about 25 mg, or about 30 mg of the ASO, siRNA, shRNA, DNA or RNA aptamer, miRNA, miRNA mimic, antimiR, DNA or RNA decoy, or CpG oligonucleotide.
E60. The capsule of any one of embodiments E53 to E59, wherein the capsule comprises:
10 mg of the ASO, siRNA, shRNA, DNA or RNA aptamer, miRNA, miRNA mimic, antimiR, DNA or RNA decoy, or CpG oligonucleotide and 100 mg of 5-CNAC;
20 mg of the ASO, siRNA, shRNA, DNA or RNA aptamer, miRNA, miRNA mimic, antimiR, DNA or RNA decoy, or CpG oligonucleotide and 200 mg of 5-CNAC;
5 mg of the ASO, siRNA, shRNA, DNA or RNA aptamer, miRNA, miRNA mimic, antimiR, DNA or RNA decoy, or CpG oligonucleotide and 200 mg of 5-CNAC;
10 mg of the ASO, siRNA, shRNA, DNA or RNA aptamer, miRNA, miRNA mimic, antimiR, DNA or RNA decoy, or CpG oligonucleotide and 200 mg of 5-CNAC;
25 mg of the ASO, siRNA, shRNA, DNA or RNA aptamer, miRNA, miRNA mimic, antimiR, DNA or RNA decoy, or CpG oligonucleotide and 200 mg of 5-CNAC; or,
30 mg of the ASO, siRNA, shRNA, DNA or RNA aptamer, miRNA, miRNA mimic, antimiR, DNA or RNA decoy, or CpG oligonucleotide and 200 mg of 5-CNAC.
E61. The capsule of any one of embodiments E53 to E60, further containing a therapeutic agent, e.g., a small molecule.
E62. A method of reducing expression levels and/or activity of a target gene in a subject in need thereof comprising administering a capsule of any one of embodiments E53 to E61 to the subject.
E63. A capsule of any one of embodiments E53 to E61 for use as a medicament to reduce expression levels and/or activity of a target gene in a subject.
E64. A method to manufacture a capsule of any one of embodiments E53 to E61 comprising: (i) dry blending a first composition comprising an ASO, siRNA, shRNA, DNA or RNA aptamer, miRNA, miRNA mimic, antimiR, DNA or RNA decoy, or CpG oligonucleotide and a second composition comprising 5-CNAC; and, (ii) encapsulating the resulting dry blend of step (i) in a capsule.

### EXAMPLES

### Example 1

### CIVI 008: Subcutaneous or Oral (Capsule) Repeat-Dose Toxicity and Toxicokinetic Study of CIVI 008 in the Cynomolgus Monkeys Using SNAC as Carrier

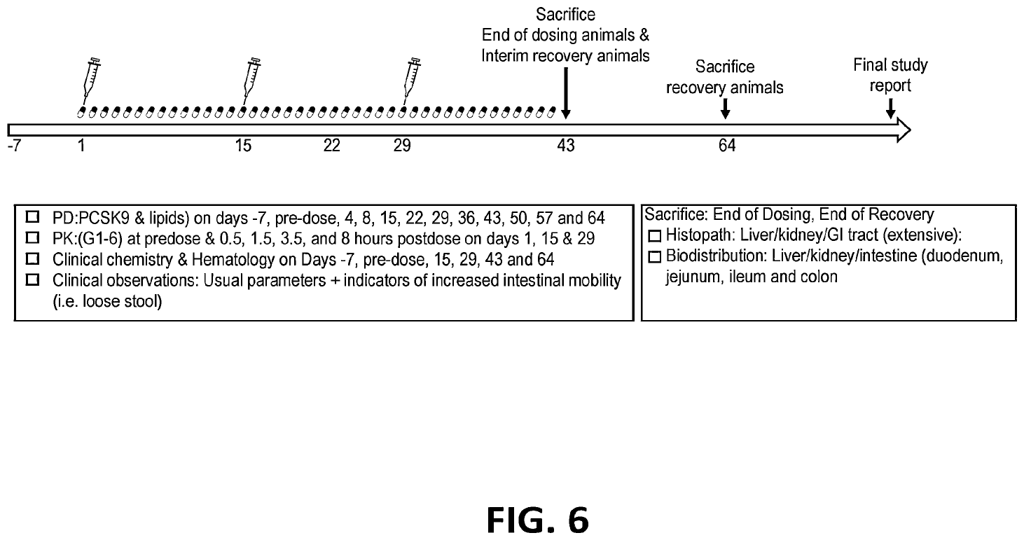

The objectives of this study were to evaluate the toxicity and reversibility of CIVI 008 and to determine the pharmacology, plasma exposure and target organ accumulation of CIVI 008 when administered daily by oral capsule to the Cynomolgus monkey of Mauritian origin for 42 days compared to administration by the subcutaneous route administered once every two weeks (investigated previously). The test article, CIVI 008, is an antisense oligonucleotide targeting PCSK9 formulated with salcaprozate sodium [SNAC, Sodium 8-[(2-hydroxybenzoyl)amino]octanoate], Manufacturer: abcr GmbH, Im Schlehert 10, 76187 Karlsruhe, Germany, Catalogue Number: AB 304409], which has previously been shown to be capable of increasing the oral bioavailability of peptides in animals and man. The purpose of this study was to provide information on the capsule dosing of CIVI 008 for further clinical trials in humans. A schematic description of the study is provided in FIG. 6.

### Group Assignment, Study Design and Dose Levels

### Subcutaneous and Oral Capsule

TABLE 2
Experimental conditions

| | | | Number | | Number of animals | | | |
|---|---|---|---|---|---|---|---|---|
| | | | of | Dosing | toxicity | | Recovery | |
| Group | Admin. Route | Dose level^{A, B} | Capsules | days | Male | Female | Male | Female |
| 1 | Subcutaneous | 3 mg/kg CIVI 008 in DPBS^{A} | — | 1, 15 & 29 | 2 | 2 | — | — |
| 2 | Oral capsule | 10 mg CIVI 008/100 mg SNAC | 1 | Daily × 42 | 2 | 2 | 1 | 1 |
| 3 | Oral capsule | 20 mg CIVI 008/200 mg SNAC | 2 | Daily × 42 | 2 | 2 | 1 | 1 |
| 4 | Oral capsule | 20 mg CIVI 008 | 2 | Daily × 42 | 1 | 1 | | |
| 5 | Oral capsule | 200 mg SNAC | 2 | Daily × 42 | 1 | 1 | | |
| 6 | Oral capsule | 0 | 2 | Daily × 42 | 1 | 1 | | |

^{A}Group 1 animals were dosed 3 mg/kg of CIVI 008 via the subcutaneous route, using the most recent body weight of each animal. For administration, the calculated dose of CIVI 008 was dissolved in a sterile solution of Dulbecco's Phosphate-Buffered Saline (DPBS) to a volume of 1 mL/kg
^{B}Groups 2 to 6 animals were dosed orally with group designated capsules, using either 1 capsule (Groups 2) or two capsules (Groups 3 to 6). The capsule(s) were administered directly into the lower part of the stomach by a catheter and expelled with air. Immediately after administration of the capsule approximately 5 mL of water was administered to aid dissolution.

Oral Capsule: Capsules containing CIVI 008 (10 mg)/SNAC (100 mg) were manufactured as uniform dry blend formulations, that were filled into Size 4 hard shell gelatin capsules that were enterically coated post fill. Formulation of the CIVI 008 drug product as an enteric capsule is justified since CIVI 008 drug substance is known to be sensitive to acidic degradation. Encapsulation within Size 4 capsules (Closed Length 14.3 mm×External Diameter 5.05 mm) was selected in order to facilitate passage of the intact capsule through the monkey pyloric sphincter.

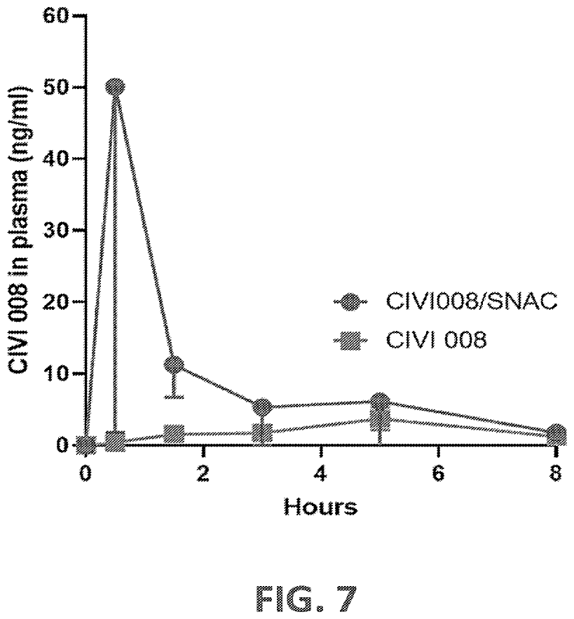

Pharmacokinetic and Biodistribution results: As shown in FIG. 7, dosing of animals by two capsules each containing 10 mg CIVI 008 and 100 mg SNAC, led to measurable concentrations of CIVI 008 in the plasma, with mean Tmax being achieved within 30 min of dosing. Consistent with SNAC being necessary for the uptake of CIVI 008, very little CIVI 008 were noted in the plasma of control animals dosed with a single capsule containing 20 mg CIVI 008 without the SNAC carrier.

CIVI 008 is a GalNac conjugated LNA-gapmer. CIVI 008 detected in plasma samples up to 1.5 hours post-dose showed the parent compound retention time in HLPC analyses, demonstrating that the drug was being absorbed intact from the gut. Samples from later time points showed a broader peak, indicative of a mixture of parent compound and metabolites, i.e., oligonuclotides with incomplete sugar moiety.

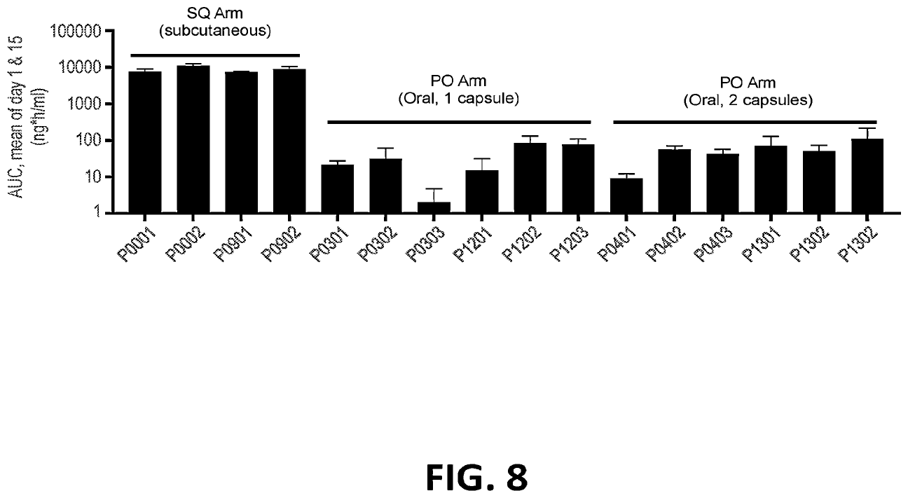

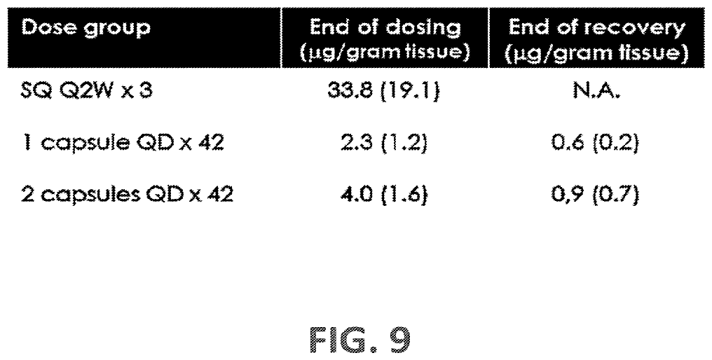

When dosed orally, CIVI 008 is subject to first-pass effects in the liver, which—due to the presence of the liver targeting GalNAc moiety—should lead to rapid liver absorption. At doses below the absorption capacity of the liver, much of the absorbed drug would thus be expected to accumulate in the liver and with very little drug appearing in general circulation. This expectation was supported by a comparison between plasma AUC's and liver concentrations (measured at the end of the dosing period) in the SQ (subcutaneous) and PO (oral) arms. Specifically, plasma AUC's in the SQ arm was observed to be 2-3 orders of magnitude higher than AUC's in the oral dosing arms (FIG. 8), whilst liver concentrations were only approximately one order of magnitude different between the SQ and PO arms (FIG. 9). When using RNA therapeutics to inhibit targets in the liver, the change in the plasma/liver exposure ratio between oral dosing and SQ or IV dosing, thus offered the means to significantly reduce exposure of non-hepatic tissues/plasma compartment and consequently reduce potential safety issues in non-target tissues.

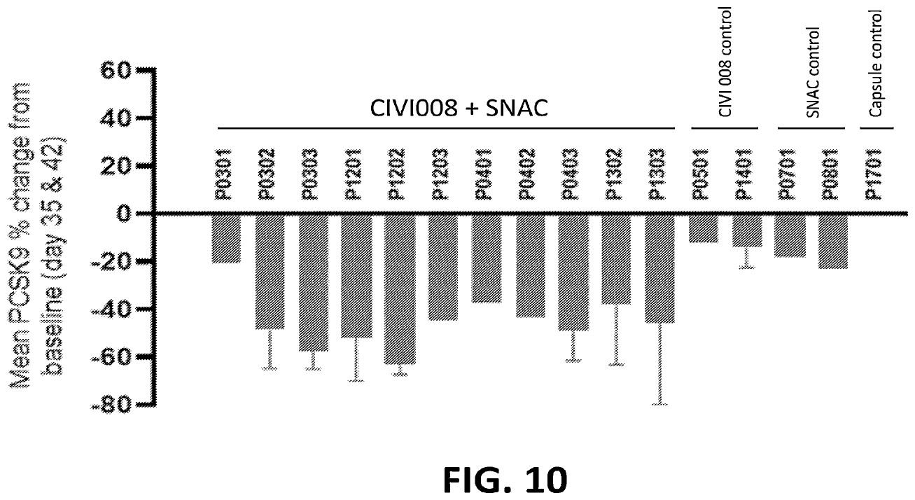

Pharmacodynamic results: Daily oral dosing of 1 or 2 capsules of CIVI 008/SNAC for 42 days, led to a measurable reductions in the primary target, PCSK9, typically being noted after two-weeks of dosing. Mean percentage reductions in PCSK9 at day 35/42 varied between animals with best responders achieving PCSK9 reductions >60% compared to baseline (FIG. 10). Mean PCSK9 reductions in control animals (CIVI 008 alone, SNAC alone and empty capsules), were comparatively small, underpinning that the significant decrease in the active arms were caused by SNAC mediated absorption of CIVI 008.

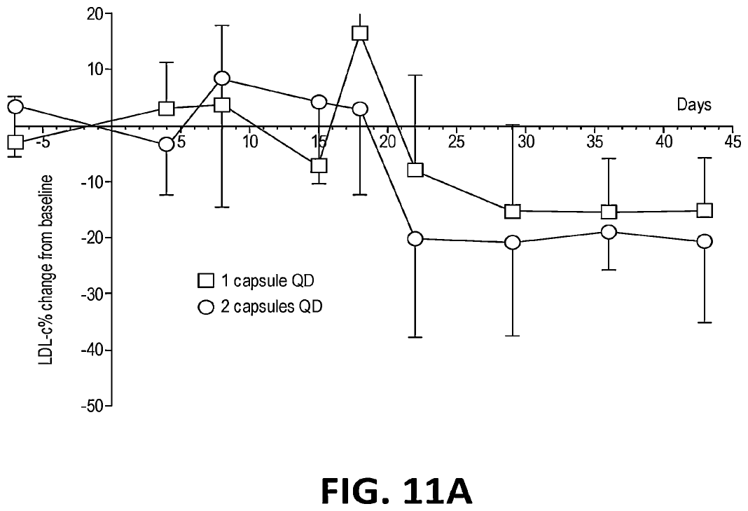

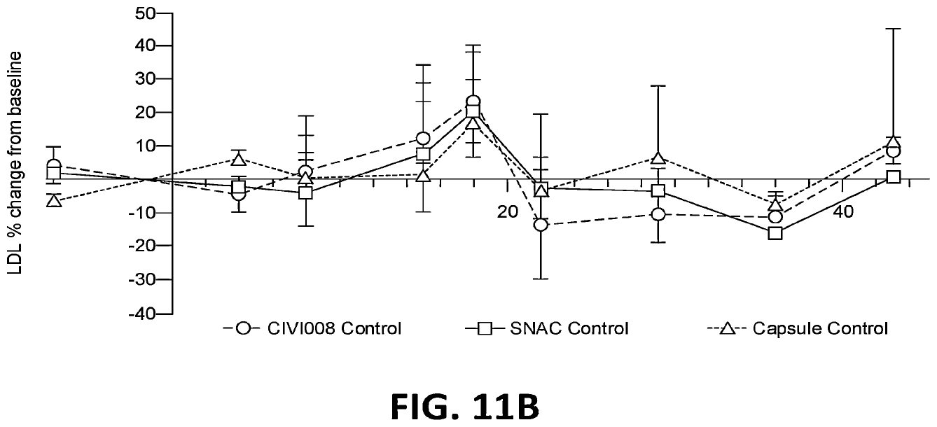

PCSK9 negatively regulates the cell surface LDL-receptor, which is responsible for cholesterol import into the liver. Reducing PCSK9 pharmacologically thus increases the amount of LDL-receptor, causing an increase in import into the liver and a reduction in plasma LDL-cholesterol. Consistent with this function of PCSK9, the CIVI 008/SNAC mediated reductions in PCSK9 led to a measurable reduction in LDL-c, starting around 3-weeks post dosing and stabilizing from week 4 forward (FIG. 11A). LDL-c reductions in control animals (CIVI 008 alone, SNAC alone and empty capsules) fluctuated around baseline values throughout the study (FIG. 11B)

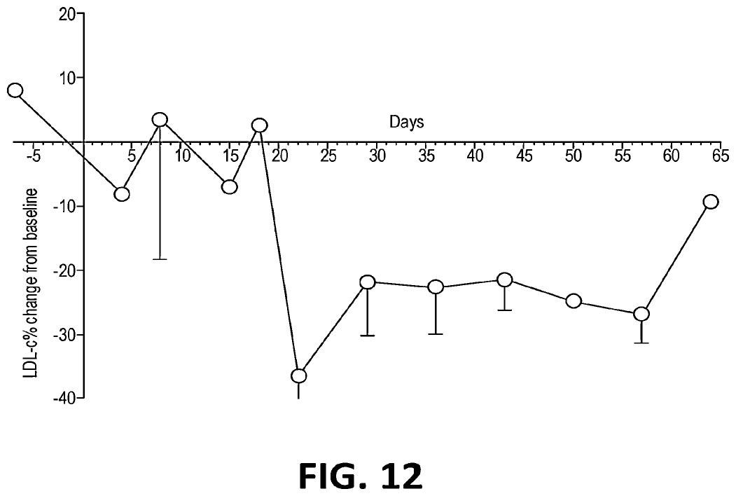

As shown in FIG. 9, the concentration of CIVI 008 declined in the liver during the recovery period, but was still present in measurable quantities at the 3-week timepoint. Consistent with the presence of drug in the liver throughout the recovery period, LDL levels at the end of dosing were maintained for two weeks post-dosing, and had yet to fully return to baseline at the end of recovery (FIG. 12).

Toxicity results: The study was conducted to GLP standards and monitored the following toxicity parameters throughout the study (pre-dose, day 14, 29, 42, and end of recovery):
Hematology: red blood cell (erythrocyte) count, hemoglobin, hematocrit, mean corpuscular volume, mean corpuscular hemoglobin, mean corpuscular hemoglobin, concentration, red cell distribution width, absolute reticulocyte count, platelet count, white blood cell (leukocyte) count, absolute neutrophil count, absolute lymphocyte count, absolute monocyte count, absolute eosinophil count, absolute basophil count, absolute large unstained cell count, blood smear.
Clinical chemistry, PCSK9 and lipids: urea nitrogen, creatinine, total protein, albumin, globulin, albumin:globulin ratio, total bilirubin, aspartate aminotransferase, alanine aminotransferase, alkaline phosphatase, gamma glutamyltransferase, creatine kinase, calcium, inorganic phosphorus, sodium, potassium, chloride, high density lipoproteins, low density lipoprotein, very low density lipoprotein, total cholesterol, triglycerides.

CIVI 008 formulated with SNAC was well tolerated with no post-dosing signs of toxicologically significant clinical observations. There was no notable changes in clinical chemistry parameters or haematological markers in any of the animals during the dosing or recovery phase.

There were no organ weight changes that suggested an effect of the drug following oral capsule administration. Also, there were no macroscopic or microscopic findings that suggested local or systemic effects of the drug at the end of dosing and end of recovery. In particular, there were no local histopathological changes in any segment of the intestinal tract (duodenum, jejunum, ileum and colon) after QD oral dosing for 42 days, despite all segment having measurable concentrations of CIVI 008.

### Example 2

### Preparation of Disodium N-(5-Chorosalicyloyl)-8-aminocaprylate Step 1 Step 2

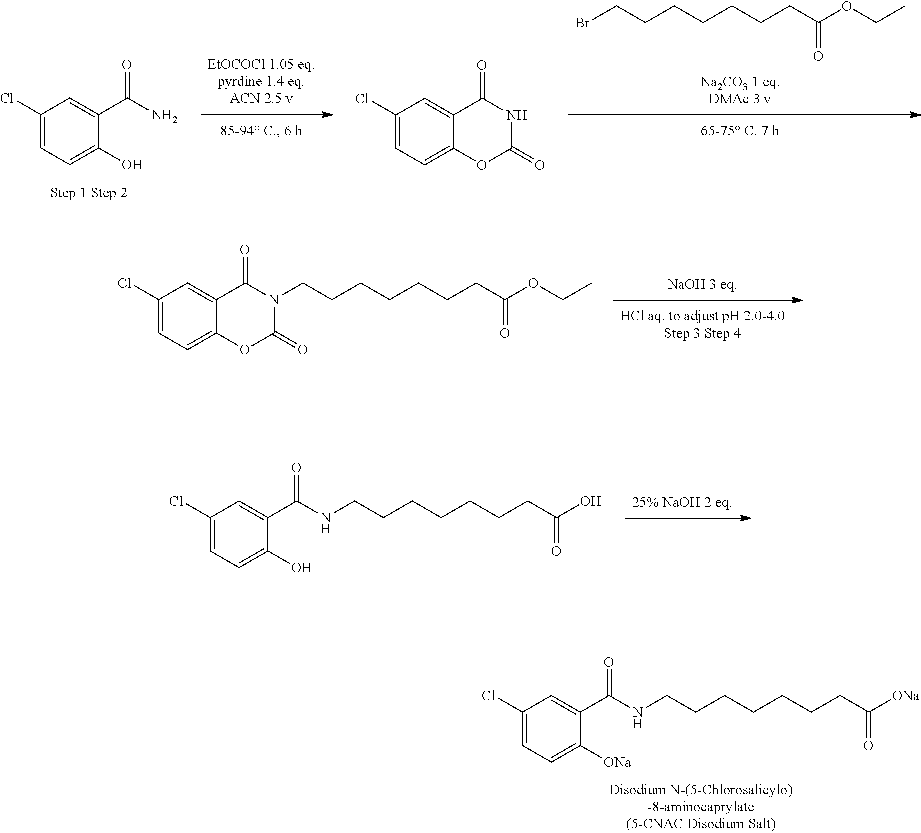

Step 1: 5-Chloro-2-hydroxy-benzamide (30.0 g, 99.9%, 1.0 equivalent), acetonitrile (90 mL, 3 volumes), pyridine 19.4 g, 1.403 equivalents were charged to a reactor and the mixture stirred at 8-16° C. for 10-30 minutes. Ethyl chloroformate (20.3 g, 1.07 equivalents) was charged to the reactor at 8-16° C. and the reaction mixture stirred at 10-18° C. for 30-60 minutes. The mixture was then heated to reflux and stirred at 80-90° C. for 4 hours. The mixture was concentrated to 3.5 volumes by distillation at temperature below 80-90° C. at reduced pressure (<1 bar). The reactor was then charged with acetonitrile (45 mL, 1.5 volumes) was again concentrated to 3.5 volumes, then allowed to cool to 18-28° C. Water (60 mL, 2 volumes) was added and the mixture stirred at 18-28° C. for 1-3 hours. The mixture was cooled to 4-8° C., the precipitate collected by filtration and the cake washed with water (30 mL, 1 volume). The wet cake was dried at 58° C. for 6 hours to afford the 6-chloro-2H-1,3-benzoxazine-2,4(3H)-dione (31.5 g, 92.6% purity) in 92.6% crude yield.

Step 2: Dry dimethylacetamide (150 mL, 3 volumes), granular sodium carbonate (25.1 g, 1.0 equivalents), 6-chloro-2H-1,3-benzoxazine-2,4(3H)-dione (50.0 g, after corrected by assay 93.4 w %, 1 equivalent) and ethyl 8-bromooctanoate (56.8 g, after corrected by assay 99.2 w %, 0.95 equivalents) and the pressure reduced to −0.3 MPa. The stirred mixture was then heated at 70° C. for 14 hours. The mixture was then cooled to 35-45° C. and the precipitate collected by filtration. The wet cake was charged to a reactor designated Reactor 1 (R1), the filtrate was charged to a second reactor, designated Reactor 2 (R2). Ethanol (60 mL) was charged to R1 and the wet cake-ethanol mixture stirred at 35-45° C. for 10-30 minutes. The mixture was filtered and the filtrate combined with that already present in R2. The stirred solution contents of R2 were cooled to 25-30° C. and water (100 mL, 2 volumes) slowly added directly to the solution. The mixture was cooled to 5-10° C. and after holding for 9.5 hours, the precipitate that formed was collected to afford ethyl 8-(6-chloro-2H-1,3-benzoxazine-2.4 (3H)-dionyl)octanoate (100 g, 97.5% purity) as a wet cake.

Step 3: Water (240 mL, 3 volumes), sodium hydroxide (30 g, 3.3 equivalents) and ethyl 8-(6-chloro-2H-1,3-benzoxazine-2.4 (3H)-dionyl)octanoate (83 g, 1.0 equivalent) were charged to a reactor (Reactor 1 (R1)) and stirred at 25° C. for 10 minutes. The stirred mixture was heated at 98° C. for 3 hours with distillation at which time the starting material had been consumed. The reaction mixture was then allowed to cool to 27° C. Water (240 mL) and HCl (66 mL, 3.5 equivalents) were charged with stirring into an adjacent reactor (Reactor 2 (R2)) and allowed to cool to 20-25° C. The saponified reaction mixture (R1) was slowly added to R2 over a period of 5 hours, with accompanied by the evolution of carbon dioxide and product precipitation. The pH of the mixture was adjusted to pH 2-3 with 50% sodium hydroxide solution and stirred at 8° C. for 3 hours. The product was collected by filtration, washed with water and dried under vacuum to afford N-(5-chlorosalicyloyl)-8-aminocaprylic acid (5-CNAC, 63 g, 95.7% purity) in 88.9% crude yield.

Step 4: N-(5-chlorosalicyloyl)-8-aminocaprylic acid (5-CNAC) (1.0 g, 1.0 equivalent), sodium hydroxide (0.26 g, 2 equivalents) and water (5 mL, 5 volumes) were combined in a reactor and stirred at 55° C. for 2 hours. The mixture was allowed to cool to 20° C. and the solution filtered to remove insoluble solids. The filtrate was concentrated below 50° C. at reduced pressure and the wet cake dried at 50° C. for 12 hours to afford Disodium N-(5-Chlorosalicyloyl)-8-aminocaprylate (1.1 g, 97.9% purity) in 96.5% crude yield.

### Example 3

### CIVI 008: Oral (Capsule) Toxicokinetic Study of CIVI 008 in the Cynomolgus Monkeys Using 5-CNAC as Carrier

The objectives of this study were to determine the ability of 5-CNAC [Disodium N-(5-Chorosalicyloyl)-8-aminocaprylate] to facilitate oral uptake of CIVI 008 and to determine the manufacture method and dosing paradigm of CIVI 008/5-CNAC capsules that provide the most efficient oral uptake of CIVI 008 when administered to Cynomolgus monkeys of Mauritian origin. The purpose of this study was to provide information on the capsule dosing of CIVI 008 for further clinical trials in humans. A schematic outline of the study is provided in FIG. 13.

Oral Capsule: A number of different capsules were manufactured as outlined below.

TABLE 3
Capsules.

| Capsule | CIVI 008 | 5-CNAC | Manufacture | Capsule |
|---|---|---|---|---|
| group | (mg) | (mg) | method | size |
| A | 10 | 100 | Dry Blend¹ | 4 |
| B | 10 | 100 | Dry Blend¹ | 0 |
| C | 20 | 200 | Dry Blend¹ | 0 |
| D | 5 | 200 | Dry Blend¹ | 0 |
| E | 10 | 200 | Dry Blend¹ | 0 |
| F | 25 | 200 | Dry Blend¹ | 0 |
| G | 30 | 200 | Dry Blend¹ | 0 |
| H | 5 | 200 | Freeze Dryed² | 0 |
| I | 10 | 200 | Freeze Dryed² | 0 |
| J | 25 | 200 | Freeze Dryed² | 0 |
| K | 30 | 200 | Freeze Dryed² | 0 |

¹Dry blended capsules were manufactured from uniform dry blended formulations, that were filled into either i) Size 4 hard shell gelatin capsules (Closed Length 14.3 mm × External Diameter 5.05 mm) and enterically coated post fill or ii) enterically coated Size 0 hard shell gelatin capsules (Closed Length 21.7 mm × External Diameter 7.6 mm)
²Freeze dried capsules were manufactured by co-dissolving CIVI 008 and 5-CNAC in [water] followed by freeze drying and filling into enterically coated Size 0 hard shell gelatin capsules

Study outline: A total of 10 Cynomolgus monkeys (5 males and 5 females) were each dosed with 2 group A capsules on Monday and Wednesday in week 1, followed by 4 days of dosing holiday. This Monday and Wednesday dosing scheme was continued in weeks 2, 3, 5, 6, 7, 8, 13, 14, 15 and 17 using 2 group B-capsules per dose occasion in week 2, and 1 capsule of groups C to K per dose occasion in their respective weeks. During the dosing period, blood samples for PK analysis were drawn pre-dose and 0.5, 1.5, 3 and 5 hours post-dosing on Mondays and Wednesdays. Samples for clinical chemistry and hematology (see example 1) were taken pre-study and end of study.

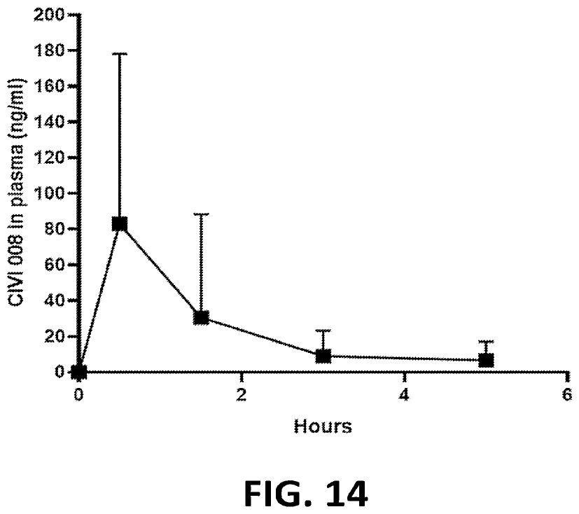

Pharmacokinetic results: As shown in FIG. 14 dosing of animals by two group A capsules, led to measurable concentrations of CIVI 008 in the plasma, with mean Tmax being achieved within 30 min of dosing.

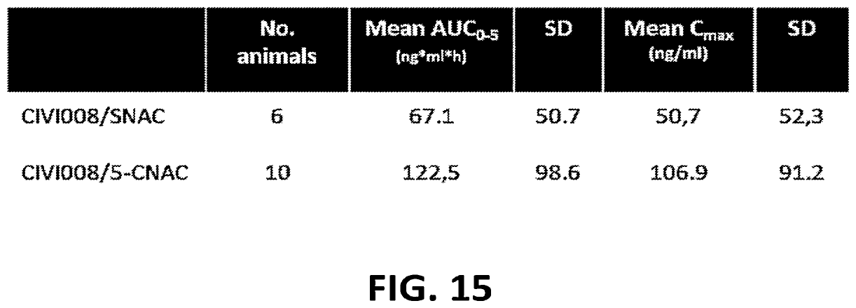

Compared to a similar dose of CIVI 008 using SNAC as carrier (Example 1, FIG. 7), the use of the 5-CNAC carrier led to an increase in both AUC₀₋₅ as well as Cₘₐₓ (FIG. 15), indicating that 5-CNAC is the more efficient of the two carriers in facilitating oral uptake of CIVI 008.

Capsules used in the study were enterically coated to facilitate pH dependent release of CIVI 008/5-CNAC in the intestine. Increasing the size of the capsules from size 4 (group A) to size 0 (group B) didn't change the mean plasma PK profile of CIVI 008 or the mean Tmax, indicating that the larger size 0 capsules are able to transit from the stomach with the same kinetics as the smaller size 4 capsules.

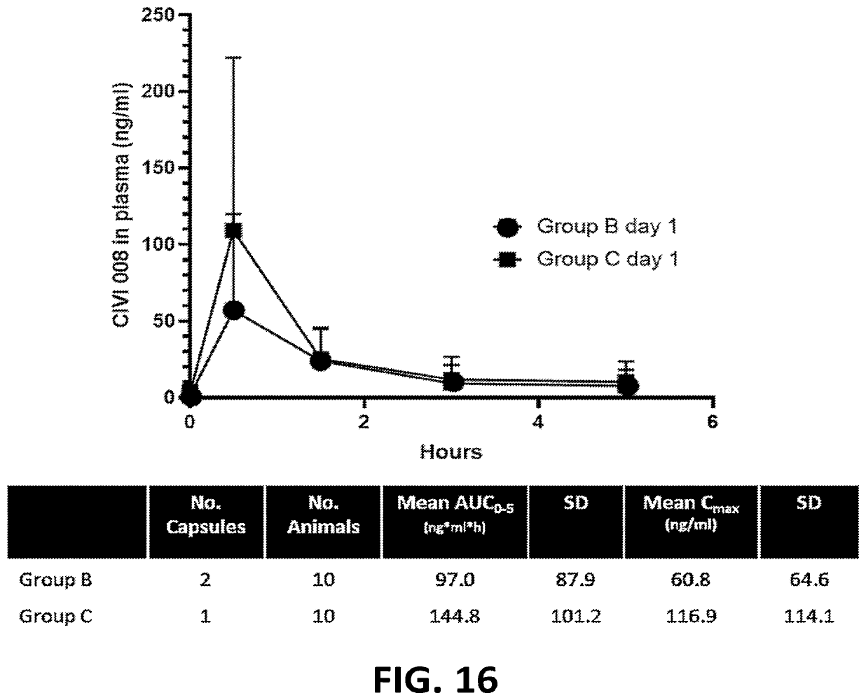

When delivered as a capsule, the co-formulation creates a high local concentration of each constituent at the landing site in the intestine, which is important for the ability of the carrier to facilitate uptake of the co-formulated drug. Consequently, delivering a similar amount of drug/carrier in either 1 or 2 capsules might affect the absorption efficiency of the drug. As shown in FIG. 16, this appears to be the case. Whilst the kinetics of the plasma PK profiles were very similar, a single capsule containing 20 mg CIVI 008/200 mg 5-CNAC (group C capsules) caused a higher Cₘₐₓ and AUC₀₋₅ than the similar amount of drug/carrier administered in 2 capsules each containing half the amount of drug/carrier.

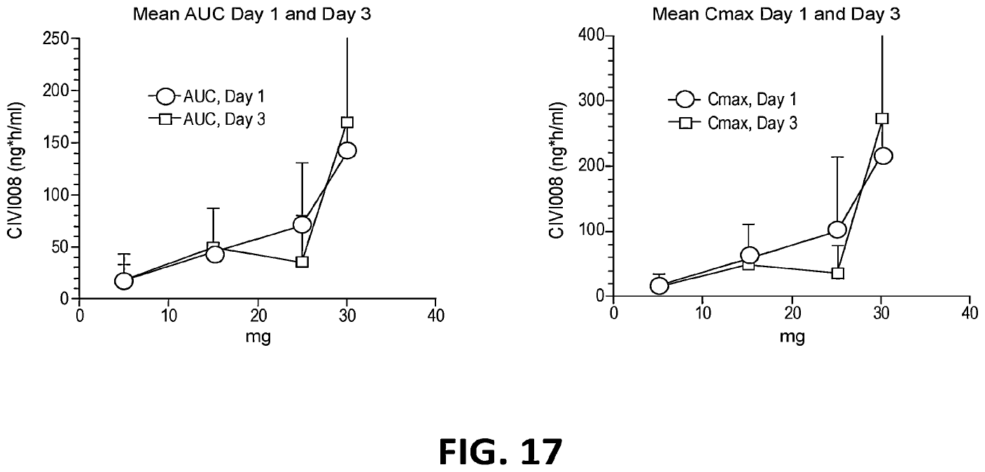

Increasing the dose of CIVI 008 at a constant dose of 5-CNAC, increased AUC and Cₘₐₓ dose proportionally up to 25 mg. Increasing the dose further lead to more than dose proportional increases in AUC and Cₘₐₓ, indicating that liver uptake was reaching saturation (FIG. 17).

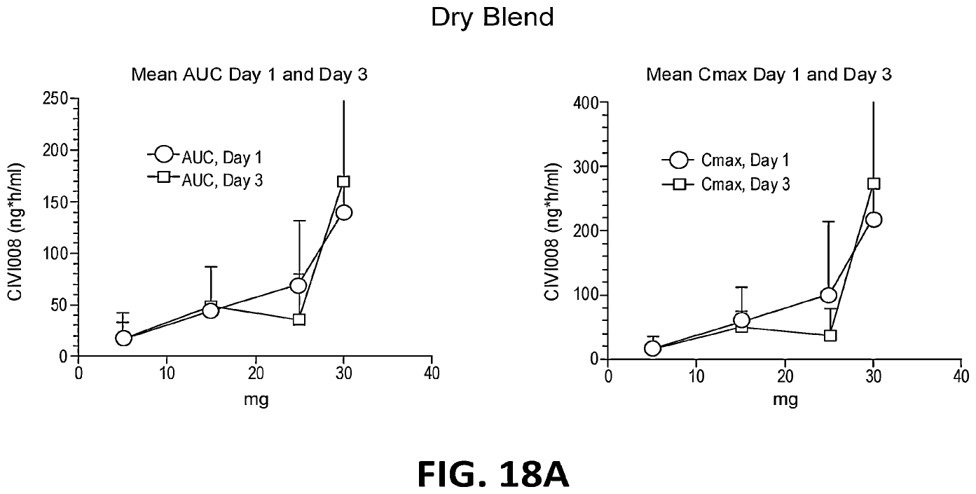

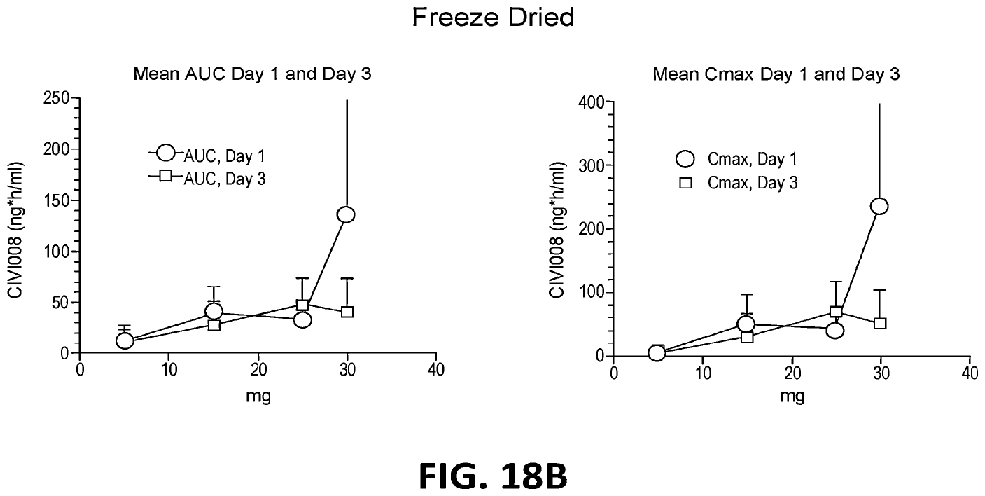

Changing the manufacture method from dry blending of CIVI 008 and 5-CNAC (FIG. 18A) to co-dissolution of CIVI 008 and 5-CNAC followed by freeze-drying (FIG. 18B), did not appear to affect intestinal absorption of the CIVI 008 drug. Thus, very similar AUC and Cₘₐₓ profiles of CIVI 008 were observed over the dose range 5 mg to 30 mg when capsules were manufactured by either method.

### Example 4

### CIVI 008: Oral (Capsule) Pharmacology Study of CIVI 008 in the Cynomolgus Monkeys Using 5-CNAC as Carrier

The objectives of this study were to determine the ability of 5-CNAC [disodium N-(5-chorosalicyloyl)-8-aminocaprylate] to reduce plasma PCSK9 when dosed once daily for 7 weeks. The half-life of CIVI 008 in the liver of non-human primates is between 2 to 3 weeks, so after 7-weeks of dosing liver concentrations are expected to reach >80% of its steady state level.

Study outline: Dry blended capsules (20 mg CIVI 008/200 mg 5-CNAC) were manufactured from uniform dry blended formulations, that were filled into enterically coated Size 0, hard shell gelatin capsules. A total of 10 Cynomolgus monkeys (5 males and 5 females) were each administered a single capsule by oral gavage once daily for 49 days. Two animals (a male and a female), which did not receive treatment, were included in the study as reference animals. During the dosing period, blood samples for PK analysis were drawn pre-dose and 0.5, 1.5, 3 and 5 hours post-dosing at study start and at day 49. Blood samples for PCSK9 and lipid analysis were drawn on week −2 and −1 pre-study, pre-dose on Day 1 and weekly thereafter for the duration of the study. Samples for clinical chemistry, coagulation and hematology were taken on week −2 and −1 pre-study, and on Day 22 and 49.

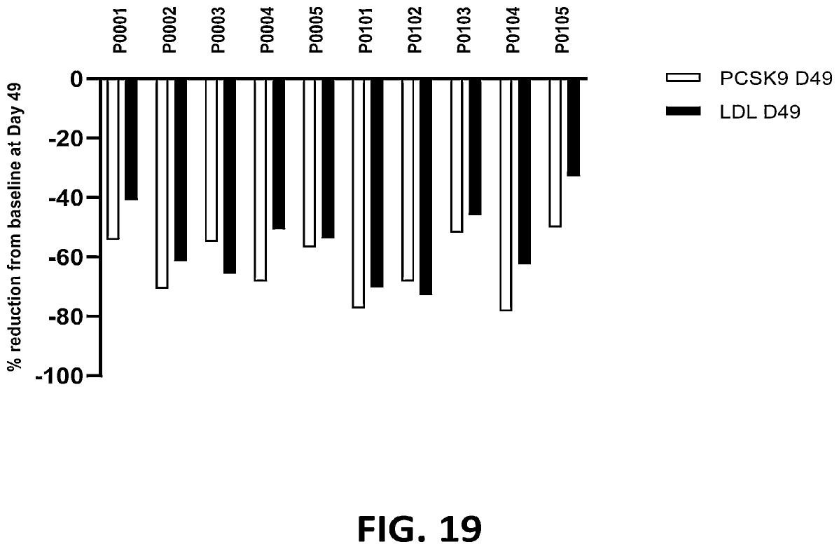

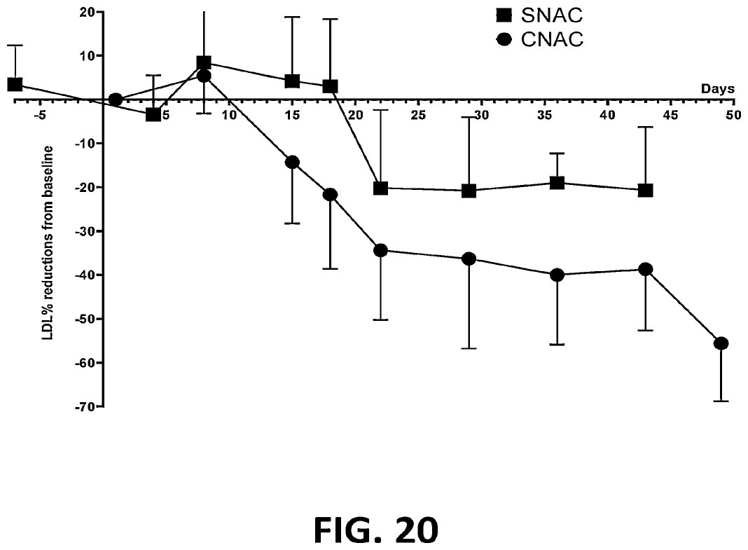

Pharmacodynamic results: FIG. 19 shows the reductions in PCSK9 and LDL at day 49. Significant reductions in PCSK-9 and LDL were apparent in all the animals, with the highest responders achieving reductions from baseline in plasma LDL of >70%. PCSK9 started to drop as early as day 8 and both PCSK9 and LDL were clearly reduced in the majority of animals at day 15. Compared with the previous experiment using SNAC as the carrier for CIVI 008 (Example 1, FIG. 11A), dosing with 5-CNAC formulated CIVI 008, caused a more rapid and more substantial drop in LDL (FIG. 20), indicating that 5-CNAC was substantially more efficient than SNAC in facilitating oral uptake of CIVI 008.

### Example 5

### CIVI 008: Oral (Capsule) PK Study of CIVI 008 without GalNAc in the Cynomolgus Monkeys Using 5-CNAC as Carrier

As shown in previous Examples 1 and 3, orally dosed CIVI 008 with GalNAc is rapidly internalized in the liver by the GalNAc receptor, with very little CIVI 008 escaping into general circulation at doses below that required for saturation of the GalNAc receptor. In many instances the objective is to target genes that are expressed in organs other than the liver. Potentially this can be achieved by using either un-conjugated oligos or oligos with targeting ligands other than GalNAc, both of which designs should increase systemic exposure and hence accumulation in the target organ(s). The objective of this study was to determine the systemic exposure of orally administered CIVI 008 without the GalNAc conjugate, and to compare the exposure to that of a similar oral dose of CIVI 008 with GalNAc.

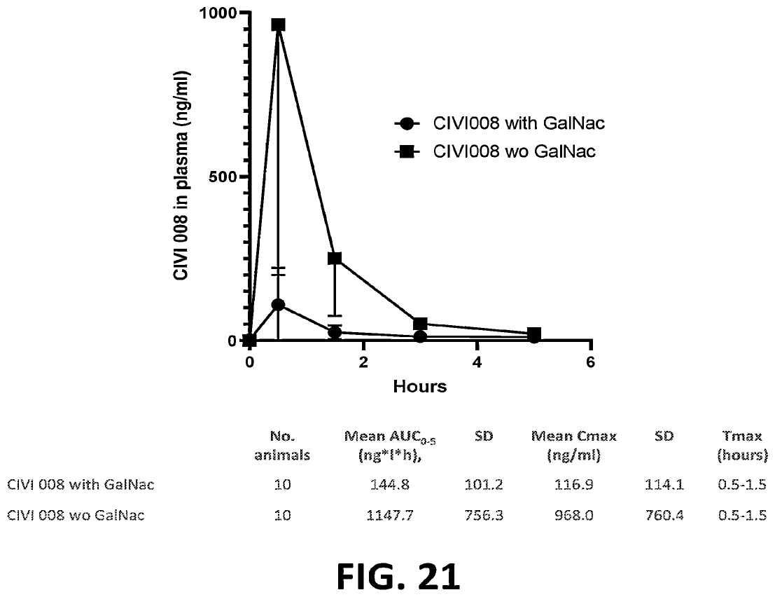

Study outline: Dry blended capsules (20 mg CIVI 008 without GalNAc/200 mg 5-CNAC) were manufactured from uniform dry blended formulations, that were filled into enterically coated Size 0, hard shell gelatin capsules. A total of 10 Cynomolgus monkeys (5 males and 5 females) were each administered a single capsule by oral gavage, and blood samples for PK analysis were drawn pre-dose and 0.5, 1.5, 3 and 5 hours post-dosing. Results: As shown in FIG. 21 dosing of animals by 20 mg CIVI 008 without GalNAc, led to measurable concentrations of CIVI 008 in the plasma. The plasma profile was very similar to that of CIVI 008 with GalNAc, with Tmax occurring within 0.5 to 1.5 hours post dosing. As expected, however, the Cₘₐₓ and AUC were significantly larger (˜8 fold) than the corresponding values for a similar dose of CIVI 008 with GalNac, indicating that oligo design (i.e. absence of conjugate/choice of conjugate) may be used to improve targeting of non-hepatic tissues by the oral route.

### Example 6

### Oral (Capsule) PK Study of an Oligo Against Factor VII, with or without GalNAc, in the Cynomolgus Monkeys Using 5-CNAC as Carrier

The objective of this study was to demonstrate 5-CNAC's ability to increase oral uptake of antisense oligos in general.

Study outline: Dry blended capsules (20 mg Factor FVII with or without GalNac/200 mg 5-CNAC) were manufactured from uniform dry blended formulations, that were filled into enterically coated Size 0, hard shell gelatin capsules. A total of 10 Cynomolgus monkeys (5 males and 5 females) were each administered a single capsule of either oligo by oral gavage, and blood samples for PK analysis were drawn pre-dose and 0.5, 1.5, 3 and 5 hours post-dosing.

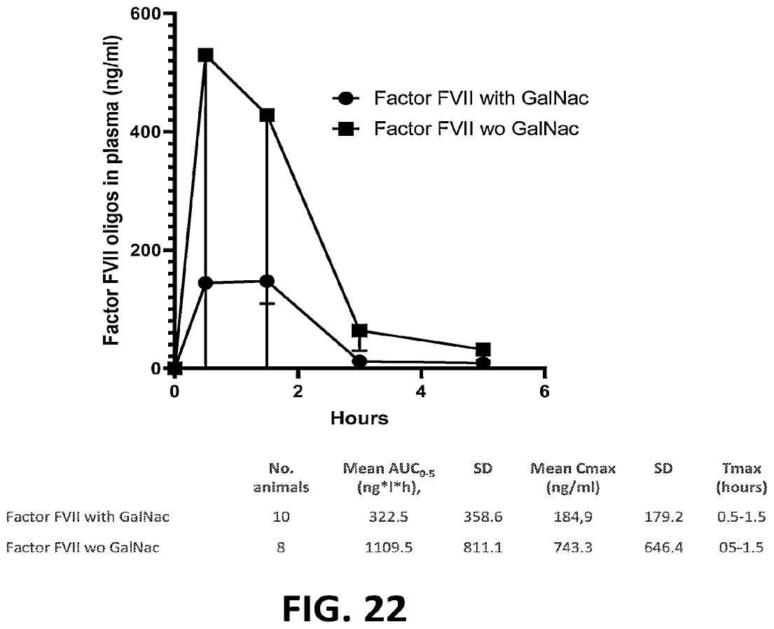

Results: As shown in FIG. 22 dosing of animals by 20 mg of either of the two FVII oligos, led to measurable concentrations in the plasma, and with Tmax occurring within 0.5 to 1.5 hours post dosing. Similar to the observation with CIVI 008, with and without GalNac, the AUC and Cₘₐₓ for the unconjugated Factor FVII oligo, were significantly higher (˜3.5 fold) than for the GalNac conjugated Factor FVII oligo. Furthermore, PK parameters were quite similar to those of the corresponding CIVI 008 oligos, with and without GalNac, supporting the ability of 5-CNAC to facilitate oral dosing of therapeutic oligos in general.

0.5 hr samples showed the parent compound, 1.5 hr samples showed a mixture of parent compound and loss of carbohydrate moiety. 3 hr and 5 hr samples showed test item mixtures with progressive loss of carbohydrate moiety

TABLE 4
Conjugated and unconjugated oligonucleotides.

| | free acid | | | |
|---|---|---|---|---|
| | [Da] | SEQ | | Sequence modification patterns |
| | Na⁺ salt | ID | Length | [s: thiosulfate linkage; b: modified nucleobase (LNA); |
| Compound | [Da] | NO | [nt] | d: DNA; (GalNAc3)(NHC6): GalNAc moiety] |
| Factor VII | 5.911, 20 | 158 | 13 | (GalNAc3)(NHC6)sGbsCbsdAsdCsdCsdAsdCsdGsdGsdTsCbsCbsAb |
| ASO with | 6.196, 97 | | | |
| GalNAc | | | | |
| moiety | | | | |
| Factor VII | 4.279, 48 | 158 | 13 | GbsCbsdAsdCsdCsdAsdCsdGsdGsdTsCbsCbsAb |
| ASO | 4.543, 26 | | | |
| without | | | | |
| GalNAc | | | | |
| moity | | | | |
| CIVI 008 | 6.896, 02 | 134 | 16 | (GalNAc3)(NHC6)sAbsAbsTbsdGsdCsdTsdAsdCsdAsdAsdAsdAsdCsCbsCbsAb |
| with | 7.247, 73 | | | |
| GalNAc | | | | |
| moiety | | | | |
| CIVI 008 | 5.264, 29 | 134 | 16 | AbsAbsTbsdGsdCsdTsdAsdCsdAsdAsdAsdAsdCsCbsCbsAb |
| without | 5.594, 02 | | | |
| GalNAc | | | | |
| moiety | | | | |

### INCORPORATION BY REFERENCE

The contents of all cited references (including literature references, patents, patent applications, and websites) that may be cited throughout this application are hereby expressly incorporated by reference in their entirety for any purpose, as are the references cited therein, in the versions publicly available on Dec. 10, 2021. Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on Apr. 22, 2021.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method for increasing (i) oral uptake, (ii) biological effect or therapeutic effect, and/or (iii) circulating plasma levels, of a therapeutic oligonucleotide, comprising co-administering the therapeutic oligonucleotide and N-(5-chlorosalicyloyl)-8-aminocaprylic acid (5-CNAC).

2. The method of claim 1, wherein the (i) oral uptake, (ii) biological effect or therapeutic effect, and/or (ii) circulating plasma levels is increased by at least about 10%, about 20%, about 30%, about 40%, about 50%, about 60%, about 70%, about 80%, about 90%, about 100%, about 125%, about 150%, about 175%, or about 200% compared to oral uptake, biological effect or therapeutic effect and/or circulating plasma levels observed when the therapeutic oligonucleotide is administered without 5-CNAC or co-administered with N-(8-[2-hydroxybenzoyl]amino)caprylic acid (SNAC).

3. An oligonucleotide composition comprising a therapeutic oligonucleotide, and 5-CNAC or a salt thereof, wherein the oligonucleotide composition is formulated for delivery to the gastrointestinal tract.

4. The oligonucleotide composition of claim 3, wherein the salt of 5-CNAC is selected from the group consisting of a sodium salt, a potassium salt, a calcium salt, and any combination thereof, and wherein the salt of 5-CNAC is a monosodium salt or a disodium salt.

5. The oligonucleotide composition of claim 3, wherein the therapeutic oligonucleotide is an antisense oligonucleotide (ASO), short interference RNA (siRNA), small hairpin RNA (shRNA), DNA and/or RNA aptamer, micro RNA (miRNA), anti-micro RNA (antimiR), CpG oligonucleotide, or DNA and/or RNA decoy.

6. A method of manufacturing the oligonucleotide composition of claim 3 comprising admixing (i) a therapeutic oligonucleotide selected from the group consisting of an ASO, a siRNA, a shRNA, a DNA or RNA aptamer, a miRNA, a miRNA mimic, an antimiR, a DNA or RNA decoy, and CpG oligonucleotide; and, (ii) 5-CNAC or a salt thereof, wherein the salt is a monosodium or disodium salt.

7. The method of claim 6, wherein the admixing comprises dry blending.

8. The method of claim 7, further comprising encapsulating the resulting dry blend of step in a capsule.

9. A tablet or capsule comprising an oligonucleotide composition comprising (i) a therapeutic oligonucleotide selected from the group consisting of an ASO, a siRNA, a shRNA, a DNA or RNA aptamer, a miRNA, a miRNA mimic, an antimiR, a DNA or RNA decoy, and CpG oligonucleotide; and, (ii) 5-CNAC or a salt thereof, wherein the salt is a monosodium or disodium salt.

10. The tablet or capsule of claim 9, wherein the tablet or capsule comprises an enteric coating, a pH sensitive coating, or a combination thereof.

11. The tablet or capsule of claim 9, wherein the tablet or capsule has a weight between 10 mg and 500 mg.

12. The tablet or capsule of claim 9, wherein the tablet or capsule comprises 1 mg to 500 mg of therapeutic oligonucleotide.

13. A method of treating a disease or condition in a subject in need thereof comprising administering an effective amount of a oligonucleotide composition of claims 3 to 5 or the tablet or capsule of claims 9 to 12 to the subject.

14. The method of claim 13, wherein the oligonucleotide composition, table, or capsule is administered orally.

15. The method of claim 13, wherein the oligonucleotide composition, tablet, or capsule is administered as a single dose or multiple doses.

16. The method of claim 13, wherein the oligonucleotide composition, tablet, or capsule is administered at least 5, 10, 15, 20, 25, 30, 35, 40, 45, 50, 55 or 60 minutes prior to a meal.

17. The method of claims 1, 2, 6-8, or 13-15, oligonucleotide composition of claims 3 to 5, or tablet or capsule of claims 9-12, wherein the therapeutic oligonucleotide is selected from the group consisting of 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™), ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimderlirsen, cobitolimod, cobomarsen, CODA-001 (NEXAGON™), cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™), fazisiran, fesomersen, fitusiran, fomivirsen (VITRAVENE™), frenlosirsen, gataparsen, givosiran (GIVLAARI™), GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS-FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX-H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™), oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™), pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SB010, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™) vupanorsen, vutrisiran, WVE-003, WVE-004, WVEN-531, zilebesiran, and zilganersen.
